SEQUENCE LISTING (571 sequences)

SEQ ID NO: 1 - protein, 140 aa, Clostridium perfringens
NPSLIRSESWQVYEGNEANLLDGDDNTGVWYKTLNGDTSLAGEFIGLDLGKEIKLDGIRFVIGKNGGGSSDKWNKFKLEYSLDNESWTTIKEYDKTGAPAGKDVIEESFETPISAKYIRLTNMENINKWLTFSEFAIVSD

SEQ ID NO: 2 - protein, 140 aa, synthetic construct
  VARIANT 11..14: note = can be any amino acid
  VARIANT 32..38: note = can be any amino acid
  VARIANT 125..129: note = can be any amino acid
NPSLIRSESWXXXXGNEANLLDGDDNTGVWYXXXXXXXSLAGEFIGLDLGKEIKLDGIRFVIGKNGGGSSDKWNKFKLEYSLDNESWTTIKEYDKTGAPAGKDVIEESFETPISAKYIRLTNLEXXXXXLTFSEFAIVSD

SEQ ID NO: 3 - protein, 143 aa, synthetic construct
  VARIANT 11..14: note = can be any amino acid
  VARIANT 32..38: note = can be any amino acid
  VARIANT 125..129: note = can be any amino acid
DPTLIHTPGWXXXXGSEADLLDGDDSTGVEYXXXXXXXSLAGEFIGLDLGEVVEVGGIHFVIGADGGGSSDKWTRFRLEYSLDGESWTTIREYDHTGAPAGQDVIDEDFETPISAQYIRLTNLEXXXXXLTFSEFAIVSDELE

SEQ ID NO: 4 - protein, 8 aa, synthetic construct
  VARIANT 1: note = or S
  VARIANT 4: note = or R
  VARIANT 5: note = or S
  VARIANT 6: note = or E
  VARIANT 7: note = or S
TLIHTPGW

SEQ ID NO: 5 - protein, 17 aa, synthetic construct
  VARIANT 2: note = or N or T
  VARIANT 4: note = or S
  VARIANT 5: note = or N or S or A
  VARIANT 12: note = or N or T
  VARIANT 16: note = or W or A
GSEADLLDGDDSTGVEY

SEQ ID NO: 6 - protein, 12 aa, synthetic construct
  VARIANT 2: note = or V
SLAGEFIGLDLG

SEQ ID NO: 7 - protein, 9 aa, synthetic construct
  VARIANT 2: note = or V
  VARIANT 3: note = or R or Y or N
  VARIANT 8: note = or K or R
  VARIANT 9: note = or N
GIHFVIGAD

SEQ ID NO: 8 - protein, 11 aa, synthetic construct
  VARIANT 4: note = or N or S
  VARIANT 5: note = or K
  VARIANT 7: note = or K
DKWTRFRLEYS

SEQ ID NO: 9 - protein, 9 aa, synthetic construct
  VARIANT 5: note = or K or H or Q
  VARIANT 9: note = or K or R or Q
WTTIREYDH

SEQ ID NO: 10 - protein, 8 aa, synthetic construct
  VARIANT 1: note = or K
  VARIANT 5: note = or E
  VARIANT 7: note = or S
QDVIDEDF

SEQ ID NO: 11 - protein, 9 aa, synthetic construct
  VARIANT 1: note = or K or R
QYIRLTNLE

SEQ ID NO: 12 - protein, 10 aa, synthetic construct
  VARIANT 8: note = or V
LTFSEFAIVS

SEQ ID NO: 13-16 - skipped (4 entries)

SEQ ID NO: 17 - protein, 5 aa, synthetic construct
  VARIANT 1: note = or D
GGGSS

SEQ ID NO: 18 - protein, 6 aa, synthetic construct
TGAPAG

SEQ ID NO: 19 - protein, 6 aa, synthetic construct
  VARIANT 1: note = or K or S
  VARIANT 2: note = or E
  VARIANT 3: note = or I or T
  VARIANT 4: note = or K or P or S
  VARIANT 5: note = or L
  VARIANT 6: note = or D
EVVEVG

SEQ ID NO: 20 - protein, 5 aa, synthetic construct
  VARIANT 3: note = or N
  VARIANT 4: note = or S
LDGES

SEQ ID NO: 21 - protein, 6 aa, synthetic construct
  VARIANT 5: note = or E
ETPISA

SEQ ID NO: 22 - protein, 8 aa, synthetic construct
TLIHTPGW

SEQ ID NO: 23 - protein, 17 aa, synthetic construct
GSEADLLDGDDSTGVEY

SEQ ID NO: 24 - protein, 12 aa, synthetic construct
SLAGEFIGLDLG

SEQ ID NO: 25 - protein, 9 aa, synthetic construct
GIHFVIGAD

SEQ ID NO: 26 - protein, 11 aa, synthetic construct
DKWTRFRLEYS

SEQ ID NO: 27 - protein, 9 aa, synthetic construct
WTTIREYDH

SEQ ID NO: 28 - protein, 8 aa, synthetic construct
QDVIDEDF

SEQ ID NO: 29 - protein, 9 aa, synthetic construct
QYIRLTNLE

SEQ ID NO: 30 - protein, 10 aa, synthetic construct
LTFSEFAIVS

SEQ ID NO: 31 - protein, 6 aa, synthetic construct
EVVEVG

SEQ ID NO: 32 - protein, 5 aa, synthetic construct
GGGSS

SEQ ID NO: 33 - protein, 5 aa, synthetic construct
LDGES

SEQ ID NO: 34 - protein, 6 aa, synthetic construct
ETPISA

SEQ ID NO: 35 - protein, 4 aa, synthetic construct
DELE

SEQ ID NO: 36 - protein, 5 aa, synthetic construct
GGGGG

SEQ ID NO: 37 - protein, 4 aa, synthetic construct
GGGG

SEQ ID NO: 38 - DNA, 6128 bp, synthetic construct
  source 1..6128: mol_type = other DNA
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacaacccttctttaattcgttctgaatcctgggaagacatcaaagggaatgaagccaatttattagatggagacgataacaccggtgtttggtatttcaacgaagttttctacgaatctcttgcaggagaattcattggattggacttaggtaaggaaattaaattggatggtattcgttttgttattggtaagaatggaggcggtagttccgacaaatggaacaaattcaagttggagtactccctggataacgaaagttggactactatcaaagaatacgacaagacaggggctcctgcagggaaagatgttattgaagaatccttcgagactcccatttccgctaagtacattcgtctgactaatctggaagacaaaatcctgttcctgacttttagtgagtttgcaattgtgtctgacggtggaggtggcagcggcggtggtggctcgggtggagggtgctgagctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 39 - protein, 115 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGGGLNDIFEAQKIEWHE

SEQ ID NO: 40 - protein, 368 aa, synthetic construct
MGSSHHHHHHSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGLYFQGSKGEELFTGVVPILVELDGDVNGHKFSVSGEGEGDATYGKLTLKFICTTGKLPVPWPTLVTTFAYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKIEGRGGKPIPNPLLGLDST

SEQ ID NO: 41 - protein, 15 aa, synthetic construct
GGGGSGGGGSGGGGS

SEQ ID NO: 42 - protein, 4 aa, synthetic construct
EDIK

SEQ ID NO: 43 - protein, 7 aa, synthetic construct
FNEVFYE

SEQ ID NO: 44 - protein, 5 aa, synthetic construct
DKILF

SEQ ID NO: 45 - protein, 4 aa, synthetic construct
LVIP

SEQ ID NO: 46 - protein, 7 aa, synthetic construct
IIVEEFT

SEQ ID NO: 47 - protein, 5 aa, synthetic construct
YNFID

SEQ ID NO: 48 - protein, 4 aa, synthetic construct
NVEQ

SEQ ID NO: 49 - protein, 7 aa, synthetic construct
ILVEEFP

SEQ ID NO: 50 - protein, 5 aa, synthetic construct
NEVPQ

SEQ ID NO: 51 - protein, 4 aa, synthetic construct
PVVT

SEQ ID NO: 52 - protein, 7 aa, synthetic construct
IVLTDQA

SEQ ID NO: 53 - protein, 5 aa, synthetic construct
LAQDP

SEQ ID NO: 54 - DNA, 87 bp, synthetic construct
  source 1..87: mol_type = other DNA
atactcaacaccggtggaatcgtctccatctaataaatcggcttcggacccnnnnnngacnnnccagccgggggtatggattaaggt

SEQ ID NO: 55 - DNA, 100 bp, synthetic construct
  source 1..100: mol_type = other DNA
tatcgacgaagatttcgagactcccatctccgctcagtacatccgtttaactaatttagaannnnnnnnnnnnnnnttaacttttagtgagtttgcaatc

SEQ ID NO: 56 - DNA, 100 bp, synthetic construct
  source 1..100: mol_type = other DNA
gatggagacgattccaccggtgttgagtatnnnnnnnnnnnnnnnnnnnnntctcttgcaggagaattcatcggattagacttaggtgaggtagtagaag

SEQ ID NO: 57 - DNA, 200 bp, synthetic construct
  source 1..200: mol_type = other DNA
gatgggagtctcgaaatcttcgtcgataacatcttgccctgcaggagcccctgtatggtcgtattcacggatagtagtccaactttcgccatctaaggagtactctaaacggaaacgagtccatttgtcggaactaccgcctccatcagcaccgataacaaaatggataccgccgacttctactacctcacctaagtcta

SEQ ID NO: 58 - protein, 160 aa, synthetic construct
GSSHHHHHHDPTLIHTPGWWVPGSEADLLDGDDSTGVEYVHYPVQSSLAGEFIGLDLGEVVEVGGIHFVIGADGGGSSDKWTRFRLEYSLDGESWTTIREYDHTGAPAGKDVIDEDFETPISAQYIRLTNLEGPWHGLTFSEFAIVSDELEDYKDDDDKG

SEQ ID NO: 59 - skipped

SEQ ID NO: 60 - protein, 7 aa, synthetic construct
VHYPVQS

SEQ ID NO: 61 - protein, 5 aa, synthetic construct
GPWHG

SEQ ID NO: 62 - protein, 159 aa, synthetic construct
GSSHHHHHHDPTLIHTPGWNVTVGSEADLLDGDDSTGVEYYITTSSLAGEFIGLDLGEVVEVGGIHFVIGADGGGSSDKWTRFRLEYSLDGESWTTIREYDHTGAPAGQDVIDEDFETPISAQYIRLTNLEEPGSHLTFSEFAIVSDELEDYKDDDDKG

SEQ ID NO: 63 - protein, 4 aa, synthetic construct
NVTV

SEQ ID NO: 64 - protein, 5 aa, synthetic construct
YITTS

SEQ ID NO: 65 - protein, 5 aa, synthetic construct
EPGSH

SEQ ID NO: 66 - protein, 158 aa, synthetic construct
HHHHHHDPTLIHTPGWVVAAGSEADLLDGDDSTGVEYPQFQHQVSLAGEFIGLDLGEVVEVGGIHFVIGADGGGSSDKWTRFRLEYSLDGESWTTIREYDHTGAPAGQDVIDEDFETPITAQYIRLTNLEAFQPGLTFSEFAIVSDELEDYKDDDDKG

SEQ ID NO: 67 - protein, 4 aa, synthetic construct
VVAA

SEQ ID NO: 68 - protein, 7 aa, synthetic construct
PQFQHQV

SEQ ID NO: 69 - protein, 5 aa, synthetic construct
AFQPG

SEQ ID NO: 70 - protein, 145 aa, synthetic construct
NPSLIRSESWGNEANLLDGDDNTGVWYSDHPWFNSLAGEFIGLDLGKEIKLDGIRFVIGKNGGGSSDKWNKFKLEYSLDNESWTTIKEYDKTGAPAGKDVIEESFETPISAKYIRLTNLEYSFRFLTFSEFAIVSDDYKDDDDKG

SEQ ID NO: 71 - protein, 7 aa, synthetic construct
SDHPWFN

SEQ ID NO: 72 - protein, 5 aa, synthetic construct
YSFRF

SEQ ID NO: 73 - protein, 157 aa, synthetic construct
GSSHHHHHHNPSLIRSESWPVYGNEANLLDGDDNTGVWYYSSGTYFSLAGEFIGLDLGKEIKLDGIRFVIGKNGGGSSDKWNKFKLEYSLDNESWTTIKEYDKTGAPAGKDVIEESFETPISAKYIRLTNLELKYYGLTFSEFAIVSDDYKDDDDKG

SEQ ID NO: 74 - skipped

SEQ ID NO: 75 - protein, 7 aa, synthetic construct
YSSGTYF

SEQ ID NO: 76 - protein, 5 aa, synthetic construct
LKYYG

SEQ ID NO: 77 - protein, 157 aa, synthetic construct
GSSHHHHHHNPSLIRSESWLAFGNEANLLDGDDNTGVWYYDVAWISEDRNNRDASLAGEFIGLDLGKEIKLDGIRFVIGKNGGGSSDKWNKFKLEYSLDNESWTTIKEYDKTGAPAGKDVIEESFETPISAKYIRLTNLESVIPILTFSEFAIVSDG

SEQ ID NO: 78 - skipped

SEQ ID NO: 79 - protein, 15 aa, synthetic construct
YDVAWISEDRNNRDA

SEQ ID NO: 80 - protein, 5 aa, synthetic construct
SVIPI

SEQ ID NO: 81 - protein, 148 aa, synthetic construct
NPSLIRSESWAENGNEANLLDGDDNTGVWYNEDDHFGSLAGEFIGLDLGKEIKLDGIRFVIGKNGGGSSDKWNKFKLEYSLDNESWTTIKEYDKTGAPAGKDVIEESFETPISAKYIRLTNLEPRHDTLTFSEFAIVSDDYKDDDDKG

SEQ ID NO: 82 - skipped

SEQ ID NO: 83 - protein, 7 aa, synthetic construct
NEDDHFG

SEQ ID NO: 84 - protein, 5 aa, synthetic construct
PRHDT

SEQ ID NO: 85 - protein, 148 aa, synthetic construct
NPSLIRSESWVGKGNEANLLDGDDNTGVWYDEDNHFGSLAGEFIGLDLGKEIKLDGIRFVIGKNGGGSSDKWNKFKLEYSLDNESWTTIKEYDKTGAPAGKDVIEESFETPISAKYIRLTNLEYQTRELTFSEFAIVSDDYKDDDDKG

SEQ ID NO: 86 - skipped

SEQ ID NO: 87 - protein, 7 aa, synthetic construct
DEDNHFG

SEQ ID NO: 88 - protein, 5 aa, synthetic construct
YQTRE

SEQ ID NO: 89 - protein, 148 aa, synthetic construct
NPSLIRSESWVASGNEANLLDGDDNTGVWYKFGHEWESLAGEFIGLDLGKEIKLDGIRFVIGKNGGGSSDKWNKFKLEYSLDNESWTTIKEYDKTGAPAGKDVIEESFETPISAKYIRLTNLERKYIFLTFSEFAIVSDDYKDDDDKG

SEQ ID NO: 90 - skipped

SEQ ID NO: 91 - protein, 7 aa, synthetic construct
KFGHEWE

SEQ ID NO: 92 - protein, 5 aa, synthetic construct
RKYIF

SEQ ID NO: 93 - protein, 148 aa, synthetic construct
NPSLIRSESWVFFGNEANLLDGDDNTGVWYFTAGNKQSLAGEFIGLDLGKEIKLDGIRFVIGKNGGGSSDKWNKFKLEYSLDNESWTTIKEYDKTGAPAGKDVIEESFETPISAKYIRLTNLEEHQYLLTFSEFAIVSDDYKDDDDKG

SEQ ID NO: 94 - skipped

SEQ ID NO: 95 - protein, 7 aa, synthetic construct
FTAGNKQ

SEQ ID NO: 96 - protein, 5 aa, synthetic construct
EHQYL

SEQ ID NO: 97 - protein, 148 aa, synthetic construct
NPSLIRSESWVFFGNEANLLDGDDNTGVWYAGGHADDSLAGEFIGLDLGKEIKLDGIRFVIGKNGGGSSDKWNKFKLEYSLDNESWTTIKEYDKTGAPAGKDVIEESFETPISAKYIRLTNLETGFNYLTFSEFAIVSDDYKDDDDKG

SEQ ID NO: 98 - skipped

SEQ ID NO: 99 - protein, 7 aa, synthetic construct
AGGHADD

SEQ ID NO: 100 - protein, 5 aa, synthetic construct
TGFNY

SEQ ID NO: 101 - protein, 148 aa, synthetic construct
NPSLIRSESWVLVGNEANLLDGDDNTGVWYRASAWYNSLAGEFIGLDLGKEIKLDGIRFVIGKNGGGSSDKWNKFKLEYSLDNESWTTIKEYDKTGAPAGKDVIEESFETPISAKYIRLTNLEEIRSVLTFSEFAIVSDDYKDDDDKG

SEQ ID NO: 102 - skipped

SEQ ID NO: 103 - protein, 7 aa, synthetic construct
RASAWYN

SEQ ID NO: 104 - protein, 5 aa, synthetic construct
EIRSV

SEQ ID NO: 105 - protein, 142 aa, synthetic construct
NPSLIRSESWPYSYGNEANLLDGDDNTGVWYNSLAGEFIGLDLGKEIKLDGIRFVIGKNGGGSSDKWNKFKLEYSLDNESWTTIKEYDKTGAPAGKDVIEESFETPISAKYIRLTNLEAPGNLTFSEFAIVSDDYKDDDDKG

SEQ ID NO: 106 - protein, 4 aa, synthetic construct
PYSY

SEQ ID NO: 107 - skipped

SEQ ID NO: 108 - protein, 4 aa, synthetic construct
APGN

SEQ ID NO: 109 - protein, 145 aa, synthetic construct
NPSLIRSESWDEWWGNEANLLDGDDNTGVWYYYDDSLAGEFIGLDLGKEIKLDGIRFVIGKNGGGSSDKWNKFKLEYSLDNESWTTIKEYDKTGAPAGKDVIEESFETPISAKYIRLTNLEDIKYLTFSEFAIVSDDYKDDDDKG

SEQ ID NO: 110 - protein, 4 aa, synthetic construct
DEWW

SEQ ID NO: 111 - protein, 4 aa, synthetic construct
YYDD

SEQ ID NO: 112 - protein, 4 aa, synthetic construct
DIKY

SEQ ID NO: 113 - protein, 147 aa, synthetic construct
NPSLIRSESWHKAHGNEANLLDGDDNTGVWYWAHKKSLAGEFIGLDLGKEIKLDGIRFVIGKNGGGSSDKWNKFKLEYSLDNESWTTIKEYDKTGAPAGKDVIEESFETPISAKYIRLTNLEYVEKELTFSEFAIVSDDYKDDDDKG

SEQ ID NO: 114 - protein, 4 aa, synthetic construct
HKAH

SEQ ID NO: 115 - protein, 5 aa, synthetic construct
WAHKK

SEQ ID NO: 116 - protein, 5 aa, synthetic construct
YVEKE

SEQ ID NO: 117 - protein, 147 aa, synthetic construct
NPSLIRSESWLDNQGNEANLLDGDDNTGVWYEKNDYSLAGEFIGLDLGKEIKLDGIRFVIGKNGGGSSDKWNKFKLEYSLDNESWTTIKEYDKTGAPAGKDVIEESFETPISAKYIRLTNLETIITELTFSVFAIVSDDYKDDDDKG

SEQ ID NO: 118 - protein, 4 aa, synthetic construct
LDNQ

SEQ ID NO: 119 - protein, 5 aa, synthetic construct
EKNDY

SEQ ID NO: 120 - protein, 5 aa, synthetic construct
TIITE

SEQ ID NO: 121 - protein, 147 aa, synthetic construct
NPSLIRSESWVIWVGNEANLLDGDDNTGVWYNFEYYSLAGEFIGLDLGKEIKLDGIRFVIGKNGGGSSDKWNKFKLEYSLDNESWTTIKEYDKTGAPAGKDVIEESFETPISAKYIRLTNLEFVYGVLTFSEFAIVSDDYKDDDDKG

SEQ ID NO: 122 - protein, 4 aa, synthetic construct
VIWV

SEQ ID NO: 123 - protein, 5 aa, synthetic construct
NFEYY

SEQ ID NO: 124 - protein, 5 aa, synthetic construct
FVYGV

SEQ ID NO: 125 - protein, 157 aa, synthetic construct
GSSHHHHHHNPSLIRSESWEPWAGNEANLLDGDDNTGVWYLGLYGYSLAGEFIGLDLGKEIKLDGIRFVIGKNGGGSSDKWNKFKLEYSLDNESWTTIKEYDKTGAPAGKDVIEESFETPISAKYIRLTNLEKVFIWLTFSEFAIVSDDYKDDDDKG

SEQ ID NO: 126 - protein, 4 aa, synthetic construct
EPWA

SEQ ID NO: 127 - protein, 6 aa, synthetic construct
LGLYGY

SEQ ID NO: 128 - protein, 5 aa, synthetic construct
KVFIW

SEQ ID NO: 129 - protein, 149 aa, synthetic construct
MGCSHHHHHHNPSLIRSESWFIKKGNEANLLDGDDNTGVWYWLPNYFSLAGEFIGLDLGKEIKLDGIRFVIGKNGGGSSDKWNKFKLEYSLDNESWTTIKEYDKTGAPAGKDVIEESFETPISAKYIRLTNLETHYFNLTFSEFAIVSD

SEQ ID NO: 130 - protein, 4 aa, synthetic construct
FIKK

SEQ ID NO: 131 - protein, 6 aa, synthetic construct
WLPNYF

SEQ ID NO: 132 - protein, 5 aa, synthetic construct
THYFN

SEQ ID NO: 133 - protein, 148 aa, synthetic construct
NPSLIRSESWQVFYGNEANLLDGDDNTGVWYYGKDDWSLAGEFIGLDLGKEIKLDGIRFVIGKNGGGSSDKWNKFKLEYSLDNESWTTIKEYDKTGAPAGKDVIEESFETPISAKYIRLTNLEPWWSYLTFSEFAIVSDDYKDDDDKG

SEQ ID NO: 134 - protein, 4 aa, synthetic construct
QVFY

SEQ ID NO: 135 - protein, 6 aa, synthetic construct
YGKDDW

SEQ ID NO: 136 - protein, 5 aa, synthetic construct
PWWSY

SEQ ID NO: 137 - protein, 163 aa, synthetic construct
MGSSHHHHHHNPSLIRSESWVRWEGNEANLLDGDDNTGVWYALWNHLSLAGEFIGLDLGKEIKLDGIRFVIGKNGGGSSDKWNKFKLEYSLDNESWTTIKEYDKTGAPAGKDVIEESFETPISAKYIRLTNLESQYSDLTFSEFAIVSDGGGGSGGGGSGGGC

SEQ ID NO: 138 - protein, 4 aa, synthetic construct
VRWE

SEQ ID NO: 139 - protein, 6 aa, synthetic construct
ALWNHL

SEQ ID NO: 140 - protein, 5 aa, synthetic construct
SQYSD

SEQ ID NO: 141 - protein, 148 aa, synthetic construct
NPSLIRSESWPFIIGNEANLLDGDDNTGVWYIPSHHFSLAGEFIGLDLGKEIKLDGIRFVIGKNGGGSSDKWNKFKLEYSLDNESWTTIKEYDKTGAPAGKDVIEESFETPISAKYIRLTNLEFKYTYLTFSEFAIVSDDYKDDDDKG

SEQ ID NO: 142 - protein, 4 aa, synthetic construct
PFII

SEQ ID NO: 143 - protein, 6 aa, synthetic construct
IPSHHF

SEQ ID NO: 144 - protein, 5 aa, synthetic construct
FKYTY

SEQ ID NO: 145 - protein, 148 aa, synthetic construct
NPSLIRSESWIWAIGNEANLLDGDDNTGVWYIPKEKKSLAGEFIGLDLGKEIKLDGIRFVIGKNGGGSSDKWNKFKLEYSLDNESWTTIKEYDKTGAPAGKDVIEESFETPISAKYIRLTNLELQISYLTFSEFAIVSDDYKDDDDKG

SEQ ID NO: 146 - protein, 4 aa, synthetic construct
IWAI

SEQ ID NO: 147 - protein, 6 aa, synthetic construct
IPKEKK

SEQ ID NO: 148 - protein, 5 aa, synthetic construct
LQISY

SEQ ID NO: 149 - protein, 148 aa, synthetic construct
NPSLIRSESWKITWGNEANLLDGDDNTGVWYLNVFKKSLAGEFIGLDLGKEIKLDGIRFVIGKNGGGSSDKWNKFKLEYSLDNESWTTIKEYDKTGAPAGKDVIEESFETPISAKYIRLTNLEYEHSELTFSEFAIVSDDYKDDDDKG

SEQ ID NO: 150 - protein, 4 aa, synthetic construct
KITW

SEQ ID NO: 151 - protein, 6 aa, synthetic construct
LNVFKK

SEQ ID NO: 152 - protein, 5 aa, synthetic construct
YEHSE

SEQ ID NO: 153 - protein, 148 aa, synthetic construct
NPSLIRSESWIFWSGNEANLLDGDDNTGVWYYKYPKQSLAGEFIGLDLGKEIKLDGIRFVIGKNGGGSSDKWNKFKLEYSLDNESWTTIKEYDKTGAPAGKDVIEESFETPISAKYIRLTNLEVVETWLTFSEFAIVSDDYKDDDDKG

SEQ ID NO: 154 - protein, 4 aa, synthetic construct
IFWS

SEQ ID NO: 155 - protein, 6 aa, synthetic construct
YKYPKQ

SEQ ID NO: 156 - protein, 5 aa, synthetic construct
VVETW

SEQ ID NO: 157 - protein, 148 aa, synthetic construct
NPSLIRSESWHVIVGNEANLLDGDDNTGVWYISEFAWSLAGEFIGLDLGKEIKLDGIRFVIGKNGGGSSDKWNKFKLEYSLDNESWTTIKEYDKTGAPAGKDVIEESFETPISAKYIRLTNLEGKYVFLTFSEFAIVSDDYKDDDDKG

SEQ ID NO: 158 - protein, 4 aa, synthetic construct
HVIV

SEQ ID NO: 159 - protein, 6 aa, synthetic construct
ISEFAW

SEQ ID NO: 160 - protein, 5 aa, synthetic construct
GKYVF

SEQ ID NO: 161 - protein, 148 aa, synthetic construct
NPSLIRSESWFRDHGNEANLLDGDDNTGVWYFALVNTSLAGEFIGLDLGKEIKLDGIRFVIGKNGGGSSDKWNKFKLEYSLDNESWTTIKEYDKTGAPAGKDVIEESFETPISAKYIRLTNLEHHFSDLTFSEFAIVSDDYKDDDDKG

SEQ ID NO: 162 - protein, 4 aa, synthetic construct
FRDH

SEQ ID NO: 163 - protein, 6 aa, synthetic construct
FALVNT

SEQ ID NO: 164 - protein, 5 aa, synthetic construct
HHFSD

SEQ ID NO: 165 - protein, 148 aa, synthetic construct
  VARIANT 4: note = any amino acid
NPSXIRSESWPESFGNEANLLDGDDNTGVWYFALINSSLAGEFIGLDLGKEIKLDGIRFVIGKNGGGSSDKWNKFKLEYSLDNESWTTIKEYDKTGAPAGKDVIEESFETPISAKYIRLTNLEVNNDPLTFSEFAIVSDDYKDDDDKG

SEQ ID NO: 166 - protein, 4 aa, synthetic construct
PESF

SEQ ID NO: 167 - protein, 6 aa, synthetic construct
FALINS

SEQ ID NO: 168 - protein, 5 aa, synthetic construct
VNNDP

SEQ ID NO: 169 - protein, 148 aa, synthetic construct
NPSLIRSESWPTKFGNEANLLDGDDNTGVWYFWGFWKSLAGEFIGLDLGKEIKLDGIRFVIGKNGGGSSDKWNKFKLEYSLDNESWTTIKEYDKTGAPAGKDVIEESFETPISAKYIRLTNLEPSINYLTFSEFAIVSDDYKDDDDKG

SEQ ID NO: 170 - protein, 4 aa, synthetic construct
PTKF

SEQ ID NO: 171 - protein, 6 aa, synthetic construct
FWGFWK

SEQ ID NO: 172 - protein, 5 aa, synthetic construct
PSINY

SEQ ID NO: 173 - protein, 148 aa, synthetic construct
NPSLIRSESWHENAGNEANLLDGDDNTGVWYFALINGSLAGEFIGLDLGKEIKLDGIRFVIGKNGGGSSDKWNKFKLEYSLDNESWTTIKEYDKTGAPAGKDVIEESFETPISAKYIRLTNLERHHQQLTFSEFAIVSDDYKDDDDKG

SEQ ID NO: 174 - protein, 4 aa, synthetic construct
HENA

SEQ ID NO: 175 - protein, 6 aa, synthetic construct
FALING

SEQ ID NO: 176 - protein, 5 aa, synthetic construct
RHHQQ

SEQ ID NO: 177 - protein, 148 aa, synthetic construct
NPSLIRSESWGSGHGNEANLLDGDDNTGVWYYIVINKSLAGEFIGLDLGKEIKLDGIRFVIGKNGGGSSDKWNKFKLEYSLDNESWTTIKEYDKTGAPAGKDVIEESFETPISAKYIRLTNLERGVYVLTFSEFAIVSDDYKDDDDKG

SEQ ID NO: 178 - protein, 4 aa, synthetic construct
GSGH

SEQ ID NO: 179 - protein, 6 aa, synthetic construct
YIVINK

SEQ ID NO: 180 - protein, 5 aa, synthetic construct
RGVYV

SEQ ID NO: 181 - protein, 148 aa, synthetic construct
NPSLIRSESWPRSNGNEANLLDGDDNTGVWYFAILNISLAGEFIGLDLGKEIKLDGIRFVIGKNGGGSSDKWNKFKLEYSLDNESWTTIKEYDKTGAPAGKDVIEESFETPISAKYIRLTNLEEQAYTLTFSEFAIVSDDYKDDDDKG

SEQ ID NO: 182 - protein, 4 aa, synthetic construct
PRSN

SEQ ID NO: 183 - protein, 6 aa, synthetic construct
FAILNI

SEQ ID NO: 184 - protein, 5 aa, synthetic construct
EQAYT

SEQ ID NO: 185 - protein, 166 aa, synthetic construct
MGSSHHHHHHNPSLIRSESWIIRDGNEANLLDGDDNTGVWYPKIFSHSLAGEFIGLDLGKEIKLDGIRFVIGKNGGGSSDKWNKFKLEYSLDNESWTTIKEYDKTGAPAGKDVIEESFETPISAKYIRLTNLEWRGVALTFSEFAIVSDGGGLNDIFEAQKIEWHE

SEQ ID NO: 186 - protein, 4 aa, synthetic construct
IIRD

SEQ ID NO: 187 - protein, 6 aa, synthetic construct
PKIFSH

SEQ ID NO: 188 - protein, 5 aa, synthetic construct
WRGVA

SEQ ID NO: 189 - protein, 159 aa, synthetic construct
GSSHHHHHHNPSLIRSESWETWTGNEANLLDGDDNTGVWYFKKNFHVASLAGEFIGLDLGKEIKLDGIRFVIGKNGGGSSDKWNKFKLEYSLDNESWTTIKEYDKTGAPAGKDVIEESFETPISAKYIRLTNLESHFRALTFSEFAIVSDDYKDDDDKG

SEQ ID NO: 190 - protein, 4 aa, synthetic construct
ETWT

SEQ ID NO: 191 - protein, 8 aa, synthetic construct
FKKNFHVA

SEQ ID NO: 192 - protein, 5 aa, synthetic construct
SHFRA

SEQ ID NO: 193 - protein, 150 aa, synthetic construct
NPSLIRSESWTYVIGNEANLLDGDDNTGVWYVLNKLKKKSLAGEFIGLDLGKEIKLDGIRFVIGKNGGGSSDKWNKFKLEYSLDNESWTTIKEYDKTGAPAGKDVIEESFETPISAKYIRLTNLEEKFDNLTFSEFAIVSDDYKDDDDKG

SEQ ID NO: 194 - protein, 4 aa, synthetic construct
TYVI

SEQ ID NO: 195 - protein, 8 aa, synthetic construct
VLNKLKKK

SEQ ID NO: 196 - protein, 5 aa, synthetic construct
EKFDN

SEQ ID NO: 197 - protein, 150 aa, synthetic construct
NPSLIRSESWVFIIGNEANLLDGDDNTGVWYLLHKLTKTSLAGEFIGLDLGKEIKLDGIRFVIGKNGGGSSDKWNKFKLEYSLDNESWTTIKEYDKTGAPAGKDVIEESFETPISAKYIRLTNLEHAKKWLTFSEFAIVSDDYKDDDDKG

SEQ ID NO: 198 - protein, 4 aa, synthetic construct
VFII

SEQ ID NO: 199 - protein, 8 aa, synthetic construct
LLHKLTKT

SEQ ID NO: 200 - protein, 5 aa, synthetic construct
HAKKW

SEQ ID NO: 201 - protein, 150 aa, synthetic construct
NPSLIRSESWVVIDGNEANLLDGDDNTGVWYFTDYWIVESLAGEFIGLDLGKEIKLDGIRFVIGKNGGGSSDKWNKFKLEYSLDNESWTTIKEYDKTGAPAGKDVIEESFETPISAKYIRLTNLEKTNEFLTFSEFAIVSDDYKDDDDKG

SEQ ID NO: 202 - protein, 4 aa, synthetic construct
VVID

SEQ ID NO: 203 - protein, 8 aa, synthetic construct
FTDYWIVE

SEQ ID NO: 204 - protein, 5 aa, synthetic construct
KTNEF

SEQ ID NO: 205 - protein, 150 aa, synthetic construct
NPSLIRSESWAWWIGNEANLLDGDDNTGVWYALKVFNYWSLAGEFIGLDLGKEIKLDGIRFVIGKNGGGSSDKWNKFKLEYSLDNESWTTIKEYDKTGAPAGKDVIEESFETPISAKYIRLTNLEPSLTDLTFSEFAIVSDDYKDDDDKG

SEQ ID NO: 206 - protein, 4 aa, synthetic construct
AWWI

SEQ ID NO: 207 - protein, 8 aa, synthetic construct
ALKVFNYW

SEQ ID NO: 208 - protein, 5 aa, synthetic construct
PSLTD

SEQ ID NO: 209 - protein, 151 aa, synthetic construct
NPSLIRSESWAQTNGNEANLLDGDDNTGVWYVAVVTHIKFSLAGEFIGLDLGKEIKLDGIRFVIGKNGGGSSDKWNKFKLEYSLDNESWTTIKEYDKTGAPAGKDVIEESFETPISAKYIRLTNLEHAYKNLTFSEFAIVSDDYKDDDDKG

SEQ ID NO: 210 - protein, 4 aa, synthetic construct
AQTN

SEQ ID NO: 211 - protein, 9 aa, synthetic construct
VAVVTHIKF

SEQ ID NO: 212 - protein, 5 aa, synthetic construct
HAYKN

SEQ ID NO: 213 - protein, 149 aa, synthetic construct
GSSHHHHHHNPSLIRSESWSIYKGNEANLLDGDDNTGVWYTGKLDKSYNSLAGEFIGLDLGKEIKLDGIRFVIGKNGGGSSDKWNKFKLEYSLDNESWTTIKEYDKTGAPAGKDVIEESFETPISAKYIRLTNLEFKTLTFSEFAIVSD

SEQ ID NO: 214 - protein, 4 aa, synthetic construct
SIYK

SEQ ID NO: 215 - protein, 9 aa, synthetic construct
TGKLDKSYN

SEQ ID NO: 216 - skipped

SEQ ID NO: 217 - protein, 152 aa, synthetic construct
NPSLIRSESWWRPTGNEANLLDGDDNTGVWYAKIYFYYWDISLAGEFIGLDLGKEIKLDGIRFVIGKNGGGSSDKWNKFKLEYSLDNESWTTIKEYDKTGAPAGKDVIEESFETPISAKYIRLTNLERHEKSLTFSEFAIVSDDYKDDDDKG

SEQ ID NO: 218 - protein, 4 aa, synthetic construct
WRPT

SEQ ID NO: 219 - protein, 10 aa, synthetic construct
AKIYFYYWDI

SEQ ID NO: 220 - protein, 5 aa, synthetic construct
RHEKS

SEQ ID NO: 221 - protein, 153 aa, synthetic construct
NPSLIRSESWDDVHGNEANLLDGDDNTGVWYFHYYFRSSKKHSLAGEFIGLDLGKEIKLDGIRFVIGKNGGGSSDKWNKFKLEYSLDNESWTTIKEYDKTGAPAGKDVIEESFETPISAKYIRLTNLEKYSLELTFSEFAIVSDDYKDDDDKG

SEQ ID NO: 222 - protein, 4 aa, synthetic construct
DDVH

SEQ ID NO: 223 - protein, 11 aa, synthetic construct
FHYYFRSSKKH

SEQ ID NO: 224 - protein, 5 aa, synthetic construct
KYSLE

SEQ ID NO: 225 - protein, 157 aa, synthetic construct
GSSHHHHHHNPSLIRSESWINLFGNEANLLDGDDNTGVWYGILYTVSLEYSKIYSLAGEFIGLDLGKEIKLDGIRFVIGKNGGGSSDKWNKFKLEYSLDNESWTTIKEYDKTGAPAGKDVIEESFETPISAKYIRLTNLEKDTTFLTFSEFAIVSDG

SEQ ID NO: 226 - protein, 4 aa, synthetic construct
INLF

SEQ ID NO: 227 - protein, 14 aa, synthetic construct
GILYTVSLEYSKIY

SEQ ID NO: 228 - protein, 5 aa, synthetic construct
KDTTF

SEQ ID NO: 229 - protein, 158 aa, synthetic construct
GSSHHHHHHNPSLIRSESWVGSKGNEANLLDGDDNTGVWYPWFPKAIFFKNREFGSLAGEFIGLDLGKEIKLDGIRFVIGKNGGGSSDKWNKFKLEYSLDNESWTTIKEYDKTGAPAGKDVIEESFETPISAKYIRLTNLEYVSVILTFSEFAIVSDG

SEQ ID NO: 230 - protein, 4 aa, synthetic construct
VGSK

SEQ ID NO: 231 - protein, 15 aa, synthetic construct
PWFPKAIFFKNREFG

SEQ ID NO: 232 - protein, 5 aa, synthetic construct
YVSVI

SEQ ID NO: 233 - protein, 147 aa, synthetic construct
NPSLIRSESWQFSNGNEANLLDGDDNTGVWYDNNDHFGSLAGEFIGLDLGKEIKLDGIRFVIGKNGGGSSDKWNKFKLEYSLDNESWTTIKEYDKTGAPAGKDVIEESFETPISAKYIRLTNLEVIYLTFSEFAIVSDDYKDDDDKG

SEQ ID NO: 234 - protein, 4 aa, synthetic construct
QFSN

SEQ ID NO: 235 - protein, 7 aa, synthetic construct
DNNDHFG

SEQ ID NO: 236 - skipped

SEQ ID NO: 237 - protein, 157 aa, synthetic construct
GSSHHHHHHNPSLIRSESWHTYNGNEANLLDGDDNTGVWYNNNSWFSSLAGEFIGLDLGKEIKLDGIRFVIGKNGGGSSDKWNKFKLEYSLDNESWTTIKEYDKTGAPAGKDVIEESFETPISAKYIRLTNLEAKNNLTFSEFAIVSDDYKDDDDKG

SEQ ID NO: 238 - protein, 4 aa, synthetic construct
HTYN

SEQ ID NO: 239 - protein, 7 aa, synthetic construct
NNNSWFS

SEQ ID NO: 240 - protein, 4 aa, synthetic construct
AKNN

SEQ ID NO: 241 - protein, 157 aa, synthetic construct
GSSHHHHHHNPSLIRSESWPGVIGNEANLLDGDDNTGVWYEDKIWFNSLAGEFIGLDLGKEIKLDGIRFVIGKNGGGSSDKWNKFKLEYSLDNESWTTIKEYDKTGAPAGKDVIEESFETPISAKYIRLTNLEAKYNLTFSEFAIVSDDYKDDDDKG

SEQ ID NO: 242 - protein, 4 aa, synthetic construct
PGVI

SEQ ID NO: 243 - protein, 7 aa, synthetic construct
EDKIWFN

SEQ ID NO: 244 - protein, 4 aa, synthetic construct
AKYN

SEQ ID NO: 245 - protein, 157 aa, synthetic construct
GSSHHHHHHNPSLIRSESWPYLDGNEANLLDGDDNTGVWYTGRNPFNSLAGEFIGLDLGKEIKLDGIRFVIGKNGGGSSDKWNKFKLEYSLDNESWTTIKEYDKTGAPAGKDVIEESFETPISAKYIRLTNLEAQNNLTFSEFAIVSDDYKDDDDKG

SEQ ID NO: 246 - protein, 4 aa, synthetic construct
PYLD

SEQ ID NO: 247 - protein, 7 aa, synthetic construct
TGRNPFN

SEQ ID NO: 248 - protein, 4 aa, synthetic construct
AQNN

SEQ ID NO: 249 - protein, 157 aa, synthetic construct
GSSHHHHHHNPSLIRSESWPHNEGNEANLLDGDDNTGVWYWDRSVFNSLAGEFIGLDLGKEIKLDGIRFVIGKNGGGSSDKWNKFKLEYSLDNESWTTIKEYDKTGAPAGKDVIEESFETPISAKYIRLTNLEPQYNLTFSEFAIVSDDYKDDDDKG

SEQ ID NO: 250 - protein, 4 aa, synthetic construct
PHNE

SEQ ID NO: 251 - protein, 7 aa, synthetic construct
WDRSVFN

SEQ ID NO: 252 - protein, 4 aa, synthetic construct
PQYN

SEQ ID NO: 253 - protein, 157 aa, synthetic construct
GSSHHHHHHNPSLIRSESWKWVSGNEANLLDGDDNTGVWYDGNHWFNSLAGEFIGLDLGKEIKLDGIRFVIGKNGGGSSDKWNKFKLEYSLDNESWTTIKEYDKTGAPAGKDVIEESFETPISAKYIRLTNLESQHNLTFSEFAIVSDDYKDDDDKG

SEQ ID NO: 254 - protein, 4 aa, synthetic construct
KWVS

SEQ ID NO: 255 - protein, 7 aa, synthetic construct
DGNHWFN

SEQ ID NO: 256 - protein, 4 aa, synthetic construct
SQHN

SEQ ID NO: 257 - protein, 157 aa, synthetic construct
GSSHHHHHHNPSLIRSESWPSTSGNEANLLDGDDNTGVWYENKSWFNSLAGEFIGLDLGKEIKLDGIRFVIGKNGGGSSDKWNKFKLEYSLDNESWTTIKEYDKTGAPAGKDVIEESFETPISAKYIRLTNLETRYNLTFSEFAIVSDDYKDDDDKG

SEQ ID NO: 258 - protein, 4 aa, synthetic construct
PSTS

SEQ ID NO: 259 - protein, 7 aa, synthetic construct
ENKSWFN

SEQ ID NO: 260 - protein, 4 aa, synthetic construct
TRYN

SEQ ID NO: 261 - protein, 148 aa, synthetic construct
NPSLIRSESWVIKKGNEANLLDGDDNTGVWYSHKLWFNSLAGEFIGLDLGKEIKLDGIRFVIGKNGGGSSDKWNKFKLEYSLDNESWTTIKEYDKTGAPAGKDVIEESFETPISAKYIRLTNLEVRDNLTFSEFAIVSDDYKDDDDKG

SEQ ID NO: 262 - protein, 4 aa, synthetic construct
VIKK

SEQ ID NO: 263 - protein, 7 aa, synthetic construct
SHKLWFN

SEQ ID NO: 264 - protein, 4 aa, synthetic construct
VRDN

SEQ ID NO: 265 - protein, 148 aa, synthetic construct
NPSLIRSESWRGHPGNEANLLDGDDNTGVWYRNHHVFSSLAGEFIGLDLGKEIKLDGIRFVIGKNGGGSSDKWNKFKLEYSLDNESWTTIKEYDKTGAPAGKDVIEESFETPISAKYIRLTNLEPSNNLTFSEFAIVSDDYKDDDDKG

SEQ ID NO: 266 - protein, 4 aa, synthetic construct
RGHP

SEQ ID NO: 267 - protein, 7 aa, synthetic construct
RNHHVFS

SEQ ID NO: 268 - protein, 4 aa, synthetic construct
PSNN

SEQ ID NO: 269 - protein, 148 aa, synthetic construct
NPSLIRSESWPDLGGNEANLLDGDDNTGVWYAEHTVYNSLAGEFIGLDLGKEIKLDGIRFVIGKNGGGSSDKWNKFKLEYSLDNESWTTIKEYDKTGAPAGKDVIEESFETPISAKYIRLTNLEARENLTFSEFAIVSDDYKDDDDKG

SEQ ID NO: 270 - protein, 4 aa, synthetic construct
PDLG

SEQ ID NO: 271 - protein, 7 aa, synthetic construct
AEHTVYN

SEQ ID NO: 272 - protein, 4 aa, synthetic construct
AREN

SEQ ID NO: 273 - protein, 148 aa, synthetic construct
NPSLIRSESWVIDAGNEANLLDGDDNTGVWYAGKNWFNSLAGEFIGLDLGKEIKLDGIRFVIGKNGGGSSDKWNKFKLEYSLDNESWTTIKEYDKTGAPAGKDVIEESFETPISAKYIRLTNLEAGVNLTFSEFAIVSDDYKDDDDKG

SEQ ID NO: 274 - protein, 4 aa, synthetic construct
VIDA

SEQ ID NO: 275 - protein, 7 aa, synthetic construct
AGKNWFN

SEQ ID NO: 276 - protein, 4 aa, synthetic construct
AGVN

SEQ ID NO: 277 - protein, 148 aa, synthetic construct
NPSLIRSESWHGDAGNEANLLDGDDNTGVWYDNKNWWNSLAGEFIGLDLGKEIKLDGIRFVIGKNGGGSSDKWNKFKLEYSLDNESWTTIKEYDKTGAPAGKDVIEESFETPISAKYIRLTNLEARQNLTFSEFAIVSDDYKDDDDKG

SEQ ID NO: 278 - protein, 4 aa, synthetic construct
HGDA

SEQ ID NO: 279 - protein, 7 aa, synthetic construct
DNKNWWN

SEQ ID NO: 280 - protein, 4 aa, synthetic construct
ARQN

SEQ ID NO: 281 - protein, 148 aa, synthetic construct
NPSLIRSESWPDDQGNEANLLDGDDNTGVWYWLKNWYASLAGEFIGLDLGKEIKLDGIRFVIGKNGGGSSDKWNKFKLEYSLDNESWTTIKEYDKTGAPAGKDVIEESFETPISAKYIRLTNLEPRNNLTFSEFAIVSDDYKDDDDKG

SEQ ID NO: 282 - protein, 4 aa, synthetic construct
PDDQ

SEQ ID NO: 283 - protein, 7 aa, synthetic construct
WLKNWYA

SEQ ID NO: 284 - protein, 4 aa, synthetic construct
PRNN

SEQ ID NO: 285 - protein, 148 aa, synthetic construct
NPSLIRSESWTVPWGNEANLLDGDDNTGVWYNGKSIFNSLAGEFIGLDLGKEIKLDGIRFVIGKNGGGSSDKWNKFKLEYSLDNESWTTIKEYDKTGAPAGKDVIEESFETPISAKYIRLTNLEAEFNLTFSEFAIVSDDYKDDDDKG

SEQ ID NO: 286 - protein, 4 aa, synthetic construct
TVPW

SEQ ID NO: 287 - protein, 7 aa, synthetic construct
NGKSIFN

SEQ ID NO: 288 - protein, 4 aa, synthetic construct
AEFN

SEQ ID NO: 289 - protein, 148 aa, synthetic construct
NPSLIRSESWTNALGNEANLLDGDDNTGVWYNKKHWFSSLAGEFIGLDLGKEIKLDGIRFVIGKNGGGSSDKWNKFKLEYSLDNESWTTIKEYDKTGAPAGKDVIEESFETPISAKYIRLTNLERGQNLTFSEFAIVSDDYKDDDDKG

SEQ ID NO: 290 - protein, 4 aa, synthetic construct
TNAL

SEQ ID NO: 291 - protein, 7 aa, synthetic construct
NKKHWFS

SEQ ID NO: 292 - protein, 4 aa, synthetic construct
RGQN

SEQ ID NO: 293 - protein, 148 aa, synthetic construct
NPSLIRSESWYANSGNEANLLDGDDNTGVWYDESDHYGSLAGEFIGLDLGKEIKLDGIRFVIGKNGGGSSDKWNKFKLEYSLDNESWTTIKEYDKTGAPAGKDVIEESFETPISAKYIRLTNLEKPVILTFSEFAIVSDDYKDDDDKG

SEQ ID NO: 294 - protein, 4 aa, synthetic construct
YANS

SEQ ID NO: 295 - protein, 7 aa, synthetic construct
DESDHYG

SEQ ID NO: 296 - protein, 4 aa, synthetic construct
KPVI

SEQ ID NO: 297 - protein, 148 aa, synthetic construct
NPSLIRSESWDRPTGNEANLLDGDDNTGVWYTDLDHFGSLAGEFIGLDLGKEIKLDGIRFVIGKNGGGSSDKWNKFKLEYSLDNESWTTIKEYDKTGAPAGKDVIEESFETPISAKYIRLTNLEWGKILTFSEFAIVSDDYKDDDDKG

SEQ ID NO: 298 - protein, 4 aa, synthetic construct
DRPT

SEQ ID NO: 299 - protein, 7 aa, synthetic construct
TDLDHFG

SEQ ID NO: 300 - protein, 4 aa, synthetic construct
WGKI

SEQ ID NO: 301 - protein, 148 aa, synthetic construct
NPSLIRSESWGVNYGNEANLLDGDDNTGVWYNENNHYGSLAGEFIGLDLGKEIKLDGIRFVIGKNGGGSSDKWNKFKLEYSLDNESWTTIKEYDKTGAPAGKDVIEESFETPISAKYIRLTNLEYDDTLTFSEFAIVSDDYKDDDDKG

SEQ ID NO: 302 - protein, 4 aa, synthetic construct
GVNY

SEQ ID NO: 303 - protein, 7 aa, synthetic construct
NENNHYG

SEQ ID NO: 304 - protein, 4 aa, synthetic construct
YDDT

SEQ ID NO: 305 - protein, 148 aa, synthetic construct
NPSLIRSESWRSPVGNEANLLDGDDNTGVWYNKNDHFGSLAGEFIGLDLGKEIKLDGIRFVIGKNGGGSSDKWNKFKLEYSLDNESWTTIKEYDKTGAPAGKDVIEESFETPISAKYIRLTNLEYFHTLTFSEFAIVSDDYKDDDDKG

SEQ ID NO: 306 - protein, 4 aa, synthetic construct
RSPV

SEQ ID NO: 307 - protein, 7 aa, synthetic construct
NKNDHFG

SEQ ID NO: 308 - protein, 4 aa, synthetic construct
YFHT

SEQ ID NO: 309 - protein, 148 aa, synthetic construct
NPSLIRSESWLLALGNEANLLDGDDNTGVWYTDQDHFGSLAGEFIGLDLGKEIKLDGIRFVIGKNGGGSSDKWNKFKLEYSLDNESWTTIKEYDKTGAPAGKDVIEESFETPISAKYIRLTNLEAVKWLTFSEFAIVSDDYKDDDDKG

SEQ ID NO: 310 - protein, 4 aa, synthetic construct
LLAL

SEQ ID NO: 311 - protein, 7 aa, synthetic construct
TDQDHFG

SEQ ID NO: 312 - protein, 4 aa, synthetic construct
AVKW

SEQ ID NO: 313 - protein, 148 aa, synthetic construct
NPSLIRSESWLPQEGNEANLLDGDDNTGVWYNYFDIAFSLAGEFIGLDLGKEIKLDGIRFVIGKNGGGSSDKWNKFKLEYSLDNESWTTIKEYDKTGAPAGKDVIEESFETPISAKYIRLTNLEEIVILTFSEFAIVSDDYKDDDDKG

SEQ ID NO: 314 - protein, 4 aa, synthetic construct
LPQE

SEQ ID NO: 315 - protein, 7 aa, synthetic construct
NYFDIAF

SEQ ID NO: 316 - protein, 4 aa, synthetic construct
EIVI

SEQ ID NO: 317 - protein, 148 aa, synthetic construct
NPSLIRSESWTQFIGNEANLLDGDDNTGVWYYDLKHQYSLAGEFIGLDLGKEIKLDGIRFVIGKNGGGSSDKWNKFKLEYSLDNESWTTIKEYDKTGAPAGKDVIEESFETPISAKYIRLTNLEKILPLTFSEFAIVSDDYKDDDDKG

SEQ ID NO: 318 - protein, 4 aa, synthetic construct
TQFI

SEQ ID NO: 319 - protein, 7 aa, synthetic construct
YDLKHQY

SEQ ID NO: 320 - protein, 4 aa, synthetic construct
KILP

SEQ ID NO: 321 - protein, 148 aa, synthetic construct
NPSLIRSESWPVYRGNEANLLDGDDNTGVWYNSWAEKFSLAGEFIGLDLGKEIKLDGIRFVIGKNGGGSSDKWNKFKLEYSLDNESWTTIKEYDKTGAPAGKDVIEESFETPISAKYIRLTNLELEYGLTFSEFAIVSDDYKDDDDKG

SEQ ID NO: 322 - protein, 4 aa, synthetic construct
PVYR

SEQ ID NO: 323 - protein, 7 aa, synthetic construct
NSWAEKF

SEQ ID NO: 324 - protein, 4 aa, synthetic construct
LEYG

SEQ ID NO: 325 - protein, 148 aa, synthetic construct
NPSLIRSESWLVIYGNEANLLDGDDNTGVWYPKNWSSYSLAGEFIGLDLGKEIKLDGIRFVIGKNGGGSSDKWNKFKLEYSLDNESWTTIKEYDKTGAPAGKDVIEESFETPISAKYIRLTNLETVILLTFSEFAIVSDDYKDDDDKG

SEQ ID NO: 326 - protein, 4 aa, synthetic construct
LVIY

SEQ ID NO: 327 - protein, 7 aa, synthetic construct
PKNWSSY

SEQ ID NO: 328 - protein, 4 aa, synthetic construct
TVIL

SEQ ID NO: 329 - protein, 148 aa, synthetic construct
NPSLIRSESWQLEGGNEANLLDGDDNTGVWYFALIAINSLAGEFIGLDLGKEIKLDGIRFVIGKNGGGSSDKWNKFKLEYSLDNESWTTIKEYDKTGAPAGKDVIEESFETPISAKYIRLTNLEVKYDLTFSEFAIVSDDYKDDDDKG

SEQ ID NO: 330 - protein, 4 aa, synthetic construct
QLEG

SEQ ID NO: 331 - protein, 7 aa, synthetic construct
FALIAIN

SEQ ID NO: 332 - protein, 4 aa, synthetic construct
VKYD

SEQ ID NO: 333 - protein, 145 aa, synthetic construct
HHHHHHYPSLIRSESWARPVGNEANLLDGDDNTGVWYHKKKSKISLAGEFIGLDLGKEIKLDGIRFVIGKNGGGSSDKWNKFKLEYSLDNESWTTIKEYDKTGAPAGKDVIEESFETPISAKYIRLTNLEVNSPLTFSEFAIVSD

SEQ ID NO: 334 - protein, 4 aa, synthetic construct
ARPV

SEQ ID NO: 335 - protein, 7 aa, synthetic construct
HKKKSKI

SEQ ID NO: 336 - protein, 4 aa, synthetic construct
VNSP

SEQ ID NO: 337 - protein, 148 aa, synthetic construct
GSSHHHHHHNPSLIRSESWAYDTGNEANLLDGDDNTGVWYIYDSDEKSLAGEFIGLDLGKEIKLDGIRFVIGKNGGGSSDKWNKFKLEYSLDNESWTTIKEYDKTGAPAGKDVIEESFETPISAKYIRLTNLEYYHYLTFSEFAIVSD

SEQ ID NO: 338 - protein, 4 aa, synthetic construct
AYDT

SEQ ID NO: 339 - protein, 7 aa, synthetic construct
IYDSDEK

SEQ ID NO: 340 - protein, 4 aa, synthetic construct
YYHY

SEQ ID NO: 341 - protein, 168 aa, synthetic construct
MGSSHHHHHHNPSLIRSESWALVYGNEANLLDGDDNTGVWYKFKYWAVSLAGEFIGLDLGKEIKLDGIRFVIGKNGGGSSDKWNKFKLEYSLDNESWTTIKEYDKTGAPAGKDVIEESFETPISAKYIRLTNLETEYKWGLTFSEFAIVSDGGGLNDIFEAQKIEWHE

SEQ ID NO: 342 - protein, 4 aa, synthetic construct
ALVY

SEQ ID NO: 343 - protein, 7 aa, synthetic construct
KFKYWAV

SEQ ID NO: 344 - protein, 6 aa, synthetic construct
TEYKWG

SEQ ID NO: 345 - protein, 159 aa, synthetic construct
GSSHHHHHHNPSLIRSESWTPYDGNEANLLDGDDNTGVWYKTLNGDTSLAGEFIGLDLGKEIKLDGIRFVIGKNGGGSSDKWNKFKLEYSLDNESWTTIKEYDKTGAPAGKDVIEESFETPISAKYIRLTNLEYVLRWWLTFSEFAIVSDDYKDDDDKG

SEQ ID NO: 346 - protein, 4 aa, synthetic construct
TPYD

SEQ ID NO: 347 - protein, 7 aa, synthetic construct
KTLNGDT

SEQ ID NO: 348 - protein, 6 aa, synthetic construct
YVLRWW

SEQ ID NO: 349 - protein, 150 aa, synthetic construct
NPSLIRSESWVVRFGNEANLLDGDDNTGVWYINGWWWSSLAGEFIGLDLGKEIKLDGIRFVIGKNGGGSSDKWNKFKLEYSLDNESWTTIKEYDKTGAPAGKDVIEESFETPISAKYIRLTNLENTRVHYLTFSEFAIVSDDYKDDDDKG

SEQ ID NO: 350 - protein, 4 aa, synthetic construct
VVRF

SEQ ID NO: 351 - protein, 7 aa, synthetic construct
INGWWWS

SEQ ID NO: 352 - protein, 6 aa, synthetic construct
NTRVHY

SEQ ID NO: 353 - protein, 150 aa, synthetic construct
NPSLIRSESWVWFAIGNEANLLDGDDNTGVWYFTKTWSESLAGEFIGLDLGKEIKLDGIRFVIGKNGGGSSDKWNKFKLEYSLDNESWTTIKEYDKTGAPAGKDVIEESFETPISAKYIRLTNLELFNNPLTFSEFAIVSDDYKDDDDKG

SEQ ID NO: 354 - protein, 5 aa, synthetic construct
VWFAI

SEQ ID NO: 355 - protein, 7 aa, synthetic construct
FTKTWSE

SEQ ID NO: 356 - protein, 5 aa, synthetic construct
LFNNP

SEQ ID NO: 357 - protein, 161 aa, synthetic construct
GSSHHHHHHDPTLIHTPGWAVGDGSEADLLDGDDSTGVEYVIVWQWDSLAGEFIGLDLGEVVEVGGIHFVIGADGGGSSDKWTRFRLEYSLDGESWTTIREYDHTGAPAGQDVIDEDFETPISAQYIRLTNLEVHIQGLTFSEFAIVSDELEDYKDDDDKG

SEQ ID NO: 358 - protein, 4 aa, synthetic construct
AVGD

SEQ ID NO: 359 - protein, 7 aa, synthetic construct
VIVWQWD

SEQ ID NO: 360 - protein, 5 aa, synthetic construct
VHIQG

SEQ ID NO: 361 - protein, 161 aa, synthetic construct
GSSHHHHHHDPTLIHTPGWDVIAGSEADLLDGDDSTGVEYPIPYQGWSLAGEFIGLDLGEVVEVGGIHFVIGADGGGSSDKWTRFRLEYSLDGESWTTIREYDHTGAPAGQDGIDEDFETPISAQYIRLTNLEWAGDNLTFSEFAIVSDELEDYKDDDDKG

SEQ ID NO: 362 - protein, 4 aa, synthetic construct
DVIA

SEQ ID NO: 363 - protein, 7 aa, synthetic construct
PIPYQGW

SEQ ID NO: 364 - protein, 5 aa, synthetic construct
WAGDN

SEQ ID NO: 365 - protein, 161 aa, synthetic construct
GSSHHHHHHDPTLIHTPGWDVYGGSEADLLDGDDSTGVEYVVIWEVGSLAGEFIGLDLGEVVEVGGIHFVIGADGGGSSDKWTRFRLEYSLDGESWTTIREYDHTGAPAGQDVIDEDFETPISAQYIRLTNLEGQTQQLTFSEFAIVSDELEDYKDDDDKG

SEQ ID NO: 366 - protein, 4 aa, synthetic construct
DVYG

SEQ ID NO: 367 - protein, 7 aa, synthetic construct
VVIWEVG

SEQ ID NO: 368 - protein, 5 aa, synthetic construct
GQTQQ

SEQ ID NO: 369 - protein, 161 aa, synthetic construct
GSSHHHHHHDPTLIHTPGWEVSIGSEADLLDGDDSTGVEYVVIWEVNSLAGEFIGLDLGEVVEVGGIHFVIGADGGGSSDKWTRFRLEYSLDGESWTTIREYDHTGAPAGQDVIDEDFETPISAQYIRLTNLEYVISQLTFSEFAIVSDELEDYKDDDDKG

SEQ ID NO: 370 - protein, 4 aa, synthetic construct
EVSI

SEQ ID NO: 371 - protein, 7 aa, synthetic construct
VVIWEVN

SEQ ID NO: 372 - protein, 5 aa, synthetic construct
YVISQ

SEQ ID NO: 373 - protein, 160 aa, synthetic construct
SSHHHHHHDPTLIHTPGWIVDDGSEADLLDGDDSTGVEYFAYPAYISLAGEFIGLDLGEVVEVGGIHFVIGADGGGSSDKWTRFRLEYSLDGESWITIREYDHTGAPAGQDVIDEDFETPISAQYIRLTNLEELDTELTFSEFAIVSDELEDYKDDDDKG

SEQ ID NO: 374 - protein, 4 aa, synthetic construct
IVDD

SEQ ID NO: 375 - protein, 7 aa, synthetic construct
FAYPAYI

SEQ ID NO: 376 - protein, 5 aa, synthetic construct
ELDTE

SEQ ID NO: 377 - protein, 161 aa, synthetic construct
GSSHHHHHHDPTLIHTPGWLVIPGSEADLLDGDDSTGVEYIIVEEFTSLAGEFIGLDLGEVVEVGGIHFVIGADGGGSSDKWTRFRLEYSLDGESWTTIREYDHTGAPAGQDVIDEDFETPISAQYIRLTNLEYNFIDLTFSEFAIVSDELEDYKDDDDKG

SEQ ID NO: 378 - protein, 161 aa, synthetic construct
GSSHHHHHHDPTLIHTPGWNVEQGSEADLLDGDDSTGVEYILVEEFPSLAGEFIGLDLGEVVEVGGIHFVIGADGGGSSDKWTRFRLEYSLDGESWTTIREYDHTGAPAGQDVIDEDFETPISAQYIRLTNLENEVPQLTFSEFAIVSDELEDYKDDDDKG

SEQ ID NO: 379 - protein, 161 aa, synthetic construct
GSSHHHHHHDPTLIHTPGWNVSDGSEADLLDGDDSTGVEYVLIWQWDSLAGEFIGLDLGEVVEVGGIHFVIGADGGGSSDKWTRFRLEYSLDGESWTTIREYDHTGAPAGQDVIDEDFETPISAQYIRLTNLEQQQDELTFSEFAIVSDELEDYKDDDDKG

SEQ ID NO: 380 - protein, 4 aa, synthetic construct
NVSD

SEQ ID NO: 381 - protein, 7 aa, synthetic construct
VLIWQWD

SEQ ID NO: 382 - protein, 5 aa, synthetic construct
QQQDE

SEQ ID NO: 383 - protein, 161 aa, synthetic construct
GSSHHHHHHDPTLIHTPGWPVVTGSEADLLDGDDSTGVEYIVLTDQASLAGEFIGLDLGEVVEVGGINFVIGADGGGSSDKWTRFRLEYSLDGESWTTIREYDHTGAPAGQDVIDEDFETPISAQYIRLTNLELAQDPLTFSEFAIVSDELEDYKDDDDKG

SEQ ID NO: 384 - protein, 157 aa, synthetic construct
HHHHHHDPTLIHTPGWQVFNGSEADLLDGDDSTGVEYPHWTPHQSLAGEFIGLDLGEVVEVGGIHFVIGADGGGSSDKWTRFRLEYSLDGESWTTIREYDHKGPCRARCYRRRFQTPISAQYIRLTNLEDYQEILTFSEFAIVSDELEDYKDDDDKG

SEQ ID NO: 385 - protein, 4 aa, synthetic construct
QVFN

SEQ ID NO: 386 - protein, 7 aa, synthetic construct
PHWTPHQ

SEQ ID NO: 387 - protein, 5 aa, synthetic construct
DYQEI

SEQ ID NO: 388 - protein, 161 aa, synthetic construct
GSSHHHHHHDPTLIHTPGWQVGDGSEADLLDGDDSTGVEYITWIWLDSLAGEFIGLDLGEVVEVGGIHFVIGADGGGSSDKWTRFRLEYSLDGESWTTIREYDHTGAPAGQDVIDEDFETPISAQYIRLTNLEWDQESLTFSEFAIVSDELEDYKDDDDKG

SEQ ID NO: 389 - protein, 4 aa, synthetic construct
QVGD

SEQ ID NO: 390 - protein, 7 aa, synthetic construct
ITWIWLD

SEQ ID NO: 391 - protein, 5 aa, synthetic construct
WDQES

SEQ ID NO: 392 - protein, 161 aa, synthetic construct
GSSHHHHHHDPTLIHTPGWRSNLGSEADLLDGDDSTGVEYIIITDYESLAGEFIGLDLGEVVEVGGINFVIGADGGGSSDKWTRFRLEYSLDGESWTTIREYDHTGAPAGQDVIDEDFETPISAQYIRLTNLETAYFTLTFSEFAIVSDELEDYKDDDDKG

SEQ ID NO: 393 - protein, 4 aa, synthetic construct
RSNL

SEQ ID NO: 394 - protein, 7 aa, synthetic construct
IIITDYE

SEQ ID NO: 395 - protein, 5 aa, synthetic construct
TAYFT

SEQ ID NO: 396 - protein, 161 aa, synthetic construct
GSSHHHHHHDPTLIHTPGWSVAGGSEADLLDGDDSTSVEYPQPLVGTSLAGEFIGLDLGEVVEVGGIHFVIGADGGGSSDKWTRFRLEYSLDGESWTTIREYDHTGAPAGQDVIDEDFETPISAQYIRLTNLEWAADALTFSEFAIVSDELEDYKDDDDKG

SEQ ID NO: 397 - protein, 4 aa, synthetic construct
SVAG

SEQ ID NO: 398 - protein, 7 aa, synthetic construct
PQPLVGT

SEQ ID NO: 399 - protein, 5 aa, synthetic construct
WAADA

SEQ ID NO: 400 - protein, 158 aa, synthetic construct
HHHHHHDPTLIHTPGWTVAEGSEADLLDGDDSTGVEYVIIHEFTSLAGEFIGLDLGEVVEVGGIHFVIGADGGGSSDKWTRFRLEYSLDGESWTTIREYDHTGAPAGQDVIDEDFETPISAQYIRLTNLEHDPGVLTFSEFAIVSDELEDYKDDDDKG

SEQ ID NO: 401 - protein, 4 aa, synthetic construct
TVAE

SEQ ID NO: 402 - protein, 7 aa, synthetic construct
VIIHEFT

SEQ ID NO: 403 - protein, 5 aa, synthetic construct
HDPGV

SEQ ID NO: 404 - protein, 161 aa, synthetic construct
  VARIANT 159: note = can be any amino acid
GSSHHHHHHDPTLIHTPGWTVDIGSEADLLDGDDSTGVEYPPATPYASLAGEFIGLDLGEVVEVGGIHFVIGADGGGSSDKWTRFRLEYSLDGESWTTIREYDHTGAPAGQDVIDEDFETPISAQYIRLTNLESHGDILTFSEFAIVSDELEDYKDDDXKG

SEQ ID NO: 405 - protein, 4 aa, synthetic construct
TVDI

SEQ ID NO: 406 - protein, 7 aa, synthetic construct
PPATPYA

SEQ ID NO: 407 - protein, 5 aa, synthetic construct
SHGDI

SEQ ID NO: 408 - protein, 161 aa, synthetic construct
GSSHHHHHHDPTLIHTPGWYVAAGSEADLLDGDDSTGVEYVIIWEPQSLAGEFIGLDLGEVVEVGGIHFVIGADGGGSSDKWTRFRLEYSLDGESWTTIREYDHTGAPAGQDVIDEDFETPISAQYIRLTNLEYIHNWLTFSEFAIVSDELEDYKDDDDKG

SEQ ID NO: 409 - protein, 4 aa, synthetic construct
YVAA

SEQ ID NO: 410 - protein, 7 aa, synthetic construct
VIIWEPQ

SEQ ID NO: 411 - protein, 5 aa, synthetic construct
YIHNW

SEQ ID NO: 412 - protein, 158 aa, synthetic construct
HHHHHHDPTLIHTPGWYVVTGSEADLLDGDDSTGVEYPYIIQGLSLAGEFIGLDLGEVVEVGGIHFVIGADGGGSSDKWTRFRLEYSLDGESWTTIREYDHTGAPAGQDVIDEDFETPISAQYIRLTNLEYHQFHLTFSEFAIVSDELEDYKDDDDKG

SEQ ID NO: 413 - protein, 4 aa, synthetic construct
YVVT

SEQ ID NO: 414 - protein, 7 aa, synthetic construct
PYIIQGL

SEQ ID NO: 415 - protein, 5 aa, synthetic construct
YHQFH

SEQ ID NO: 416 - protein, 161 aa, synthetic construct
GSSHHHHHHDPTLIHTPGWAVIHGSEADLLDGDDSTGVEYWWLSVAVSLAGEFIGLDLGEVVEVGGIHFVIGADGGGSSDKWTRFRLEYSLDGESWTTIREYDHTGAPAGQDVIDEDFETPISAQYIRLTNLEFYHPYLTFSEFAIVSDELEDYKDDDDKG

SEQ ID NO: 417 - protein, 4 aa, synthetic construct
AVIH

SEQ ID NO: 418 - protein, 7 aa, synthetic construct
WWLSVAV

SEQ ID NO: 419 - protein, 5 aa, synthetic construct
FYHPY

SEQ ID NO: 420 - protein, 160 aa, synthetic construct
  VARIANT 139: note = can be any amino acid
SSHHHHHHDPTLIHTPGWAVYVGSEADLLDGDDSTGVEYWWTATGDSLAGEFIGLDLGKVVEVGGIHFVIGADGGGSSDKWTRFRLEYSLDGESWTTIREYDHTGAPAGQDVIDEDFETPISAQYIRLTNLENVDQSLXFSEFAIVSDELEDYKDDDDKG

SEQ ID NO: 421 - protein, 4 aa, synthetic construct
AVYV

SEQ ID NO: 422 - protein, 7 aa, synthetic construct
WWTATGD

SEQ ID NO: 423 - protein, 5 aa, synthetic construct
NVDQS

SEQ ID NO: 424 - protein, 161 aa, synthetic construct
GSSHHHHHHDPTLIHTPGWEVNYGSEADLLDGDDSTGVEYFTWAYGTSLAGEFIGLDLGEVVEVGGIHFVIGADGGGSSDKWTRFRLEYSLDGESWTTIREYDHTGAPAGQDVIDEDFETPISAQYIRLTNLENSHWWLTFSEFAIVSDELEDYKDDDDKG

SEQ ID NO: 425 - protein, 4 aa, synthetic construct
EVNY

SEQ ID NO: 426 - protein, 7 aa, synthetic construct
FTWAYGT

SEQ ID NO: 427 - protein, 5 aa, synthetic construct
NSHWW

SEQ ID NO: 428 - protein, 161 aa, synthetic construct
GSSHHHHHHDPTLIHTPGWFVHWGSEADLLDGDDSTGVEYWVWHSFTSLAGEFIGLDLGEVVEVGGIHFVIGADGGGSSDKWTRFRLEYSLDGESWTTIREYDHTGAPAGQDVIDEDFETPISAQYIRLTNLEHQGDALTFSEFAIVSDELEDYKDDDDKG

SEQ ID NO: 429 - protein, 4 aa, synthetic construct
FVHW

SEQ ID NO: 430 - protein, 7 aa, synthetic construct
WVWHSFT

SEQ ID NO: 431 - protein, 5 aa, synthetic construct
HQGDA

SEQ ID NO: 432 - protein, 161 aa, synthetic construct
GSSHHHHHHDPTLIHTPGWFVSDGSEADLLDGDDSTGVEYIYPYSYLSLAGEFIGLDLGEVVEVGGIHFVIGADGGGSSDKWTRFRLEYSLDGESWTTIREYDHTGAPAGQDVIDEDFETPISAQYIRLTNLEPTHWILTFSEFAIVSDELEDYKDDDDKG

SEQ ID NO: 433 - protein, 4 aa, synthetic construct
FVSD

SEQ ID NO: 434 - protein, 7 aa, synthetic construct
IYPYSYL

SEQ ID NO: 435 - protein, 5 aa, synthetic construct
PTHWI

SEQ ID NO: 436 - protein, 161 aa, synthetic construct
  VARIANT 43: note = can be any amino acid
GSSHHHHHHDPTLIHTPGWFVVPGSEADLLDGDDSTGVEYFLXPGWTSLAGEFIGLDLGEVVEVGGIHFVIGADGGGSSDKWTRFRLEYSLDGESWTTIREYDHTGAPAGQDVIDEDFETPISAQYIRLTNLEDPPVWLTFSEFAIVSDELEDYKDDDDKG

SEQ ID NO: 437 - protein, 4 aa, synthetic construct
FVVP

SEQ ID NO: 438 - protein, 7 aa, synthetic construct
  VARIANT 3: note = can be any amino acid
FLXPGWT

SEQ ID NO: 439 - protein, 5 aa, synthetic construct
DPPVW

SEQ ID NO: 440 - protein, 161 aa, synthetic construct
GSSHHHHHHDPTLIHTPGWIVVAGSEADLLDGDDSTGVEYFQVPGWTSLAGEFIGLDLGEVVEVGGIHFVIGADGGGSSDKWTRFRLEYSLDGESWTTIREYDHTGAPAGQDVIDEDFETPISAQYIRLTNLEDPPVWLTFSEFAIVSDELEDYKDDDDKG

SEQ ID NO: 441 - protein, 4 aa, synthetic construct
IVVA

SEQ ID NO: 442 - protein, 7 aa, synthetic construct
FQVPGWT

SEQ ID NO: 443 - protein, 5 aa, synthetic construct
DPPVW

SEQ ID NO: 444 - protein, 158 aa, synthetic construct
HHHHHHDPTLIHTPGWDVNFGSEADLLDGDDSTGVEYPQIGHYISLAGEFIGLDLGEVVEVGGIHFVIGADGGGSSDKWTRFRLEYSLDGESWTTIREYDHTGAPAGQDVIDEDFETPISAQYIRLTNLESFVHPLTFSEFAIVSDELEDYKDDDDKG

SEQ ID NO: 445 - protein, 4 aa, synthetic construct
DVNF

SEQ ID NO: 446 - protein, 7 aa, synthetic construct
PQIGHYI

SEQ ID NO: 447 - protein, 5 aa, synthetic construct
SFVHP

SEQ ID NO: 448 - protein, 158 aa, synthetic construct
HHHHHHDPTLIHTPGWLVANGSEADLLDGDDSTGVEYPFAHHQISLAGEFIGLDLGEVVEVGGIHFVIGADGGGSSDKWTRFRLEYSLDGESWTTIREYDHTGAPAGQDVIDEDFETPISAQYIRLTNLESFIYPLTFSEFAIVSDELEDYKDDDDKG

SEQ ID NO: 449 - protein, 4 aa, synthetic construct
LVAN

SEQ ID NO: 450 - protein, 7 aa, synthetic construct
PFAHHQI

SEQ ID NO: 451 - protein, 5 aa, synthetic construct
SFIYP

SEQ ID NO: 452 - protein, 158 aa, synthetic construct
HHHHHHDPTLIHTPGWNVAPGSEADLLDGDDSTGIEYFHIGHHISLAGEFIGLDLGEVVEVGGIHFVIGADGGGSSDKWTRFRLEYSLDGESWTTIREYDHTGAPAGQDVIDEDFETPISAQYIRLTNLEPFTWPLTFSEFAIVSDELEDYKDDDDKG

SEQ ID NO: 453 - protein, 4 aa, synthetic construct
NVAP

SEQ ID NO: 454 - protein, 7 aa, synthetic construct
FHIGHHI

SEQ ID NO: 455 - protein, 5 aa, synthetic construct
PFTWP

SEQ ID NO: 456 - protein, 156 aa, synthetic construct
HHHHHHDPTLIHTPGWNVTVGSEADLLDGDDSTGVEYYITTSSLAGEFIGLDLGEVVEVGGIHFVIGADGGGSSDKWTRFRLEYSLDGESWTTIREYDHTGAPAGQDVIDEDFETPISAQYIRLTNLEEPGSHLTFSEFAIVSDELEDYKDDDDKG

SEQ ID NO: 457 - protein, 4 aa, synthetic construct
NVTV

SEQ ID NO: 458 - protein, 5 aa, synthetic construct
YITTS

SEQ ID NO: 459 - protein, 5 aa, synthetic construct
EPGSH

SEQ ID NO: 460 - protein, 158 aa, synthetic construct
HHHHHHDPTLIHTPGWPVSFGSEADLLDGDDSTGVEYTFHGHHVSLAGEFIGLDLGEVVEVGGIHFVIGADGGGSSDKWTRFRLEYSLDGESWTTIREYDHTGAPAGQDVIDEDFETPISAQYIRLTNLEHFVYPLTFSEFAIVSDELEDYKDDDDKG

SEQ ID NO: 461 - protein, 4 aa, synthetic construct
PVSF

SEQ ID NO: 462 - protein, 7 aa, synthetic construct
TFHGHHV

SEQ ID NO: 463 - protein, 5 aa, synthetic construct
HFVYP

SEQ ID NO: 464 - protein, 158 aa, synthetic construct
HHHHHHDPTLIHTPGWQVNDGSEADLLDGDDSTGVEYPVYQHHVSLAGEFIGLDLGEVVEVGGIHFVIGADGGGSSDKWTRFRLEYSLDGESWTTIREYDHTGAPAGQDVIDEDFETPIPAQYIRLTNLEDFQYPLTFSEFAIVSDELEDYKDDDDKG

SEQ ID NO: 465 - protein, 4 aa, synthetic construct
QVND

SEQ ID NO: 466 - protein, 7 aa, synthetic construct
PVYQHHV

SEQ ID NO: 467 - protein, 5 aa, synthetic construct
DFQYP

SEQ ID NO: 468 - protein, 158 aa, synthetic construct
HHHHHHDPTLIHTPGWTVAIGSEADLLDGDDSTGVEYPQYAHTISLAGEFIGLDLGEVVEVGGIHFVIGADGGGSSDKWTRFRLEYSLDGESWTTIREYDHTGAPAGQDVIDEDFETPISAQYIRLTNLEQFIYPLTFSEFAIVSDELEDYKDDDDKG

SEQ ID NO: 469 - protein, 4 aa, synthetic construct
TVAI

SEQ ID NO: 470 - protein, 7 aa, synthetic construct
PQYAHTI

SEQ ID NO: 471 - protein, 5 aa, synthetic construct
QFIYP

SEQ ID NO: 472 - protein, 158 aa, synthetic construct
HHHHHHDPTLIHTPGWTVSPGSEADLLDGDDSTGVEYPLGWHAVSLAGEFIGLDLGEVVEVGGIHFVIGADGGGSSDKWTRFRLEYSLDGESWTTIREYDHTGAPAGQDVIDEDFETPISAQYIRLTNLEPFHPGLTFSEFAIVSDELEDYKDDDDKG

SEQ ID NO: 473 - protein, 4 aa, synthetic construct
TVSP

SEQ ID NO: 474 - protein, 7 aa, synthetic construct
PLGWHAV

SEQ ID NO: 475 - protein, 5 aa, synthetic construct
PFHPG

SEQ ID NO: 476 - protein, 158 aa, synthetic construct
HHHHHHDPTLIHTPGWYVAGGSEADLLDGDDSTGVEYPHYLHTISLAGEFIGLDLGEVVEVGGIHFVIGADGGGSSDKWTRFRLEYSLDGESWTTIREYDHTGAPAGQDVIDEDFETPISAQYIRLTNLESFTYPLTFSEFAIVSDELEDYKDDDDKG

SEQ ID NO: 477 - protein, 4 aa, synthetic construct
YVAG

SEQ ID NO: 478 - protein, 7 aa, synthetic construct
PHYLHTI

SEQ ID NO: 479 - protein, 5 aa, synthetic construct
SFTYP

SEQ ID NO: 480 - protein, 158 aa, synthetic construct
HHHHHHDPTLIHTPGWYVGTGSEADLLDGDDSTGVEYPHIAHTISLAGEFIGLDLGEVVEVGGIHFVIGADGGGSSDKWTRFRLEYSLDGESWTTIREYDHTGAPAGQDVIDEDFETPISAQYIRLTNLEEFQYPLTFSEFAIVSDELEDYKDDDDKG

SEQ ID NO: 481 - protein, 4 aa, synthetic construct
YVGT

SEQ ID NO: 482 - protein, 7 aa, synthetic construct
PHIAHTI

SEQ ID NO: 483 - protein, 5 aa, synthetic construct
EFQYP

SEQ ID NO: 484 - protein, 158 aa, synthetic construct
HHHHHHDPTLIHTPGWAVTTGSEADLLDGDDSTGVEYPFVHHQVSLAGEFIGLDLGEVVEVGGIHFVIGADGGGSSDKWTRFRLEYSLDGESWTTIREYDHTGAPAGQDVIDEDFETPISAQYIRLTNLEHFIFPLTFSEFAIVSDELEDYKDDDDKG

SEQ ID NO: 485 - protein, 4 aa, synthetic construct
AVTT

SEQ ID NO: 486 - protein, 7 aa, synthetic construct
PFVHHQV

SEQ ID NO: 487 - protein, 5 aa, synthetic construct
HFIFP

SEQ ID NO: 488 - protein, 158 aa, synthetic construct
HHHHHHDPTLIHTPGWEVEIGSEADLLDGDDSTGVEYPNYDHQISLAGEFIGLDLGEVVEVGGIHFVIGADGGGSSDKWTRFRLEYSLDGESWTTIREYDHTGAPAGQDVIDEDFETPISAQYIRLTNLEQFQPGLTFSEFAIVSDELEDYKDDDDKG

SEQ ID NO: 489 - protein, 4 aa, synthetic construct
EVEI

SEQ ID NO: 490 - protein, 7 aa, synthetic construct
PNYDHQI

SEQ ID NO: 491 - protein, 5 aa, synthetic construct
QFQPG

SEQ ID NO: 492 - protein, 158 aa, synthetic construct
HHHHHHDPTLIHTPGWFVHPGSEADLLDGDDSTGVEYVDTYQLYSLAGEFIGLDLGEVVEVGGIHFVSGADGGGSSDKWTRFRLEYSLDGESWTTIREYDHTGAPAGQDVIDEDFETPITAQYIRLTNLESFPSHLTFSEFAIVSDELEDYKDDDDKG

SEQ ID NO: 493 - protein, 4 aa, synthetic construct
FVHP

SEQ ID NO: 494 - protein, 7 aa, synthetic construct
VDTYQLY

SEQ ID NO: 495 - protein, 5 aa, synthetic construct
SFPSH

SEQ ID NO: 496 - protein, 156 aa, synthetic construct
HHHHHHDPTLIHTPGWLVHNGSEADLLDGDDSTGVEYVPWTALSSLAGEFIGLDLGEVVEVGGIHFVTDGGGSSDKWTRFRLEYSLDGESWTTIREYDHTGAPAGQDVIDEDFETPISAQYIRLTNLEASQSYLTFSEFAIVSDELEDYKDDDDKG

SEQ ID NO: 497 - protein, 4 aa, synthetic construct
LVHN

SEQ ID NO: 498 - protein, 7 aa, synthetic construct
VPWTALS

SEQ ID NO: 499 - protein, 5 aa, synthetic construct
ASQSY

SEQ ID NO: 500 - protein, 158 aa, synthetic construct
HHHHHHDPTLIHTPGWPVLIGSEADLLDGDDSTGVEYPFHGHHLSLAGEFIGLDLGEVVEVGGIHFVIGADGGGSSDKWTRFRLEYSLDGESWTTIREYDHTGAPAGQDVIDEDFETPISAQYIRLTNLEYFVHPLTFSEFAIVSDELEDYKDDDDKG

SEQ ID NO: 501 - protein, 4 aa, synthetic construct
PVLI

SEQ ID NO: 502 - protein, 7 aa, synthetic construct
PFHGHHL

SEQ ID NO: 503 - protein, 5 aa, synthetic construct
YFVHP

SEQ ID NO: 504 - protein, 158 aa, synthetic construct
HHHHHHDPTLIHTPGWQVHNGSEADLLDGDDSTGVEYLHSEPLESLAGEFIGLDLGEVVEVGGIHFVSGADGGGSSDKWTRFRLEYSLDGESWTTIREYDHTGAPAGQDVIDEDFETPISAQYIRLTNLEDATISLTFSEFAIVSDELEDYKDDDDKG

SEQ ID NO: 505 - protein, 4 aa, synthetic construct
QVHN

SEQ ID NO: 506 - protein, 7 aa, synthetic construct
LHSEPLE

SEQ ID NO: 507 - protein, 5 aa, synthetic construct
DATIS

SEQ ID NO: 508 - protein, 158 aa, synthetic construct
HHHHHHDPTLIHTPGWQVHPGSEADLLDGDDSTGVEYPQVHHHLSLAGEFIGLDLGEVVEVGGIHFVIGADGGGSSDKWTRFRLEYSLDGESWTTIREYDHTGAPAGQDVIDEDFETPISAQYIRLTNLEDFTFPLTFSEFAIVSDELEDYKDDDDKG

SEQ ID NO: 509 - protein, 4 aa, synthetic construct
QVHP

SEQ ID NO: 510 - protein, 7 aa, synthetic construct
PQVHHHL

SEQ ID NO: 511 - protein, 5 aa, synthetic construct
DFTFP

SEQ ID NO: 512 - protein, 158 aa, synthetic construct
HHHHHHDPTLIHTPGWQVITGSEADLLDGDDSTGVEYWNWATQISLAGEFIGLDLGEVVEVGGIHFVIGADGGGSSDKWTRFRLEYSLDGESWTTIREYDHTGAPAGQDVIDEDFETPISAQYIRLTNLENQVSTLTFSEFAIVSDELEDYKDDDDKG

SEQ ID NO: 513 - protein, 4 aa, synthetic construct
QVIT

SEQ ID NO: 514 - protein, 7 aa, synthetic construct
WNWATQI

SEQ ID NO: 515 - protein, 5 aa, synthetic construct
NQVST

SEQ ID NO: 516 - protein, 158 aa, synthetic construct
HHHHHHDPTLIHTPGWSVYEGSEADLLDGDDSTGVEYPQFHHSISLAGEFIGLDLGEVVEVGGIHFVIGADGGGSSDKWTRFRLEYSLDGESWTTIREYDHTGAPAGQDVIDEDFETPISAQYIRLTNLEDFVYPLTFSEFAIVSDELEDYKDDDDKG

SEQ ID NO: 517 - protein, 4 aa, synthetic construct
SVYE

SEQ ID NO: 518 - protein, 7 aa, synthetic construct
PQFHHSI

SEQ ID NO: 519 - protein, 5 aa, synthetic construct
DFVYP

SEQ ID NO: 520 - protein, 158 aa, synthetic construct
HHHHHHDPTLIHTPGWVVDPGSEADLLDGDDSTGVEYTFHGHHVSLAGEFIGLDLGEVVEVGGIHFVIGADGGGSSDKWTRFRLEYSLDGESWTTIREYDHTGAPAGQDVIDEDFETPISAQYIRLTNLEHFVYPLTFSEFAIVSDELEDYKDDDDKG

SEQ ID NO: 521 - protein, 4 aa, synthetic construct
VVDP

SEQ ID NO: 522 - protein, 7 aa, synthetic construct
TFHGHHV

SEQ ID NO: 523 - protein, 5 aa, synthetic construct
HFVYP

SEQ ID NO: 524 - protein, 158 aa, synthetic construct
HHHHHHDPTLIHTPGWEVTVGSEADLLDGDDSTGVEYLFIHDHVSLAGEFIGLDLGEVVEVGGIHFVIGADGGGSSDKWTRFRLEYSLDGESWTTIREYDHTGAPAGQDVIDEDFETPISAQYIRLTNLEFLYIHLTFSEFAIVSDELEDYKDDDDKG

SEQ ID NO: 525 - protein, 4 aa, synthetic construct
EVTV

SEQ ID NO: 526 - protein, 7 aa, synthetic construct
LFIHDHV

SEQ ID NO: 527 - protein, 5 aa, synthetic construct
FLYIH

SEQ ID NO: 528 - protein, 158 aa, synthetic construct
HHHHHHDPTLIHTPGWLVLSGSEADLLDGDDSTGVEFVWNDWGQSLAGEFIGLDLGEVVEVGGIHFVIGADGGGSSDKWTRFRLEYSLDGESWTTIREYDHTGAPAGQHVIDEDFETAISAQYIRLTNLEILTDHLTFSELAIVSDELEDYKDDDDKG

SEQ ID NO: 529 - protein, 4 aa, synthetic construct
LVLS

SEQ ID NO: 530 - protein, 7 aa, synthetic construct
VWNDWGQ

SEQ ID NO: 531 - protein, 5 aa, synthetic construct
ILTDH

SEQ ID NO: 532 - protein, 158 aa, synthetic construct
HHHHHHDPTLIHTPGWNVQEGSEADLLDGDDSTGVEYQVLHIHVSLAGEFIGLDLGEVVEVGGIHFVIGADGGGSSDKWTRFRLEYSLDGESWTTIREYDHTGAPAGQDVIDEDFETPISAQYIRLTNLEAYLQPLTFSEFAIVSDELEDYKDDDDKG

SEQ ID NO: 533 - protein, 4 aa, synthetic construct
NVQE

SEQ ID NO: 534 - protein, 7 aa, synthetic construct
QVLHIHV

SEQ ID NO: 535 - protein, 5 aa, synthetic construct
AYLQP

SEQ ID NO: 536 - protein, 158 aa, synthetic construct
HHHHHHDPTLIHTPGWVVYGGSEADLLDGDDSTGVEYLVPYLSWSLAGEFIGLDLGEVVEVGGIHFVIGADGGGSSDKWTRFRLEYSLDGESWTTIREYDHTGAPAGQDVIDEDFETPISAQYIRLTNLEGYSAYLTFSEFAIVSDELEDYKDDDDKG

SEQ ID NO: 537 - protein, 4 aa, synthetic construct
VVYG

SEQ ID NO: 538 - protein, 7 aa, synthetic construct
LVPYLSW

SEQ ID NO: 539 - protein, 5 aa, synthetic construct
GYSAY

SEQ ID NO: 540 - protein, 158 aa, synthetic construct
HHHHHHDPTLIHTPGWYVPFGSEADLLDGDDSTGVEYYPTLHNVSLAGEFIGLDLGEVVEVGGIHFVIGADGGGSSDKWTRFRLEYSLDGESWTTIREYDHTGAPAGQDVIDEDFETPISAQYIRLTNLEFYIGYLTFSEFAIVSDELEDYKDDDDKG

SEQ ID NO: 541 - protein, 4 aa, synthetic construct
YVPF

SEQ ID NO: 542 - protein, 7 aa, synthetic construct
YPTLHNV

SEQ ID NO: 543 - protein, 5 aa, synthetic construct
FYIGY

SEQ ID NO: 544 - protein, 158 aa, synthetic construct
HHHHHHDPTLIHTPGWYVPFGSEADLLDGDDSTGVEYYPTLRSVSLAGEFIGLDLGEVVEVGGIHFVIGADGGGSSDKWTRFRLEYSLDGESWTTIREYDHTGAPAGQDVIDEDFETPISAQYIRLTNLEFYIGYLTFSEFAIVSDELEDYKDDDDKG

SEQ ID NO: 545 - protein, 4 aa, synthetic construct
YVPF

SEQ ID NO: 546 - protein, 7 aa, synthetic construct
YPTLRSV

SEQ ID NO: 547 - protein, 5 aa, synthetic construct
FYIGY

SEQ ID NO: 548 - protein, 158 aa, synthetic construct
HHHHHHDPTLIHTPGWEVWFGSEADLLDGDDSTGVEYFTAQAQPSLAGEFIGLDLGEVVEVGGIHFVIGADGGGSSDKWTRFRLEYSLDGESWTTIREYDHTGAPAGQDVIDEDFETPISAQYIRLTNLEGSVWYLTFSEFAIVSDELEDYKDDDDKG

SEQ ID NO: 549 - protein, 4 aa, synthetic construct
EVWF

SEQ ID NO: 550 - protein, 7 aa, synthetic construct
FTAQAQP

SEQ ID NO: 551 - protein, 5 aa, synthetic construct
GSVWY

SEQ ID NO: 552 - protein, 158 aa, synthetic construct
HHHHHHDPTLIHTPGWFVFAGSEADLLDGDDSTGVEYTWVNWPVSLAGEFIGLDLGEVVEVGGIHFVIGADGGGSSDKWTRFRLEYSLDGESWTTIREYDHTGAPAGQDVIDEDFETPISAQYIRLTNLEGHFTSLTFSEFAIVSDELEDYKDDDDKG

SEQ ID NO: 553 - protein, 4 aa, synthetic construct
FVFA

SEQ ID NO: 554 - protein, 7 aa, synthetic construct
TWVNWPV

SEQ ID NO: 555 - protein, 5 aa, synthetic construct
GHFTS

SEQ ID NO: 556 - protein, 158 aa, synthetic construct
HHHHHHDPTLIHTPGWIVISGSEADLLDGDDSTGVEYYAFDYWNSLAGEFIGLDLGEVVEVGGIHFVIGADGGGSSDKWTRFRLEYSLDGESWTTIREYDHTGAPAGQDVIDEDFETPISAQYIRLTNLEFTTYILTFSEFAIVSDELEDYKDDDDKG

SEQ ID NO: 557 - protein, 4 aa, synthetic construct
IVIS

SEQ ID NO: 558 - protein, 7 aa, synthetic construct
YAFDYWN

SEQ ID NO: 559 - protein, 5 aa, synthetic construct
FTTYI

SEQ ID NO: 560 - protein, 158 aa, synthetic construct
HHHHHHDPTLIHTPGWLVLSGSEADLLDGDDSTGVEYWTIQSQPSLAGEFIGLDLGEVVEVGGIHFVIGADGGGSSDKWTRFRLEYSLDGESWTTIREYDHTGAPAGQDVIDEDFETPISAQYIRLTNLEFSGVILTFSEFAIVSDELEDYKDDDDKG

SEQ ID NO: 561 - protein, 4 aa, synthetic construct
LVLS

SEQ ID NO: 562 - protein, 7 aa, synthetic construct
WTIQSQP

SEQ ID NO: 563 - protein, 5 aa, synthetic construct
FSGVI

SEQ ID NO: 564 - protein, 158 aa, synthetic construct
HHHHHHDPTLIHTPGWNVAAGSEADLLDGDDSTGVEYYSAHWWVSLAGEFIGLDLGEVVEVGGIHFVIGADGGGSSDKWTRFRLEYSLDGESWTTIREYDHTGAPAGQDVIDEDFETPISAQYIRLTNLEHVVGFLTFSEFAIVSDELEDYKDDDDKG

SEQ ID NO: 565 - protein, 4 aa, synthetic construct
NVAA

SEQ ID NO: 566 - protein, 7 aa, synthetic construct
YSAHWWV

SEQ ID NO: 567 - protein, 5 aa, synthetic construct
HVVGF

SEQ ID NO: 568 - protein, 158 aa, synthetic construct
HHHHHHDPTLIHTPGWWVFQGSEADLLDGDDSTGVEYFHPHLPFSLAGEFIGLDLGEVVEVGGIHFVIGADGGGSSDKWTRFRLEYSLDGESWTTIREYDHTGAPAGQDVIDEDFETPISAQYIRLTNLETTFFQLTFSEFAIVSDELEDYKDDDDKG

SEQ ID NO: 569 - protein, 4 aa, synthetic construct
WVFQ

SEQ ID NO: 570 - protein, 7 aa, synthetic construct
FHPHLPF

SEQ ID NO: 571 - protein, 5 aa, synthetic construct
TTFFQ

(19) United States
(12) Patent Application Publication
(10) Pub. No.: US 2026/0193328 A1
(43) Pub. Date: Jul. 9, 2026

(54) THERMOSTABLE BINDING SCAFFOLDS

(71) Applicant: Nectagen, Inc., Kansas City, KS (US)

(72) Inventors: Richard J. SUDERMAN, Shawnee, KS (US); David M. CHAO, Kansas City, MO (US)

(21) Appl. No.: 19/133,337

(22) PCT Filed: Nov. 28, 2023
(86) PCT No.: PCT/US2023/081399

§ 371 Date: May 28, 2025

Related U.S. Application Data
(60) Provisional application No. 63/428,344, filed on Nov. 28, 2022.

(51) Int. Cl. C07K 16/14; C07K 1/22

(52) U.S. Cl. CPC C07K 16/14; C07K 1/22; C07K 2317/92; C07K 2317/94; C07K 2318/00

(57) ABSTRACT

The present invention features thermostable protein binding scaffolds containing framework regions and variable loop regions that can be mutagenized to bind a desired target. The scaffolds are derived from the Carbohydrate Binding Module Family 32 (CBM32) protein domain of Clostridium perfringens hyaluronidase (NagH). The robust framework of the scaffolds described herein allows for the development of custom, high performance affinity chromatography resins compatible with the harsh conditions of process-scale applications that can be adaptable to a wide diversity of target substrates.

### STATEMENT REGARDING FEDERALLY SPONSORED RESEARCH

This invention was made with government support under Grant No. 1 R43 GM143942-01, awarded by the National Institutes of Health. The government has certain rights in the invention.

### SEQUENCE LISTING

This application contains a Sequence Listing which has been filed electronically in Extensible Markup Language (XML) format and is hereby incorporated by reference in its entirety. Said XML copy, created on Nov. 22, 2023, is named 51027-005WO2_Sequence_Listing_11_22_23.XML and is 526,678 bytes in size.

### BACKGROUND OF THE INVENTION

Affinity chromatography (AC) with target-specific, immobilized capture agents is an established method of protein purification. In this technique, a capture agent, such as a protein, nucleic acid, or small molecule, is coupled to a solid support, which can then be used to isolate a protein of interest from a complex mixture. The technique has been widely used at the laboratory scale for single-step purifications of diverse target proteins, including enzymes, transcription factors, growth factors and antibodies.

Use of protein-based capture agents for AC in industrial applications has been less widespread because currently available approaches are incompatible with the temperatures, pH extremes, and solvents often needed for process-scale purification or are useful for only a limited number of targets. An exception is the purification of kg-quantities of antibodies with AC resins based on Staphylococcal Protein A. The development of Protein A resins highlights the use of protein engineering to improve the robustness of AC resins as well as some remaining limitations. Early versions of resins with wild-type Protein A captured antibodies with high selectivity and capacity from cell culture media feedstock but lost activity gradually after multiple cycles of cleaning in place with sodium hydroxide. Mutagenesis of Protein A yielded variants with increased resistance to sodium hydroxide treatment and higher binding capacity. Despite the widespread use of Protein A resins, they are nonetheless limited to the purification of antibodies.

For the process-scale purification of non-antibody targets, the use of AC is much less widespread than the use of Protein A to purify antibodies. For instance, non-protein, ligand-based approaches, such as small molecule substrate mimetics, are effective but are limited to specific enzyme classes and are difficult to use with a general protein of interest. Alternatively, specialized affinity resins, such as glutathione or nickel require the addition of non-native tags, which cause downstream complications for proteins intended for therapeutic use. Immuno-AC with antibody- or nanobody-based capture agents is the most generally applicable approach and has widely been used to purify a diverse range of proteins at laboratory-scale. However, immuno-AC has some limitations. In general, the conjugation of the antibody to the resin often results in heterogenous coupling because of a lack of precise control over the sites of conjugation. In addition, chromatography must be performed under oxidizing conditions in order to preserve the disulfide bonds essential for maintaining antibody structure. Elution of the target also usually requires low or high pH conditions that are incompatible with some target proteins. For process-scale applications, the chief limitation of immuno-AC resins is their sensitivity to the sodium hydroxide solutions which are preferred for cleaning-in-place procedures. Because of these limitations, new capture agents are needed that can perform under a variety of extreme conditions necessary for robust target purification.

### SUMMARY OF THE INVENTION

In one aspect, the invention features a protein scaffold that includes framework regions and loop regions. The protein scaffold has the structure:
A-F1-L1-F2-L2-F3-L3-F4-L4-F5-L5-F6-L6-F7-L7-F8-L8-F9-B, wherein
each of F1-F9 correspond to framework regions 1-9;
each of L1-L8 correspond to loop regions 1-8;
A and B are each independently, absent or include at least one amino acid;
F1 includes the sequence of: (T/S)-LI-(H/R)-(T/S)-(P/E)-(G/S)-W (SEQ ID NO: 4) or a sequence having one or two amino acid insertions, deletions, substitution mutations, or a combination thereof (e.g., one or two substitution mutations) relative to SEQ ID NO: 4;
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: G-(S/N/T)-E-(A/S)-(D/N/S/A)-LLDGDD-(S/N/T)-TGV-(E/W/A)-Y (SEQ ID NO: 5) or a sequence having one or two amino acid insertions, deletions, substitution mutations, or a combination thereof (e.g., one or two substitution mutations) relative to SEQ ID NO: 5;
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: S-(L/V)-AGEFIGLDLG (SEQ ID NO: 6) or a sequence having one or two amino acid insertions, deletions, substitution mutations, or a combination thereof (e.g., one or two substitution mutations) relative to SEQ ID NO: 6;
L3 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F4 includes the sequence of: G-(I/V)-(H/R/Y/N)-FVIG-(A/K/R)-(D/N) (SEQ ID NO: 7) or a sequence having one or two amino acid insertions, deletions, substitution mutations, or a combination thereof (e.g., one or two substitution mutations) relative to SEQ ID NO: 7;
L4 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F5 includes the sequence of: DKW-(T/N/S)-(R/K)-F-(R/K)-LEYS (SEQ ID NO: 8) or a sequence having one or two amino acid insertions, deletions, substitution mutations, or a combination thereof (e.g., one or two substitution mutations) relative to SEQ ID NO: 8;
L5 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F6 includes the sequence of: WTTI-(R/K/H/Q)-EYD-(H/K/R/Q) (SEQ ID NO: 9) or a sequence having one or two amino acid insertions, deletions, substitution mutations, or a combination thereof (e.g., one or two substitution mutations) relative to SEQ ID NO: 9;
L6 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F7 includes the sequence of: (Q/K)-DVI-(D/E)-E-(D/S)-F (SEQ ID NO: 10) or a sequence having one or two amino acid insertions, deletions, substitution mutations, or a combination thereof (e.g., one or two substitution mutations) relative to SEQ ID NO: 10;
L7 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F8 includes the sequence of: (Q/K/R)-YIRLTNLE (SEQ ID NO: 11) or a sequence having one or two amino acid insertions, deletions, substitution mutations, or a combination thereof (e.g., one or two substitution mutations) relative to SEQ ID NO: 11;
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFA-(I/V)-VS (SEQ ID NO: 12) or a sequence having one or two amino acid insertions, deletions, substitution mutations, or a combination thereof (e.g., one or two substitution mutations) relative to SEQ ID NO: 12.

As described herein, a sequence having one or two amino acid insertions, deletions, substitution mutations, or a combination thereof includes a sequence having, for example, one insertion, two insertions, one deletion, two deletions, one substitution mutation, two substitution mutations, one insertion and one deletion, one insertion and one substitution mutation, or one deletion and one substitution mutation.

In some embodiments, the protein scaffold includes at least one mutation selected from the group consisting of N807X, S809X, R812X, S813X, E814X, S815X₁, D818X, N822X, N825X, N832X, W836X, K857X, E858X, I859X, K860X₂, L861X, D862X, R865X, K870X, N871 X, N880X, K881 X, K883X, N890X, K897X, K901X, K908X, E912X, S914X, and K922X₃ relative to SEQ ID NO: 1, wherein:
X is any amino acid except the amino acid in the equivalent position in SEQ ID NO: 1;
X₁ is any amino acid except R or S;
X₂ is any amino acid except P or K; and
X₃ is any amino acid except R or K.

In some embodiments,
F1 includes the sequence of: (T/S)-LI-(H/R)-(T/S)-(P/E)-(G/S)-W (SEQ ID NO: 4) or a sequence having one amino acid insertion, deletion, or substitution mutation (e.g., one substitution mutation) relative to SEQ ID NO: 4;
F2 includes the sequence of: G-(S/N/T)-E-(A/S)-(D/N/S/A)-LLDGDD-(S/N/T)-TGV-(E/W/A)-Y (SEQ ID NO: 5) or a sequence having one amino acid insertion, deletion, or substitution mutation (e.g., one substitution mutation) relative to SEQ ID NO: 5;
F3 includes the sequence of: S-(L/V)-AGEFIGLDLG (SEQ ID NO: 6) or a sequence having one amino acid insertion, deletion, or substitution mutation (e.g., one substitution mutation) relative to SEQ ID NO: 6;
F4 includes the sequence of: G-(I/V)-(H/R/Y/N)-FVIG-(A/K/R)-(D/N) (SEQ ID NO: 7) or a sequence having one amino acid insertion, deletion, or substitution mutation (e.g., one substitution mutation) relative to SEQ ID NO: 7;
F5 includes the sequence of: DKW-(T/N/S)-(R/K)-F-(R/K)-LEYS (SEQ ID NO: 8) or a sequence having one amino acid insertion, deletion, or substitution mutation (e.g., one substitution mutation) relative to SEQ ID NO: 8;
F6 includes the sequence of: WTTI-(R/K/H/Q)-EYD-(H/K/R/Q) (SEQ ID NO: 9) or a sequence having one amino acid insertion, deletion, or substitution mutation (e.g., one substitution mutation) relative to SEQ ID NO: 9;
F7 includes the sequence of: (Q/K)-DVI-(D/E)-E-(D/S)-F (SEQ ID NO: 10) or a sequence having one amino acid insertion, deletion, or substitution mutation (e.g., one substitution mutation) relative to SEQ ID NO: 10;
F8 includes the sequence of: (Q/K/R)-YIRLTNLE (SEQ ID NO: 11) or a sequence having one amino acid insertion, deletion, or substitution mutation (e.g., one substitution mutation) relative to SEQ ID NO: 11; and
F9 includes the sequence of: LTFSEFA-(I/V)-VS (SEQ ID NO: 12) or a sequence having one amino acid insertion, deletion, or substitution mutation (e.g., one substitution mutation) relative to SEQ ID NO: 12.

In some embodiments, F1 includes the sequence of: (T/S)-LI-(H/R)-(T/S)-(P/E)-(G/S)-W (SEQ ID NO: 4);
F2 includes the sequence of: G-(S/N/T)-E-(A/S)-(D/N/S/A)-LLDGDD-(S/N/T)-TGV-(E/W/A)-Y (SEQ ID NO: 5);
F3 includes the sequence of: S-(L/V)-AGEFIGLDLG (SEQ ID NO: 6);
F4 includes the sequence of: G-(I/V)-(H/R/Y/N)-FVIG-(A/K/R)-(D/N) (SEQ ID NO: 7);
F5 includes the sequence of: DKW-(T/N/S)-(R/K)-F-(R/K)-LEYS (SEQ ID NO: 8);
F6 includes the sequence of: WTTI-(R/K/H/Q)-EYD-(H/K/R/Q) (SEQ ID NO: 9);
F7 includes the sequence of: (Q/K)-DVI-(D/E)-E-(D/S)-F (SEQ ID NO: 10);
F8 includes the sequence of: (Q/K/R)-YIRLTNLE (SEQ ID NO: 11); and
F9 includes the sequence of: LTFSEFA-(I/V)-VS (SEQ ID NO: 12).

In some embodiments, the protein scaffold includes at least one mutation selected from the group consisting of N807X, S809X, R812X, S813X, E814X, S815X, D818X, N822X, N825X, N832X, W836X, K857X, E858X, I859X, K860X, L861X, D862X, R865X, K870X, N871 X, N880X, K881 X, K883X, N890X, K897X, K901 X, K908X, E912X, S914X, and K922× relative to SEQ ID NO: 1, wherein X is any amino acid.

In some embodiments, the protein scaffold includes at least one mutation selected from the group consisting of N807D, S809T, R812H, S813T, E814P, S815G, D818V, N822S, N825D, N832S, W836E, K857E, E858V, I859V, K860E, L861V, D862G, R865H, K870A, N871D, N880T, K881R, K883R, N890G, K897R, K901H, K908Q, E912D, S914D, and K922Q relative to SEQ ID NO: 1.

In some embodiments, the at least one mutation is K870X and/or N890X. In some embodiments, the at least one mutation is K870A and/or N890G. In some embodiments, the at least one mutation is K870A. In some embodiments, the at least one mutation is N890G.

In some embodiments, the protein scaffold includes at least 3 fewer lysines relative to SEQ ID NO: 1. For example, in some embodiments, the protein scaffold includes at least 3, 4, 5, 6, 7, 8, 9, or 10 fewer lysines relative to SEQ ID NO: 1. In some embodiments, the protein scaffold includes at least 6 fewer lysines relative to SEQ ID NO: 1. In some embodiments, the protein scaffold includes 9 fewer lysines relative to SEQ ID NO: 1. In some embodiments, the protein scaffold does not include any lysines.

In some embodiments, the protein scaffold includes at least 3 fewer asparagines relative to SEQ ID NO: 1. For example, in some embodiments, the protein scaffold includes at least 3, 4, 5, 6, 7, or 8 fewer asparagines relative to SEQ ID NO: 1. In some embodiments, the protein scaffold includes at least 5 fewer asparagines relative to SEQ ID NO: 1. In some embodiments, the protein scaffold includes 7 fewer asparagines relative to SEQ ID NO: 1. In some embodiments, the protein scaffold does not include any asparagines.

In some embodiments, A and B are each independently, absent or at least one amino acid. For example, each of A and B may each be independently, absent. In some embodiments, A and B are each independently, at least 1, 2, 3, 4, 5, 6, 7, 8, 9, 10, 11, 12, 13, 14, 15, 16, 17, 18, 19, 20, 30, 40, 50, 60, 70, 80, 90, 100, 200, 30, 400, 500, 600, 700, 800, 900, 1,000 or more amino acids. In some embodiments, A and B are each independently, from 0 to 1,000 amino acids, e.g., from 1 to 10 amino acids (e.g., 1, 2, 3, 4, 5, 6, 7, 8, 9, or 10 amino acids), from 10 to 100 amino acids (e.g., 10, 20, 30, 40, 50, 60, 70, 80, 90, or 100 amino acids, or from 100 to 1,000 amino acids (e.g., 100, 200, 300, 400, 500, 600, 700, 800, 900, or 1,000 amino acids).

In some embodiments, A and B are each independently, absent or from 1 amino acid to 20 amino acids (e.g., 1, 2, 3, 4, 5, 6, 7, 8, 9, 10, 11, 12, 13, 14, 15, 16, 17, 18, 19, or 20 amino acids).

In some embodiments, each of L1-L8 is independently, from 1 amino acid to 20 amino acids (e.g., 1, 2, 3, 4, 5, 6, 7, 8, 9, 10, 11, 12, 13, 14, 15, 16, 17, 18, 19, or 20 amino acids).

In some embodiments, each of L1-L8 is, independently, from 1 amino acid to 10 amino acids (e.g., 1, 2, 3, 4, 5, 6, 7, 8, 9, or 10 amino acids). In some embodiments, each of L1-L8 is, independently, from 3 amino acids to 10 amino acids. In some embodiments, each of L1-L8 is, independently, from 3 amino acids to 8 amino acids.

In some embodiments, L1 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L1 is from 0 to 5 amino acids (e.g., from 1 to 5 amino acids, e.g., 0, 1, 2, 3, 4, or 5 amino acids).

In some embodiments, L2 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L2 is from 1 amino acid to 16 amino acids (e.g., from 4 to 16 amino acids, e.g., 1, 2, 3, 4, 5, 6, 7, 8, 9, 10, 11, 12, 13, 14, 15, or 16 amino acids).

In some embodiments, L3 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L3 is 6 amino acids.

In some embodiments, L4 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L4 is from 0 to 5 amino acids (e.g., from 1 to 5 amino acids, e.g., 0, 1, 2, 3, 4, or 5 amino acids).

In some embodiments, L5 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L5 is 5 amino acids.

In some embodiments, L6 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L6 is from 3 to 6 amino acids (e.g., 3, 4, 5, or 6 amino acids).

In some embodiments, L7 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L7 is 4 or 5 amino acids.

In some embodiments, L8 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L8 is from 4 to 6 amino acids (e.g., 4, 5, or 6 amino acids).

In some embodiments, L1 is 4 amino acids. In some embodiments, L2 is 7 amino acids. In some embodiments, L8 is 5 amino acids. In some embodiments, L1 is 4 amino acids, L2 is 7 amino acids, and/or L8 is 5 amino acids. In some embodiments, L1 is 4 amino acids, L2 is 7 amino acids, and L8 is 5 amino acids.

In some embodiments, L1 includes the sequence of: X₁X₂X₃X₄ (SEQ ID NO: 13), wherein each of X₁-X₄ is, independently, any amino acid. In some embodiments, X₂ is V.

In some embodiments, L2 includes the sequence of: X₁X₂X₃X₄X₅X₆X₇ (SEQ ID NO: 14), wherein each of X₁-X₇ is, independently, any amino acid.

In some embodiments, L8 includes the sequence of: X₁X₂X₃X₄X₅ (SEQ ID NO: 15), wherein each of X₁-X₅ is, independently, any amino acid.

In some embodiments, L4 includes the sequence of: X₁X₂X₃X₄X₅X₆X₇ (SEQ ID NO: 14), wherein each of X₁-X₇ is, independently, any amino acid.

In some embodiments, L6 includes the sequence of: X₁X₂X₃X₄X₅X₆(SEQ ID NO: 16), wherein each of X₁-X₆ is, independently, any amino acid.

In some embodiments, L8 includes at least two amino acids. In some embodiments, L8 includes at least one amino acid.

In some embodiments, L4 includes the sequence of: (G/D)-GGSS (SEQ ID NO: 17) or GDT or a sequence having one or two amino acid insertions, deletions, substitution mutations, or a combination thereof (e.g., one or two substitution mutations) relative to SEQ ID NO: 17 or GDT.

In some embodiments, L6 includes the sequence of TGAPAG (SEQ ID NO: 18) or a sequence having one or two amino acid insertions, deletions, substitution mutations, or a combination thereof (e.g., one or two substitution mutations) relative to SEQ ID NO: 18.

In some embodiments, L4 includes the sequence of: (G/D)-GGSS (SEQ ID NO: 17) or GDT; and L6 includes the sequence of TGAPAG (SEQ ID NO: 18).

In some embodiments, L3 includes the sequence of: (E/K/S)-(V/E)-(V/I/T)-(E/K/P/S)-(V/L)-(G/D) (SEQ ID NO: 19) or a sequence having one or two amino acid insertions, deletions, substitution mutations, or a combination thereof (e.g., one or two substitution mutations) relative to SEQ ID NO: 19.

In some embodiments, L5 includes the sequence of: LD-(G/N)-(E/S)-S(SEQ ID NO: 20) or a sequence having one or two amino acid insertions, deletions, substitution mutations, or a combination thereof (e.g., one or two substitution mutations) relative to SEQ ID NO: 20.

In some embodiments, L7 includes at least one amino acid.

In some embodiments, L7 includes the sequence of ETPI-(S/E)-A (SEQ ID NO: 21) or a sequence having one or two amino acid insertions, deletions, substitution mutations, or a combination thereof (e.g., one or two substitution mutations) relative to SEQ ID NO: 21.

In some embodiments, L3 includes the sequence of: (E/K/S)-(V/E)-(V/I/T)-(E/K/P/S)-(V/L)-(G/D) (SEQ ID NO: 19) or a sequence having one or two amino acid insertions, deletions, substitution mutations, or a combination thereof (e.g., one or two substitution mutations) relative to SEQ ID NO: 19; L5 includes the sequence of: LD-(G/N)-(E/S)-S(SEQ ID NO: 20) or a sequence having one or two amino acid insertions, deletions, substitution mutations, or a combination thereof (e.g., one or two substitution mutations) relative to SEQ ID NO: 20; and L7 includes the sequence of ETPI-(S/E)-A (SEQ ID NO: 21) or a sequence having one or two amino acid insertions, deletions, substitution mutations, or a combination thereof (e.g., one or two substitution mutations) relative to SEQ ID NO: 21.

In some embodiments, A includes the sequence of (D/N/H)-P. In some embodiments, A includes the sequence of DP.

In some embodiments, B includes the sequence of DELE (SEQ ID NO: 35).

In some embodiments, F1 includes the sequence of: TLIHTPGW (SEQ ID NO: 22) or a sequence having one or two amino acid insertions, deletions, substitution mutations, or a combination thereof (e.g., one or two substitution mutations) relative to SEQ ID NO: 22;
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: GSEADLLDGDDSTGVEY (SEQ ID NO: 23) or a sequence having one or two amino acid insertions, deletions, substitution mutations, or a combination thereof (e.g., one or two substitution mutations) relative to SEQ ID NO: 23;
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: SLAGEFIGLDLG (SEQ ID NO: 24) or a sequence having one or two amino acid insertions, deletions, substitution mutations, or a combination thereof (e.g., one or two substitution mutations) relative to SEQ ID NO: 24;
L3 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F4 includes the sequence of: GIHFVIGAD (SEQ ID NO: 25) or a sequence having one or two amino acid insertions, deletions, substitution mutations, or a combination thereof (e.g., one or two substitution mutations) relative to SEQ ID NO: 25;
L4 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F5 includes the sequence of: DKWTRFRLEYS (SEQ ID NO: 26) or a sequence having one or two amino acid insertions, deletions, substitution mutations, or a combination thereof (e.g., one or two substitution mutations) relative to SEQ ID NO: 26;
L5 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F6 includes the sequence of: WTTIREYDH (SEQ ID NO: 27) or a sequence having one or two amino acid insertions, deletions, substitution mutations, or a combination thereof (e.g., one or two substitution mutations) relative to SEQ ID NO: 27;
L6 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F7 includes the sequence of: QDVIDEDF (SEQ ID NO: 28) or a sequence having one or two amino acid insertions, deletions, substitution mutations, or a combination thereof (e.g., one or two substitution mutations) relative to SEQ ID NO: 28;
L7 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F8 includes the sequence of: QYIRLTNLE (SEQ ID NO: 29) or a sequence having one or two amino acid insertions, deletions, substitution mutations, or a combination thereof (e.g., one or two substitution mutations) relative to SEQ ID NO: 29;
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFAIVS (SEQ ID NO: 30) or a sequence having one or two amino acid insertions, deletions, substitution mutations, or a combination thereof (e.g., one or two substitution mutations) relative to SEQ ID NO: 30.

In some embodiments, F1 includes the sequence of: TLIHTPGW (SEQ ID NO: 22);
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: GSEADLLDGDDSTGVEY (SEQ ID NO: 23);
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: SLAGEFIGLDLG (SEQ ID NO: 24);
L3 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F4 includes the sequence of: GIHFVIGAD (SEQ ID NO: 25);
L4 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F5 includes the sequence of: DKWTRFRLEYS (SEQ ID NO: 26);
L5 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F6 includes the sequence of: WTTIREYDH (SEQ ID NO: 27);
L6 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F7 includes the sequence of: QDVIDEDF (SEQ ID NO: 28);
L7 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F8 includes the sequence of: QYIRLTNLE (SEQ ID NO: 29);
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFAIVS (SEQ ID NO: 30).

In some embodiments, F1 comprises the sequence of: TLIHTPGW (SEQ ID NO: 22) or a sequence having one or two amino acid insertions, deletions, substitution mutations, or a combination thereof (e.g., one or two substitution mutations) relative to SEQ ID NO: 22;
L1 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 comprises the sequence of: GSEADLLDGDDSTGVEY (SEQ ID NO: 23) or a sequence having one or two amino acid insertions, deletions, substitution mutations, or a combination thereof (e.g., one or two substitution mutations) relative to SEQ ID NO: 23;
L2 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 comprises the sequence of: SLAGEFIGLDLG (SEQ ID NO: 24) or a sequence having one or two amino acid insertions, deletions, substitution mutations, or a combination thereof (e.g., one or two substitution mutations) relative to SEQ ID NO: 24;
L3 comprises the sequence of: EVVEVG (SEQ ID NO: 31) or a sequence having one or two amino acid insertions, deletions, substitution mutations, or a combination thereof (e.g., one or two substitution mutations) relative to SEQ ID NO: 31;
F4 comprises the sequence of: GIHFVIGAD (SEQ ID NO: 25) or a sequence having one or two amino acid insertions, deletions, substitution mutations, or a combination thereof (e.g., one or two substitution mutations) relative to SEQ ID NO: 25
L4 comprises the sequence of: GGGSS (SEQ ID NO: 32) or a sequence having one or two amino acid insertions, deletions, substitution mutations, or a combination thereof (e.g., one or two substitution mutations) relative to SEQ ID NO: 32;
F5 comprises the sequence of: DKWTRFRLEYS (SEQ ID NO: 26) or a sequence having one or two amino acid insertions, deletions, substitution mutations, or a combination thereof (e.g., one or two substitution mutations) relative to SEQ ID NO: 26;
L5 comprises the sequence of: LDGES (SEQ ID NO: 33) or a sequence having one or two amino acid insertions, deletions, substitution mutations, or a combination thereof (e.g., one or two substitution mutations) relative to SEQ ID NO: 33;
F6 comprises the sequence of: WTTIREYDH (SEQ ID NO: 27) or a sequence having one or two amino acid insertions, deletions, substitution mutations, or a combination thereof (e.g., one or two substitution mutations) relative to SEQ ID NO: 27;
L6 comprises the sequence of: TGAPAG (SEQ ID NO: 18) or a sequence having one or two amino acid insertions, deletions, substitution mutations, or a combination thereof (e.g., one or two substitution mutations) relative to SEQ ID NO: 18;
F7 comprises the sequence of: QDVIDEDF (SEQ ID NO: 28) or a sequence having one or two amino acid insertions, deletions, substitution mutations, or a combination thereof (e.g., one or two substitution mutations) relative to SEQ ID NO: 28;
L7 comprises the sequence of: ETPISA (SEQ ID NO: 34) or a sequence having one or two amino acid insertions, deletions, substitution mutations, or a combination thereof (e.g., one or two substitution mutations) relative to SEQ ID NO: 34;
F8 comprises the sequence of: QYIRLTNLE (SEQ ID NO: 29) or a sequence having one or two amino acid insertions, deletions, substitution mutations, or a combination thereof (e.g., one or two substitution mutations) relative to SEQ ID NO: 29;
L8 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 comprises the sequence of: LTFSEFAIVS (SEQ ID NO: 30) or a sequence having one or two amino acid insertions, deletions, substitution mutations, or a combination thereof (e.g., one or two substitution mutations) relative to SEQ ID NO: 30.

In some embodiments, F1 comprises the sequence of: TLIHTPGW (SEQ ID NO: 22) or a sequence having one amino acid insertion, deletion, or substitution mutation (e.g., one substitution mutation) relative to SEQ ID NO: 22;
L1 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 comprises the sequence of: GSEADLLDGDDSTGVEY (SEQ ID NO: 23) or a sequence having one amino acid insertion, deletion, or substitution mutation (e.g., one substitution mutation) relative to SEQ ID NO: 23;
L2 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 comprises the sequence of: SLAGEFIGLDLG (SEQ ID NO: 24) or a sequence having one amino acid insertion, deletion, or substitution mutation (e.g., one substitution mutation) relative to SEQ ID NO: 24;
L3 comprises the sequence of: EVVEVG (SEQ ID NO: 31) or a sequence having one amino acid insertion, deletion, or substitution mutation (e.g., one substitution mutation) relative to SEQ ID NO: 31;
F4 comprises the sequence of: GIHFVIGAD (SEQ ID NO: 25) or a sequence having one amino acid insertion, deletion, or substitution mutation (e.g., one substitution mutation) relative to SEQ ID NO: 25;
L4 comprises the sequence of: GGGSS (SEQ ID NO: 32) or a sequence having one amino acid insertion, deletion, or substitution mutation (e.g., one substitution mutation) relative to SEQ ID NO: 32;
F5 comprises the sequence of: DKWTRFRLEYS (SEQ ID NO: 26) or a sequence having one amino acid insertion, deletion, or substitution mutation (e.g., one substitution mutation) relative to SEQ ID NO: 26;
L5 comprises the sequence of: LDGES (SEQ ID NO: 33) or a sequence having one amino acid insertion, deletion, or substitution mutation (e.g., one substitution mutation) relative to SEQ ID NO: 33;
F6 comprises the sequence of: WTTIREYDH (SEQ ID NO: 27) or a sequence having one amino acid insertion, deletion, or substitution mutation (e.g., one substitution mutation) relative to SEQ ID NO: 27;
L6 comprises the sequence of: TGAPAG (SEQ ID NO: 18) or a sequence having one amino acid insertion, deletion, or substitution mutation (e.g., one substitution mutation) relative to SEQ ID NO: 18;
F7 comprises the sequence of: QDVIDEDF (SEQ ID NO: 28) or a sequence having one amino acid insertion, deletion, or substitution mutation (e.g., one substitution mutation) relative to SEQ ID NO: 28;
L7 comprises the sequence of: ETPISA (SEQ ID NO: 34) or a sequence having one amino acid insertion, deletion, or substitution mutation (e.g., one substitution mutation) relative to SEQ ID NO: 34;
F8 comprises the sequence of: QYIRLTNLE (SEQ ID NO: 29) or a sequence having one amino acid insertion, deletion, or substitution mutation (e.g., one substitution mutation) relative to SEQ ID NO: 29;
L8 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 comprises the sequence of: LTFSEFAIVS (SEQ ID NO: 30) or a sequence having one amino acid insertion, deletion, or substitution mutation (e.g., one substitution mutation) relative to SEQ ID NO: 30.

In some embodiments, F1 includes the sequence of: TLIHTPGW (SEQ ID NO: 22);
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: GSEADLLDGDDSTGVEY (SEQ ID NO: 23);
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: SLAGEFIGLDLG (SEQ ID NO: 24);
L3 includes the sequence of: EVVEVG (SEQ ID NO: 31);
F4 includes the sequence of: GIHFVIGAD (SEQ ID NO: 25);
L4 includes the sequence of: GGGSS (SEQ ID NO: 32);
F5 includes the sequence of: DKWTRFRLEYS (SEQ ID NO: 26);
L5 includes the sequence of: LDGES (SEQ ID NO: 33);
F6 includes the sequence of: WTTIREYDH (SEQ ID NO: 27);
L6 includes the sequence of: TGAPAG (SEQ ID NO: 18);
F7 includes the sequence of: QDVIDEDF (SEQ ID NO: 28);
L7 includes the sequence of: ETPISA (SEQ ID NO: 34);
F8 includes the sequence of: QYIRLTNLE (SEQ ID NO: 29);
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFAIVS (SEQ ID NO: 30).

In some embodiments, F1 includes the sequence of: TLIHTPGW (SEQ ID NO: 22);
L1 includes the sequence of: X₁X₂X₃X₄ (SEQ ID NO: 13), wherein each of X₁-X₄ is, independently, any amino acid;
F2 includes the sequence of: GSEADLLDGDDSTGVEY (SEQ ID NO: 23);
L2 includes the sequence of: X₁X₂X₃X₄X₅X₆X₇ (SEQ ID NO: 14), wherein each of X₁-X₇ is, independently, any amino acid;
F3 includes the sequence of: SLAGEFIGLDLG (SEQ ID NO: 24);
L3 includes the sequence of: EVVEVG (SEQ ID NO: 31);
F4 includes the sequence of: GIHFVIGAD (SEQ ID NO: 25);
L4 includes the sequence of: GGGSS (SEQ ID NO: 32);
F5 includes the sequence of: DKWTRFRLEYS (SEQ ID NO: 26);
L5 includes the sequence of: LDGES (SEQ ID NO: 33);
F6 includes the sequence of: WTTIREYDH (SEQ ID NO: 27);
L6 includes the sequence of: TGAPAG (SEQ ID NO: 18);
F7 includes the sequence of: QDVIDEDF (SEQ ID NO: 28);
L7 includes the sequence of: ETPISA (SEQ ID NO: 34);
F8 includes the sequence of: QYIRLTNLE (SEQ ID NO: 29);
L8 includes the sequence of: X₁X₂X₃X₄X₅ (SEQ ID NO: 15), wherein each of X₁-X₅ is, independently, any amino acid; and
F9 includes the sequence of: LTFSEFAIVS (SEQ ID NO: 30).

In some embodiments, A includes the sequence of: DP;
F1 includes the sequence of: TLIHTPGW (SEQ ID NO: 22);
L1 includes the sequence of: X₁X₂X₃X₄ (SEQ ID NO: 13), wherein each of X₁-X₄ is, independently, any amino acid;
F2 includes the sequence of: GSEADLLDGDDSTGVEY (SEQ ID NO: 23);
L2 includes the sequence of: X₁X₂X₃X₄X₅X₆X₇ (SEQ ID NO: 14), wherein each of X₁-X₇ is, independently, any amino acid;
F3 includes the sequence of: SLAGEFIGLDLG (SEQ ID NO: 24);
L3 includes the sequence of: EVVEVG (SEQ ID NO: 31);
F4 includes the sequence of: GIHFVIGAD (SEQ ID NO: 25);
L4 includes the sequence of: GGGSS (SEQ ID NO: 32);
F5 includes the sequence of: DKWTRFRLEYS (SEQ ID NO: 26);
L5 includes the sequence of: LDGES (SEQ ID NO: 33);
F6 includes the sequence of: WTTIREYDH (SEQ ID NO: 27);
L6 includes the sequence of: TGAPAG (SEQ ID NO: 18);
F7 includes the sequence of: QDVIDEDF (SEQ ID NO: 28);
L7 includes the sequence of: ETPISA (SEQ ID NO: 34);
F8 includes the sequence of: QYIRLTNLE (SEQ ID NO: 29);
L8 includes the sequence of: X₁X₂X₃X₄X₅ (SEQ ID NO: 15), wherein each of X₁-X₅ is, independently, any amino acid;
F9 includes the sequence of: LTFSEFAIVS (SEQ ID NO: 30); and B includes the sequence of: DELE (SEQ ID NO: 35).

In some embodiments, L1 includes the sequence of X₁X₂X₃X₄ (SEQ ID NO: 13), wherein each of X₁, X₃, and X₄ is, independently, any amino acid, and X₂ is V.

In another aspect, featured is a protein scaffold that includes a polypeptide having at least 80% (e.g., at least 85%, 90%, 95%, 97%, or 99%) sequence identity to SEQ ID NO: 3. In some embodiments, the polypeptide includes the sequence of SEQ ID NO: 3. In some embodiments, the polypeptide does not include the sequence of SEQ ID NO: 1. In some embodiments, the polypeptide does not include the sequence of SEQ ID NO: 2.

In some embodiments, the polypeptide includes at least one mutation selected from the group consisting of N807X, S809X, R812X, S813X, E814X, S815X₁, D818X, N822X, N825X, N832X, W836X, K857X, E858X, I859X, K860X₂, L861X, D862X, R865X, K870X, N871 X, N880X, K881 X, K883X, N890X, K897X, K901X, K908X, E912X, S914X, and K922X₃ relative to SEQ ID NO: 1, wherein:
X is any amino acid except the amino acid in the equivalent position in SEQ ID NO: 1;
X₁ is any amino acid except R or S;
X₂ is any amino acid except P or K; and
X₃ is any amino acid except R or K.

In some embodiments of any of the above aspects, the protein scaffold further includes a mutation that adds a cysteine residue. In some embodiments, the protein scaffold includes a first mutation that adds a first cysteine residue and a second mutation that adds a second cysteine residue. In some embodiments, the first cysteine residue and the second cysteine residue form a disulfide bond under oxidizing conditions.

In some embodiments, the protein scaffold comprises at least one mutation selected from the group consisting of F806C, P808C, S845C, L855C, V858C, V861C, K878C, W879C, L884C, L888C, A904C, P905C, A906GC, G907C, I924C, L926C, N928C, L936C, I943C, L948C.

In some embodiments, the protein scaffold comprises at least two or more mutations selected from the group consisting of F806C, P808C, S845C, L855C, V858C, V861C, K878C, W879C, L884C, L888C, A904C, P905C, A906GC, G907C, I924C, L926C, N928C, L936C, I943C, L948C.

In some embodiments, the protein scaffold comprises a pair of cysteine mutations selected from the group consisting of K878C and G907C, K878C and A904C, V861C and I943C, P905C and L855C, S845C and L936C, W879C and N928C, L884C and L926C, F806C and L948C, V858C and L888C, K878C and G907C, K878C and A906GC, S845C and N928C, K878C and A904C, P808C and I943C, V861C and I924C, P808C and V861C, and I943C and L855C.

In some embodiments, the pair of cysteine mutations is selected from the group consisting of K878C and G907C, K878C and A904C, S845C and L936C, W879C and N928C, W879C and N928C, L884C and L926C, V858C and L888C, K878C and G907C, and K878C and A906GC (i.e., the substitution of alanine 906 with glycine and cysteine).

In some embodiments of any of the above aspects, the protein scaffold further includes a tag covalently attached to the scaffold.

In some embodiments, the tag is an affinity tag (e.g., a polyhistidine tag, e.g., 4, 5, 6, 7, 8, 9, or 10 histidines), an epitope tag, a covalent tag, or a protein tag.

In some embodiments, the tag is attached to the N-terminus or the C-terminus of the scaffold.

In some embodiments, the scaffold is conjugated to a functional group. In some embodiments, the functional group includes biotin, streptavidin or a derivative of streptavidin, a polyethylene glycol moiety, a fluorescent dye, an enzyme, a radioactive moiety, a lanthanide, or a lanthanide binding motif.

In some embodiments, the scaffold is conjugated to a lanthanide or a lanthanide binding motif. In some embodiments, the lanthanide is terbium.

In some embodiments, the scaffold is conjugated to a radioactive moiety. In some embodiments, the radioactive moiety is an a or p emitter.

In some embodiments, the functional group is conjugated to a sulfhydryl group or a primary amine.

In another aspect, featured is a polynucleotide encoding a protein scaffold as described herein, e.g., of any of the above embodiments. In some embodiments, the polynucleotide is a ribonucleotide. In some embodiments, the polynucleotide is a deoxyribonucleotide.

In another aspect, featured is a vector that includes a polynucleotide as described herein.

In another aspect, featured is a cell that includes a polynucleotide encoding the protein scaffold or a vector that includes the polynucleotide.

In another aspect, featured is a method of producing a protein scaffold as described herein, e.g., of any of the above embodiments. The method includes the steps of (a) providing a cell transformed with a polynucleotide encoding the protein scaffold or a vector that includes the polynucleotide; (b) culturing the transformed cell under conditions for expressing the polynucleotide, wherein the culturing results in expression of the protein scaffold. The method may further include (c) isolating the protein scaffold or using the protein scaffold to bind a target.

In another aspect, featured is a particle that includes the protein scaffold of any of the above embodiments. In some embodiments, the particle is a magnetic particle.

In another aspect, featured is a resin that includes a plurality of the particles, e.g., containing the protein scaffold.

In another aspect, featured is a column (e.g., a chromatography column) containing the particles or the resin, e.g., conjugated to the scaffold.

In another aspect, featured is a method of purifying a target molecule from a plurality of molecules. The method includes (a) providing a sample that includes a mixture of the target molecule and the plurality of molecules; (b) contacting the sample with the protein scaffold of any one of the above embodiments, wherein the scaffold specifically binds to the target molecule; and (c) separating the target molecule bound to the protein scaffold from the plurality of molecules.

In some embodiments, the step of separating includes immobilizing the protein scaffold.

In some embodiments, the protein scaffold is conjugated to a particle. In some embodiments, the particle includes a magnetic bead. In some embodiments, the protein scaffold is conjugated to a resin or monolith including a plurality of the particles.

### Definitions

The Carbohydrate Binding Module Family 32 (CBM32) scaffold of SEQ ID NO: 1 is derived from a single protein domain of Clostridium perfringens hyaluronidase (NagH), a multi-domain enzyme consisting of 1627 amino acids. Amino acid residue 1 of SEQ ID NO: 1 corresponds to amino acid residue 807 of NagH, and amino acid residue 140 of SEQ ID NO: 1 corresponds to amino acid residue 946 of NagH. Amino acid positions and mutations described herein generally relate to the position on the corresponding full length NagH unless otherwise specified.

The term “constant region,” as used herein, generally refers to a region of a binding scaffold that does not include the variable loop regions involved in target binding. For example, a constant region may include a framework region (e.g., F1-F9) or a loop region (e.g., L3-L7) that is not one of the three loops mutagenized for target binding (e.g., L1, L2, and L8). A constant region may have sequence variability.

The term “non-naturally occurring amino acid,” as used herein, means non-proteinogenic amino acids. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylornithine, L-α-methylproline, L-α-methylthreonine, L-α-methyltyrosine, L-N-methyl-homophenylalanine, N—(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-p-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

As used herein, the term “percent (%) identity” refers to the percentage of amino acid residues of a candidate sequence, e.g., a protein scaffold, that are identical to the amino acid residues of a reference sequence, e.g., a wild-type CBM32 polypeptide, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

$$100 \times ( fraction of A / B )$$

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

### BRIEF DESCRIPTION OF THE DRAWINGS

FIG. 1 is a schematic drawing showing an outline of the protein engineering campaign to produce a member of the nC-B class of nanoCLAMPs. The starting nanoCLAMP was anti-SUMO clone SMT3-A1, a member of the nC-A class of nanoCLAMPs. SMT3A1 was mutated over 7 rounds. At the conclusion of each round, the performance of clone(s) combining different mutations from the round was assessed by DSF and SEC. The end product is clone P2788, whose constant regions served as the basis for the nC-B class of nanoCLAMPs.

FIG. 2 is a space filled model of P2788, an example of the nC-B class of nanoCLAMP with mutations in P2788 mapped on to CBM-32-3 crystal structure and the sequence of the constant region of the nC-A class of nanoCLAMPs. The constant regions of clone P2788 are the basis for the nC-B class of nanoCLAMPs. Side chains of mutated positions are labeled in green; side of chains of variable loops are shown in red. Other side chains are shown in light gray. Backbone residues are shown in dark gray. The alignment compares the constant regions of the nC-A and nC-B classes of nanoCLAMPs. Residues denoted with black, bolded text represent nC-A positions where at least one mutation was tested (top row). Mutations were tested for 58% of positions in the constant regions (72 out of 124). Residues denoted with bolded text represent mutations in P2788 (bottom row). In P2788, 24% of positions in the constant regions are mutated relative to nC-A (30 out of 124).

FIG. 3 is a model showing the superimposition of the crystal structures for CBM32-2 (basis for the nC-A class of nanoCLAMPs) and the AlphaFold model for P2788 (basis for the nC-B class). CBM32-2 (PDB accession 2W1Q) and P2788 were aligned with jFATCAT (rigid) on the RCSB server, resulting in a high TM-score (0.95). Backbone deviations are apparent and expected for the loops, which have different amino acid sequences.

FIG. 4 is a graph showing differential scanning fluorimetry analysis of SMT3-A1 (nC-A class) and P2788 (nC-B class). Both clones show classically shaped melting curves with low initial fluorescence. The 30 mutations in P2788 increase its Tₘ by 24° C. relative to SMT3-A1.

FIGS. 5A-5F are gels and a graph showing protease resistance of nanoCLAMPs of the nC-A and nC-B classes. FIG. 5A shows SDS-PAGE analysis of SMT3-A1 (nC-A class) and P2788 (same variable loops as SMT3-A1 but with constant regions of the nC-B class) after exposure to 16 hr incubation with trypsin or chymotrypsin. FIGS. 5B-5D show SDS-PAGE analysis of time course tryptic digestions of SMT3-A1, P2788 and P2808. P2788 and P2808 have the same constant regions (nC-B class), but different loops. P2788 and P2808 were resistant to tryptic digest for over 20 hours. FIG. 5E shows quantitative densitometry analysis of the time course-stained gels. FIG. 5F shows SDS-PAGE analysis of members of the nC-A class of nanoCLAMPs (SMT3-A1) and nC-B class (P2788, P2808, P2809, and P2811) following 16 h tryptic digestions.

FIG. 6 is a set of size exclusion chromatograms showing monodispersity and melting temperature analysis of anti-SUMO nanoCLAMPs of the nC-B class. Size exclusion chromatography (left panel) and differential scanning fluorescence (right panel) of nanoCLAMPs P2808, P2809 and P2811.

FIG. 7 is a graph showing dynamic binding capacity of SMT3-A1 resin (nC-A class) and P2808 resin (nC-B class). Breakthrough curves were generated by loading a solution of 0.2 mg/ml Sumo-GFP in PBS onto 0.6 ml of packed resin in a column (3 cm height×5 mm ID) at a flowrate of 0.5 ml/min and measuring the fluorescence of the eluate. The percent fluorescence of the load was calculated by diving the eluate fluorescence by the load fluorescence. The dynamic binding capacity (DBC) was calculated with the following formula: DBC=(Vₓ−V_{delay})*c/(Vᵣₑₛᵢₙ). Vₓ is the volume of eluate collected, V_{delay} is the elution volume of the load under non-binding conditions, c=concentration of target in load, Vᵣₑₛᵢₙ is the volume of the packed resin in the column. The P2808 resin has a dynamic binding capacity of 10 mg/ml resin (240 nmol/ml resin).

FIGS. 8A and 8B are gels showing performance of P2808 resin in single step affinity chromatography purification of GFP-SUMO from spiked lysates in resin-limiting and protein-limiting scenarios. Identical columns were loaded under conditions that were 33% above (FIG. 8A) or 58% below (FIG. 8B) the columns dynamic binding capacity. E. coli lysates with spiked-in target protein (SUMO-GFP) were used as the load. After loading and washing, bound proteins were eluted with 3 M imidazole pH 8. Total protein loaded on SDS-PAGE in FIG. 8A: Lysate=32 μg, Spiked Lysate=34 μg, FT=47 μg, Eluate=6 μg; FIG. 8B: Lysate=17 μg, Spiked Lysate=17 μg, FT=21 μg, Wash=NA, Eluate=3 μg. Metrics of purifications in FIGS. 8A and 8B are tabulated in Table 6.

FIG. 9 is a graph showing the effect of sodium hydroxide treatment on binding capacity of nanoCLAMP capture agents of the nC-A and nC-B class. The binding capacities of resins with capture agents of the nC-A class (SMT3-A1, P1519, P1533) and the nC-B class (P2808, P2809, P2811) were determined after each of 22 cycles of purification of GFP-SUMO from a spiked E. coli lysate, followed by washing, eluting, and cleaning in place with 0.1 M NaOH (10 min contact time). The % of starting binding capacity was determined by dividing the eluate fluorescence with the load fluorescence. The selectivity was determined by analysis of the eluates on SDS-PAGE (FIG. 13).

FIGS. 10A-10D are graphs and gels showing the effect of organic solvent and autoclaving on the binding capacity of resins made with nanoCLAMPs of the nC-B class. Resins P2808, P2809, and P2811 (nC-B class) and Resin SMT3-A1 (nC-A class) were incubated in 100% DMF for 2 h (FIGS. 10A and 10B) or autoclaved (105-minute liquid steam cycle, including 30 min exposure to 120° C. and 20 p.s.i.) (FIGS. 10C and 10D) and then re-equilibrated in fresh buffer and tested in affinity chromatography purification of SUMO-GFP from spiked E. coli lysate. Binding capacity % of untreated was determined by dividing the eluate fluorescence by the control (non-treated) eluate fluorescence. Specificity was determined by Coomassie staining of SDS-PAGE (FIGS. 10B and 10D).

FIG. 11 is a graph showing kinetic thermal stability of nC-A (SMT3-A1) and nC-B (P2808, P2809, and P2811) nanoCLAMPs. nanoCLAMPs were heat treated, cooled and centrifuged. The supernatant was tested for binding activity by biolayer interferometry. The percent of starting response was measured as the amplitude of binding divided by that obtained by the control sample (held at 20° C. during heat treatments).

FIG. 12 is a gel showing static binding capacity of P2808 resin. Affinity resin prepared with P2808 (nC-B class) was incubated with a spiked E. coli lysate; washed; eluted with 3 M imidazole, pH 8; buffer exchanged; and quantified by A280.

FIG. 13 is a set of gels showing the effect of sodium hydroxide treatment on specificity of nanoCLAMP capture agents of the nC-A and nC-B class. nC-A (SMT3-A1, P1519, P1533) and nC-B (P2808, P2809, P2811) Sumo-binding nanoCLAMPs were covalently conjugated to 6% cross-linked agarose resin and then used to purify a Sumo-GFP fusion from a crude E. coli lysate. Each cycle consisted of a crude Sumo-GFP-spiked sample load, wash, elution with 3M Imidazole (collected), wash, 0.1 M NaOH regeneration (10 min contact time per cycle), and a 5 min refolding wash. The target protein in the eluate was quantified by fluorescence spectroscopy (FIG. 9), and the percent yield calculated by dividing the fluorescence by the initial eluate fluorescence. The purity of the eluted target from each cycle was assessed by SDS-PAGE and stained with Coomassie. Cycle number is shown for each lane, L=Load, M=marker. The prominent band in the eluates of each gel is SUMO-GFP (42 kD).

FIGS. 14A and 14B are a graph and a gel testing the stability of Resin P2808 (nC-B class) through >20 low pH elution cycles. SUMO-GFP was spiked into crude E. coli lysate, loaded onto P2808 resin, washed, and eluted with 0.1 M Citrate, pH 2.5, followed by regeneration with 0.1 N NaOH with 1 min contact time per cycle, and a re-equilibration wash for 5 min. FIG. 14A shows the target protein in the eluate, which was quantified by densitometry of the Coomassie stained SDS-PAGE gel of FIG. 14B because the fluorescence of the eluate was destroyed by the low pH. Percent yield was calculated by dividing the band density by the initial eluate band density. The purity of the eluted target from each cycle was assessed by SDS-PAGE of FIG. 14B.

FIG. 15 is a graph showing nanoCLAMPs stably binding terbium (Tb). SMT3-A1 (nC-A class), P2808 (nC-B class), and a negative control protein (recombinant SMT3) were incubated with CaCl₂) or TbCl₃ overnight, and then buffer exchanged to remove unbound metals. The buffer exchanged proteins were analyzed by time resolved fluorescence 24 h post buffer exchange (Ex/Em: 350 nm/544 nm), 200μ-sec delay.

FIG. 16 is a model showing the front, back, top, and bottom faces of the nC-B class of nanoCLAMP. A, B, F1-9, and L1-L8 are mapped on clone P2808 sequence and 3D-modeled to illustrate the locations of each region of the scaffold.

FIG. 17 is a model showing an alignment of the P2808, an example of the nC-B class, in which Loop 1 is replaced with a (G₄S)₃ sequence or is removed.

FIG. 18 is a model showing an alignment of the nC-B nanoCLAMP P2808 in which Loop 2 is replaced with a (G₄S)₃ sequence or is removed.

FIG. 19 is a model showing an alignment of the nC-B nanoCLAMP P2808 in which Loop 4 is replaced with a (G₄S)₃ sequence or is removed.

FIG. 20 is a model showing an alignment of the nC-B nanoCLAMP P2808 in which Loop 6 is replaced with a (G₄S)₃ sequence or GG.

FIG. 21 is a model showing the nC-B nanoCLAMP P2808 in which Loop 8 is replaced with a GGGGG (SEQ ID NO: 36), GGGG (SEQ ID NO: 37), GGG, GG, or G, or is removed.

FIG. 22 is a model showing an alignment of the nC-B nanoCLAMP P2808 in which Loop 8 is replaced with a (G4S)₃ sequence or is removed.

FIG. 23 is a model showing an alignment of the nC-B nanoCLAMP P2808 in which Loop 3 is replaced with a (G₄S)₃ sequence or is removed.

FIG. 24 is a model showing an alignment of the nC-B nanoCLAMP P2808 in which Loop 5 is replaced with a (G₄S)₃ sequence or is removed.

FIG. 25 is a model showing the nC-B nanoCLAMP P2808 in which Loop 7 is replaced with a GGGGG (SEQ ID NO: 36), GGGG (SEQ ID NO: 37), GGG, GG, or G, or is removed.

FIG. 26 is a model showing an alignment of the nC-B nanoCLAMP P2808 in which Loop 8 is replaced with a (G4S)₃ sequence or G.

FIG. 27 is a gel showing introduction of artificial disulfide bonds into clones P2808 and P2960. SDS-PAGE analysis of P2808 and P2960 variants, mutated to contain pairs of adjacent Cysteines, under oxidizing and reducing conditions. Purified proteins were treated with SDS sample buffer containing (first lane of each set) or lacking (second lane of each set) reducing agent (DTT). The presence of faster migrating species indicates disulfide bonding in samples lacking DTT, likely due to more compact folding and a smaller hydrodynamic radius. P2808 and P2960 contain no Cys residues and migrate at the same rate in oxidizing and reducing sample buffer, as expected. BSA, which contains 17 disulfide bonds, is included as a control for the activity of the reducing agent (DTT).

FIG. 28 is a graph showing that artificial disulfide improves thermal stability of P3015 by 9° C. The graph shows differential scanning fluorescence (DSF) analysis of melting temperature of reduced and oxidized P3015.

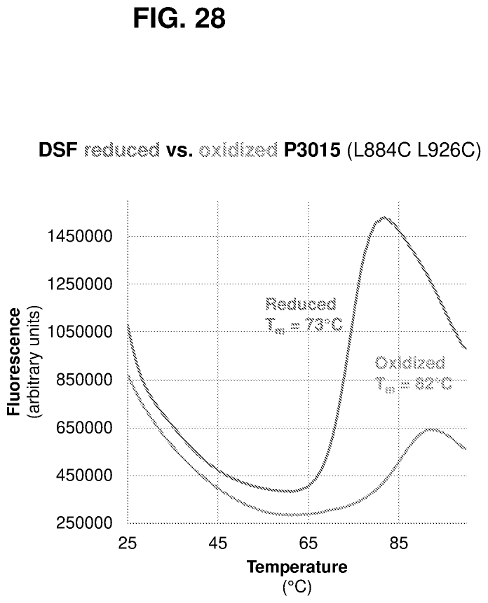

### DETAILED DESCRIPTION

Immunoaffinity chromatography is an established laboratory-scale technique for the isolation of target proteins with high yield and purity. However, properties of antibodies and nanobodies often make immunoaffinity chromatography incompatible with conditions typical of many industrial scale processes. To overcome these limitations, the present invention features an antibody-mimetic scaffold, called a nanoCLAMP, that can be used in process-scale affinity chromatography. The 16 kD antibody mimetic is based on a bacterial, cysteine-free, p-sandwich protein with a structure analogous to immunoglobulin variable domains. Like antibodies and other antibody mimetics, the first generation of nanoCLAMPs generally showed high selectivity and affinity but also suffered from sensitivity to high temperature, digestion by proteases, and inactivation by alkali. The present invention solves this problem by engineering a plurality of mutations in in the nanoCLAMP scaffold to improve the general robustness of nanoCLAMPs and resistance to extreme conditions.

This mutated scaffold serves as the basis for an improved nanoCLAMP class, called the nC-B class. Phage display was used to generate hundreds of nC-B capture agents recognizing diverse targets. The resulting immunoaffinity capture agents typically had a K_{d} of <80 nM, a Tₘ of >70° C. and a t_{1/2} in 0.1 mg/ml trypsin of >20 hours. The nC-B capture agents also maintained their binding capacity and selectivity over 20 purification cycles, each including 10 minutes of cleaning in place with 0.1 M NaOH. Affinity chromatography resins made with nC-B capture agents supported efficient single-step purifications from crude mixtures. Target proteins could be eluted with either 3 M imidazole, pH 8 or 0.1 M sodium citrate, pH 2.5. Furthermore, affinity chromatography resins with nC-B capture agents remained functional after exposure to 100% DMF and autoclaving. The robust nanoCLAMP scaffold described herein allows for the development of custom, high performance affinity chromatography resins compatible with the harsh conditions of process-scale applications that can be adaptable to a wide diversity of target substrates.

### Protein Scaffolds

The scaffolds described herein are derived from the Carbohydrate Binding Module Family 32 (CBM32) protein domain of Clostridium perfringens hyaluronidase (NagH), a multi-domain enzyme consisting of 1627 amino acids. Amino acid residue 1 of SEQ ID NO: 1 corresponds to amino acid residue 807 of NagH, and amino acid residue 140 of SEQ ID NO: 1 corresponds to amino acid residue 946 of NagH. Amino acid positions and mutations described herein generally relate to the position on the corresponding full length NagH unless otherwise specified. The WT sequence of CBM32 is shown below:

CBM32 SEQ
(SEQ ID NO: 1)
NPSLIRSESWQVYEGNEANLLDGDDNTGVWYKTLNGDTSLAGEFIGLDL
GKEIKLDGIRFVIGKNGGGSSDKWNKFKLEYSLDNESWTTIKEYDKTGA
PAGKDVIEESFETPISAKYIRLINMENINKWLTFSEFAIVSD

Previous work identified a scaffold in which three or five loop regions (L1, L2, and L8; or L1, L2, L4, L6, and L8) were mutagenized to form binders to diverse protein targets instead of carbohydrates, a property not expected for a carbohydrate binding module. In some embodiments, the protein scaffold does not retain carbohydrate binding activity, e.g., of the native CBM scaffold. Loop L1 corresponds to residues 817-820, loop L2 corresponds to residues 838-844, and loop L8 corresponds to residues 931-935. The original scaffold (nC-A) is shown below and contains a single M929L mutation relative to SEQ ID NO: 1. X denotes a variable loop residue, and each X may independently be any residue.

nC-A Scaffold sequence
(SEQ ID NO: 2)
NPSLIRSESWXXXXGNEANLLDGDDNTGVWYXXXXXXXSLAGEFIGLDL
GKEIKLDGIRFVIGKNGGGSSDKWNKFKLEYSLDNESWTTIKEYDKTGA
PAGKDVIEESFETPISAKYIRLTNLEXXXXXLTFSEFAIVSD

The current scaffold (nC-B) described herein is based on the exemplary scaffold of SEQ ID NO: 3 shown below. X denotes a variable loop residue, and each X may independently be any residue.

nC-B Scaffold sequence full length
(SEQ ID NO: 3)
DPTLIHTPGWXXXXGSEADLLDGDDSTGVEYXXXXXXXSLAGEFIGLDL
GEVVEVGGIHFVIGADGGGSSDKWTRFRLEYSLDGESWTTIREYDHTGA
PAGQDVIDEDFETPISAQYIRLTNLEXXXXXLTFSEFAIVSDELE

The protein scaffolds described herein include (e.g., consist of) framework regions (F) and loop regions (L). The scaffolds generally have the structure of:

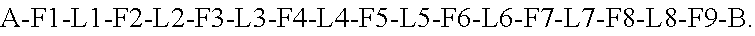

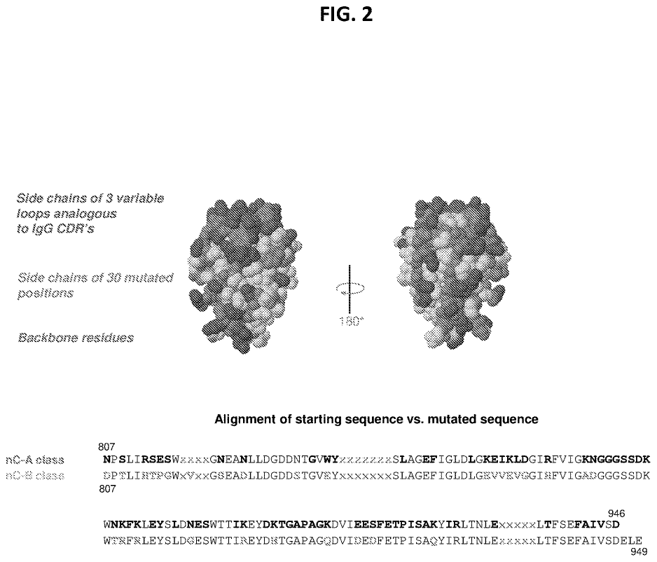

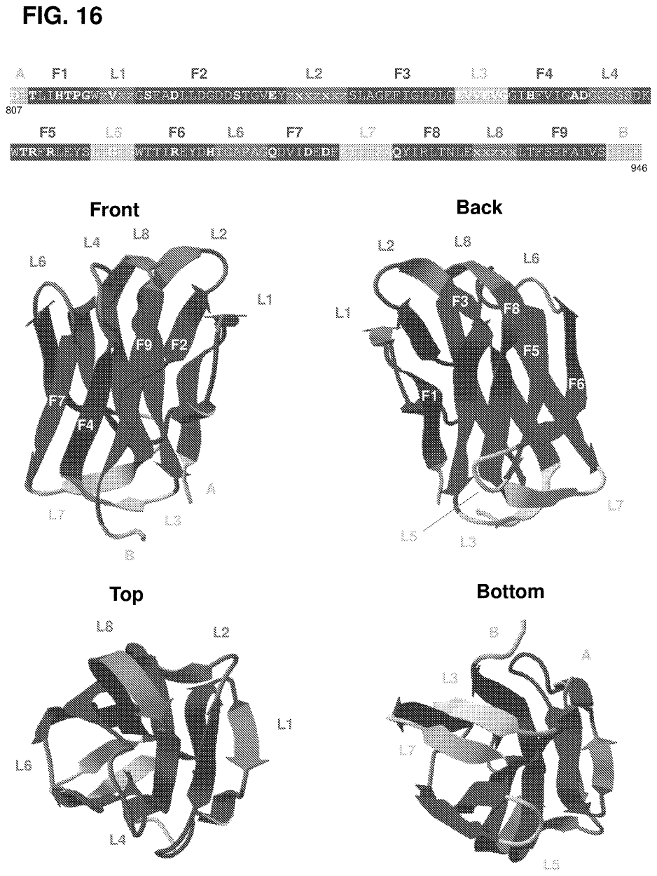

F1-F9 correspond to framework regions 1-9, and L1-L8 correspond to loop regions 1-8. Framework regions and loop regions were selected based on where beta strands turn to loops or where beta strands show a sharp turn from the plane of the strand's beta sheet (see FIGS. 2 and 16). The N- and C-termini of the scaffold, A and B, may each independently, be present (e.g., contain one or more amino acids) or absent.
F1 includes the sequence of: (T/S)-LI-(H/R)-(T/S)-(P/E)-(G/S)-W (SEQ ID NO: 4) or a sequence having one or two amino acid insertions, deletions, substitution mutations, or a combination thereof (e.g., one or two substitution mutations) relative to SEQ ID NO: 4;
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: G-(S/N/T)-E-(A/S)-(D/N/S/A)-LLDGDD-(S/N/T)-TGV-(E/W/A)-Y (SEQ ID NO: 5) or a sequence having one or two amino acid insertions, deletions, substitution mutations, or a combination thereof (e.g., one or two substitution mutations) relative to SEQ ID NO: 5;
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: S-(L/V)-AGEFIGLDLG (SEQ ID NO: 6) or a sequence having one or two amino acid insertions, deletions, substitution mutations, or a combination thereof (e.g., one or two substitution mutations) relative to SEQ ID NO: 6;
L3 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F4 includes the sequence of: G-(I/V)-(H/R/Y/N)-FVIG-(A/K/R)-(D/N) (SEQ ID NO: 7) or a sequence having one or two amino acid insertions, deletions, substitution mutations, or a combination thereof (e.g., one or two substitution mutations) relative to SEQ ID NO: 7;
L4 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F5 includes the sequence of: DKW-(T/N/S)-(R/K)-F-(R/K)-LEYS (SEQ ID NO: 8) or a sequence having one or two amino acid insertions, deletions, substitution mutations, or a combination thereof (e.g., one or two substitution mutations) relative to SEQ ID NO: 8;
L5 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F6 includes the sequence of: WTTI-(R/K/H/Q)-EYD-(H/K/R/Q) (SEQ ID NO: 9) or a sequence having one or two amino acid insertions, deletions, substitution mutations, or a combination thereof (e.g., one or two substitution mutations) relative to SEQ ID NO: 9;
L6 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F7 includes the sequence of: (Q/K)-DVI-(D/E)-E-(D/S)-F (SEQ ID NO: 10) or a sequence having one or two amino acid insertions, deletions, substitution mutations, or a combination thereof (e.g., one or two substitution mutations) relative to SEQ ID NO: 10;
L7 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F8 includes the sequence of: (Q/K/R)-YIRLTNLE (SEQ ID NO: 11) or a sequence having one or two amino acid insertions, deletions, substitution mutations, or a combination thereof (e.g., one or two substitution mutations) relative to SEQ ID NO: 11;
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFA-(I/V)-VS (SEQ ID NO: 12) or a sequence having one or two amino acid insertions, deletions, substitution mutations, or a combination thereof (e.g., one or two substitution mutations) relative to SEQ ID NO: 12.

As described herein, a sequence having one or two amino acid insertions, deletions, substitution mutations, or a combination thereof includes a sequence having, for example, one insertion, two insertions, one deletion, two deletions, one substitution mutation, two substitution mutations, one insertion and one deletion, one insertion and one substitution mutation, or one deletion and one substitution mutation.

In some embodiments, the protein scaffold includes at least one mutation selected from the group consisting of N807X, S809X, R812X, S813X, E814X, S815X, D818X, N822X, N825X, N832X, W836X, K857X, E858X, I859X, K860X, L861X, D862X, R865X, K870X, N871 X, N880X, K881 X, K883X, N890X, K897X, K901 X, K908X, E912X, S914X, and K922X relative to SEQ ID NO: 1, wherein X is any amino acid.

In some embodiments, the protein scaffold includes at least one mutation selected from the group consisting of N807X, S809X, R812X, S813X, E814X, S815X₁, D818X, N822X, N825X, N832X, W836X, K857X, E858X, I859X, K860X₂, L861X, D862X, R865X, K870X, N871 X, N880X, K881 X, K883X, N890X, K897X, K901X, K908X, E912X, S914X, and K922X₃ relative to SEQ ID NO: 1, wherein:
X is any amino acid except the amino acid in the equivalent position in SEQ ID NO: 1;
X₁ is any amino acid except R or S;
X₂ is any amino acid except P or K; and
X₃ is any amino acid except R or K.

In some embodiments,
F1 includes the sequence of: (T/S)-LI-(H/R)-(T/S)-(P/E)-(G/S)-W (SEQ ID NO: 4) or a sequence having one amino acid insertion, deletion, or substitution mutation (e.g., one substitution mutation) relative to SEQ ID NO: 4;
F2 includes the sequence of: G-(S/N/T)-E-(A/S)-(D/N/S/A)-LLDGDD-(S/N/T)-TGV-(E/W/A)-Y (SEQ ID NO: 5) or a sequence having one amino acid insertion, deletion, or substitution mutation (e.g., one substitution mutation) relative to SEQ ID NO: 5;
F3 includes the sequence of: S-(L/V)-AGEFIGLDLG (SEQ ID NO: 6) or a sequence having one amino acid insertion, deletion, or substitution mutation (e.g., one substitution mutation) relative to SEQ ID NO: 6;
F4 includes the sequence of: G-(I/V)-(H/R/Y/N)-FVIG-(A/K/R)-(D/N) (SEQ ID NO: 7) or a sequence having one amino acid insertion, deletion, or substitution mutation (e.g., one substitution mutation) relative to SEQ ID NO: 7;
F5 includes the sequence of: DKW-(T/N/S)-(R/K)-F-(R/K)-LEYS (SEQ ID NO: 8) or a sequence having one amino acid insertion, deletion, or substitution mutation (e.g., one substitution mutation) relative to SEQ ID NO: 8;
F6 includes the sequence of: WTTI-(R/K/H/Q)-EYD-(H/K/R/Q) (SEQ ID NO: 9) or a sequence having one amino acid insertion, deletion, or substitution mutation (e.g., one substitution mutation) relative to SEQ ID NO: 9;
F7 includes the sequence of: (Q/K)-DVI-(D/E)-E-(D/S)-F (SEQ ID NO: 10) or a sequence having one amino acid insertion, deletion, or substitution mutation (e.g., one substitution mutation) relative to SEQ ID NO: 10;
F8 includes the sequence of: (Q/K/R)-YIRLTNLE (SEQ ID NO: 11) or a sequence having one amino acid insertion, deletion, or substitution mutation (e.g., one substitution mutation) relative to SEQ ID NO: 11; and
F9 includes the sequence of: LTFSEFA-(I/V)-VS (SEQ ID NO: 12) or a sequence having one amino acid insertion, deletion, or substitution mutation (e.g., one substitution mutation) relative to SEQ ID NO: 12.

In some embodiments,
F1 includes the sequence of: (T/S)-LI-(H/R)-(T/S)-(P/E)-(G/S)-W (SEQ ID NO: 4);
F2 includes the sequence of: G-(S/N/T)-E-(A/S)-(D/N/S/A)-LLDGDD-(S/N/T)-TGV-(E/W/A)-Y (SEQ ID NO: 5);
F3 includes the sequence of: S-(L/V)-AGEFIGLDLG (SEQ ID NO: 6);
F4 includes the sequence of: G-(I/V)-(H/R/Y/N)-FVIG-(A/K/R)-(D/N) (SEQ ID NO: 7);
F5 includes the sequence of: DKW-(T/N/S)-(R/K)-F-(R/K)-LEYS (SEQ ID NO: 8);
F6 includes the sequence of: WTTI-(R/K/H/Q)-EYD-(H/K/R/Q) (SEQ ID NO: 9);
F7 includes the sequence of: (Q/K)-DVI-(D/E)-E-(D/S)-F (SEQ ID NO: 10);
F8 includes the sequence of: (Q/K/R)-YIRLTNLE (SEQ ID NO: 11); and
F9 includes the sequence of: LTFSEFA-(I/V)-VS (SEQ ID NO: 12).

In some embodiments, the protein scaffold includes at least one mutation selected from the group consisting of N807X, S809X, R812X, S813X, E814X, S815X, D818X, N822X, N825X, N832X, W836X, K857X, E858X, I859X, K860X, L861X, D862X, R865X, K870X, N871 X, N880X, K881 X, K883X, N890X, K897X, K901 X, K908X, E912X, S914X, and K922X relative to SEQ ID NO: 1, wherein X is any amino acid.

In some embodiments, the protein scaffold includes at least one mutation selected from the group consisting of N807D, S809T, R812H, S813T, E814P, S815G, D818V, N822S, N825D, N832S, W836E, K857E, E858V, I859V, K860E, L861V, D862G, R865H, K870A, N871D, N880T, K881R, K883R, N890G, K897R, K901H, K908Q, E912D, S914D, and K922Q relative to SEQ ID NO: 1.

In some embodiments, the at least one mutation is K870X and/or N890X. In some embodiments, the at least one mutation is K870A and/or N890G. In some embodiments, the at least one mutation is K870A. In some embodiments, the at least one mutation is N890G.

In some embodiments, the protein scaffold includes at least 3 fewer lysines relative to SEQ ID NO: 1. For example, in some embodiments, the protein scaffold includes at least 3, 4, 5, 6, 7, 8, 9, or 10 fewer lysines relative to SEQ ID NO: 1. In some embodiments, the protein scaffold includes at least 6 fewer lysines relative to SEQ ID NO: 1. In some embodiments, the protein scaffold includes 9 fewer lysines relative to SEQ ID NO: 1. In some embodiments, the protein scaffold does not include any lysines.

In some embodiments, the protein scaffold includes at least 3 fewer asparagines relative to SEQ ID NO: 1. For example, in some embodiments, the protein scaffold includes at least 3, 4, 5, 6, 7, or 8 fewer asparagines relative to SEQ ID NO: 1. In some embodiments, the protein scaffold includes at least 5 fewer asparagines relative to SEQ ID NO: 1. In some embodiments, the protein scaffold includes 7 fewer asparagines relative to SEQ ID NO: 1. In some embodiments, the protein scaffold does not include any asparagines.

In some embodiments, A and B are each independently, absent or at least one amino acid. For example, each of A and B may each be independently, absent. In some embodiments, A and B are each independently, at least 1, 2, 3, 4, 5, 6, 7, 8, 9, 10, 11, 12, 13, 14, 15, 16, 17, 18, 19, 20, 30, 40, 50, 60, 70, 80, 90, 100, 200, 30, 400, 500, 600, 700, 800, 900, 1,000 or more amino acids. In some embodiments, A and B are each independently, from 0 to 1,000 amino acids, e.g., from 1 to 10 amino acids (e.g., 1, 2, 3, 4, 5, 6, 7, 8, 9, or 10 amino acids), from 10 to 100 amino acids (e.g., 10, 20, 30, 40, 50, 60, 70, 80, 90, or 100 amino acids, or from 100 to 1,000 amino acids (e.g., 100, 200, 300, 400, 500, 600, 700, 800, 900, or 1,000 amino acids).

In some embodiments, A and B are each independently, absent or from 1 amino acid to 20 amino acids (e.g., 1, 2, 3, 4, 5, 6, 7, 8, 9, 10, 11, 12, 13, 14, 15, 16, 17, 18, 19, or 20 amino acids).

In some embodiments, each of L1-L8 is independently, from 1 amino acid to 20 amino acids (e.g., 1, 2, 3, 4, 5, 6, 7, 8, 9, 10, 11, 12, 13, 14, 15, 16, 17, 18, 19, or 20 amino acids).

In some embodiments, each of L1-L8 is, independently, from 1 amino acid to 10 amino acids (e.g., 1, 2, 3, 4, 5, 6, 7, 8, 9, or 10 amino acids). In some embodiments, each of L1-L8 is, independently, from 3 amino acids to 10 amino acids. In some embodiments, each of L1-L8 is, independently, from 3 amino acids to 8 amino acids.

In some embodiments, L1 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L1 is from 0 to 5 amino acids (e.g., from 1 to 5 amino acids, e.g., 0, 1, 2, 3, 4, or 5 amino acids).

In some embodiments, L2 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L2 is from 1 amino acid to 16 amino acids (e.g., from 4 to 16 amino acids, e.g., 1, 2, 3, 4, 5, 6, 7, 8, 9, 10, 11, 12, 13, 14, 15, or 16 amino acids).

In some embodiments, L3 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L3 is 6 amino acids.

In some embodiments, L4 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L4 is from 0 to 5 amino acids (e.g., from 1 to 5 amino acids, e.g., 0, 1, 2, 3, 4, or 5 amino acids) In some embodiments, L5 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L5 is 5 amino acids.

In some embodiments, L6 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L6 is from 3 to 6 amino acids (e.g., 3, 4, 5, or 6 amino acids).

In some embodiments, L7 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L7 is 4 or 5 amino acids.

In some embodiments, L8 is from 1 amino acid to 20 amino acids (e.g., 1, 2, 3, 4, 5, 6, 7, 8, 9, 10, 11, 12, 13, 14, 15, 16, 17, 18, 19, or 20 amino acids). In some embodiments, L8 is from 4 to 6 amino acids (e.g., 4, 5, or 6 amino acids).

In some embodiments, L1 is 4 amino acids. In some embodiments, L2 is 7 amino acids. In some embodiments, L8 is 5 amino acids. In some embodiments, L1 is 4 amino acids, L2 is 7 amino acids, and/or L8 is 5 amino acids. In some embodiments, L1 is 4 amino acids, L2 is 7 amino acids, and L8 is 5 amino acids.

In some embodiments, L1 includes the sequence of: X₁X₂X₃X₄ (SEQ ID NO: 13), wherein each of X₁-X₄ is, independently, any amino acid. In some embodiments, X₂ is V.

In some embodiments, L2 includes the sequence of: X₁X₂X₃X₄X₅X₆X₇ (SEQ ID NO: 14), wherein each of X₁-X₇ is, independently, any amino acid.

In some embodiments, L8 includes the sequence of: X₁X₂X₃X₄X₅ (SEQ ID NO: 15), wherein each of X₁-X₅ is, independently, any amino acid.

In some embodiments, L4 includes the sequence of: X₁X₂X₃X₄X₅X₆X₇ (SEQ ID NO: 14), wherein each of X₁-X₇ is, independently, any amino acid.

In some embodiments, L6 includes the sequence of: X₁X₂X₃X₄X₅X₆(SEQ ID NO: 16), wherein each of X₁-X₆ is, independently, any amino acid. In some embodiments, L8 includes at least two amino acids. In some embodiments, L8 includes at least one amino acid.

In some embodiments, L4 includes the sequence of: (G/D)-GGSS (SEQ ID NO: 17) or GDT or a sequence having one or two amino acid insertions, deletions, substitution mutations, or a combination thereof (e.g., one or two substitution mutations) relative to SEQ ID NO: 17 or GDT.

In some embodiments, L6 includes the sequence of TGAPAG (SEQ ID NO: 18) or a sequence having one or two amino acid insertions, deletions, substitution mutations, or a combination thereof (e.g., one or two substitution mutations) relative to SEQ ID NO: 18.

In some embodiments, L4 includes the sequence of: (G/D)-GGSS (SEQ ID NO: 17) or GDT; and L6 includes the sequence of TGAPAG (SEQ ID NO: 18).

In some embodiments, L3 includes the sequence of: (E/K/S)-(V/E)-(V/I/T)-(E/K/P/S)-(V/L)-(G/D) (SEQ ID NO: 19) or a sequence having one or two amino acid insertions, deletions, substitution mutations, or a combination thereof (e.g., one or two substitution mutations) relative to SEQ ID NO: 19.

In some embodiments, L5 includes the sequence of: LD-(G/N)-(E/S)-S(SEQ ID NO: 20) or a sequence having one or two amino acid insertions, deletions, substitution mutations, or a combination thereof (e.g., one or two substitution mutations) relative to SEQ ID NO: 20.

In some embodiments, L7 includes at least one amino acid.

In some embodiments, L7 includes the sequence of ETPI-(S/E)-A (SEQ ID NO: 21) or a sequence having one or two amino acid insertions, deletions, substitution mutations, or a combination thereof (e.g., one or two substitution mutations) relative to SEQ ID NO: 21.

In some embodiments, L3 includes the sequence of: (E/K/S)-(V/E)-(V/I/T)-(E/K/P/S)-(V/L)-(G/D) (SEQ ID NO: 19) or a sequence having one or two amino acid insertions, deletions, substitution mutations, or a combination thereof (e.g., one or two substitution mutations) relative to SEQ ID NO: 19; L5 includes the sequence of: LD-(G/N)-(E/S)-S(SEQ ID NO: 20) or a sequence having one or two amino acid insertions, deletions, substitution mutations, or a combination thereof (e.g., one or two substitution mutations) relative to SEQ ID NO: 20; and L7 includes the sequence of ETPI-(S/E)-A (SEQ ID NO: 21) or a sequence having one or two amino acid insertions, deletions, substitution mutations, or a combination thereof (e.g., one or two substitution mutations) relative to SEQ ID NO: 21.

In some embodiments, A includes the sequence of (D/N/H)-P. In some embodiments, A includes the sequence of DP.

In some embodiments, B includes the sequence of DELE (SEQ ID NO: 35).

In some embodiments, F1 includes the sequence of: TLIHTPGW (SEQ ID NO: 22) or a sequence having one or two amino acid insertions, deletions, substitution mutations, or a combination thereof (e.g., one or two substitution mutations) relative to SEQ ID NO: 22;
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: GSEADLLDGDDSTGVEY (SEQ ID NO: 23) or a sequence having one or two amino acid insertions, deletions, substitution mutations, or a combination thereof (e.g., one or two substitution mutations) relative to SEQ ID NO: 23;
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: SLAGEFIGLDLG (SEQ ID NO: 24) or a sequence having one or two amino acid insertions, deletions, substitution mutations, or a combination thereof (e.g., one or two substitution mutations) relative to SEQ ID NO: 24;
L3 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F4 includes the sequence of: GIHFVIGAD (SEQ ID NO: 25) or a sequence having one or two amino acid insertions, deletions, substitution mutations, or a combination thereof (e.g., one or two substitution mutations) relative to SEQ ID NO: 25;
L4 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F5 includes the sequence of: DKWTRFRLEYS (SEQ ID NO: 26) or a sequence having one or two amino acid insertions, deletions, substitution mutations, or a combination thereof (e.g., one or two substitution mutations) relative to SEQ ID NO: 26;
L5 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F6 includes the sequence of: WTTIREYDH (SEQ ID NO: 27) or a sequence having one or two amino acid insertions, deletions, substitution mutations, or a combination thereof (e.g., one or two substitution mutations) relative to SEQ ID NO: 27;
L6 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F7 includes the sequence of: QDVIDEDF (SEQ ID NO: 28) or a sequence having one or two amino acid insertions, deletions, substitution mutations, or a combination thereof (e.g., one or two substitution mutations) relative to SEQ ID NO: 28;
L7 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F8 includes the sequence of: QYIRLTNLE (SEQ ID NO: 29) or a sequence having one or two amino acid insertions, deletions, substitution mutations, or a combination thereof (e.g., one or two substitution mutations) relative to SEQ ID NO: 29;
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFAIVS (SEQ ID NO: 30) or a sequence having one or two amino acid insertions, deletions, substitution mutations, or a combination thereof (e.g., one or two substitution mutations) relative to SEQ ID NO: 30.

In some embodiments, F1 includes the sequence of: TLIHTPGW (SEQ ID NO: 22);
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: GSEADLLDGDDSTGVEY (SEQ ID NO: 23);
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: SLAGEFIGLDLG (SEQ ID NO: 24);
L3 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F4 includes the sequence of: GIHFVIGAD (SEQ ID NO: 25);
L4 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F5 includes the sequence of: DKWTRFRLEYS (SEQ ID NO: 26);
L5 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F6 includes the sequence of: WTTIREYDH (SEQ ID NO: 27);
L6 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F7 includes the sequence of: QDVIDEDF (SEQ ID NO: 28);
L7 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F8 includes the sequence of: QYIRLTNLE (SEQ ID NO: 29);
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFAIVS (SEQ ID NO: 30).

In some embodiments, F1 comprises the sequence of: TLIHTPGW (SEQ ID NO: 22) or a sequence having one or two amino acid insertions, deletions, substitution mutations, or a combination thereof (e.g., one or two substitution mutations) relative to SEQ ID NO: 22;
L1 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 comprises the sequence of: GSEADLLDGDDSTGVEY (SEQ ID NO: 23) or a sequence having one or two amino acid insertions, deletions, substitution mutations, or a combination thereof (e.g., one or two substitution mutations) relative to SEQ ID NO: 23;
L2 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 comprises the sequence of: SLAGEFIGLDLG (SEQ ID NO: 24) or a sequence having one or two amino acid insertions, deletions, substitution mutations, or a combination thereof (e.g., one or two substitution mutations) relative to SEQ ID NO: 24;
L3 comprises the sequence of: EVVEVG (SEQ ID NO: 31) or a sequence having one or two amino acid insertions, deletions, substitution mutations, or a combination thereof (e.g., one or two substitution mutations) relative to SEQ ID NO: 31;
F4 comprises the sequence of: GIHFVIGAD (SEQ ID NO: 25) or a sequence having one or two amino acid insertions, deletions, substitution mutations, or a combination thereof (e.g., one or two substitution mutations) relative to SEQ ID NO: 25;
L4 comprises the sequence of: GGGSS (SEQ ID NO: 32) or a sequence having one or two amino acid insertions, deletions, substitution mutations, or a combination thereof (e.g., one or two substitution mutations) relative to SEQ ID NO: 32;
F5 comprises the sequence of: DKWTRFRLEYS (SEQ ID NO: 26) or a sequence having one or two amino acid insertions, deletions, substitution mutations, or a combination thereof (e.g., one or two substitution mutations) relative to SEQ ID NO: 26;
L5 comprises the sequence of: LDGES (SEQ ID NO: 33) or a sequence having one or two amino acid insertions, deletions, substitution mutations, or a combination thereof (e.g., one or two substitution mutations) relative to SEQ ID NO: 33;
F6 comprises the sequence of: WTTIREYDH (SEQ ID NO: 27) or a sequence having one or two amino acid insertions, deletions, substitution mutations, or a combination thereof (e.g., one or two substitution mutations) relative to SEQ ID NO: 27;
L6 comprises the sequence of: TGAPAG (SEQ ID NO: 18) or a sequence having one or two amino acid insertions, deletions, substitution mutations, or a combination thereof (e.g., one or two substitution mutations) relative to SEQ ID NO: 18;
F7 comprises the sequence of: QDVIDEDF (SEQ ID NO: 28) or a sequence having one or two amino acid insertions, deletions, substitution mutations, or a combination thereof (e.g., one or two substitution mutations) relative to SEQ ID NO: 28;
L7 comprises the sequence of: ETPISA (SEQ ID NO: 34) or a sequence having one or two amino acid insertions, deletions, substitution mutations, or a combination thereof (e.g., one or two substitution mutations) relative to SEQ ID NO: 34;
F8 comprises the sequence of: QYIRLTNLE (SEQ ID NO: 29) or a sequence having one or two amino acid insertions, deletions, substitution mutations, or a combination thereof (e.g., one or two substitution mutations) relative to SEQ ID NO: 29;
L8 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 comprises the sequence of: LTFSEFAIVS (SEQ ID NO: 30) or a sequence having one or two amino acid insertions, deletions, substitution mutations, or a combination thereof (e.g., one or two substitution mutations) relative to SEQ ID NO: 30.

In some embodiments, F1 comprises the sequence of: TLIHTPGW (SEQ ID NO: 22) or a sequence having one amino acid insertion, deletion, or substitution mutation (e.g., one substitution mutation) relative to SEQ ID NO: 22;
L1 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 comprises the sequence of: GSEADLLDGDDSTGVEY (SEQ ID NO: 23) or a sequence having one amino acid insertion, deletion, or substitution mutation (e.g., one substitution mutation) relative to SEQ ID NO: 23;
L2 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 comprises the sequence of: SLAGEFIGLDLG (SEQ ID NO: 24) or a sequence having one amino acid insertion, deletion, or substitution mutation (e.g., one substitution mutation) relative to SEQ ID NO: 24;
L3 comprises the sequence of: EVVEVG (SEQ ID NO: 31) or a sequence having one amino acid insertion, deletion, or substitution mutation (e.g., one substitution mutation) relative to SEQ ID NO: 31;
F4 comprises the sequence of: GIHFVIGAD (SEQ ID NO: 25) or a sequence having one amino acid insertion, deletion, or substitution mutation (e.g., one substitution mutation) relative to SEQ ID NO: 25;
L4 comprises the sequence of: GGGSS (SEQ ID NO: 32) or a sequence having one amino acid insertion, deletion, or substitution mutation (e.g., one substitution mutation) relative to SEQ ID NO: 32;
F5 comprises the sequence of: DKWTRFRLEYS (SEQ ID NO: 26) or a sequence having one amino acid insertion, deletion, or substitution mutation (e.g., one substitution mutation) relative to SEQ ID NO: 26;
L5 comprises the sequence of: LDGES (SEQ ID NO: 33) or a sequence having one amino acid insertion, deletion, or substitution mutation (e.g., one substitution mutation) relative to SEQ ID NO: 33;
F6 comprises the sequence of: WTTIREYDH (SEQ ID NO: 27) or a sequence having one amino acid insertion, deletion, or substitution mutation (e.g., one substitution mutation) relative to SEQ ID NO: 27;
L6 comprises the sequence of: TGAPAG (SEQ ID NO: 18) or a sequence having one amino acid insertion, deletion, or substitution mutation (e.g., one substitution mutation) relative to SEQ ID NO: 18;
F7 comprises the sequence of: QDVIDEDF (SEQ ID NO: 28) or a sequence having one amino acid insertion, deletion, or substitution mutation (e.g., one substitution mutation) relative to SEQ ID NO: 28;
L7 comprises the sequence of: ETPISA (SEQ ID NO: 34) or a sequence having one amino acid insertion, deletion, or substitution mutation (e.g., one substitution mutation) relative to SEQ ID NO: 34;
F8 comprises the sequence of: QYIRLTNLE (SEQ ID NO: 29) or a sequence having one amino acid insertion, deletion, or substitution mutation (e.g., one substitution mutation) relative to SEQ ID NO: 29;
L8 is absent or comprises at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 comprises the sequence of: LTFSEFAIVS (SEQ ID NO: 30) or a sequence having one amino acid insertion, deletion, or substitution mutation (e.g., one substitution mutation) relative to SEQ ID NO: 30.

In some embodiments, F1 includes the sequence of: TLIHTPGW (SEQ ID NO: 22);
L1 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F2 includes the sequence of: GSEADLLDGDDSTGVEY (SEQ ID NO: 23);
L2 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids);
F3 includes the sequence of: SLAGEFIGLDLG (SEQ ID NO: 24);
L3 includes the sequence of: EVVEVG (SEQ ID NO: 31);
F4 includes the sequence of: GIHFVIGAD (SEQ ID NO: 25);
L4 includes the sequence of: GGGSS (SEQ ID NO: 32);
F5 includes the sequence of: DKWTRFRLEYS (SEQ ID NO: 26);
L5 includes the sequence of: LDGES (SEQ ID NO: 33);
F6 includes the sequence of: WTTIREYDH (SEQ ID NO: 27);
L6 includes the sequence of: TGAPAG (SEQ ID NO: 18);
F7 includes the sequence of: QDVIDEDF (SEQ ID NO: 28);
L7 includes the sequence of: ETPISA (SEQ ID NO: 34);
F8 includes the sequence of: QYIRLTNLE (SEQ ID NO: 29);
L8 is absent or includes at least one amino acid (e.g., 1 to 20 amino acids, e.g., 1, 2, 3, 4, 5, 6, 7, 8, 9, 10, 11, 12, 13, 14, 15, 16, 17, 18, 19, or 20 amino acids); and
F9 includes the sequence of: LTFSEFAIVS (SEQ ID NO: 30).

In some embodiments, F1 includes the sequence of: TLIHTPGW (SEQ ID NO: 22);
L1 includes the sequence of: X₁X₂X₃X₄ (SEQ ID NO: 13), wherein each of X₁-X₄ is, independently, any amino acid;
F2 includes the sequence of: GSEADLLDGDDSTGVEY (SEQ ID NO: 23);
L2 includes the sequence of: X₁X₂X₃X₄X₅X₆X₇ (SEQ ID NO: 14), wherein each of X₁-X₇ is, independently, any amino acid;
F3 includes the sequence of: SLAGEFIGLDLG (SEQ ID NO: 24);
L3 includes the sequence of: EVVEVG (SEQ ID NO: 31);
F4 includes the sequence of: GIHFVIGAD (SEQ ID NO: 25);
L4 includes the sequence of: GGGSS (SEQ ID NO: 32);
F5 includes the sequence of: DKWTRFRLEYS (SEQ ID NO: 26);
L5 includes the sequence of: LDGES (SEQ ID NO: 33);
F6 includes the sequence of: WTTIREYDH (SEQ ID NO: 27);
L6 includes the sequence of: TGAPAG (SEQ ID NO: 18);
F7 includes the sequence of: QDVIDEDF (SEQ ID NO: 28);
L7 includes the sequence of: ETPISA (SEQ ID NO: 34);
F8 includes the sequence of: QYIRLTNLE (SEQ ID NO: 29);
L8 includes the sequence of: X₁X₂X₃X₄X₅ (SEQ ID NO: 15), wherein each of X₁-X₅ is, independently, any amino acid; and
F9 includes the sequence of: LTFSEFAIVS (SEQ ID NO: 30).

In some embodiments, A includes the sequence of: DP;
F1 includes the sequence of: TLIHTPGW (SEQ ID NO: 22);
L1 includes the sequence of: X₁X₂X₃X₄ (SEQ ID NO: 13), wherein each of X₁-X₄ is, independently, any amino acid;
F2 includes the sequence of: GSEADLLDGDDSTGVEY (SEQ ID NO: 23);
L2 includes the sequence of: X₁X₂X₃X₄X₅X₆X₇ (SEQ ID NO: 14), wherein each of X₁-X₇ is, independently, any amino acid;
F3 includes the sequence of: SLAGEFIGLDLG (SEQ ID NO: 24);
L3 includes the sequence of: EVVEVG (SEQ ID NO: 31);
F4 includes the sequence of: GIHFVIGAD (SEQ ID NO: 25);
L4 includes the sequence of: GGGSS (SEQ ID NO: 32);
F5 includes the sequence of: DKWTRFRLEYS (SEQ ID NO: 26);
L5 includes the sequence of: LDGES (SEQ ID NO: 33);
F6 includes the sequence of: WTTIREYDH (SEQ ID NO: 27);
L6 includes the sequence of: TGAPAG (SEQ ID NO: 18);
F7 includes the sequence of: QDVIDEDF (SEQ ID NO: 28);
L7 includes the sequence of: ETPISA (SEQ ID NO: 34);
F8 includes the sequence of: QYIRLTNLE (SEQ ID NO: 29);
L8 includes the sequence of: X₁X₂X₃X₄X₅ (SEQ ID NO: 15), wherein each of X₁-X₅ is, independently, any amino acid;
F9 includes the sequence of: LTFSEFAIVS (SEQ ID NO: 30); and B includes the sequence of: DELE (SEQ ID NO: 35).

In some embodiments, L1 includes the sequence of X₁X₂X₃X₄ (SEQ ID NO: 13), wherein each of X₁, X₃, and X₄ is, independently, any amino acid, and X₂ is V.

In another aspect, featured is a protein scaffold that includes a polypeptide having at least 80% (e.g., at least 85%, 90%, 95%, 97%, or 99%) sequence identity to SEQ ID NO: 3. In some embodiments, the polypeptide includes the sequence of SEQ ID NO: 3. In some embodiments, the polypeptide does not include the sequence of SEQ ID NO: 1. In some embodiments, the polypeptide does not include the sequence of SEQ ID NO: 2.

In another aspect, featured is a polypeptide having at least 85% (e.g., at least 90%, 95%, 97%, 99%, or 100%) sequence identity to a polypeptide of Table 9 or Table 10. In some embodiments, the polypeptide includes a sequence as set forth in Table 9 or Table 10.

In some embodiments, the polypeptide includes at least one mutation selected from the group consisting of N807X, S809X, R812X, S813X, E814X, S815X₁, D818X, N822X, N825X, N832X, W836X, K857X, E858X, I859X, K860X₂, L861X, D862X, R865X, K870X, N871X, N880X, K881X, K883X, N890X, K897X, K901X, K908X, E912X, S914X, and K922X₃ relative to SEQ ID NO: 1, wherein:
X is any amino acid except the amino acid in the equivalent position in SEQ ID NO: 1;
X₁ is any amino acid except R or S;
X₂ is any amino acid except P or K; and
X₃ is any amino acid except R or K.

One of skill in the art would understand that the protein scaffolds described herein, which contain 9 framework regions (i.e., F1-F9) may be optimized or swapped according to established biophysical techniques. Accordingly, the invention also features protein scaffolds containing 7 out of 9 or 8 out of 9 framework regions described herein. Based on the detailed structural analysis of the scaffold known in the art (see, e.g., Ficko-Blean et al. J. Mol. Bio. 390: 208-220, 2009) and PDB ID 2w1q, one of skill in the art could swap out one or more beta strands or a portion thereof (e.g., more than 2 residues of a given framework region, e.g., any one of F1-F9) of the core protein fold, while still maintaining structural integrity of the overall scaffold. One could generate a phage library where each member of a library expresses a protein scaffold with loops that confer binding to a specific target and with randomized amino acids at each position in a specific beta strand. The phage library could then be selected for library members that are thermostable and maintain binding to a target by selecting the library for those members that can withstand an incubation at >55° C. without aggregation and for those members that can bind to an immobilized target. The isolated clones with these properties would represent scaffolds with a swapped out beta strand or portion thereof.

Accordingly, in some embodiments, the invention also contemplates protein scaffolds having at least 7, e.g., at least 8, of the following framework regions, wherein 7 of the 9 or 8 of the 9 framework regions have the following sequence:
F1 includes the sequence of: (T/S)-LI-(H/R)-(T/S)-(P/E)-(G/S)-W (SEQ ID NO: 4) or a sequence having one or two amino acid insertions, deletions, substitution mutations, or a combination thereof (e.g., one or two substitution mutations) relative to SEQ ID NO: 4;
F2 includes the sequence of: G-(S/N/T)-E-(A/S)-(D/N/S/A)-LLDGDD-(S/N/T)-TGV-(E/W/A)-Y (SEQ ID NO: 5) or a sequence having one or two amino acid insertions, deletions, substitution mutations, or a combination thereof (e.g., one or two substitution mutations) relative to SEQ ID NO: 5;
F3 includes the sequence of: S-(L/V)-AGEFIGLDLG (SEQ ID NO: 6) or a sequence having one or two amino acid insertions, deletions, substitution mutations, or a combination thereof (e.g., one or two substitution mutations) relative to SEQ ID NO: 6;
F4 includes the sequence of: G-(I/V)-(H/R/Y/N)-FVIG-(A/K/R)-(D/N) (SEQ ID NO: 7) or a sequence having one or two amino acid insertions, deletions, substitution mutations, or a combination thereof (e.g., one or two substitution mutations) relative to SEQ ID NO: 7;
F5 includes the sequence of: DKW-(T/N/S)-(R/K)-F-(R/K)-LEYS (SEQ ID NO: 8) or a sequence having one or two amino acid insertions, deletions, substitution mutations, or a combination thereof (e.g., one or two substitution mutations) relative to SEQ ID NO: 8;
F6 includes the sequence of: WTTI-(R/K/H/Q)-EYD-(H/K/R/Q) (SEQ ID NO: 9) or a sequence having one or two amino acid insertions, deletions, substitution mutations, or a combination thereof (e.g., one or two substitution mutations) relative to SEQ ID NO: 9;
F7 includes the sequence of: (Q/K)-DVI-(D/E)-E-(D/S)-F (SEQ ID NO: 10) or a sequence having one or two amino acid insertions, deletions, substitution mutations, or a combination thereof (e.g., one or two substitution mutations) relative to SEQ ID NO: 10;
F8 includes the sequence of: (Q/K/R)-YIRLTNLE (SEQ ID NO: 11) or a sequence having one or two amino acid insertions, deletions, substitution mutations, or a combination thereof (e.g., one or two substitution mutations) relative to SEQ ID NO: 11; and
F9 includes the sequence of: LTFSEFA-(I/V)-VS (SEQ ID NO: 12) or a sequence having one or two amino acid insertions, deletions, substitution mutations, or a combination thereof (e.g., one or two substitution mutations) relative to SEQ ID NO: 12.

In other embodiments, the invention also contemplates protein scaffolds having at least 7, e.g., at least 8, of the following framework regions, wherein 7 of the 9 or 8 of the 9 framework regions have the following sequence:
F1 includes the sequence of: TLIHTPGW (SEQ ID NO: 22) or a sequence having one or two amino acid insertions, deletions, substitution mutations, or a combination thereof (e.g., one or two substitution mutations) relative to SEQ ID NO: 22;
F2 includes the sequence of: GSEADLLDGDDSTGVEY (SEQ ID NO: 23) or a sequence having one or two amino acid insertions, deletions, substitution mutations, or a combination thereof (e.g., one or two substitution mutations) relative to SEQ ID NO: 23;
F3 includes the sequence of: SLAGEFIGLDLG (SEQ ID NO: 24) or a sequence having one or two amino acid insertions, deletions, substitution mutations, or a combination thereof (e.g., one or two substitution mutations) relative to SEQ ID NO: 24;
F4 includes the sequence of: GIHFVIGAD (SEQ ID NO: 25) or a sequence having one or two amino acid insertions, deletions, substitution mutations, or a combination thereof (e.g., one or two substitution mutations) relative to SEQ ID NO: 25;
F5 includes the sequence of: DKWTRFRLEYS (SEQ ID NO: 26) or a sequence having one or two amino acid insertions, deletions, substitution mutations, or a combination thereof (e.g., one or two substitution mutations) relative to SEQ ID NO: 26;
F6 includes the sequence of: WTTIREYDH (SEQ ID NO: 27) or a sequence having one or two amino acid insertions, deletions, substitution mutations, or a combination thereof (e.g., one or two substitution mutations) relative to SEQ ID NO: 27;
F7 includes the sequence of: QDVIDEDF (SEQ ID NO: 28) or a sequence having one or two amino acid insertions, deletions, substitution mutations, or a combination thereof (e.g., one or two substitution mutations) relative to SEQ ID NO: 28;
F8 includes the sequence of: QYIRLTNLE (SEQ ID NO: 29) or a sequence having one or two amino acid insertions, deletions, substitution mutations, or a combination thereof (e.g., one or two substitution mutations) relative to SEQ ID NO: 29; and
F9 includes the sequence of: LTFSEFAIVS (SEQ ID NO: 30) or a sequence having one or two amino acid insertions, deletions, substitution mutations, or a combination thereof (e.g., one or two substitution mutations) relative to SEQ ID NO: 30.

In other embodiments, the invention also contemplates protein scaffolds having at least 7, e.g., at least 8, of the following framework regions, wherein 7 of the 9 or 8 of the 9 framework regions have the following sequence:
F1 includes the sequence of: TLIHTPGW (SEQ ID NO: 22);
F2 includes the sequence of: GSEADLLDGDDSTGVEY (SEQ ID NO: 23);
F3 includes the sequence of: SLAGEFIGLDLG (SEQ ID NO: 24);
F4 includes the sequence of: GIHFVIGAD (SEQ ID NO: 25);
F5 includes the sequence of: DKWTRFRLEYS (SEQ ID NO: 26);
F6 includes the sequence of: WTTIREYDH (SEQ ID NO: 27);
F7 includes the sequence of: QDVIDEDF (SEQ ID NO: 28);
F8 includes the sequence of: QYIRLTNLE (SEQ ID NO: 29); and
F9 includes the sequence of: LTFSEFAIVS (SEQ ID NO: 30).

In another aspect, featured is a protein scaffold that includes a polypeptide having at least 80% (e.g., at least 85%, 90%, 95%, 97%, or 99%) sequence identity to the framework regions (F1-F9) over the region of alignment corresponding to F1-F9 of the reference sequence (e.g., SEQ ID NO: 3).

In some embodiments, the protein scaffold includes one or more non-natural amino acids. In some embodiments, one or more of the framework regions includes a non-natural amino acid. In some embodiments, one or more of the loop regions includes a non-natural amino acid.

### Cysteine Mutations and Disulfide Bridges

The protein scaffolds described herein may lack native cysteine residues. Accordingly, the scaffold may be mutagenized to introduce one or more cysteine residues into the scaffold (e.g., in one or more loop or framework regions). When two or more cysteine residues are introduced into the scaffold at nearby sites, the two cysteine residues may form a disulfide bridge, e.g., under oxidizing conditions. In some embodiments, the disulfide bridge enhances thermal stability of the protein scaffold.

In some embodiments, the protein scaffold includes a mutation that adds a cysteine residue. In some embodiments, the protein scaffold includes a first mutation that adds a first cysteine residue and a second mutation that adds a second cysteine residue. In some embodiments, the first cysteine residue and the second cysteine residue form a disulfide bond under oxidizing conditions.

In some embodiments, the protein scaffold comprises at least one mutation selected from the group consisting of F806C, P808C, S845C, L855C, V858C, V861C, K878C, W879C, L884C, L888C, A904C, P905C, A906GC, G907C, I924C, L926C, N928C, L936C, I943C, L948C.

In some embodiments, the protein scaffold comprises at least two or more mutations selected from the group consisting of F806C, P808C, S845C, L855C, V858C, V861C, K878C, W879C, L884C, L888C, A904C, P905C, A906GC, G907C, I924C, L926C, N928C, L936C, I943C, L948C.

In some embodiments, the protein scaffold comprises a pair of cysteine mutations selected from the group consisting of K878C and G907C, K878C and A904C, V861C and I943C, P905C and L855C, S845C and L936C, W879C and N928C, L884C and L926C, F806C and L948C, V858C and L888C, K878C and G907C, K878C and A906GC, S845C and N928C, K878C and A904C, P808C and I943C, V861C and I924C, P808C and V861C, and I943C and L855C.

In some embodiments, the pair of cysteine mutations is selected from the group consisting of K878C and G907C, K878C and A904C, S845C and L936C, W879C and N928C, W879C and N928C, L884C and L926C, V858C and L888C, K878C and G907C, and K878C and A906GC.

### Tags and Functional Groups

The protein scaffolds described herein may further include a tag. A tag may provide for ease of purification, detection or attachment of the protein scaffold. The tag may be covalently attached to the scaffold. In some embodiments, A and/or B of the scaffold is or includes a tag.

In some embodiments, the tag is an affinity tag (e.g., a polyhistidine tag, e.g., 4, 5, 6, 7, 8, 9, or 10 histidines, e.g., Gly-His tags, e.g., AviTag, e.g., Calmodulin-tag, e.g., polyglutamate tag, e.g., polyarginine tag, e.g., SBP-tag).

In some embodiments, the tag is an epitope tag (e.g., ALFA-tag, C-tag, iCapTag, E-tag, FLAG-tag, HA-tag, Myc-tag, NE-tag, Rho1D4-tag, S-tag, Softag 1, Softag 3, Spot-tag, T7-tag, TC tag, Ty tag, V6 tag, VSV-tag or Xpress tag).

In some embodiments, the tag is a covalent protein tag (e.g., Isopeptag, SpyTag, SnoopTag, DogTag or SdyTag).

In some embodiments, the tag is a protein tag (e.g., biotin carboxyl carrier protein tag, glutathione-S-transferase (GST) tag, green fluorescent protein (GFP) tag, HaloTag, SNAP-tag, CLIP-tag, HUH-tag, maltose binding protein tag, Nus tag, thioredoxin tag, Fc tag, Designed Intrinsically Disordered tag, CRDSAT tag, SpyCatcher, SnoopCatcher, DogCatcher, SdyCatcher, or SUMO-tag.

In some embodiments, A and/or B of the scaffold includes an affinity tag, epitope tag, covalent peptide tag, or protein tag.

In some embodiments, the tag is attached to the N-terminus or the C-terminus of the scaffold.

In some embodiments, the scaffold is conjugated to a functional group. In some embodiments, the functional group includes biotin, streptavidin or a derivative of streptavidin, a polyethylene glycol moiety, a fluorescent dye, an enzyme, a radioactive moiety, a lanthanide, or a lanthanide binding motif.

In some embodiments, the scaffold is conjugated to a lanthanide or a lanthanide binding motif. In some embodiments, the lanthanide is terbium.

In some embodiments, the scaffold is conjugated to a radioactive moiety. In some embodiments, the radioactive moiety is an a or p emitter.

In some embodiments, the functional group is conjugated to a sulfhydryl group or a primary amine (e.g., on a cysteine residue or a lysine).

### Polynucleotides, Vectors, and Cells

The protein scaffolds described herein may be encoded by a polynucleotide. In some embodiments, the polynucleotide is a ribonucleotide. In some embodiments, the polynucleotide is a deoxyribonucleotide. Also contemplated herein is a vector that includes a polynucleotide encoding the protein scaffold.

In other embodiments, featured is a cell that includes a polynucleotide encoding the protein scaffold or a vector that includes the polynucleotide. The polynucleotide or vector may include an expression element configured to drive expression of the protein scaffold. The cell may be a prokaryotic cell (e.g., E. coli). The cell may be a eukaryotic cell. In some embodiments, the eukaryotic cell is yeast cell (e.g., S. cerevisiae) or a mammalian cell (e.g., a Chinese hamster ovary (CHO) cell). In some embodiments, the protein scaffold is secreted by the cell. In some embodiments, the protein scaffold is expressed within the cell. Such a cell (e.g., E. coli) may be lysed to provide a lysate that includes the protein scaffold.

Also featured herein is a method of producing a protein scaffold as described herein. The method includes the steps of providing a cell transformed with a polynucleotide encoding the protein scaffold or a vector that includes the polynucleotide and culturing the transformed cell under conditions for expressing the polynucleotide. The culturing step results in expression of the protein scaffold. The method may further include isolating the protein scaffold or using the protein scaffold to bind a target.

### Particles, Resins, and Columns

The protein scaffolds described herein may be conjugated to a particle. In some embodiments, the particle is a magnetic particle. Also featured is a resin or monolith that includes a plurality of the particles, e.g., containing the protein scaffold. Also contemplated herein is a column (e.g., a chromatography column) containing the particles or the resin, e.g., conjugated to the scaffold.

The scaffolds and methods of use thereof can use a surface linked to the protein scaffold, which is configured to bind its target. The surface of the resin refers to a part of a support structure (e.g., a substrate) that is accessible to contact with one or more target molecules. The shape, form, materials, and modifications of the surface of the resin can be selected from a range of options depending on the application. In one embodiment, the surface of the resin is SEPHAROSE®. In one embodiment, the surface of the resin is agarose.

The surface of the resin can be substantially flat or planar. Alternatively, the surface of the resin can be rounded or contoured. Exemplary contours that can be included on a surface of the resin are wells, depressions, pillars, ridges, channels or the like.

In one embodiment, the surface of the resin is modified to contain channels, patterns, layers, or other configurations (e.g., a patterned surface). The surface can be in the form of a bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial. The surface can be a singular discrete body (e.g., a single tube, a single bead), any number of a plurality of surface bodies (e.g., a rack of 10 tubes, several beads), or combinations thereof (e.g., a tray includes a plurality of microtiter plates, a column filled with beads, a microtiter plate filed with beads).

In some embodiments, a surface can include a membrane-based resin matrix. In some embodiments, the surface of the resin includes a porous resin or a non-porous resin. Examples of porous resins can include additional agarose-based resins (e.g., cyanogen bromide activated SEPHAROSE® (GE); WORKBEADS™ 40 ACT and WORKBEADS™ 40/10000 ACT (Bioworks)), methacrylate: (Tosoh 650M derivatives etc.), polystyrene divinylbenzene (Life Tech Poros media/GE Source media), fractogel, polyacrylamide, silica, controlled pore glass, dextran derivatives, acrylamide derivatives, convective-interaction media (Sartorius), additional polymers, and combinations thereof.

In some embodiments, a surface can include one or more pores. In some embodiments, pore sizes can be from 300 to 8,000 Angstroms, e.g., 500 to 4,000 Angstroms in size.

A resin as described herein includes a plurality of particles. Examples of particle sizes are 5 μm-500 μm, 20 μm-300 μm, and 50 μm-200 μm. In some embodiments, particle size can be 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm.

A protein scaffold can be immobilized, coated on, bound to, stuck, adhered, or attached to any of the forms of surfaces described herein (e.g., bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial).

### Methods of Purification

Featured herein is a method of purifying a target molecule, e.g., from a plurality of molecules, e.g., from a crude lysate. The method includes providing a sample that includes a mixture of the target molecule and the plurality of molecules and contacting the sample with a protein scaffold as described herein. The protein scaffold may have previously been generated with loop regions that are specific to the desired target. The scaffold (e.g., the loop regions of the scaffold) specifically binds to the target molecule. The method further includes separating the target molecule bound to the protein scaffold from the plurality of molecules. In some embodiments, the step of separating includes immobilizing the protein scaffold. In some embodiments, the protein scaffold is conjugated to a particle. In some embodiments, the particle includes a magnetic bead. In some embodiments, the protein scaffold is conjugated to a resin or monolith as described herein.

The scaffolds, particles, resins, and columns described herein are amenable to single-step purifications from crude mixtures. For example, target proteins may be eluted with polyol, imidazole (e.g., 3 M imidazole, e.g., pH 8), or sodium citrate (e.g., or 0.1 M sodium citrate, e.g., pH 2.5). The scaffolds, particles, resins, and columns may be cleaned, e.g., with an alkaline substance, e.g., NaOH, e.g., 0.1 M NaOH. Furthermore, resins or columns made with a protein scaffold as described herein may remain functional after exposure to dimethylformamide (DMF), e.g., 100% DMF and/or autoclaving. These features allow reuse of the scaffold without loss of target binding capabilities.

### EXAMPLES

### Example 1. Protein Engineering of nanoCLAMP Antibody-Mimetics for Use as Affinity Chromatography Capture Agents Resistant to High Temperature, Trypsin, Low pH, Organic Solvent, and Sodium Hydroxide

Immunoaffinity chromatography is an established laboratory-scale technique for the isolation of target proteins with high yield and purity. However, properties of antibodies and nanobodies often make immunoaffinity chromatography incompatible with conditions typical of many industrial scale processes. To overcome these limitations, we have optimized an antibody-mimetic, called a nanoCLAMP, for use in process-scale affinity chromatography. The 16 kD antibody mimetic is based on a bacterial, cysteine-free, R-sandwich protein with a structure analogous to immunoglobulin variable domains. Like antibodies and other antibody mimetics, the first generation of nanoCLAMPs generally showed high selectivity and affinity but also suffered from sensitivity to high temperature, digestion by proteases, and inactivation by alkali. In this work, we address these limitations with a protein engineering campaign to improve the general robustness of nanoCLAMPs. Over 7 rounds of mutagenesis and screening, we tested 185 protein variants, with at least one mutation made in 58% of the positions in the nanoCLAMP's constant regions (72 of 124). The campaign yielded a protein with mutations in 30 of 124 positions in the constant regions and dramatically improved resistance to extreme conditions. The mutant protein served as the basis for an improved nanoCLAMP class, called the nC-B class. Phage display was then used to generate several nC-B capture agents recognizing diverse targets. The resulting immunoaffinity capture agents typically had a K_{d} of <80 nM, a Tₘ of >70° C. and a t_{1/2} in 0.1 mg/ml trypsin of >20 hours. The nC-B capture agents also maintained their binding capacity and selectivity over 20 purification cycles, each including 10 minutes of cleaning in place with 0.1 M NaOH. Affinity chromatography resins made with nC-B capture agents supported efficient single-step purifications from crude mixtures. Target proteins could be eluted with either 3 M imidazole, pH 8 or 0.1 M sodium citrate, pH 2.5. Furthermore, affinity chromatography resins with nC-B capture agents remained functional after exposure to 100% DMF and autoclaving. The robust nC-B scaffold developed in this work enables the development of custom, high performance affinity chromatography resins compatible with the harsh conditions of process-scale applications.

### INTRODUCTION

Affinity chromatography (AC) with target-specific, immobilized capture agents is an established method of protein purification. In this technique, a capture agent, such as a protein, nucleic acid, or small molecule, is coupled to a solid support, which can then be used to isolate a protein of interest from a complex mixture. The technique has been widely used at the laboratory scale for single-step purifications of diverse target proteins, including enzymes, transcription factors, growth factors and antibodies.

Use of protein-based capture agents for AC in industrial applications has been less widespread because currently available approaches are incompatible with the temperatures, pH extremes, and solvents often needed for process-scale purification or are useful for only a limited number of targets. An exception is the purification of kg-quantities of antibodies with AC resins based on Staphylococcal Protein A. The development of Protein A resins highlights the use of protein engineering to improve the robustness of AC resins as well as some remaining limitations. Early versions of resins with wild-type Protein A captured antibodies with high selectivity and capacity from cell culture media feedstock but lost activity gradually after multiple cycles of cleaning in place with sodium hydroxide. Mutagenesis of Protein A yielded variants with increased resistance to sodium hydroxide treatment and higher binding capacity. Despite the widespread use of Protein A resins, they are nonetheless limited to the purification of antibodies.

For the process-scale purification of non-antibody targets, the use of AC is much less widespread than the use of Protein A to purify antibodies. For instance, non-protein, ligand-based approaches, such as small molecule substrate mimetics, are effective but are limited to specific enzyme classes and are difficult to use with a general protein of interest. Alternatively, specialized affinity resins such as glutathione or nickel require the addition of non-native tags, which cause downstream complications for proteins intended for therapeutic use. Immuno-AC with antibody- or nanobody-based capture agents is the most generally applicable approach and has widely been used to purify a diverse range of proteins at laboratory-scale. However, immuno-AC has some limitations. In general, the conjugation of the antibody to the resin often results in heterogenous coupling because of a lack of precise control over the sites of conjugation. In addition, chromatography must be performed under oxidizing conditions in order to preserve the disulfide bonds essential for maintaining antibody structure. Elution of the target also usually requires low or high pH conditions that are incompatible with some target proteins. For process-scale applications, the chief limitation of immuno-AC resins is their sensitivity to the sodium hydroxide solutions which are preferred for cleaning-in-place procedures.

These limitations have motivated the development of AC resins based on antibody mimetics-proteins that, like antibodies, can be produced to bind specific antigens with high affinity and specificity, but are not directly derived from the immune system of animals. Examples of antibody mimetics include those based on Protein A, gamma-b crystallin, ubiquitin, cystatin, lipocalins, ankyrin repeat motifs, SH3 domains, fibronectin, OB fold domains, lamprey variable lymphocyte receptors, minibodies, miniproteins, and Kunitz domains. Most of these antibody mimetics use animal-sparing phage display for their isolation and can be produced by microbial cells. Many have a unique cysteine that supports homogeneous, site-specific coupling to sulfhydryl-reactive supports. However, few of the current antibody mimetics have been shown to enable elution near neutral pH or to be compatible with harsh conditions sometimes needed for process-scale procedures. The development of custom peptide and protein-based affinity chromatography platforms for the purification of non-antibody protein therapeutics is supported by some proprietary platforms, but the availability and technical details of these platforms is limited (e.g., Avitide, LigaTrap Technologies, Astrea Bioseparations, and Navigo Proteins).

We set out to develop a broadly available and generally applicable class of protein-based affinity capture reagents useful for industrial protein purification. Specifically, we sought to develop an AC capture agent technology with the potential to address a broad range of targets; enable single-step purifications from crude mixtures; elute targets at near neutral pH; and maintain function after exposure to high temperatures, organic solvents, proteases and pH extremes.

We previously developed an antibody mimetic based on the 16 kD, 2^{nd} Type 32 carbohydrate binding module of the hyaluronoglucosaminidase nagH from Clostridium perfringens (NagH CpCBM32-2) (Suderman et al. Protein Expr. Purif. 134:114-124, 2017). This binding module is a monomeric p-sandwich domain with variable loops comparable to the complementary determining regions of the immunoglobulin variable domain. We named these antibody mimetics nanoCLAMPs (nano Clostridial Antibody Mimetic Proteins). nanoCLAMPs have the unusual and advantageous general property of releasing bound target protein in solutions of non-denaturing polyols and ammonium sulfate at neutral pH. We have isolated nanoCLAMPs recognizing a variety of target proteins with Kd's ranging from 1 to 100 nM before affinity maturation and from 10 to 1000 pM after affinity maturation (Suderman et al. 2017). Affinity chromatography media produced with nanoCLAMPs support single-step purifications to near homogeneity as assessed by Coomassie staining. The working binding capacity ranges from 5 to 200 nmol target protein per ml of packed beads. While these first generation nanoCLAMP resins have adequate selectivity and capacity for laboratory-scale purifications, they are suboptimal for process-scale purifications because of their moderate thermostability (Tₘ ranging from 45 to 60° C.), sensitivity to protease digestion (t_{1/2}<1 h in 0.1 mg/ml trypsin), and moderate alkaline resistance (50% loss of activity after 12 cycles of incubation with 0.1 M NaOH).

We set out to improve upon the performance of the first generation of nanoCLAMPs in order to improve their general utility for the process-scale purification of targets with AC. Toward this end, we undertook a multi-round protein engineering campaign to improve upon first generation nanoCLAMPs. In each round, we made site-directed mutations at specific positions, evaluated the mutations' impact on thermostability and monodispersity, and then combined beneficial mutations to generate the basis for the next round. The end-product of the 7-round, >180 mutation campaign was a clone with significantly improved performance. We used this clone as the basis for the new “nC-B” class of nanoCLAMPs. Here we report on the campaign to develop the improved nC-B class, the generation and characterization of nC-B nanoCLAMPs against the exemplary protein yeast SUMO (SMT3), and the performance of nC-B nanoCLAMPs after repeated exposure to extreme conditions.

### Results

Approach to Improve nanoCLAMP Performance with a Multi-Round Campaign of Site-Directed Mutagenesis.

We aimed to improve the thermal, proteolytic, and alkaline stability of the first generation of nanoCLAMPs by using consensus protein design, an approach for improving the thermostability of proteins. Our initial attempts of directly synthesizing several versions of the consensus sequence were unsuccessful and yielded only aggregated or multimeric proteins. Therefore, we decided to take an incremental approach of making single mutations, determining their effect alone and in combination, and working towards an improved protein in several rounds. We focused the effort on surface residues and loops and generally sought to remove lysine, arginine and asparagine where possible. The intent of making substitutions for lysine and arginine was to reduce the number of potential sites for cleavage by trypsin. The intent of making substitutions for asparagine was to increase the protein's resistance to the alkaline solutions commonly used to sanitize industrial chromatography columns. Asparagine in certain contexts is susceptible to deamidation, and its removal has been shown to reduce the loss of protein binding activity in sodium hydroxide.

We refer to an individual nanoCLAMP as a “clone,” i.e., a specific isolate with unique binding loops. Our starting clone consisted of an anti-SMT3 nanoCLAMP, SMT3-A1 (Suderman et al. supra), which is capable of purifying SUMO-fusion proteins in a single step from complex lysates. The target of SMT3-A1 is a SUMO tag, which is widely used to improve the solubility and yield of proteins produced in E. coli, and can be cleaved to leave behind a native sequence. SMT3-A1 consists of residues 807 to 946 of the 2^{nd} Type 32 carbohydrate binding module of NagH from Clostridium perfringens with loops mutated in positions 817-820, 838-844, and 931-935 and selected for binding to yeast SUMO. Throughout the paper we number nanoCLAMP amino acids based on the sequence of NagH. To identify evolutionarily conserved amino acids, we generated a multiple sequence alignment for 20 non-redundant BLAST hits selected to cover a range of similarity. The percent identity of the orthologs ranged from 58% for Clostridium nigeriense to 43% for Coprobacillus sp. AF21-8LB (Table 1).

TABLE 1
Orthologous sequences used for consensus-based design

| | Percent identity to C. perfringens |
|---|---|
| Species of origin | CBM32-2 residues 808-943 |
| Clostridium nigeriense | 58% |
| Clostridium tertium | 52% |
| Clostridium sp. C8 | 50% |
| Clostridium sp. 7_2_43FAA | 53% |
| Clostridium chauvoei | 50% |
| Clostridium paraputrificum | 52% |
| Clostridium celatum | 51% |
| Clostridium multispecies | 51% |
| Clostridium septicum | 47% |
| Clostridium spiroforme | 45% |
| Erysipelatoclostridium ramosum | 46% |
| Bacillus mesonae | 44% |
| Tyzzerella sp. An114 | 44% |
| Bacillus sp. B-jedd | 42% |
| Bacillus niacini | 41% |
| Bacillus mesonae | 40% |
| Sporosarcina sp. ZBG7A | 39% |
| Sporosarcina sp. D27 | 40% |
| Lysinibacillus sphaericus | 40% |
| Coprobacillus sp. AF21-8LB | 43% |

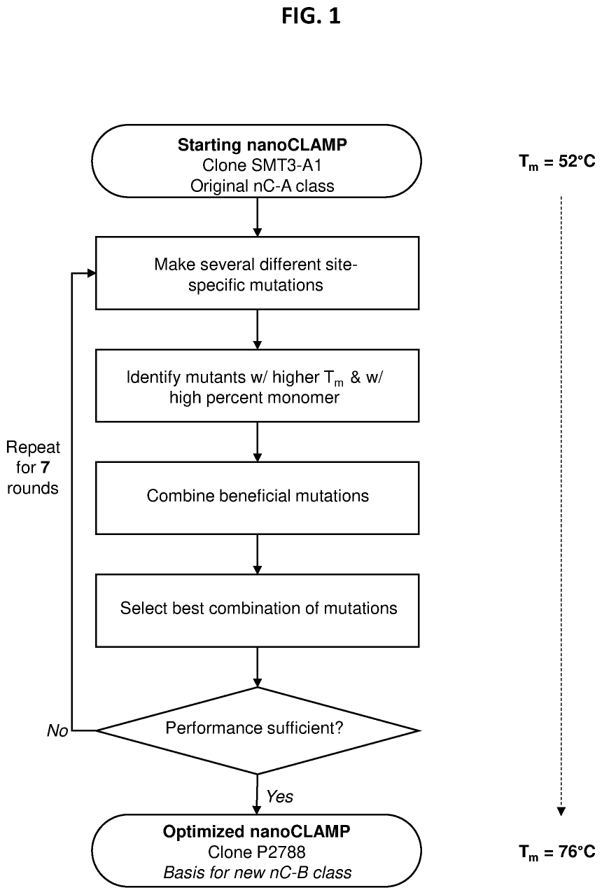

Our workflow for the protein engineering campaign is summarized in FIG. 1. We made site-specific mutations and then purified each individual mutant proteins for further biophysical assessment. For the initial assessment, we measured the melting temperature of the resulting mutants using differential scanning fluorimetry (DSF). We then assessed two parameters, melting temperature and initial fluorescence. The rationale for including low initial fluorescence as a criterion for progression is that we previously observed a rough correlation between high initial fluorescence and the presence of soluble aggregates and multimers detected by size exclusion chromatography. Initial fluorescence in DSF may be caused by the binding of fluorophore to hydrophobic patches exposed prior to unfolding.

After identifying beneficial mutations, we made several constructs with different combinations of the singly beneficial mutations, whose effects were usually, but not always, additive. As a secondary screen, we confirmed that the starting clone for each subsequent round of mutagenesis was monodisperse by size exclusion chromatography.

Optimized Protein Resulting from the Mutagenesis Campaign.

The results of 7 rounds of mutagenesis and evaluation are summarized in Table 2. The full list of mutations is listed in Table 3.

TABLE 2
Summary of mutations to improve nanoCLAMP performance

| | ID | | | | | | | |
|---|---|---|---|---|---|---|---|---|
| | number of | Total | | | | | | |
| | clone | mutations | | | | | | |
| | resulting | added in | Specific | | | | | |
| | from | round | mutations | Lysines | Arginines | Asparagines | | Variants |
| Round | round | (No.) | added in round | (No.) | (No.) | (No.) | Tₘ | Tested |
| Start | SMT3-A1 | | N/A | 10 | 3 | 8 | 52° C. | |
| 1 | P2186 | 8 | K857E, E858V, | 6 | 3 | 8 | 65° C. | 31 |
| | | | I859V, K860E, | | | | | |
| | | | L861V, D862G, | | | | | |
| | | | K870A, K922Q | | | | | |
| 2 | P2387 | 5 | R812H, R865H, | 4 | 2 | 7 | 63° C. | 28 |
| | | | N871D, K897R, | | | | | |
| | | | K908Q | | | | | |
| 3 | P2424 | 7 | N832S, K881R, | 1 | 6 | 6 | 70° C. | 33 |
| | | | K883R, K901R, | | | | | |
| | | | E912D, S914D, | | | | | |
| | | | Q922R | | | | | |
| 4 | P2550 | 7 | S809T, H812R, | 1 | 5 | 5 | 71° C. | 53 |
| | | | S813T, E814P, | | | | | |
| | | | S815G, N890G, | | | | | |
| | | | R922Q | | | | | |
| 5 | P2739 | 3 | R812H, R901H, | 1 | 4 | 5 | 74° C. | 10 |
| | | | D818V | | | | | |
| 6 | P2760 | 1 | W836E | 1 | 4 | 5 | 76° C. | 2 |
| 7 | P2788 | 4 | N807D, N822S, | 1 | 4 | 1 | 76° C. | 27 |
| | | | N825D, N880T | | | | | |
| Net | | 30 | N807D, S809T, | 9 | 1 | 7 | +22° C. | 184 |
| change | | added | R812H, S813T, | removed | added | removed | | tested |
| | | | E814P, S815G, | | | | | |
| | | | D818V, N822S, | | | | | |
| | | | N825D, N832S, | | | | | |
| | | | W836E, K857E, | | | | | |
| | | | E858V, I859V, | | | | | |
| | | | K860E, L861V, | | | | | |
| | | | D862G, R865H, | | | | | |
| | | | K870A, N871D, | | | | | |
| | | | N880T, K881R, | | | | | |
| | | | K883R, N890G, | | | | | |
| | | | K897R, K901H, | | | | | |
| | | | K908Q, E912D, | | | | | |
| | | | S914D, K922Q | | | | | |

TABLE 3
Summary of mutations and resulting biophysical properties in each round of mutagenesis

| | | | | | | Dimer | |
|---|---|---|---|---|---|---|---|
| | | | | | | SEC | |
| | | | | | Aggregated | % | Monomer |
| | | | Tm | | SEC % (7-9 | (9-11 | SEC % |
| Clone | Round | Mutations | (C.) | IF* | mL) | mL) | (12-15 ml) |
| SMT3- | 1 | | 52 | L | 0 | 15 | 85 |
| A1 | | | | | | | |
| P2100 | 1 | K857S, E858V, K860P, L861V, | 58 | L | | | |
| | | D862G | | | | | |
| P2101 | 1 | K857E, E858V, I859V, K860E, | 60 | L | | | |
| | | L861V, D862G | | | | | |
| P2102 | 1 | K857E, E858V, I859T, K860S, | 61 | L | | | |
| | | L861V, D862G | | | | | |
| P2103 | 1 | K870R | 54 | L | | | |
| P2104 | 1 | K870A | 57 | L | | | |
| P2105 | 1 | K870R, G873S, S875Δ, S876Δ | 51 | H | | | |
| P2106 | 1 | K870Δ, N871Δ, G872A, G874D, | 57 | H | | | |
| | | S876Δ | | | | | |
| P2107 | 1 | D877E, K878N | 51 | M | | | |
| P2108 | 1 | K881S | 49 | M | | | |
| P2109 | 1 | K881N | 48 | L | | | |
| P2110 | 1 | K883Q | 48 | L | | | |
| P2111 | 1 | K883C | 50 | H | | | |
| P2112 | 1 | K897R | 53 | L | | | |
| P2113 | 1 | K897H | 49 | L | | | |
| P2114 | 1 | K897Q | 49 | L | | | |
| P2115 | 1 | K897N | 50 | L | | | |
| P2116 | 1 | D900E, K901G, T902S, G903Q, | 57 | H | | | |
| | | A904S, P905G, A906S, G907Δ, | | | | | |
| | | K908Δ | | | | | |
| P2118 | 1 | K922R | 62 | M | | | |
| P2119 | 1 | K922Q | 55 | L | | | |
| P2120 | 1 | R812H, S815A | 53 | L | | | |
| P2121 | 1 | R865H | 52 | L | | | |
| P2122 | 1 | R865Y | 49 | L | | | |
| P2124 | 1 | E913A | 47 | L | | | |
| P2125 | 1 | E930V | 47 | L | | | |
| P2126 | 1 | N832T, T833G, G834D | 54 | H | | | |
| P2184 | 1 | K857E, E858V, I859V, K860E, | 64 | L | | | |
| | combo | L861V, D862G, K870A | | | | | |
| P2185 | 1 | K857E, E858V, I859V, K860E, | 71 | M | | | |
| | combo | L861V, D862G, K870A, K922R | | | | | |
| P2186 | 1 | K857E, E858V, I859V, K860E, | 65 | L | | | |
| | combo | L861V, D862G, K870A, K922Q | | | | | |
| P2187 | 1 | K857E, E858V, I859T, K860S, | 65 | L | | | |
| | combo | L861V, D862G, K870A | | | | | |
| P2188 | 1 | K857E, E858V, I859T, K860S, | 69 | M | | | |
| | combo | L861V, D862G, K870A, K922R | | | | | |
| P2189 | 1 | K857E, E858V, I859T, K860S, | 64 | L | | | |
| | combo | L861V, D862G, K870A, K922Q | | | | | |
| P2338 | 2 | K857E, E858V, I859V, K860E, | 66 | L | | | |
| | | L861V, D862G, K870A, K922Q, | | | | | |
| | | 947E, 948L, 949E | | | | | |
| P2340 | 2 | K857E, E858V, I859V, K860E, | N | | | | |
| | | L861V, D862G, K870A, K922Q | | | | | |
| P2341 | 2 | K857E, E858V, I859V, K860E, | 65 | L | | | |
| | | L861V, D862G, K870A, K922Q | | | | | |
| P2342 | 2 | K857E, E858V, I859V, K860E, | 60 | H | | | |
| | | L861V, D862G, K870A, D877A, | | | | | |
| | | K922Q | | | | | |
| P2343 | 2 | K857E, E858V, I859V, K860E, | 57 | H | | | |
| | | L861V, D862G, K870A, D877E, | | | | | |
| | | K922Q | | | | | |
| P2344 | 2 | K857E, E858V, I859V, K860E, | N | H | | | |
| | | L861V, D862G, K870A, K922Q | | | | | |
| P2345 | 2 | K857E, E858V, I859V, K860E, | 55 | L | | | |
| | | L861V, D862G, K870A, E913A, | | | | | |
| | | K922Q | | | | | |
| P2346 | 2 | K857E, E858V, I859V, K860E, | 58 | L | | | |
| | | L861V, D862G, K870A, K922Q, | | | | | |
| | | E930A | | | | | |
| P2347 | 2 | K857E, E858V, I859V, K860E, | 56 | L | | | |
| | | L861V, D862G, K870A, K922Q, | | | | | |
| | | E930V | | | | | |
| P2348 | 2 | R812H, K857E, E858V, I859V, | 64 | L | | | |
| | | K860E, L861V, D862G, K870A, | | | | | |
| | | K922Q | | | | | |
| P2349 | 2 | R812V, K857E, E858V, I859V, | 59 | H | | | |
| | | K860E, L861V, D862G, K870A, | | | | | |
| | | K922Q | | | | | |
| P2350 | 2 | R812Q, K857E, E858V, I859V, | 59 | H | | | |
| | | K860E, L861V, D862G, K870A, | | | | | |
| | | K922Q | | | | | |
| P2351 | 2 | R812A, K857E, E858V, I859V, | N | | | | |
| | | K860E, L861V, D862G, K870A, | | | | | |
| | | K922Q | | | | | |
| P2352 | 2 | K857E, E858V, I859V, K860E, | 63 | L | | | |
| | | L861V, D862G, R865H, K870A, | | | | | |
| | | K922Q | | | | | |
| P2353 | 2 | K857E, E858V, I859V, K860E, | 58 | H | | | |
| | | L861V, D862G, R865S, K870A, | | | | | |
| | | K922Q | | | | | |
| P2354 | 2 | K857E, E858V, I859V, K860E, | 57 | H | | | |
| | | L861V, D862G, R865Q, K870A, | | | | | |
| | | K922Q | | | | | |
| P2355 | 2 | K857E, E858V, I859V, K860E, | 55 | M | | | |
| | | L861V, D862G, R865A, K870A, | | | | | |
| | | K922Q | | | | | |
| P2356 | 2 | K857E, E858V, I859V, K860E, | 64 | H | | | |
| | | L861V, D862G, K870A, D900Δ, | | | | | |
| | | K901Δ, A904K, P905D, A906S, | | | | | |
| | | K908Q, K922Q | | | | | |
| P2357 | 2 | K857E, E858V, I859V, K860E, | 63 | H | | | |
| | | L861V, D862G, K870A, D900Δ, | | | | | |
| | | K901Δ, P905D, A906S, K908Q, | | | | | |
| | | K922Q | | | | | |
| P2358 | 2 | K857E, E858V, I859V, K860E, | 62 | H | | | |
| | | L861V, D862G, K870A, D900Δ, | | | | | |
| | | K901Δ, A904Q, P905D, A906S, | | | | | |
| | | K908Q, K922Q | | | | | |
| P2359 | 2 | K857E, E858V, I859V, K860E, | 61 | H | | | |
| | | L861V, D862G, K870A, D900Δ, | | | | | |
| | | K901Δ, A904Q, P905A, A906S, | | | | | |
| | | K908Q, K922Q | | | | | |
| P2360 | 2 | K857E, E858V, I859V, K860E, | 65 | H | | | |
| | | L861V, D862G, K870A, D900Δ, | | | | | |
| | | K901Δ, T902E, A904V, P905D, | | | | | |
| | | A906S, K908Q, K922Q | | | | | |
| P2361 | 2 | K857E, E858V, I859V, K860E, | N | H | | | |
| | | L861V, D862G, K870A, D900Δ, | | | | | |
| | | K901Δ, T902E, A904V, P905T, | | | | | |
| | | A906N, K908Q, K922Q | | | | | |
| P2362 | 2 | K857E, E858V, I859V, K860E, | 61 | H | | | |
| | | L861V, D862G, K870A, D900Δ, | | | | | |
| | | K901Δ, T902Q, A904V, P905S, | | | | | |
| | | A906S, K908Q, K922Q | | | | | |
| P2363 | 2 | K857E, E858V, I859V, K860E, | 65 | H | | | |
| | | L861V, D862G, K870A, D900Δ, | | | | | |
| | | K901Δ, T902Δ, G903N, A904S, | | | | | |
| | | P905T, K908Q, K922Q | | | | | |
| P2385 | 2 | R812H, K857E, E858V, I859V, | 64 | L | | | |
| | combo | K860E, L861V, D862G, R865H, | | | | | |
| | | K870A, N871D, K897R, K922Q, | | | | | |
| | | 947E, 948L, 949E | | | | | |
| P2386 | 2 | R812H, K857E, E858V, I859V, | 56 | L | | | |
| | combo | K860E, L861V, D862G, R865H, | | | | | |
| | | K870A, N871D, E885Q, K897R, | | | | | |
| | | K922Q, 947E, 948L, 949E | | | | | |
| P2387 | 2 | R812H, K857E, E858V, I859V, | 63 | L | | | |
| | combo | K860E, L861V, D862G, R865H, | | | | | |
| | | K870A, N871D, K897R, K908Q, | | | | | |
| | | K922Q,947E, 948L, 949E | | | | | |
| P2388 | 2 | R812H, K857E, E858V, I859V, | 54 | L | | | |
| | combo | K860E, L861V, D862G, R865H, | | | | | |
| | | K870A, N871D, E885Q, K897R, | | | | | |
| | | K908Q, K922Q, 947E, 948L, | | | | | |
| | | 949E | | | | | |
| P2390 | 3 | R812H, N832S, K857E, E858V, | 62 | L | | | |
| | | I859V, K860E, L861V, D862G, | | | | | |
| | | R865H, K870A, N871D, K897R, | | | | | |
| | | K908Q, K922Q, 947E, 948L, | | | | | |
| | | 949E | | | | | |
| P2391 | 3 | R812H, K857E, E858V, I859V, | 62 | L | | | |
| | | K860E, L861V, D862G, R865H, | | | | | |
| | | K870A, N871D, K897R, K908Q, | | | | | |
| | | S914D, K922Q, 947E, 948L, | | | | | |
| | | 949E | | | | | |
| P2392 | 3 | R812H, K857E, E858V, I859V, | 64 | L | | | |
| | | K860E, L861V, D862G, R865H, | | | | | |
| | | K870A, N871D, K897R, K908Q, | | | | | |
| | | E912D, K922Q, 947E, 948L, | | | | | |
| | | 949E | | | | | |
| P2393 | 3 | R812H, K857E, E858V, I859V, | 40, 60 | H | | | |
| | | K860E, L861V, D862G, R865H, | | | | | |
| | | K870A, N871D, K878Y, K897R, | | | | | |
| | | K908Q, K922Q, 947E, 948L, | | | | | |
| | | 949E | | | | | |
| P2394 | 3 | R812H, K857E, E858V, I859V, | 58 | H | | | |
| | | K860E, L861V, D862G, R865H, | | | | | |
| | | K870A, N871D, K878R, K897R, | | | | | |
| | | K908Q, K922Q, 947E, 948L, | | | | | |
| | | 949E | | | | | |
| P2395 | 3 | R812H, K857E, E858V, I859V, | 63 | H | | | |
| | | K860E, L861V, D862G, R865H, | | | | | |
| | | K870A, N871D, K878H, K897R, | | | | | |
| | | K908Q, K922Q, 947E, 948L, | | | | | |
| | | 949E | | | | | |
| P2396 | 3 | R812H, K857E, E858V, I859V, | N | | | | |
| | | K860E, L861V, D862G, R865H, | | | | | |
| | | K870A, N871D, K878V, K897R, | | | | | |
| | | K908Q, K922Q, 947E, 948L, | | | | | |
| | | 949E | | | | | |
| P2397 | 3 | R812H, K857E, E858V, I859V, | N | H | | | |
| | | K860E, L861V, D862G, R865H, | | | | | |
| | | K870A, N871D, K878N, K897R, | | | | | |
| | | K908Q, K922Q, 947E, 948L, | | | | | |
| | | 949E | | | | | |
| P2398 | 3 | R812H, K857E, E858V, I859V, | 59 | H | | | |
| | | K860E, L861V, D862G, R865H, | | | | | |
| | | K870A, N871D, K878Q, K897R, | | | | | |
| | | K908Q, K922Q, 947E, 948L, | | | | | |
| | | 949E | | | | | |
| P2399 | 3 | R812H, K857E, E858V, I859V, | 58 | H | | | |
| | | K860E, L861V, D862G, R865H, | | | | | |
| | | K870A, N871D, K878L, K897R, | | | | | |
| | | K908Q, K922Q, 947E, 948L, | | | | | |
| | | 949E | | | | | |
| P2400 | 3 | R812H, K857E, E858V, I859V, | 40, 61 | H | | | |
| | | K860E, L861V, D862G, R865H, | | | | | |
| | | K870A, N871D, K878M, K897R, | | | | | |
| | | K908Q, K922Q, 947E, 948L, | | | | | |
| | | 949E | | | | | |
| P2401 | 3 | R812H, K857E, E858V, I859V, | 65 | L | | | |
| | | K860E, L861V, D862G, R865H, | | | | | |
| | | K870A, N871D, K897R, K901R, | | | | | |
| | | K908Q, K922Q, 947E, 948L, | | | | | |
| | | 949E | | | | | |
| P2402 | 3 | R812H, K857E, E858V, I859V, | 62 | L | | | |
| | | K860E, L861V, D862G, R865H, | | | | | |
| | | K870A, N871D, K897R, K901H, | | | | | |
| | | K908Q, K922Q, 947E, 948L, | | | | | |
| | | 949E | | | | | |
| P2403 | 3 | R812H, K857E, E858V, I859V, | 56 | L | | | |
| | | K860E, L861V, D862G, R865H, | | | | | |
| | | K870A, N871D, K897R, K901G, | | | | | |
| | | K908Q, K922Q, 947E, 948L, | | | | | |
| | | 949E | | | | | |
| P2404 | 3 | R812H, K857E, E858V, I859V, | 47 | H | | | |
| | | K860E, L861V, D862G, R865H, | | | | | |
| | | K870A, N871D, K897R, K901S, | | | | | |
| | | K908Q, K922Q, 947E, 948L, | | | | | |
| | | 949E | | | | | |
| P2405 | 3 | R812H, K857E, E858V, I859V, | 59 | L | | | |
| | | K860E, L861V, D862G, R865H, | | | | | |
| | | K870A, N871D, K897R, K901Q, | | | | | |
| | | K908Q, K922Q, 947E, 948L, | | | | | |
| | | 949E | | | | | |
| P2406 | 3 | R812H, K857E, E858V, I859V, | 64 | L | | | |
| | | K860E, L861V, D862G, R865H, | | | | | |
| | | K870A, N871D, K881R, K883R, | | | | | |
| | | K897R, K908Q, K922Q, 947E, | | | | | |
| | | 948L, 949E | | | | | |
| P2407 | 3 | R812H, K857E, E858V, I859V, | 40, 58 | H | | | |
| | | K860E, L861V, D862G, R865H, | | | | | |
| | | K870A, N871D, N880T, K881R, | | | | | |
| | | F882Y, K883R, K897R, K908Q, | | | | | |
| | | K922Q, 947E, 948L, 949E | | | | | |
| P2408 | 3 | R812H, K857E, E858V, I859V, | 52 | L | | | |
| | | K860E, L861V, D862G, R865H, | | | | | |
| | | K870A, N871D, N880T, K881R, | | | | | |
| | | F882Y, K883S, K897R, K908Q, | | | | | |
| | | K922Q, 947E, 948L, 949E | | | | | |
| P2409 | 3 | R812H, K857E, E858V, I859V, | 48 | L | | | |
| | | K860E, L861V, D862G, R865H, | | | | | |
| | | K870A, N871D, N880T, K881R, | | | | | |
| | | F882Y, K883H, K897R, K908Q, | | | | | |
| | | K922Q, 947E, 948L, 949E | | | | | |
| P2410 | 3 | R812H, K857E, E858V, I859V, | 59 | H | | | |
| | | K860E, L861V, D862G, R865H, | | | | | |
| | | K870A, N871D, N880T, K881H, | | | | | |
| | | F882Y, K883R, K897R, K908Q, | | | | | |
| | | K922Q, 947E, 948L, 949E | | | | | |
| P2411 | 3 | R812H, K857E, E858V, I859V, | 49 | L | | | |
| | | K860E, L861V, D862G, R865H, | | | | | |
| | | K870A, N871D, N880T, K881H, | | | | | |
| | | F882Y, K883H, K897R, K908Q, | | | | | |
| | | K922Q, 947E, 948L, 949E | | | | | |
| P2412 | 3 | R812H, K857E, E858V, I859V, | 50 | L | | | |
| | | K860E, L861V, D862G, R865H, | | | | | |
| | | K870A, N871D, N880T, K881N, | | | | | |
| | | F882Y, K883R, K897R, K908Q, | | | | | |
| | | K922Q, E930R, 947E, 948L, | | | | | |
| | | 949E | | | | | |
| P2413 | 3 | R812H, K857E, E858V, I859V, | 45 | L | | | |
| | | K860E, L861V, D862G, R865H, | | | | | |
| | | K870A, N871D, N880T, K881E, | | | | | |
| | | F882Y, K883Q, K897R, K908Q, | | | | | |
| | | K922Q, E930R, 947E, 948L, | | | | | |
| | | 949E | | | | | |
| P2414 | 3 | R812H, K857E, E858V, I859V, | 40 | L | | | |
| | | K860E, L861V, D862G, R865H, | | | | | |
| | | K870A, N871D, N880S, K881N, | | | | | |
| | | F882Y, K883D, K897R, K908Q, | | | | | |
| | | K922Q, E930R, 947E, 948L, | | | | | |
| | | 949E | | | | | |
| P2415 | 3 | R812H, K857E, E858V, I859V, | 50 | L | | | |
| | | K860E, L861V, D862G, R865H, | | | | | |
| | | K870A, N871D, K897R, K908Q, | | | | | |
| | | K922Q, R925H, 947E, 948L, | | | | | |
| | | 949E | | | | | |
| P2416 | 3 | R812H, K857E, E858V, I859V, | 43 | L | | | |
| | | K860E, L861V, D862G, R865H, | | | | | |
| | | K870A, N871D, K897R, K908Q, | | | | | |
| | | K922Q, R925A, 947E, 948L, | | | | | |
| | | 949E | | | | | |
| P2417 | 3 | R812H, K857E, E858V, I859V, | 50 | L | | | |
| | | K860E, L861V, D862G, R865H, | | | | | |
| | | K870A, N871D, E885Q, K897R, | | | | | |
| | | K908Q, K922Q, R925A, 947E, | | | | | |
| | | 948L, 949E | | | | | |
| P2418 | 3 | R812H, K857E, E858V, I859V, | 34, 54 | H | | | |
| | | K860E, L861V, D862G, R865H, | | | | | |
| | | K870A, N871D, E885Q, K897R, | | | | | |
| | | K908Q, K922Q, R925Y, 947E, | | | | | |
| | | 948L, 949E | | | | | |
| P2419 | 3 | R812H, K857E, E858V, I859V, | 34, 56 | H | | | |
| | | K860E, L861V, D862G, R865H, | | | | | |
| | | K870A, N871D, E885Q, K897R, | | | | | |
| | | K908Q, K922Q, R925F, 947E, | | | | | |
| | | 948L, 949E | | | | | |
| P2420 | 3 | R812H, K857E, E858V, I859V, | 47 | L | | | |
| | | K860E, L861V, D862G, R865H, | | | | | |
| | | K870A, N871D, E885A, K897R, | | | | | |
| | | K908Q, K922Q, R925A, 947E, | | | | | |
| | | 948L, 949E, 947E, 948L, 949E | | | | | |
| P2423 | 3 | R812H, N832S, K857E, E858V, | 62 | L | | | |
| | combo | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922Q, 947E, | | | | | |
| | | 948L, 949E | | | | | |
| P2424 | 3 | R812H, N832S, K857E, E858V, | 70 | L | | | |
| | combo | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, 947E, | | | | | |
| | | 948L, 949E | | | | | |
| P2425 | 4 | R812H, N832S, K857E, E858V, | 59 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, F915L, E916S, | | | | | |
| | | T917G, P918V, 1919Q, S920Δ, | | | | | |
| | | K922R, 947E, 948L, 949E | | | | | |
| P2426 | 4 | R812H, N832S, K857E, E858V, | 58 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, E916S, T917G, | | | | | |
| | | P918V, 1919Q, S920Δ, K922R, | | | | | |
| | | 947E, 948L, 949E | | | | | |
| P2427 | 4 | R812H, N832S, K857E, E858V, | 56 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, F915Δ, E916L, | | | | | |
| | | T917H, P918G, 1919A, S920Q, | | | | | |
| | | K922R, 947E, 948L, 949E | | | | | |
| P2428 | 4 | R812H, N832S, K857E, E858V, | 52 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, E916H, T917G, | | | | | |
| | | P918A, 1919Q, S920Δ, K922R, | | | | | |
| | | 947E, 948L, 949E | | | | | |
| P2429 | 4 | R812H, N832S, K857E, E858V, | 58 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, E916Δ, T917G, | | | | | |
| | | P918G, S920E, K922R, 947E, | | | | | |
| | | 948L, 949E | | | | | |
| P2430 | 4 | R812H, N832S, K857E, E858V, | 58 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, F915L, E916G, | | | | | |
| | | T917G, P9181, 1919E, S920A, | | | | | |
| | | A921Q, K922Δ, 947E, 948L, | | | | | |
| | | 949E | | | | | |
| P2431 | 4 | R812H, N832S, L846V, K857E, | 70 | L | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2432 | 4 | R812H, N832S, E849D, K857E, | N | | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2433 | 4 | R812H, N832S, F850Y, K857E, | 72 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2434 | 4 | R812H, N832S, L853V, K857E, | 73 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2435 | 4 | R812H, N832S, K857E, E858V, | 63 | M | | | |
| | | I859V, K860E, L861V, D862G, | | | | | |
| | | G863N, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2436 | 4 | R812H, N832S, K857E, E858V, | 57 | L | | | |
| | | I859V, K860E, L861V, D862G, | | | | | |
| | | G863D, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2437 | 4 | R812H, N832S, K857E, E858V, | 68 | L | | | |
| | | I859V, K860E, L861V, D862G, | | | | | |
| | | I864V, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2438 | 4 | R812H, N832S, K857E, E858V, | 71 | M | | | |
| | | I859V, K860E, L861V, D862G, | | | | | |
| | | R865H, I868V, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2439 | 4 | R812H, N832S, K857E, E858V, | 72 | M | | | |
| | | I859V, K860E, L861V, D862G, | | | | | |
| | | R865H, K870A, N871D, K881R, | | | | | |
| | | K883R, L888T, K897R, K901R, | | | | | |
| | | K908Q, E912D, S914D, K922R, | | | | | |
| | | 947E, 948L, 949E | | | | | |
| P2440 | 4 | R812H, N832S, K857E, E858V, | 68 | L | | | |
| | | I859V, K860E, L861V, D862G, | | | | | |
| | | R865H, K870A, N871D, K881R, | | | | | |
| | | K883R, E891S, K897R, K901R, | | | | | |
| | | K908Q, E912D, S914D, K922R, | | | | | |
| | | 947E, 948L, 949E | | | | | |
| P2441 | 4 | R812H, N832S, K857E, E858V, | 73 | H | | | |
| | | I859V, K860E, L861V, D862G, | | | | | |
| | | R865H, K870A, N871D, K881R, | | | | | |
| | | K883R, S892N, K897R, K901R, | | | | | |
| | | K908Q, E912D, S914D, K922R, | | | | | |
| | | 947E, 948L, 949E | | | | | |
| P2442 | 4 | R812H, N832S, K857E, E858V, | 64 | L | | | |
| | | I859V, K860E, L861V, D862G, | | | | | |
| | | R865H, K870A, N871D, K881R, | | | | | |
| | | K883R, I896F, K897R, K901R, | | | | | |
| | | K908Q, E912D, S914D, K922R, | | | | | |
| | | 947E, 948L, 949E | | | | | |
| P2443 | 4 | R812H, N832S, K857E, E858V, | 59 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, 1924V, | | | | | |
| | | 947E, 948L, 949E | | | | | |
| P2444 | 4 | R812H, N832S, K857E, E858V, | 69 | H | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, T9371, | | | | | |
| | | 947E, 948L, 949E | | | | | |
| P2445 | 4 | R812H, N832S, K857E, E858V, | 70 | H | | | |
| | | I859V, K860E, L861V, D862G, | | | | | |
| | | R865H, K870A, N871E, K881R, | | | | | |
| | | K883R, K897R, K901R, K908Q, | | | | | |
| | | E912D, S914D, K922R, 947E, | | | | | |
| | | 948L, 949E | | | | | |
| P2446 | 4 | R812H, N832S, K857E, E858V, | 74 | H | | | |
| | | I859V, K860E, L861V, D862G, | | | | | |
| | | R865H, K870A, N871Q, K881R, | | | | | |
| | | K883R, K897R, K901R, K908Q, | | | | | |
| | | E912D, S914D, K922R, 947E, | | | | | |
| | | 948L, 949E | | | | | |
| P2447 | 4 | R812H, N832S, K857E, E858V, | 68 | H | | | |
| | | I859V, K860E, L861V, D862G, | | | | | |
| | | R865H, K870A, N871S, K881R, | | | | | |
| | | K883R, K897R, K901R, K908Q, | | | | | |
| | | E912D, S914D, K922R, 947E, | | | | | |
| | | 948L, 949E | | | | | |
| P2448 | 4 | R812H, N832S, K857E, E858V, | 71 | M | | | |
| | | I859V, K860E, L861V, D862G, | | | | | |
| | | R865H, K870A, N871D, G872A, | | | | | |
| | | K881R, K883R, K897R, K901R, | | | | | |
| | | K908Q, E912D, S914D, K922R, | | | | | |
| | | 947E, 948L, 949E | | | | | |
| P2449 | 4 | R812H, G829D, N832S, K857E, | 71 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2450 | 4 | R812H, G829N, N832S, K857E, | 70 | M | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2451 | 4 | R812H, N832S, W836E, Y837F, | 74 | H | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2452 | 4 | R812H, N832S, W836V, Y837F, | 73 | M | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2453 | 4 | R812H, N832S, W836A, Y837F, | 72 | H | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2454 | 4 | R812H, N832S, W836E, | 75 | H | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2455 | 4 | R812H, N832S, W836V, | 74 | M | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2456 | 4 | R812H, N832S, W836A, | 74 | M | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2458 | 4 | R812H, N832S, K857E, E858V, | 64 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, F915L, K922R, | | | | | |
| | | 947E, 948L, 949E | | | | | |
| P2459 | 4 | R812H, N832S, K857E, E858V, | 73 | H | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, F9411, | | | | | |
| | | 947E, 948L, 949E | | | | | |
| P2460 | 4 | R812H, S815H, N832S, K857E, | 70 | M | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2461 | 4 | R812H, S815H, N832S, E849D, | 72 | H | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2462 | 4 | R812H, N832S, G834D, K857E, | 72 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2463 | 4 | R812H, N832S, G834D, K857E, | 40, 72 | H | | | |
| | | E858V, I859V, K860E, L861V, | | | | | |
| | | D862G, R865H, K870R, | | | | | |
| | | N871D, K881R, K883R, K897R, | | | | | |
| | | K901R, K908Q, E912D, S914D, | | | | | |
| | | K922R, 947E, 948L, 949E | | | | | |
| P2464 | 4 | R812H, N832S, K857E, E858V, | 70 | H | | | |
| | | I859V, K860E, L861V, D862G, | | | | | |
| | | R865H, K870A, N871D, K881R, | | | | | |
| | | K883R, L888V, K897R, K901R, | | | | | |
| | | K908Q, E912D, S914D, K922R, | | | | | |
| | | 947E, 948L, 949E | | | | | |
| P2465 | 4 | R812H, N832S, K857E, E858V, | N | H | | | |
| | | I859V, K860E, L861V, D862G, | | | | | |
| | | R865H, K870A, N871D, K881R, | | | | | |
| | | K883R, L888S, K897R, K901R, | | | | | |
| | | K908Q, E912D, S914D, K922R, | | | | | |
| | | 947E, 948L, 949E | | | | | |
| P2466 | 4 | R812H, N832S, K857E, E858V, | 66 | M | | | |
| | | I859V, K860E, L861V, D862G, | | | | | |
| | | R865H, K870A, N871D, K881R, | | | | | |
| | | K883R, L888D, K897R, K901R, | | | | | |
| | | K908Q, E912D, S914D, K922R, | | | | | |
| | | 947E, 948L, 949E | | | | | |
| P2467 | 4 | R812H, N832S, K857E, E858V, | 62 | M | | | |
| | | I859V, K860E, L861V, D862G, | | | | | |
| | | R865H, K870A, N871D, K881R, | | | | | |
| | | K883R, L888H, K897R, K901R, | | | | | |
| | | K908Q, E912D, S914D, K922R, | | | | | |
| | | 947E, 948L, 949E | | | | | |
| P2468 | 4 | R812H, N832S, K857E, E858V, | 69 | M | | | |
| | | I859V, K860E, L861V, D862G, | | | | | |
| | | R865H, K870A, N871D, K881R, | | | | | |
| | | K883R, S892D, K897R, K901R, | | | | | |
| | | K908Q, E912D, S914D, K922R, | | | | | |
| | | 947E, 948L, 949E | | | | | |
| P2469 | 4 | R812H, N832S, K857E, E858V, | 72 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, S920E, K922R, | | | | | |
| | | 947E, 948L, 949E | | | | | |
| P2470 | 4 | R812H, N832S, K857E, E858V, | 69 | L | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, 1943V, | | | | | |
| | | 947E, 948L, 949E | | | | | |
| P2471 | 4 | R812H, N832S, K857E, E858V, | 73 | H | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, V944E, | | | | | |
| | | 947E, 948L, 949E | | | | | |
| P2472 | 4 | R812H, N832S, K857E, E858V, | 70 | H | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, A942R, | | | | | |
| | | V944E, 947E, 948L, 949E | | | | | |
| P2473 | 4 | R812H, N832S, K857E, E858V, | 72 | H | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922R, D946H, | | | | | |
| | | 947E, 948L, 949E | | | | | |
| P2474 | 4 | R812H, N832S, W836F, K857E, | 73 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2475 | 4 | R812H, N832S, W836Y, | 73 | H | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922R, 947E, 948L, 949E | | | | | |
| P2476 | n | R812H, N832S, G834C, K857E, | 87 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | S939C, 947E, 948L, 949E | | | | | |
| P2477 | n | R812H, N832S, K857E, E858V, | 59 | H | | | |
| | | I859V, K860E, L861V, D862G, | | | | | |
| | | R865H, K870A, N871D, K881R, | | | | | |
| | | K883R, I896C, K897R, K901R, | | | | | |
| | | K908Q, E912D, S914D, F915C, | | | | | |
| | | K922R, 947E, 948L, 949E | | | | | |
| P2478 | n | R812H, N832S, F850C, K857E, | 93 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | T927C, 947E, 948L, 949E | | | | | |
| P2479 | n | R812H, N832S, K857E, E858V, | 61 | H | | | |
| | | I859V, K860E, L861V, D862G, | | | | | |
| | | I864C, R865H, K870A, N871D, | | | | | |
| | | K881R, K883R, K897R, K901R, | | | | | |
| | | K908Q, E912D, S914D, K922R, | | | | | |
| | | 1943C, 947E, 948L, 949E | | | | | |
| P2480 | n | R812H, N832S, K857E, E858V, | 71 | H | | | |
| | | I859V, K860E, L861V, D862G, | | | | | |
| | | R865H, V867C, K870A, N871D, | | | | | |
| | | K881R, K883R, K897R, K901R, | | | | | |
| | | K908Q, E912D, S914D, K922R, | | | | | |
| | | S939C, 947E, 948L, 949E | | | | | |
| P2481 | n | R812H, N832S, K857E, E858V, | 58 | H | | | |
| | | I859V, K860E, L861V, D862G, | | | | | |
| | | R865H, K870A, N871D, K881R, | | | | | |
| | | K883R, Y886C, K897R, K901R, | | | | | |
| | | K908Q, E912D, S914D, K922R, | | | | | |
| | | 1924C, 947E, 948L, 949E | | | | | |
| P2482 | 4 | P808A, R812H, N832S, K857E, | 71 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922R, | | | | | |
| | | 947E, 948L, 949E | | | | | |
| P2495 | n | R812H, N832S, K857E, E858V, | 60 | H | | | |
| | | I859V, K860E, L861V, D862G, | | | | | |
| | | R865H, K870A, N871Q, K881R, | | | | | |
| | | K883R, K897R, K901R, K908Q, | | | | | |
| | | E912D, S914D, K922Q, 947E, | | | | | |
| | | 948L, 949E | | | | | |
| P2496 | n | R812H, N832S, W836V, | 63 | M | | | |
| | | K857E, E858V, I859V, K860E, | | | | | |
| | | L861V, D862G, R865H, K870A, | | | | | |
| | | N871D, K881R, K883R, K897R, | | | | | |
| | | K901R, K908Q, E912D, S914D, | | | | | |
| | | K922Q, 947E, 948L, 949E | | | | | |
| P2497 | n | R812H, N832S, G834C, K857E, | 62, 77 | H | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922Q, | | | | | |
| | | S939C, 947E, 948L, 949E | | | | | |
| P2498 | n | R812H, N832S, K857E, E858V, | 61 | H | | | |
| | | I859V, K860E, L861V, D862G, | | | | | |
| | | R865H, K870A, N871D, K881R, | | | | | |
| | | K883R, K897R, K901R, K908Q, | | | | | |
| | | E912D, S914D, K922Q, F9411, | | | | | |
| | | 947E, 948L, 949E | | | | | |
| P2499 | n | P808A, R812H, N832S, K857E, | 60 | M | | | |
| | | E858V, I859V, K860E, L861V, | | | | | |
| | | D862G, R865H, K870A, N871D, | | | | | |
| | | K881R, K883R, K897R, K901R, | | | | | |
| | | K908Q, E912D, S914D, K922Q, | | | | | |
| | | 947E, 948L, 949E | | | | | |
| P2543 | n | E817S, D818V, I819A, K820S, | N | | | | |
| | | F838K, E840H, V841H, F842H, | | | | | |
| | | Y843W, D931A, K932A, 1933P, | | | | | |
| | | L934A, F935H | | | | | |
| P2549 | 4 | S809T, R812H, S813T, E814P, | 71 | L | | | |
| | combo | S815G, N832S, K857E, E858V, | | | | | |
| | | I859V, K860E, L861V, D862G, | | | | | |
| | | R865H, K870A, N871D, K881R, | | | | | |
| | | K883R, N890G, K897R, K901R, | | | | | |
| | | K908Q, E912D, S914D, K922Q, | | | | | |
| | | 947E, 948L, 949E | | | | | |
| P2550 | 4 | S809T, S813T, E814P, S815G, | 78 | L | 0 | 25 | 75 |
| | combo | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901R, | | | | | |
| | | K908Q, E912D, S914D, K922Q, | | | | | |
| | | 947E, 948L, 949E | | | | | |
| P2551 | 5 | S809T, S813T, E814P, S815G, | 83 | M | 10 | 30 | 60 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901R, | | | | | |
| | | K908Q, E912D, S914D, K922R, | | | | | |
| | | 947E, 948L, 949E | | | | | |
| P2719 | 5 | S809T, S813T, E814P, S815G, | 75 | L | 0 | 15 | 85 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2720 | 5 | S809T, S813T, E814P, S815G, | 75 | L | 0 | 20 | 80 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901R, | | | | | |
| | | K908Q, S914D, K922Q, 947E, | | | | | |
| | | 948L, 949E | | | | | |
| P2721 | 5 | S809T, S813T, E814P, S815G, | 77 | M | 2 | 23 | 75 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901R, | | | | | |
| | | K908Q, E912D, K922Q, 947E, | | | | | |
| | | 948L, 949E | | | | | |
| P2722 | 5 | S809T, S813T, E814P, S815G, | 71 | L | 0 | 10 | 90 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901H, | | | | | |
| | | K908Q, K922Q, 947E, 948L, | | | | | |
| | | 949E | | | | | |
| P2723 | 5 | S809T, S813T, E814P, S815G, | 76 | M | 0 | 30 | 60 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901R, | | | | | |
| | | K908Q, K922Q, 947E, 948L, | | | | | |
| | | 949E | | | | | |
| P2724 | 5 | S809T, S813T, E814P, S815G, | 76 | L | 0 | 15 | 85 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901H, | | | | | |
| | | K908Q, E912D, K922Q, 947E, | | | | | |
| | | 948L, 949E | | | | | |
| P2733 | n | S809T, R812H, S813T, E814P, | 62 | L | 0 | 50 | 50 |
| | | S815G, E817D, D818V, I819N, | | | | | |
| | | K820H, N832S, F838T, N839L, | | | | | |
| | | E8401, V841T, F842D, Y843T, | | | | | |
| | | E844Q, K857E, E858V, I859V, | | | | | |
| | | K860E, L861V, D862G, R865N, | | | | | |
| | | K870A, N871D, K881R, K883R, | | | | | |
| | | N890G, K897R, K901R, K908Q, | | | | | |
| | | E912D, S914D, P918R, K922Q, | | | | | |
| | | D931F, K932M, 1933A, L934P, | | | | | |
| | | F935M, 947E, 948L, 949E | | | | | |
| P2734 | n | S809T, S813T, E814P, S815G, | 58 | L | 5 | 45 | 50 |
| | | E817D, D818V, I819N, K820H, | | | | | |
| | | N832S, F838T, N839L, E8401, | | | | | |
| | | V841T, F842D, Y843T, E844Q, | | | | | |
| | | K857E, E858V, I859V, K860E, | | | | | |
| | | L861V, D862G, R865N, K870A, | | | | | |
| | | N871D, K881R, K883R, N890G, | | | | | |
| | | K897R, K901H, K908Q, E912D, | | | | | |
| | | S914D, P918R, K922Q, D931F, | | | | | |
| | | K932M, 1933A, L934P, F935M, | | | | | |
| | | 947E, 948L, 949E | | | | | |
| P2735 | n | S809T, R812H, S813T, E814P, | 57 | | 5 | 50 | 45 |
| | | S815G, E817D, D818V, I819N, | | | | | |
| | | K820H, N832S, F838T, N839L, | | | | | |
| | | E8401, V841T, F842D, Y843T, | | | | | |
| | | E844Q, K857E, E858V, I859V, | | | | | |
| | | K860E, L861V, D862G, R865N, | | | | | |
| | | K870A, N871D, K881R, K883R, | | | | | |
| | | N890G, K897R, K901H, K908Q, | | | | | |
| | | E912D, S914D, P918R, K922Q, | | | | | |
| | | D931F, K932M, 1933A, L934P, | | | | | |
| | | F935M, 947E, 948L, 949E | | | | | |
| P2736 | n | S809T, R812H, S813T, E814P, | 57 | L | 5 | 45 | 50 |
| | | S815G, E817D, D818V, I819N, | | | | | |
| | | K820H, N832S, F838T, N839L, | | | | | |
| | | E8401, V841T, F842D, Y843T, | | | | | |
| | | E844Q, K857E, E858V, I859V, | | | | | |
| | | K860E, L861V, D862G, R865N, | | | | | |
| | | K870A, N871D, N890G, K897R, | | | | | |
| | | K901H, K908Q, E912D, S914D, | | | | | |
| | | P918R, K922Q, D931F, K932M, | | | | | |
| | | 1933A, L934P, F935M, 947E, | | | | | |
| | | 948L, 949E | | | | | |
| P2737 | 5 | S809T, R812H, S813T, E814P, | 75 | L | 0 | 15 | 85 |
| | | S815G, D818V, N832S, K857E, | | | | | |
| | | E858V, I859V, K860E, L861V, | | | | | |
| | | D862G, R865H, K870A, N871D, | | | | | |
| | | K881R, K883R, N890G, K897R, | | | | | |
| | | K901R, K908Q, E912D, S914D, | | | | | |
| | | K922Q, 947E, 948L, 949E | | | | | |
| P2738 | 5 | S809T, S813T, E814P, S815G, | 74 | L | 0 | 15 | 85 |
| | | D818V, N832S, K857E, E858V, | | | | | |
| | | I859V, K860E, L861V, D862G, | | | | | |
| | | R865H, K870A, N871D, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2739 | 5 | S809T, R812H, S813T, E814P, | 74 | L | 0 | 10 | 90 |
| | combo | S815G, D818V, N832S, K857E, | | | | | |
| | | E858V, I859V, K860E, L861V, | | | | | |
| | | D862G, R865H, K870A, N871D, | | | | | |
| | | K881R, K883R, N890G, K897R, | | | | | |
| | | K901H, K908Q, E912D, S914D, | | | | | |
| | | K922Q, 947E, 948L, 949E | | | | | |
| P2759 | 6 | S809T, R812H, S813T, E814P, | 73 | L | 0 | 10 | 90 |
| | | S815G, D818V, N832S, | | | | | |
| | | W836A, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2760 | 6 | S809T, R812H, S813T, E814P, | 76 | L | 0 | 10 | 90 |
| | combo | S815G, D818V, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2761 | 7 | N807D, S809T, R812H, S813T, | 75 | L | 0 | 20 | 80 |
| | | E814P, S815G, D818V, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2762 | 7 | N807H, S809T, R812H, S813T, | 74 | L | 0 | 20 | 80 |
| | | E814P, S815G, D818V, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2763 | 7 | S809T, R812H, S813T, E814P, | 75 | L | 0 | 20 | 80 |
| | | S815G, D818V, N822S, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2764 | 7 | S809T, R812H, S813T, E814P, | 75 | L | 0 | 20 | 80 |
| | | S815G, D818V, N822T, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2765 | 7 | S809T, R812H, S813T, E814P, | 74 | L | 0 | 20 | 80 |
| | | S815G, D818V, N825D, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2766 | 7 | S809T, R812H, S813T, E814P, | 73 | L | 0 | 30 | 70 |
| | | S815G, D818V, N825S, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2767 | 7 | S809T, R812H, S813T, E814P, | 73 | L | 0 | 30 | 70 |
| | | S815G, D818V, N825A, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2768 | 7 | S809T, R812H, S813T, E814P, | 72 | L | 0 | 30 | 70 |
| | | S815G, D818V, G829N, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2769 | 7 | S809T, R812H, S813T, E814P, | 71 | L | 0 | 30 | 70 |
| | | S815G, D818V, G829D, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2770 | 7 | S809T, R812H, S813T, E814P, | 75 | L | 0 | 20 | 80 |
| | | S815G, D818V, N822S, A824S, | | | | | |
| | | N825D, N832S, W836E, | | | | | |
| | | K857E, E858V, I859V, K860E, | | | | | |
| | | L861V, D862G, R865H, K870A, | | | | | |
| | | N871D, K881R, K883R, N890G, | | | | | |
| | | K897R, K901H, K908Q, E912D, | | | | | |
| | | S914D, K922Q, 947E, 948L, | | | | | |
| | | 949E | | | | | |
| P2771 | 7 | S809T, R812H, S813T, E814P, | 75 | L | 0 | 20 | 80 |
| | | S815G, D818V, N832T, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2772 | 7 | S809T, R812H, S813T, E814P, | 71 | L | 0 | 20 | 80 |
| | | S815G, D818V, N832V, | | | | | |
| | | W836E, K857E, E858V, I859V, | | | | | |
| | | K860E, L861V, D862G, R865H, | | | | | |
| | | K870A, N871D, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2773 | 7 | S809T, R812H, S813T, E814P, | 73 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871G, G872D, G873Δ, | | | | | |
| | | G874Δ, S875Δ, S876T, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2774 | 7 | S809T, R812H, S813T, E814P, | 75 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871D, G872D, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2775 | 7 | S809T, R812H, S813T, E814P, | 76 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871D, N880T, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2776 | 7 | S809T, R812H, S813T, E814P, | 76 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871D, N880S, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2777 | 7 | S809T, R812H, S813T, E814P, | 72 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871D, N880L, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2778 | 7 | S809T, R812H, S813T, E814P, | 69 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | D889S, N890G, S892D, K897R, | | | | | |
| | | K901H, K908Q, E912D, S914D, | | | | | |
| | | K922Q, 947E, 948L, 949E | | | | | |
| P2779 | 7 | S809T, R812H, S813T, E814P, | 70 | L | 0 | 20 | 80 |
| | | S815G, D818V, N832S, | | | | | |
| | | W836E, K857E, E858V, I859V, | | | | | |
| | | K860E, L861V, D862G, R865H, | | | | | |
| | | K870A, N871D, K881R, K883R, | | | | | |
| | | K897R, K901H, K908Q, E912D, | | | | | |
| | | S914D, K922Q, 947E, 948L, | | | | | |
| | | 949E | | | | | |
| P2781 | 7 | S809T, R812H, S813T, E814P, | 74 | L | 0 | 20 | 80 |
| | combo | S815G, D818V, N822S, N832S, | | | | | |
| | | K857E, E858V, I859V, K860E, | | | | | |
| | | L861V, D862G, R865H, K870A, | | | | | |
| | | N871D, N880T, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2782 | 7 | S809T, R812H, S813T, E814P, | 73 | L | 0 | 20 | 80 |
| | combo | S815G, D818V, N825D, N832S, | | | | | |
| | | K857E, E858V, I859V, K860E, | | | | | |
| | | L861V, D862G, R865H, K870A, | | | | | |
| | | N871D, N880T, K881R, K883R, | | | | | |
| | | N890G, K897R, K901H, K908Q, | | | | | |
| | | E912D, S914D, K922Q, 947E, | | | | | |
| | | 948L, 949E | | | | | |
| P2783 | 7 | S809T, R812H, S813T, E814P, | 74 | L | 0 | 10 | 90 |
| | combo | S815G, D818V, N822S, N825D, | | | | | |
| | | N832S, K857E, E858V, I859V, | | | | | |
| | | K860E, L861V, D862G, R865H, | | | | | |
| | | K870A, N871D, N880T, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2784 | 7 | N807D, S809T, R812H, S813T, | 73 | L | 0 | 10 | 90 |
| | combo | E814P, S815G, D818V, N822S, | | | | | |
| | | N825D, N832S, K857E, E858V, | | | | | |
| | | I859V, K860E, L861V, D862G, | | | | | |
| | | R865H, K870A, N871D, N880T, | | | | | |
| | | K881R, K883R, N890G, K897R, | | | | | |
| | | K901H, K908Q, E912D, S914D, | | | | | |
| | | K922Q, 947E, 948L, 949E | | | | | |
| P2785 | 7 | S809T, R812H, S813T, E814P, | 76 | L | 0 | 20 | 80 |
| | combo | S815G, D818V, N822S, N832S, | | | | | |
| | | W836E, K857E, E858V, I859V, | | | | | |
| | | K860E, L861V, D862G, R865H, | | | | | |
| | | K870A, N871D, N880T, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2786 | 7 | S809T, R812H, S813T, E814P, | 74 | L | 0 | 20 | 80 |
| | combo | S815G, D818V, N825D, N832S, | | | | | |
| | | W836E, K857E, E858V, I859V, | | | | | |
| | | K860E, L861V, D862G, R865H, | | | | | |
| | | K870A, N871D, N880T, K881R, | | | | | |
| | | K883R, N890G, K897R, K901H, | | | | | |
| | | K908Q, E912D, S914D, K922Q, | | | | | |
| | | 947E, 948L, 949E | | | | | |
| P2787 | 7 | S809T, R812H, S813T, E814P, | 75 | L | 0 | 20 | 80 |
| | combo | S815G, D818V, N822S, N825D, | | | | | |
| | | N832S, W836E, K857E, E858V, | | | | | |
| | | I859V, K860E, L861V, D862G, | | | | | |
| | | R865H, K870A, N871D, N880T, | | | | | |
| | | K881R, K883R, N890G, K897R, | | | | | |
| | | K901H, K908Q, E912D, S914D, | | | | | |
| | | K922Q, 947E, 948L, 949E | | | | | |
| P2788 | 7 | N807D, S809T, R812H, S813T, | 76 | L | 0 | 10 | 90 |
| | combo | E814P, S815G, D818V, N822S, | | | | | |
| | | N825D, N832S, W836E, | | | | | |
| | | K857E, E858V, I859V, K860E, | | | | | |
| | | L861V, D862G, R865H, K870A, | | | | | |
| | | N871D, N880T, K881R, K883R, | | | | | |
| | | N890G, K897R, K901H, K908Q, | | | | | |
| | | E912D, S914D, K922Q, 947E, | | | | | |
| | | 948L, 949E | | | | | |

N = no usable data
*IF = Initial fluorescence in DSF assay

Criteria for L, M, H in IF assay: L=IF below 30% of amplitude of unfolding peak, M=30% to 50% of amplitude of unfolding peak, H=above 50% of amplitude of unfolding peak
The volumes listed for Aggregated, Dimer, and Monomer SEC % correspond to elution volumes from a Superdex 75 SEC column.

Rounds 1 through 4 focused on increasing Tₘ. Rounds 5 and 6 focused on removing potential protease cleavage sites while maintaining Tₘ and included many reversions to past rounds. Round 7 focused on removing remaining asparagines while maintaining Tₘ. Overall, mutations were tested for 58% of the amino acids in the constant regions (72 of 124 positions). The clone resulting from 7 rounds of mutagenesis is designated P2788. In all, P2788 contains 30 mutations, representing approximately 24% of the positions in the constant regions, and includes a three-residue, C-terminal extension of residues from CBM32-2. The resulting mutations are broadly distributed throughout the primary sequence as shown in an alignment with the original sequence (FIG. 2) and throughout the 3-D structure when mapped to the CBM32-2 crystal structure (PDB accession 2W1Q). Although the mutants all possessed the same binding loops as the initial clone, we expected and observed a gradual decline in target binding with an increasing number of mutations, many of which were adjacent to the binding loops. We speculate that the loss of binding was caused by shifts in the conformation of the binding loops (data not shown). Because our intent was to improve the stability of the nanoCLAMP and then isolate new binders, our workflow did not include a screen for SUMO binding.

The number of lysines and arginines representing potential trypsin cleavage sites was reduced from 11 in the constant regions of the starting protein (clone SMT3-A1) to 5 in the constant regions of the resulting protein (clone P2788). Three of the remaining arginines (R881, R897 and R925) are expected to be involved in salt bridges as identified by the ESBRI algorithm (Costantini et al. ESBRI: a web server for evaluating salt bridges in proteins. Bioinformation 3:137-138, 2008). For these residues, we were unable to identify any substitution mutations that did not destabilize the proteins. For another position, K883, substitution with arginine was beneficial, but several additional substitutions either resulted in a greater than 10° C. decrease in melting temperature or high initial fluorescence in DSF. For one remaining position, K878, which is universally conserved in the alignment, 10 of 10 substitution mutations resulted in proteins with high initial fluorescence in DSF. In the wild-type NagH CpCBM32-2 structure, K878 Nζ forms hydrogen bonds with the carbonyl oxygens of P905 and G907 as determined by the RING 2.0 algorithm (Piovesan, et al. Nucleic Acids Res 44:W367-374, 2016). For asparagine, the number was reduced from 8 in the original clone (SMT3-A1) to 1 in the mutated clone. The remaining asparagine N928 is universally conserved in the consensus alignment and buried in the 3D structure. N928 Nδ² forms hydrogen bonds with the carbonyl oxygens of S845 and L846 as determined by the RING 2.0 algorithm. We chose not to attempt substitution mutations with N928 because of the low likelihood of deamidation based on its sequence context and the likely challenge of finding a substitution with a beneficial effect.

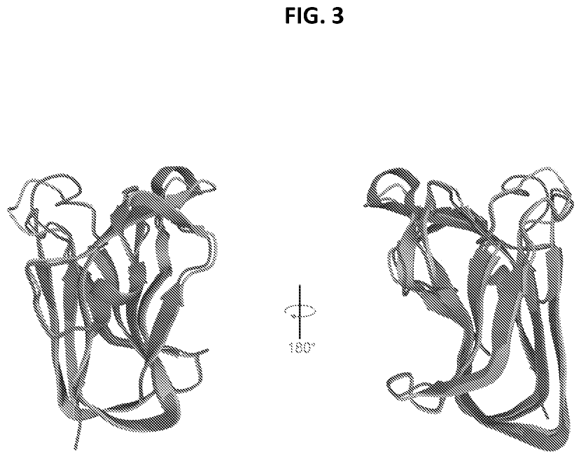

We next used AlphaFold to predict the 3-D structure of P2788 in order to assess the likelihood of gross changes in 3D-structure (Jumper et al. Nature 596:583-589, 2021). A 3D-alignment of the crystal structure of CBM32-2 and the predicted structure of P2788 was performed with the jFATCAT(rigid) algorithm (FIG. 3). As expected with conservative substitutions, the high degree of similarity and the use of templates by the AlphaFold algorithm, the predicted structure of the constant regions of P2788 does not show gross deviations from the solved crystal structure of NagH CpCBM32-2. As expected, because of the differences in amino acid sequence, the variable loops show deviations, especially for the longest 838-844 loop. The overall similarity of the solved CBM32-2 structure versus the predicted structure of P2788 is high, even with the differences in loop sequences (TM-score=0.95).

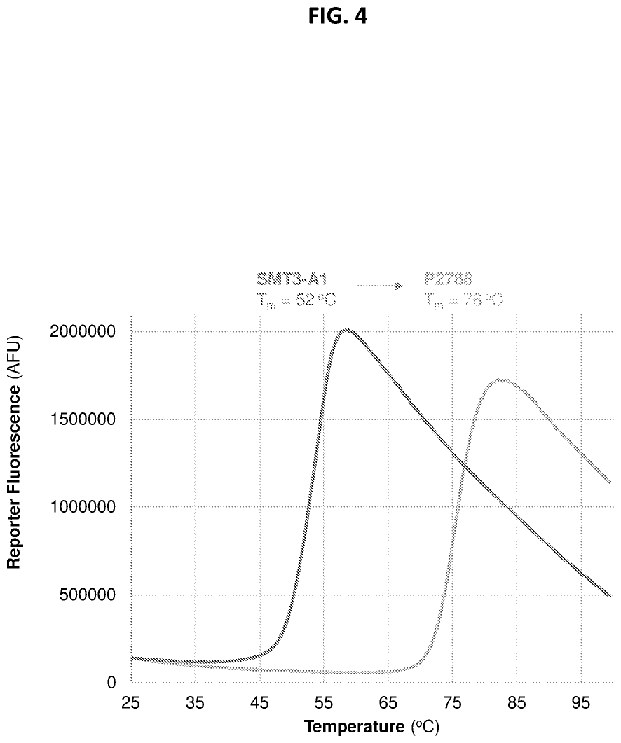

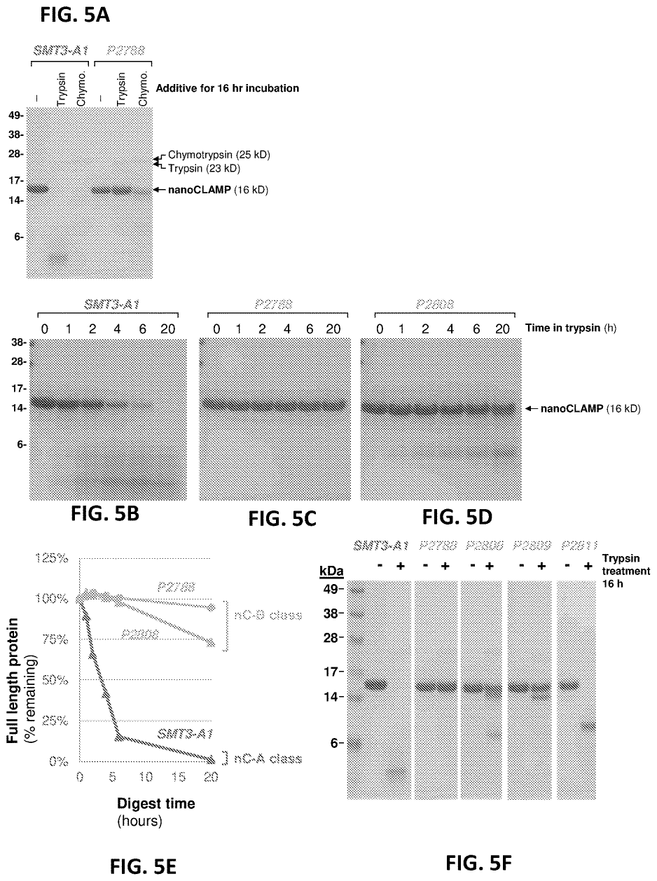

Compared with the starting protein, the Tₘ increased by 24° C. from 52° C. to 76° C. (FIG. 4). We next tested P2788's resistance to digestion by trypsin. Following a 16-hour digestion in 0.1 mg/ml trypsin, no full length SMT3-A1 remained whereas no apparent digestion of P2788 had occurred as assessed by SDS-PAGE (FIG. 5A). A time course of trypsin digestion determined that the t_{1/2} increased from 3 hours for SMT3-A1 to >16 hours for P2788 (FIGS. 5C-5E). While our protein engineering campaign deleted only a few surface-exposed chymotrypsin-cleavage sites, we also tested resistance to chymotrypsin as a reflection of general stability. Following a 16-hour digestion with 0.1 mg/ml chymotrypsin, about a fifth of P2788 appeared to remain full-length while SMT3-A1 was completely digested (FIG. 5A).

We next sought to determine whether a new class of nanoCLAMPs based on the constant regions of P2788 could confer these properties to newly isolated clones. We call the P2788-derived class the “nC-B class” (nanoCLAMP-B with the identifier “B” referring to the next variant of the original class of nanoCLAMPs). The first generation of nanoCLAMPs represented by SMT3-A1 and others is referred to as the “nC-A class” (nanoCLAMP-A with the identifier “A” referring to the first class of nanoCLAMPs). For clarity, the nC-A class of nanoCLAMPs encompasses the first published nanoCLAMPs (Suderman et al. supra). Relative to NagH CpCBM32-2, nanoCLAMPs of the nC-A class have a M929L mutation that removes a methionine as well as amino acid differences in the variable loops.

Phage Display Library Panning for Improved SUMO Capture Agents of the nC-B Class of nanoCLAMPs.

To confirm that the optimized constant regions of the nC-B class can support the general isolation of high affinity binders with improved thermal, proteolytic, and alkaline stability, we sought to isolate new nanoCLAMP binders containing the nC-B constant regions and a diversity of variable loops. We first constructed a phage display library with randomized binding loops in the context of the nC-B constant regions and panned the library for binders to yeast SUMO (SMT3). This library has the same three variable loops with randomized residues as the previous library from which SMT3-A1 was isolated but uses the nC-B constant regions instead of the nC-A constant regions. Degenerate oligonucleotides constructed with phosphoramidite trimers were designed so that the variable regions encoded all amino acids except cysteine (omitted to avoid heterogeneous coupling to multiple cysteines), methionine (omitted to avoid the risk of inactivating oxidation), and lysine and arginine (omitted to avoid the addition of a trypsin-cleavage sites). Position 818 was held constant with a valine, the wild-type amino acid, because valine and another small hydrophobic amino acid isoleucine, appeared in one quarter of nanoCLAMPs from previous screens. The resulting library contained over 10¹⁰ variants of nC-B nanoCLAMPs.

Following the third round of panning of this library, we randomly selected 96 clones and screened them for target binding by semELISA, which yielded 93 confirmed positives. Of these, 40 were sequenced to identify 18 unique nanoCLAMP SUMO binders. These binders were subcloned into a bacterial expression vector, expressed, purified by immobilized metal affinity chromatography (IMAC), and confirmed to be over 90% pure as estimated by SDS-PAGE (data not shown). We then evaluated the purified nanoCLAMPs' ability to function as affinity capture agents in a medium-throughput, small-scale depletion assay. In this assay, the nanoCLAMPs were conjugated to cross-linked agarose under denaturing conditions, refolded on the resin, incubated with SUMO, and the quantity of unbound SUMO measured by A₂₈₀.

The purified nanoCLAMPs were then screened for monodispersity by size exclusion chromatography and Tₘ by DSF. Of the 18, seven (38%) were over 90% monomer. Of these, five had a melting temperature of greater than 73° C., with four having melting temperatures greater than 99° C. (Table 4). The initial results with the SUMO test case suggest that the nC-B constant regions generally support the isolation of clones with high monodispersity and thermostability.

TABLE 4
Yield and proportion of monomer for anti-SUMO nanoCLAMPs

| | Approximate proportion monomer | Tₘ | |
|---|---|---|---|
| Clone | (% of total area by SEC) | (° C.) by DSF | |
| P2808 | >90% | 73° | C. |
| P2809 | >90% | >99° | C. |
| P2811 | >90% | >99° | C. |
| P2815 | >90% | 48° | C. |
| P2807 | >90% | >99° | C. |
| P2816 | >90% | >99° | C. |
| P2817 | >90% | 49° | C. |
| P2803 | 70-90% | 79° | C. |
| P2804 | 70-90% | 46° | C. |
| P2810 | 70-90% | >99° | C. |
| P2820 | 70-90% | 83° | C. |
| P2805 | <70% | 45° | C. |
| P2806 | <70% | 83° | C. |
| P2812 | <70% | 57° | C. |
| P2813 | <70% | >99° | C. |
| P2814 | <70% | 70° | C. |
| P2818 | <70% | 79° | C. |
| P2819 | <70% | 66° | C. |

Characterization of Binding Affinity, Thermostability and Protease-Resistance of nanoCLAMP Capture Agents of the nC-B Class.

We chose three nC-B nanoCLAMPs (P2808, P2809, and P2811) for further characterization. These were selected to provide a diverse sample of binding loops. For the three clones, Loop 817-820 and Loop 931-935 did not have any apparent similarity. Except for V818, whose identity was fixed in the library, there were no identities in any positions of these loops. For Loop 838-844, clones P2808 and P2809 are identical in 5 of 7 positions while clone 2811 shows no identities with either.

All three nanoCLAMPs were produced in E. coli with yields >150 mg/liter of shaken E. coli culture and used for biophysical characterization experiments.

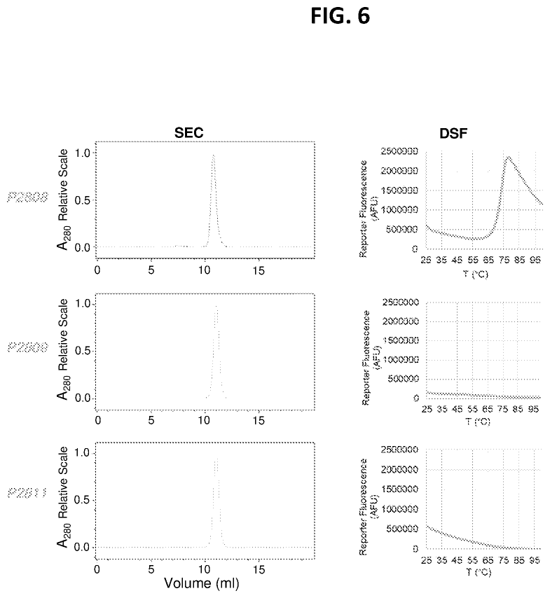

We first checked the quaternary structure of nanoCLAMPs by size exclusion chromatography to confirm that subsequent results could be interpreted without confounding avidity effects from higher order multimers or aggregates. All three clones migrated as monodisperse monomers (FIG. 6). To rank these nanoCLAMPs by their affinity for SUMO, we used biolayer interferometry to measure their dissociation constants, which ranged from 5 to 80 nM (Table 5). We then measured the melting temperature of the nanoCLAMPs using DSF. P2808 had an apparent Tₘ of 73° C. P2809 and P811 had flat-line DSF curves with no melting transition apparent between 25° and 99° C. (FIG. 6). This observation suggests that the Tₘ for these nanoCLAMPs exceeds the quantifiable range for this assay. We corroborated this observation with a functional binding assay to assess kinetic thermostability. In this assay, we incubated samples of each nanoCLAMP at different temperatures, cooled and centrifuged the solutions, and then measured the binding activity remaining in the supernatant by biolayer interferometry. In this test, we define T₅₀ as the temperature of a 5-minute heat challenge after which 50% of binding activity is irreversibly lost. The T₅₀ ranged from ~85° C. for clone P2808 to >100° C. for clones P2809 and P2811.

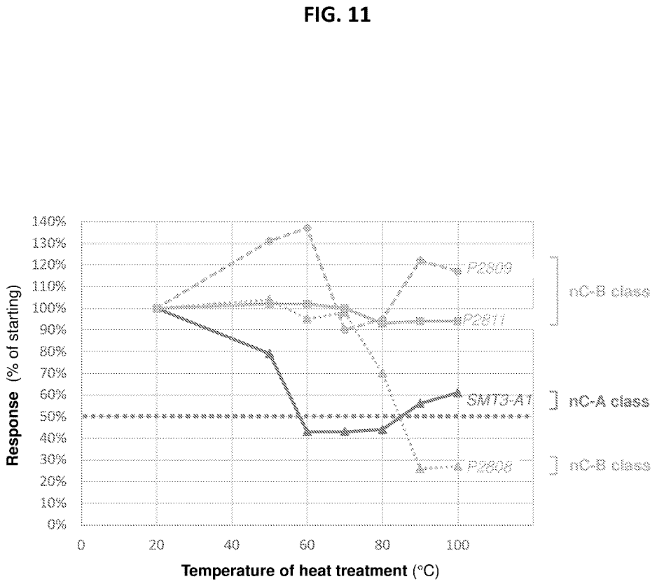

The rank order and values are consistent with the Tₘ measured by DSF (FIG. 11). Clones P2809 and P2811, both with Tₘ's >100° C., maintained greater than 90% activity after incubation at boiling temperatures for 5 minutes. The maintenance of binding activity indicates that the nanoCLAMPs remained in solution after heat treatment and did not irreversibly aggregate or precipitate. Taken together with the DSF data, the kinetic thermostability measurements suggest that these two nanoCLAMPs may remain folded up to, and possibly above, 99° C.

TABLE 5
Tₘ, T₅₀, K_{d}, trypsin resistance of selected anti-SUMO nanoCLAMPs

| | | Loop | | | | Loop | | | | | |
|---|---|---|---|---|---|---|---|---|---|---|---|
| | | seq. | SEQ | Loop | SEQ | seq. | SEQ | | | | |
| | | 817- | ID | seq. | ID | 931- | ID | | | K_{d} | Trypsin |
| Clone | Class | 820 | NO: | 838-844 | NO: | 935 | NO: | Tₘ | T₅₀ | SUMO | resist. |
| SMT3- | nC-A | EDIK | 42 | FNEVFYE | 43 | DKILF | 44 | 52° C. | ~58° C. | 163 nM | − |
| A1 | | | | | | | | | | | |
| P2788 | nC-B | EDIK | 42 | FNEVFYE | 43 | DKILF | 44 | ~76° C. | N/A | N/A | + |
| P2808 | nC-B | LVIP | 45 | IIVEEFT | 46 | YNFID | 47 | 73° C. | ~85° C. | 7 nM | + |
| P2809 | nC-B | NVEQ | 48 | ILVEEFP | 49 | NEVPQ | 50 | >99° C. | >100° C. | 80 nM | + |
| P2811 | nC-B | PVVT | 51 | IVLTDQA | 52 | LAQDP | 53 | >99° C. | >100° C. | 5 nM | − |

We next characterized the trypsin-resistance of the three clones. Clones P2808 and P2809 were highly resistant to digestion with 0.1 mg/ml trypsin, while clone P2811 was less resistant (FIG. 5F). FIGS. 5B-5D show a time course of a tryptic digest comparing nanoCLAMPs with different combinations of constant regions and variable loops to understand the contribution of each component to trypsin resistance. We tested the original clone SMT3-A1 (nC-A constant regions and original variable loops), P2788 (nC-B constant regions with the original variable loops from SMT3-A1), and P2808 (nC-B constant regions with newly isolated variable loops). Both P2788 and P2808 show a t_{1/2} of >20 hours in 0.1 mg/ml trypsin, compared with ~4 hours for the original SMT3-A1 clone. Together with the observations of P2809 and P2811, these results indicate that trypsin-resistance depends upon the sequences of both the variable loops and constant regions. The observation of trypsin-resistance for 3 of 4 clones isolated with diverse loop sequences and identical constant regions indicates that the nC-B constant regions can be generally used to isolate trypsin-resistant clones, in at least a first test-case.

Measurement of Performance Parameters with Affinity Chromatography Using Capture Agents of the nC-B Class of nanoCLAMPs.

For brevity, we will subsequently refer to affinity chromatography resins made with capture agents of the nC-B class of nanoCLAMPs as “nC-B affinity resins.” As a test case for the utility of nC-B affinity resins, we used P2808 as a capture agent for more detailed studies. To generate the affinity resin, the P2808 protein was expressed, purified by IMAC under denaturing conditions, conjugated to sulfhydryl-reactive 6% cross-linked agarose resin, and then refolded by rinsing with buffered saline. We prepared a test mixture of crude E. coli lysates spiked with a SUMO-GFP fusion protein, optimized purification conditions and assessed the resins' selectivity and binding capacity.

In pilot experiments, we observed that polyol-elution at neutral pH was still possible, as with the original nanoCLAMP (Suderman et al. supra), but with qualitatively lower speed and yield (data not shown). As an alternative, we tested elution of the target protein with molar concentrations of imidazole, which has been used successfully for disrupting protein-peptide interactions and antibody-Protein A interactions.

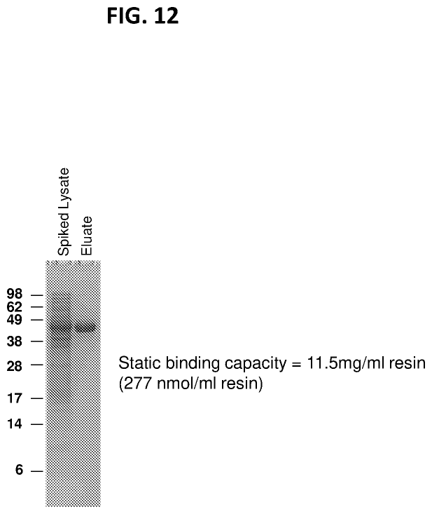

A buffer with 3 M imidazole at pH 8 quickly and completely eluted SUMO from P2808 resin. As shown in FIG. 12, after incubation for 1 hour with an excess of target protein in a spiked E. coli lysate, the bound target was washed and eluted with a purity of over 90% as estimated by densitometric analysis of Coomassie stained SDS-PAGE. The static binding capacity under these conditions was 11.5 mg/ml resin (277 nmol/ml). In subsequent experiments, we found that imidazole elution worked consistently with P2808, P2809, and P2811, as well as three different AC resins targeting SUMO, mCherry, and GFP with capture agents of the nC-A class. The observations suggest that imidazole elution is a general property of both class of nanoCLAMPs.

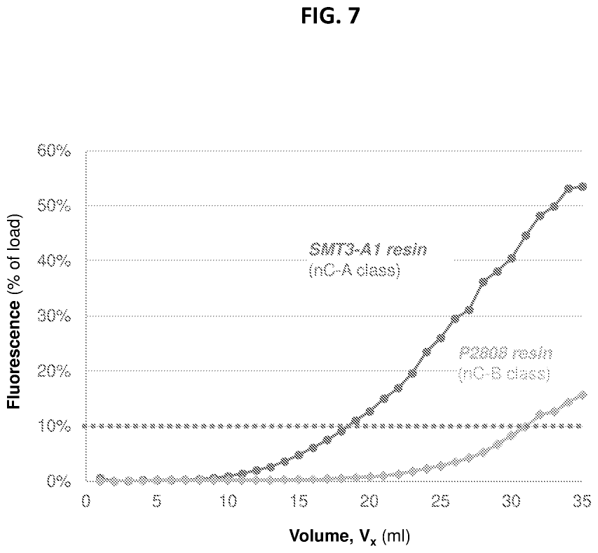

With elution conditions established, we next measured the dynamic binding capacity of P2808 resin using a 0.6 ml packed column (0.5 cm ID×3.06 cm) at a constant flowrate of 0.5 ml/min. We utilized the fluorescence of the SUMO-GFP target to determine the QB₁₀, or the volume at which the eluate's fluorescence equaled 10% of the load's fluorescence (see Materials and Methods for calculations). The dynamic binding capacity of P2808 resin under these conditions was 10 mg/ml resin (240 nmol/ml resin) (FIG. 7). This capacity represents an approximate 70% increase over our original SMT3-A1 resin.

We next tested the efficiency of the SUMO affinity resin P2808 resin under flow conditions. A 0.6 ml packed column and a flowrate of 0.5 ml/min (linear flowrate=153 cm/h) were used to test two regimes.

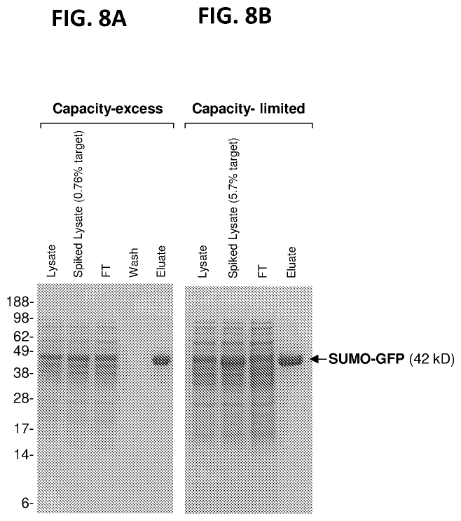

In the capacity-excess regime, SUMO was spiked into an E. coli lysate at a low concentration (SUMO-GFP=0.76% of total protein by weight), loaded at 42% of the column's dynamic capacity (36% of its static binding capacity). As assessed by densitometry of a Coomassie-stained SDS-PAGE gel, the purity was >90%, and the yield was 90%. (FIG. 8A).

In the capacity-limited regime, SUMO-GFP was spiked into an E. coli lysate at a high concentration (SUMO-GFP=5.7% of total protein by weight) and loaded at 133% of the column's dynamic binding capacity (116% of its static binding capacity) (FIG. 8B). The purity and yield were comparable to the capacity-excess regime, and both estimated to be >90%. The performance metrics for both purifications are summarized in Table 6.

TABLE 6
Metrics for AC purification of SUMO-GFP with
P2808 resin loaded at different capacities

| | Capacity-excess | | Capacity-limited | |
|---|---|---|---|---|
| % of Dynamic binding | 42% | | 133% | |
| capacity¹ | | | | |
| % of Static binding capacity² | 36% | | 116% | |
| Total protein in Load (mix of | 333 | mg | 140 | mg |
| E. coli lysate and SUMO) | | | | |
| Target (SUMO-GFP) in Load | 2.5 mg (0.76% of total protein) | | 8 mg (5.7% of total protein) | |
| Concentration of target in | 0.025 | mg/ml | 0.2 | mg/ml |
| Load | | | | |
| Volume of Load | 100 | ml | 40 | ml |
| Flow rate | 0.5 ml/min (0.44 CV/min) | | 0.5 ml/min. (0.44 CV/min | |
| Linear flow rate | 153 | cm/h | 153 | cm/h |
| Residence time | 1.2 | min | 1.2 | min |
| Recovered target | 2.47 | mg | 7.6 | mg |

| | | |
|---|---|---|
| Purity by densitometry | >90% | >90% |
| Fold purification | 120-fold | 16-fold |
| Yield | >90% | >90% |

¹DBC = 10 mg/ml resin (see FIG. 7)
²SBC = 11.5 mg/ml resin (see FIG. 12)

Compatibility of nC-B Affinity Resins with Repeated Cycles of NaOH Cleaning.

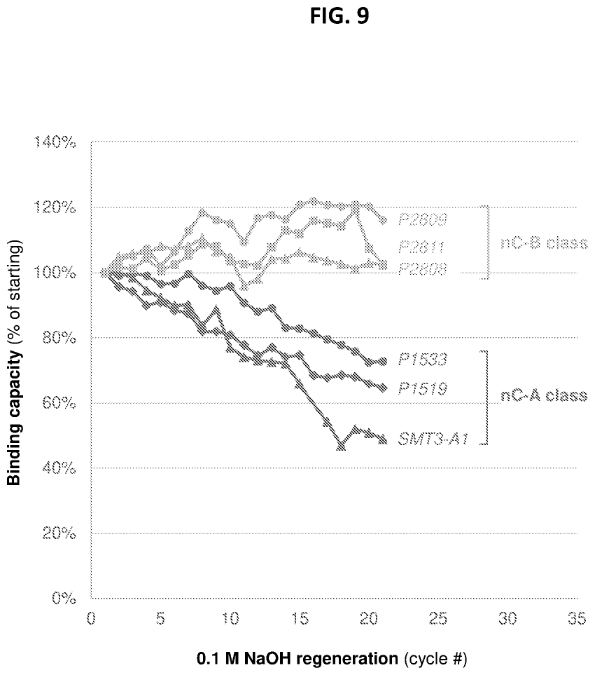

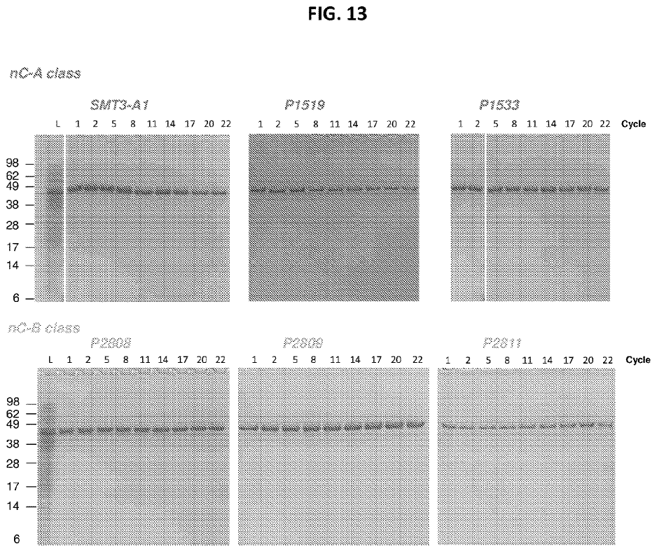

For large-scale production of industrial proteins or biologics, the re-use and cleaning of columns in place reduces manufacturing costs and maintains consistent performance. Sodium hydroxide solutions are commonly used for cleaning-in-place procedures, so we tested the general compatibility of the new nanoCLAMP resins with sodium hydroxide treatment. We tested 3 resins made with nC-A nanoCLAMPs and 3 resins made with nC-B nanoCLAMPs. Each cycle consisted of loading a SUMO-GFP target spiked into an E. coli lysate; a wash step, elution with 3 M imidazole pH 8; 10 minutes of contact time with 0.1 M NaOH; and then a 5-minute re-equilibration step. The eluates were collected and analyzed for purity by SDS PAGE, and the purified target quantified by fluorescence spectroscopy (FIGS. 9 and 13). In the first few cycles, we were surprised to observe an improvement in binding capacity of 5 to 20% with nC-B resins. It is possible that the increase in binding results from the removal of inhibitory material by NaOH. For all three nC-B resins, the binding capacity plateaued over 20 cycles and remained at or above 100% of starting capacity. In contrast, we observed a steady reduction in binding capacity by 25% to 50% with all three nC-A resins. Taken together, this data indicates that nanoCLAMPs of the nC-B class can generally serve as capture agents for resins capable of single-step affinity purification of targets to homogeneity. These resins are also compatible with cleaning-in-place protocols using 0.1 M sodium hydroxide for over 20 cycles, without loss of binding capacity or specificity. Further, in practice, cleaning-in-place cycles are not usually performed after each run, so the expected lifetime likely exceeds 100 cycles with the assumption of sanitation every 5^{th} run.

Compatibility of nC-B Affinity Resins with Repeated Cycles of low pH Elution.

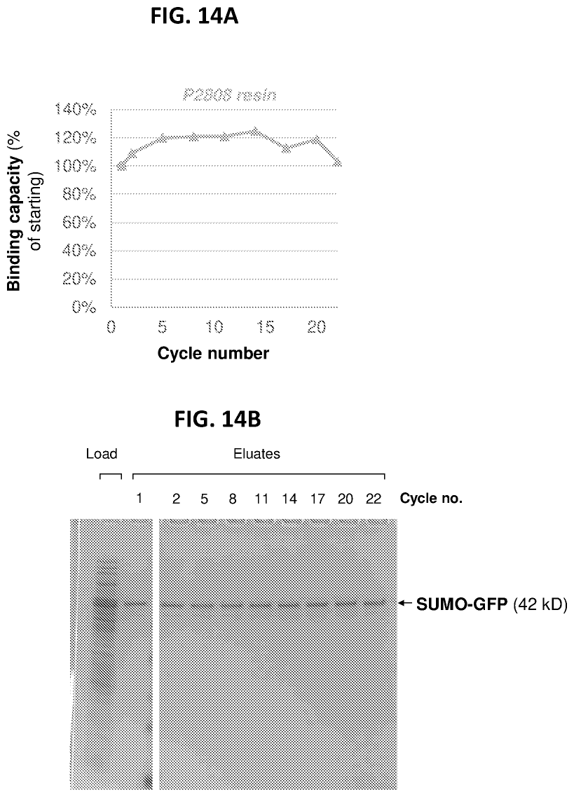

Because elution with 3 M imidazole might be suboptimal for some applications, we also tested elution with a citrate buffer at pH 2.5. In these experiments, the resin was also cleaned with NaOH in between each cycle for 1 minute before re-equilibrating the resin with buffered saline. The nanoCLAMP maintained 100% of its binding capacity and specificity over more than 20 cycles of loading, elution, and regeneration (FIGS. 14A and 14B).

Compatibility of nC-B Affinity Resins with Organic Solvent and Autoclaving.

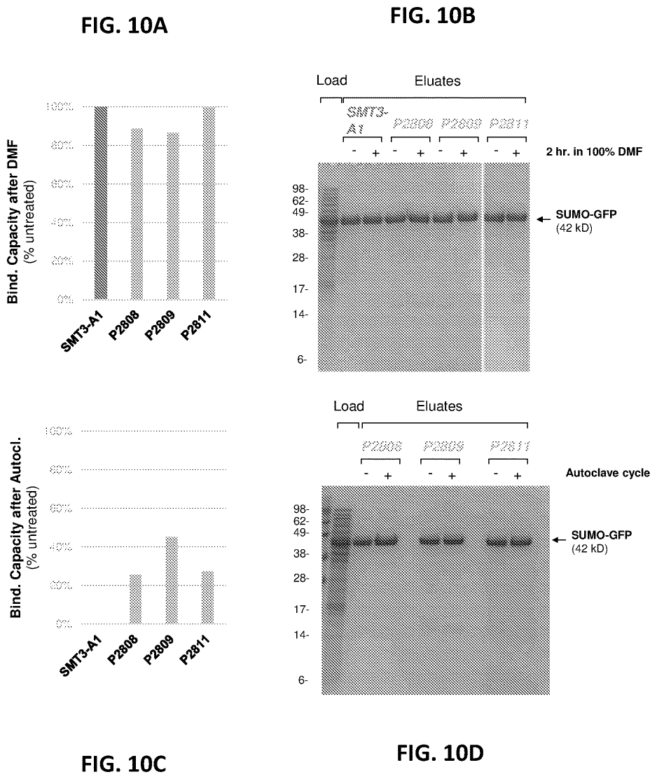

We next tested the ability of the nC-B resins and the original SMT3-A1 resin to resist more extreme conditions. First, we tested the resins' ability to recover selective binding capacity after exposure to 100% DMF. We incubated nanoCLAMP resin in 100% DMF for 2 hours, re-equilibrated with buffered saline, and then measured binding capacity. Of the four resins tested (the original SMT3-A1 resin and P2808 resin, P2809 resin, and P2811 resin), all retained over 85% binding capacity after treatment with DMF (FIG. 10A) and maintained their apparent selectivity as assessed qualitatively by SDS-PAGE (FIG. 10B).

Because the nC-B resins were robust to the broad range of conditions tested so far, we decided to determine whether the resins could also retain binding and specificity after autoclaving. We autoclaved the resin with a 105-minute liquid steam cycle, including 30-minute exposure to 120° C. and 20 p.s.i., re-equilibrated at room temperature, and then measured binding capacity. The resin made with the nC-A nanoCLAMP (SMT3-A1) did not bind any detectable target protein after autoclaving. In contrast, the resins made with the nC-B nanoCLAMPs (P2808, P2809, and P2811) retained 25% to 45% binding capacity with specificity comparable to controls (FIGS. 10C and 10D). The SMT3-A1 eluates were not analyzed in FIG. 10D because there was not enough protein in the autoclaved sample to prepare a normalized aliquot to compare to the control. To our knowledge, affinity chromatography resins based on nC-B nanoCLAMPs are the first protein-based affinity resins shown to retain significant binding capacity and specificity after autoclaving.

### Discussion

We report on a protein engineering campaign yielding an improved class of nanoCLAMPs suitable as capture agents suitable for process-scale affinity chromatography. Similar to Protein A resins or antibody-based immunoaffinity resins, resins made with the new nC-B class of nanoCLAMPs support single step purifications from complex mixtures with high yield and fold-purification. Similar to Protein A resins, but unlike antibody-based resins, nC-B resins are compatible with NaOH cleaning-in-place, can be produced by bacterial expression of the capture reagent, and lack cysteine residues. Unlike either Protein A or antibody-based resins, nC-B resins are distinct in having been shown to exhibit resistance to boiling temperatures, trypsin, and organic solvent. Key performance parameters of nC-B resins and supporting results are summarized in Table 7.

TABLE 7
Attributes of affinity chromatography resins made with the nC-B class of nanoCLAMPs

| | Supporting results from test case with anti- |
|---|---|
| Attribute | SUMO nanoCLAMPs* |
| Efficient purification | Purification to >90% in a single step |
| High dynamic binding capacity | 240 nmol/ml (residence time of 1.2 min) |
| High thermal stability | Tₘ > 99° C., resistant to boiling and autoclaving |
| High trypsin resistance | t_{1/2} > 16 hours in 0.1 mg/ml trypsin |
| High resistance to organic solvent | Full recovery of binding activity after 2 hr in 100% |
| | DMF |
| High stability in NaOH | Full recovery of binding activity after >20 cycles, |
| | each including 10 min incubation with 0.1M |
| | NaOH |
| Activity-preserving options for elution buffers | 100% yield after elution with imidazole pH 8 or |
| | citrate pH 2.5 |
| Tolerance to oxidizing and reducing conditions | No cysteines or methionines in the protein |
| Low-cost production | 150-300 mg nanoCLAMP per liter of shaken |
| | E. coli culture |

*Affinity resins based on Sulfolink crosslinked beaded agarose (Thermo)

This work provides a test case supporting the potential of nC-B nanoCLAMP resins to extend Protein A-like levels of performance to a broad range of proteins beyond antibodies. nC-B resins' efficiency, low cost-of-manufacture and reusability have the potential to reduce the total cost of manufacture for process-scale purifications. In addition, nanoCLAMPs' general compatibility with high temperature, organic solvent and pH extremes may enable industrial applications where extreme conditions are required.

The improved stability and performance of nC-B nanoCLAMPs also support their use in applications beyond immunoaffinity chromatography. nanoCLAMPs have been used successfully in bioelectric and electrochemical sensors. The conditions at the surface of a biosensor represent a challenging environment where the improved stability of nC-B nanoCLAMPs may be enabling. For example, conjugation of nanoCLAMPs to surfaces in DMF allows the use of reaction conditions compatible with reagents that have low solubility in aqueous buffers.

The performance characteristics demonstrated for the exemplary nanoCLAMPs in this work support further exploration of nanoCLAMPs' potential to substitute for other capture agents in a broad range of applications, especially those procedures requiring tight, selective or reversible binding and exposure to extreme conditions.

### Materials and Methods

### Cloning of SMT3-A1 Mutants

The plasmid pET(SMT3-A1) containing nanoCLAMP SMT3-A1 was mutated by inverse PCR (Ochman et al. Genetics 120:621-623, 1988) by amplifying the plasmid with forward and reverse primers containing the mutation(s) of interest with 15-bp overlapping 5′ ends, purifying the amplicon, In-Fusion cloning the ends back together (In Fusion HD Cloning Kit, Takara), and transforming chemically competent NEc1 E. coli (BL21(DE3) derivative with slyDD(His151-His196) from Nectagen, Inc.). Plasmids were purified by Qiagen miniprep kit (Qiagen) and mutations were verified by sequencing the purified plasmids by Sanger sequencing (Genewiz). Glycerol stocks of the plasmids in NEc1 cells were prepared for seeding expression cultures. Constructs for conjugating to Sulfolink resin (Thermo) coded for the nanoCLAMP with an N-terminal 6-His tag and a 13-amino acid C-terminal GS-linker followed by a Cys. Constructs for expressing nanoCLAMPs for biophysical characterization lacked the GS-linker and the C-terminal Cys to avoid dimerization issues due to disulfides.

pET(SMT3-A1) Sequence
(SEQ ID NO: 38)
TTCTTGAAGACGAAAGGGCCTCGTGATACGCCTATTTTTATAGGTTAATGTCATGATAATAATGGTTTCTTAGACGT
CAGGTGGCACTTTTCGGGGAAATGTGCGCGGAACCCCTATTTGTTTATTTTTCTAAATACATTCAAATATGTATCCG
CTCATGAGACAATAACCCTGATAAATGCTTCAATAATATTGAAAAAGGAAGAGTATGAGTATTCAACATTTCCGTGT
CGCCCTTATTCCCTTTTTTGCGGCATTTTGCCTTCCTGTTTTTGCTCACCCAGAAACGCTGGTGAAAGTAAAAGATG
CTGAAGATCAGTTGGGTGCACGAGTGGGTTACATCGAACTGGATCTCAACAGCGGTAAGATCCTTGAGAGTTTTCGC
CCCGAAGAACGTTTTCCAATGATGAGCACTTTTAAAGTTCTGCTATGTGGCGCGGTATTATCCCGTGTTGACGCCGG
GCAAGAGCAACTCGGTCGCCGCATACACTATTCTCAGAATGACTTGGTTGAGTACTCACCAGTCACAGAAAAGCATC
TTACGGATGGCATGACAGTAAGAGAATTATGCAGTGCTGCCATAACCATGAGTGATAACACTGCGGCCAACTTACTT
CTGACAACGATCGGAGGACCGAAGGAGCTAACCGCTTTTTTGCACAACATGGGGGATCATGTAACTCGCCTTGATCG
TTGGGAACCGGAGCTGAATGAAGCCATACCAAACGACGAGCGTGACACCACGATGCCTGCAGCAATGGCAACAACGT
TGCGCAAACTATTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCCGGTGAGCGTGG
GTCTCGCGGTATCATTGCAGCACTGGGGCCAGATGGTAAGCCCTCCCGTATCGTAGTTATCTACACGACGGGGAGTC
AGGCAACTATGGATGAACGAAATAGACAGATCGCTGAGATAGGTGCCTCACTGATTAAGCATTGGTAACTGTCAGAC
CAAGTTTACTCATATATACTTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGAGCGCAGATACCAAATACTGT
CCTTCTAGTGTAGCCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATCCCCTGATTCTGTGGATAACC
GTATTACCGCCTTTGAGTGAGCTGATACCGCTCGCCGCAGCCGAACGACCGAGCGCAGCGAGTCAGTGAGCGAGGAA
GCGGAAGAGCGCCTGATGCGGTATTTTCTCCTTACGCATCTGTGCGGTATTTCACACCGCATATATGGTGCACTCTC
AGTACAATCTGCTCTGATGCCGCATAGTTAAGCCAGTATACACTCCGCTATCGCTACGTGACTGGGTCATGGCTGCG
CCCCGACACCCGCCAACACCCGCTGACGCGCCCTGACGGGCTTGTCTGCTCCCGGCATCCGCTTACAGACAAGCTGT
GACCGTCTCCGGGAGCTGCATGTGTCAGAGGTTTTCACCGTCATCACCGAAACGCGCGAGGCAGCTGCGGTAAAGCT
CATCAGCGTGGTCGTGAAGCGATTCACAGATGTCTGCCTGTTCATCCGCGTCCAGCTCGTTGAGTTTCTCCAGAAGC
GTTAATGTCTGGCTTCTGATAAAGCGGGCCATGTTAAGGGCGGTTTTTTCCTGTTTGGTCACTGATGCCTCCGTGTA
AGGGGGATTTCTGTTCATGGGGGTAATGATACCGATGAAACGAGAGAGGATGCTCACGATACGGGTTACTGATGATG
AACATGCCCGGTTACTGGAACGTTGTGAGGGTAAACAACTGGCGGTATGGATGCGGCGGGACCAGAGAAAAATCACT
CAGGGTCAATGCCAGCGCTTCGTTAATACAGATGTAGGTGTTCCACAGGGTAGCCAGCAGCATCCTGCGATGCAGAT
CCGGAACATAATGGTGCAGGGCGCTGACTTCCGCGTTTCCAGACTTTACGAAACACGGAAACCGAAGACCATTCATG
TTGTTGCTCAGGTCGCAGACGTTTTGCAGCAGCAGTCGCTTCACGTTCGCTCGCGTATCGGTGATTCATTCTGCTAA
CCAGTAAGGCAACCCCGCCAGCCTAGCCGGGTCCTCAACGACAGGAGCACGATCATGCGCACCCGTGGCCAGGACCC
AACGCTGCCCGAGATGCGCCGCGTGCGGCTGCTGGAGATGGCGGACGCGATGGATATGTTCTGCCAAGGGTTGGTTT
GCGCATTCACAGTTCTCCGCAAGAATTGATTGGCTCCAATTCTTGGAGTGGTGAATCCGTTAGCGAGGTGCCGCCGG
CTTCCATTCAGGTCGAGGTGGCCCGGCTCCATGCACCGCGACGCAACGCGGGGAGGCAGACAAGGTATAGGGCGGCG
CCTACAATCCATGCCAACCCGTTCCATGTGCTCGCCGAGGCGGCATAAATCGCCGTGACGATCAGCGGTCCAGTGAT
CGAAGTTAGGCTGGTAAGAGCCGCGAGCGATCCTTGAAGCTGTCCCTGATGGTCGTCATCTACCTGCCTGGACAGCA
TGGCCTGCAACGCGGGCATCCCGATGCCGCCGGAAGCGAGAAGAATCATAATGGGGAAGGCCATCCAGCCTCGCGTC
GCGAACGCCAGCAAGACGTAGCCCAGCGCGTCGGCCGCCATGCCGGCGATAATGGCCTGCTTCTCGCCGAAACGTTT
GGTGGCGGGACCAGTGACGAAGGCTTGAGCGAGGGCGTGCAAGATTCCGAATACCGCAAGCGACAGGCCGATCATCG
TCGCGCTCCAGCGAAAGCGGTCCTCGCCGAAAATGACCCAGAGCGCTGCCGGCACCTGTCCTACGAGTTGCATGATA
AAGAAGACAGTCATAAGTGCGGCGACGATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACATTAATTGCGTTGCGCTCACTGCCCGCTT
TCCAGTCGGGAAACCTGTCGTGCCAGCTGCATTAATGAATCGGCCAACGCGCGGGGAGAGGCGGTTTGCGTATTGGG
CGCCAGGGTGGTTTTTCTTTTCACCAGTGAGACGGGCAACAGCTGATTGCCCTTCACCGCCTGGCCCTGAGAGAGTT
GCAGCAAGCGGTCCACGCTGGTTTGCCCCAGCAGGCGAAAATCCTGTTTGATGGTGGTTAACGGCGGGATATAACAT
GAGCTGTCTTCGGTATCGTCGTATCCCACTACCGAGATATCCGCACCAACGCGCAGCCCGGACTCGGTAATGGCGCG
CATTGCGCCCAGCGCCATCTGATCGTTGGCAACCAGCATCGCAGTGGGAACGATGCCCTCATTCAGCATTTGCATGG
TTTGTTGAAAACCGGACATGGCACTCCAGTCGCCTTCCCGTTCCGCTATCGGCTGAATTTGATTGCGAGTGAGATAT
TTATGCCAGCCAGCCAGACGCAGACGCGCCGAGACAGAACTTAATGGGCCCGCTAACAGCGCGATTTGCTGGTGACC
CAATGCGACCAGATGCTCCACGCCCAGTCGCGTACCGTCTTCATGGGAGAAAATAATACTGTTGATGGGTGTCTGGT
CAGAGACATCAAGAAATAACGCCGGAACATTAGTGCAGGCAGCTTCCACAGCAATGGCATCCTGGTCATCCAGCGGA
TAGTTAATGATCAGCCCACTGACGCGTTGCGCGAGAAGATTGTGCACCGCCGCTTTACAGGCTTCGACGCCGCTTCG
TTCTACCATCGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCCACGCGGTTG
GGAATGTAATTCAGCTCCGCCATCGCCGCTTCCACTTTTTCCCGCGTTTTCGCAGAAACGTGGCTGGCCTGGTTCAC
CACGCGGGAAACGGTCTGATAAGAGACACCGGCATACTCTGCGACATCGTATAACGTTACTGGTTTCACATTCACCA
CCCTGAATTGACTCTCTTCCGGGCGCTATCATGCCATACCGCGAAAGGTTTTGCGCCATTCGATGGTGTCCGGGATC
TCGACGCTCTCCCTTATGCGACTCCTGCATTAGGAAGCAGCCCAGTAGTAGGTTGAGGCCGTTGAGCACCGCCGCCG
CAAGGAATGGTGCATGCAAGGAGATGGCGCCCAACAGTCCCCCGGCCACGGGGCCTGCCACCATACCCACGCCGAAA
CAAGCGCTCATGAGCCCGAAGTGGCGAGCCCGATCTTCCCCATCGGTGATGTCGGCGATATAGGCGCCAGCAACCGC
ACCTGTGGCGCCGGTGATGCCGGCCACGATGCGTCCGGCGTAGAGGATCGAGATCTCGATCCCGCGAAATTAATACG
ACTCACTATAGGGGAATTGTGAGCGGATAACAATTCCCCTCTAGAAATAATTTTGTTTAACTTTAAGAAGGAGATAT
ACCATGGGCAGCAGCCATCATCATCATCATCACAACCCTTCTTTAATTCGTTCTGAATCCTGGGAAGACATCAAAGG
GAATGAAGCCAATTTATTAGATGGAGACGATAACACCGGTGTTTGGTATTTCAACGAAGTTTTCTACGAATCTCTTG
CAGGAGAATTCATTGGATTGGACTTAGGTAAGGAAATTAAATTGGATGGTATTCGTTTTGTTATTGGTAAGAATGGA
GGCGGTAGTTCCGACAAATGGAACAAATTCAAGTTGGAGTACTCCCTGGATAACGAAAGTTGGACTACTATCAAAGA
ATACGACAAGACAGGGGCTCCTGCAGGGAAAGATGTTATTGAAGAATCCTTCGAGACTCCCATTTCCGCTAAGTACA
TTCGTCTGACTAATCTGGAAGACAAAATCCTGTTCCTGACTTTTAGTGAGTTTGCAATTGTGTCTGACGGTGGAGGT
GGCAGCGGCGGTGGTGGCTCGGGTGGAGGGTGCTGAGCTGCTAACAAAGCCCGAAAGGAAGCTGAGTTGGCTGCTGC
CACCGCTGAGCAATAACTAGCATAACCCCTTGGGGCCTCTAAACGGGTCTTGAGGGGTTTTTTGCTGAAAGGAGGAA
CTATATCCGGATATCCCGCAAGAGGCCCGGCAGTACCGGCATAACCAAGCCTATGCCTACAGCATCCAGGGTGACGG
TGCCGAGGATGACGATGAGCGCATTGTTAGATTTCATACACGGTGCCTGACTGCGTTAGCAATTTAACTGTGATAAA
CTACCGCATTAAAGCTTATCGATGATAAGCTGTCAAACATGAGAA

Expression and Purification of nanoCLAMPs Under Denaturing Conditions for Conjugation to Affinity Chromatography Resin (1 l Scale)

Glycerol stocks of NEc1 cells harboring nanoCLAMP expression vectors (described above) were used to inoculate 3 ml starter cultures of 2×YT/2% glucose (Glu)/100 mg/ml Carbenicillin (CB) and grown overnight at 37° C., 250 rpm. The overnight cultures were diluted 1:100 into 300 ml of Novagen Overnight Express Instant TB Medium/1% glycerol/CB and incubated 24 h, 30° C., 250 rpm. Cells were pelleted at 10 k×g, 10 min, 4° C., and lysed with 30 ml 100 mM NaH₂PO₄, 10 mM Tris, 6 M GuHCl (QAB) pH 8.5, plus 1 mM TCEP (QAB-TCEP, pH 8.5) using a Polytron to homogenize. The insoluble material was pelleted at 15 k×g, 20 min, 15° C., and the cleared supernatant applied to Ni Sepharose 6 Fast Flow (Cytivia) and incubated rotating for 1 h to overnight. The beads were transferred to a column and washed with 3 CV QAB-TCEP, pH 8.5, then 3 CV QAB, pH 8.5. The protein was eluted with QAB, pH 8.5+250 mM imidazole and quantified by A280. The purity of the eluted protein was measured by SDS-PAGE on 12% NuPAGE Bis-Tris gels and Coomassie staining with Gel-Code Blue (after removing the GuHCl by cold ethanol precipitation). Yields for nanoCLAMPs were typically 150-300 mg/L culture, and purity was typically greater than 90%.

The purified, denatured nanoCLAMPs in QAB, pH 8.5 were reduced with 2 mM TCEP if used after storage and conjugated to Sulfolink cross-linked, 6% beaded agarose (Thermo). Briefly, the resin was equilibrated with QAB, pH 8.5+5 mM EDTA and transferred to a column. The nanoCLAMP was adjusted to 8 mg/ml in a volume 2× the volume of the Sulfolink resin, and then incubated with the resin with rotation for 30 min. at room temperature. The resin was allowed to settle for 15 min., and the column drained to the top of the resin bed. The column was washed with QAB, pH 8.5 and then incubated with 50 mM L-Cys in QAB, pH 8.5 to quench for 15 min. with rotation. The column was allowed to settle, drained, and washed again with 6 M GuHCl, 20 mM Tris, (QCB) pH 8. Finally, the nanoCLAMP was refolded on the resin by rinsing with 6 CV of 20 mM MOPS, 150 mM NaCl (MBS), pH 6.5+1 mM CaCl₂).

Determination of Static Binding Capacity of nanoCLAMP Resins

A spiked E. coli lysate was prepared by pelleting the equivalent of OD₆₀₀=8 culture, discarding the supernatant and lysing the cells with BPER at 4 ml per g pellet, 20 min at room temperature with rotation. The insoluble material was removed by centrifugation, and the cleared lysate adjusted to a total protein concentration of approximately 1.87 mg/ml, 20% BPER in PBS, pH 7.4. The target protein, SUMO-GFP (described above) was spiked into the lysate to a final concentration of 0.025 to 0.2 mg/ml, depending on the application with a highly concentrated stock so that the total protein concentration remained unchanged. The spiked lysate was then incubated with 10 ml of the nanoCLAMP resin (packed volume) in a total volume of 1.4 ml, rotating at 4° C. for 1 h. The resin was precipitated by centrifugation and transferred to a small column. The resin was washed 4 times with 400 ml PBS, pH 7.4, and then eluted with 3 M imidazole, pH 8. The eluates were buffer exchanged twice with Zeba columns (7 kD MWCO, Thermo), and quantified by A₂₈₀ or fluorescence using an iD5 plate reader.

Expression and Purification of nanoCLAMPs for Biophysical Characterization

Glycerol stocks of NEc1 cells harboring nanoCLAMP expression vectors (described above) were used to inoculate 3 ml starter cultures of 2×YT/2% glucose (Glu)/100 mg/ml Carbenicillin (CB) and grown overnight at 37° C., 250 rpm. The overnight cultures were diluted 1:100 into 35 ml of Novagen Overnight Express Instant TB Medium/1% glycerol/CB and incubated 24 h, 30° C., 250 rpm. Cells were pelleted and lysed with QCB, pH 8, and insoluble material removed by centrifugation at 15 k×g, 20 min, 15° C. The cleared lysate was incubated with Ni Sepharose 6 Fast Flow (Cytivia) for >1 h rotating, room temperature, then transferred to 2 ml columns. The columns were washed with 6×1 ml QCB, pH 8, then refolded with 11 ml of 20 mM MOPS, 150 mM NaCl (MBS), 1 mM CaCl₂), pH 8. The nanoCLAMPs were eluted with MBS, 1 mM CaCl₂), 250 mM imidazole, pH 8, buffer exchanged to remove the imidazole using Zeba 7 MWCO desalting columns, and normalized to 1 mg/ml in MBS, 1 mM CaCl₂), pH 6.5.

### Expression and Purification of Target Proteins for Panning and Affinity Chromatography

To prepare a target protein for panning the library NL-26, we prepared a biotinylated yeast SUMO construct (B-SUMO; P1068) in a pET expression vector and transformed into BL21(DE3) E. coli harboring a constitutively expressed biotin ligase, BirA. An overnight starter culture was diluted 1:100 into 500 ml Novagen Overnight Express Instant TB Medium/1% glycerol/CB/CAM including 5 mM Biotin and incubated 24 h, 30° C., 250 rpm. Following the induction, the cells were pelleted and the media discarded. The pellet was frozen at −80° C., thawed on ice and resuspended at 5 ml/g pellet in MBS, pH 7.4+Pierce Protease Inhibitor Tablet Mini and sonicated on ice for 10 min. at 50% duty cycle. Biotin was added to 100 μM and the lysed cells incubated at 37° C., 30 min, 250 rpm to drive biotinylation to completion. The lysate was cleared by centrifugation at 30 k×g, 20 min, 4° C. and the supernatant transferred to a 2.25 ml SMT3-A1 resin (Nectagen, Inc) packed column at 1 ml/min. The resin was washed with 25 ml MBS, pH 7.4 and the protein eluted with polyol elution buffer (PEB): 10 mM Tris, 1 mM EDTA, 0.75M ammonium sulfate, 40% propylene glycol, pH 7.9. The protein was desalted 2× into 50 mM Tris, pH 8 and stored as a 50% glycerol stock at −20° C.

B-SUMO sequence (P1068)
(SEQ ID NO: 39)
MSDSEVNQEAKPEVKPEVKPETHINLKVSDGSSEIFFKIKKTTPLRRLM
EAFAKRQGKEMDSLRFLYDGIRIQADQTPEDLDMEDNDIIEAHREQIGG
GGGLNDIFEAQKIEWHE

To facilitate quantification of affinity chromatography target protein in eluates, we prepared a SUMO-GFP fusion (P10126RDG-1) that we could track by fluorescence spectroscopy. Briefly, the pET expression construct was transformed into NEc1 E. coli (Nectagen, Inc) and an overnight culture diluted 1:100 into 1 L Novagen Overnight Express Instant TB Medium/1% glycerol/CB and grown 24 h at 30° C., 250 rpm. The cells were pelleted, the media removed, and the cells lysed with BPER with Universal Nuclease (Thermo). The insoluble material was removed by centrifugation and the cleared supernatant loaded onto a 5 ml Ni Sepharose 6 Fast Flow column (Cytivia) at 1.5 ml/min, and the resin washed with 100 ml 50 mM NaH₂PO₄, 300 mM NaCl, 20 mM imidazole, pH 8, and then eluted with the same buffer with 250 mM imidazole. Protein purity of both B-SUMO and SUMO-GFP was assessed by SDS-PAGE using 12% NuPAGE, BisTris, MES running buffer under reducing conditions and stained with GelCode Blue (Thermo).

SUMO-GFP fusion (P10126RDG-1)
(SEQ ID NO: 40)
MGSSHHHHHHSDSEVNQEAKPEVKPEVKPETHINLKVSDGSSEIFFKIK
KTTPLRRLMEAFAKRQGKEMDSLRFLYDGIRIQADQTPEDLDMEDNDII
EAHREQIGGLYFQGSKGEELFTGVVPILVELDGDVNGHKFSVSGEGEGD
ATYGKLTLKFICTTGKLPVPWPTLVTTFAYGLQCFARYPDHMKQHDFFK
SAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGITHG
MDELYKIEGRGGKPIPNPLLGLDST

### Analysis of Monodispersity by Size Exclusion Chromatography

Purified nanoCLAMPs were diluted to a final concentration of 0.18 mg/ml in MBS, 1 mM CaCl₂), pH 6.5, centrifuged at 20 k×g, 2 min 4° C., and the supernatants transferred to a clean tube. The samples were loaded into a 125 μl sample loop and injected onto a Superdex 75 10/300 GL column (GE Healthcare Life Sciences, Pittsburgh, PA) equilibrated in MBS, 1 mM CaCl₂), pH 6.5 at a flowrate of 0.65 ml/min. The column was calibrated with Bio-Rad Gel Filtration Standard per manufacturer's instructions.

### Determination of Melting Temperature by Differential Scanning Fluorimetry

The melting temperature of purified nanoCLAMPs was determined using GloMelt Thermal Shift Protein Stability Kit (Biotium) per manufacturer's instructions. Briefly, purified nanoCLAMPs were adjusted to 1 mg/ml in MBS, 1 mM CaCl₂), pH 6.5 and diluted in half with 2× GloMelt (Biotium) and aliquoted to 386 well plate and sealed with optical film. The plate was then heated in a Quantstudio 5 qPCR machine using SYBR Green reporter with no passive reference. The heating profile was 25° C. for 2 min; ramp at 0.05° C./sec to 99° C.; 99° C. for 2 min. Tₘ is defined as the inflection point in the unfolding curve.

Determination of Protease Stability by Digestion with Trypsin and Chymotrypsin

Digestions were performed by incubating the nanoCLAMPs at 0.25 mg/ml in a 20 μl reaction containing 0.1 mg/ml trypsin (Roche Cat 11418475001) or chymotrypsin (Roche Cat 11418467001) diluted in 1 mM HCl, such that the final HCl concentration in the reaction was 0.1 mM. CaCl₂) was added to the reaction to 10 mM. The protein and remaining diluent buffer was MBS, pH 6.5. The reaction was incubated at 37° C. for the indicated times and stopped by adding 2 ml 10× Protease Arrest (G-Biosciences), analyzed on SDS-PAGE (12% NuPAGE, Bis Tris, in MES buffer) in SDS sample buffer with reducing agent, and stained with GelCode Blue (Thermo). Densitometry was carried out using GelAnalyzer software to measure relative staining intensities of the full-length band.

Phage Display Library NL-26 Construction for nC-B Class

The pCombX phagemid template p2799 (Table 8), contained the N-terminal and C-terminal constant regions of the nC-B class separated by a stuffer region containing HindIII and SpeI cut sites. This template was digested with HindIII and SpeI, gel purified, and the plasmid region amplified with degenerate primers 1957T R and 1960T F, which added the N and C-terminal part of nC-B as well as the randomized loops L1 and L8, respectively. The primers listed with a T indicate they are degenerate primers constructed using phosphoramidite trimer mixes (Glen Research) of oligos (IDT) containing all amino acids except Cys, Met, Lys, and Arg. The short internal region of the P2788 was amplified using primers 1958T F and 1959 R, which added and randomized Loop L2. PCR was carried out with ClonAmp HiFi PCR Mix, according to manufacturer's instructions (Takara Bio, Mountain View, CA). The reaction cycle was 98° C. for 10 sec, 65° C. for 10 sec, and 72° C. for 30 sec, repeated 30 times. These two amplicons, which contained overlapping ends, were gel purified and cloned together by Gibson Assembly (described below), creating the nC-B construct with 3 variable loops: Loop L1 (3 residues—817,819, 820), Loop L2 (7 residues, 838-844), and Loop L8 (5 residues, 931-935), for a total of 15 variable residues in 3 loops.

TABLE 8
Primer Sequences

| | SEQ ID | |
|---|---|---|
| Primer | NO: | Sequence (5′-3′) |
| 1957T R | 54 | ATACTCAACACCGGTGGAATCGTCTCCATCTAATAAATCGGCTTCGGAC |
| | | CCNNNNNNGACNNNCCAGCCGGGGGTATGGATTAAGGT |
| 1960T F | 55 | TATCGACGAAGATTTCGAGACTCCCATCTCCGCTCAGTACATCCGTTTAA |
| | | CTAATTTAGAANNNNNNNNNNNNNNNTTAACTTTTAGTGAGTTTGCAATC |
| 1958T F | 56 | GATGGAGACGATTCCACCGGTGTTGAGTATNNNNNNNNNNNNNNNNNN |
| | | NNNTCTCTTGCAGGAGAATTCATCGGATTAGACTTAGGTGAGGTAGTAG |
| | | AAG |
| 1959 R | 57 | GATGGGAGTCTCGAAATCTTCGTCGATAACATCTTGCCCTGCAGGAGCC |
| | | CCTGTATGGTCGTATTCACGGATAGTAGTCCAACTTTCGCCATCTAAGGA |
| | | GTACTCTAAACGGAAACGAGTCCATTTGTCGGAACTACCGCCTCCATCA |
| | | GCACCGATAACAAAATGGATACCGCCGACTTCTACTACCTCACCTAAGT |
| | | CTA |

NNN = randomized codon

To clone the library components, 10 μg of the large amplicon and 7.86 μg of the short amplicon were combined in a 2 ml reaction containing 1000 μl of Gibson Assembly Master Mix (2X) (NEB) and incubated at 50° C. for 30 min and then put on ice. The ligated DNA was then purified and concentrated in one Nucleospin Gel and PCR Cleanup Kit (Machery Nagel) and eluted in 45 μl EB. The DNA was then desalted on a VSWP 0.025 μm membrane (EMD Millipore) on ddH₂O for 40 min with a water change at 20 min. The desalted DNA was then adjusted to 100 ng/μl with ddH₂O and used to electroporate electrocompetent TG1 cells (Lucigen). Approximately 50 μl of DNA was added to 1.25 ml ice cold TG1 cells and pipetted up and down 4 times to mix on ice, after which 25 μl aliquots were transferred to 50 electroporation cuvettes (with 1 mm gaps) on ice. The cells were electroporated, and immediately quenched with 975 μl recovery media (Lucigen), pooled, and incubated at 37° C., 250 rpm for 1 h. To titer the library, 10 μl of recovered culture was serially diluted in 2×YT and 10 μl of each dilution spotted on 2×YT/glu/carb and incubated at 30° C. overnight. The remaining library was expanded to 3 L 2×YT/glu/carb and amplified overnight at 30° C., 250 rpm. The next day, the library was pelleted at 10 k×g, 10 min, 4° C. and the media discarded. The pellet was re-suspended to an OD₆₀₀ of 75 in 2×YT/2% glucose/18% glycerol, aliquoted and stored at −80° C.

Panning of nanoCLAMP Library NL-26 (nC-B Library)

For the first round of panning, 2.7 L of 2×YT medium with 2% glucose and 100 mg/ml carbenicillin (2×YT/Glu/CB) was inoculated with 3.6 ml of the NL-26 library glycerol stock (OD₆₀₀=75), to an OD₆₀₀ of approximately 0.1 and grown at 37° C., 250 rpm until the OD₆₀₀ reached 0.52. The library was infected by adding helper phage VCSM13 (Stratagene, Cat #200251) to 750 ml of culture at an MOI of 20 phage/cell, and incubating at 37° C., 100 rpm for 30 min, then 250 rpm for an additional 30 min. The cells were pelleted at 7500×g for 10 min, and the media discarded. The cells were resuspended in 1.2 L 2×YT/CB, 70 μg/ml kanamycin (KAN), and incubated 15 h at 30° C., 250 rpm. The cells were combined, and 100 ml was centrifuged at 10 k×g for 10 min. The phage containing supernatant was transferred to clean tubes and precipitated by adding 37.5 ml of 5×PEG/NaCl (20% polyethylene glycol 6000/2.5 M NaCl), and incubated on ice for 25 min. The phage was pelleted at 13 k×g, 25 min and the supernatant discarded. The phage was resuspended in 10 ml 20 mM NaH₂PO₄, 150 mM NaCl, pH 7.4 (PBS), then centrifuged at 15 k×g for 15 min to remove insoluble material. The phage was precipitated a second time by adding % volume 5×PEG/NaCl, incubated on ice for 5 min, and pelleted at 13 k×g, 10 min at 4° C. The phage pellet was resuspended in 3 ml PBS and quantified by absorbance at 268 nm (A₂₆₈=1 for a solution of 5×1012 phage/ml).

Two sets of 100 μl of Dynabeads MyOne Streptavidin T1 (ThermoFisher Scientific) magnetic beads slurry were washed 2×1 ml with PBS-T (PBS with 0.05% Tween 20), applying magnet in between washes to remove the supernatant, and then blocked in 1 ml of 2% dry milk solution in PBS with 0.05% Tween 20 (2% M-PBS-T) for 1 h, rotating, at room temperature. To preclear the phage against beads alone, 1 ml of phage was prepared at a concentration of 2×10¹³ phage/ml in 2% M-PBS-T, the block removed from the first set of beads, and the phage added to the beads and incubated 1 h, rotating. The magnet was applied, and the precleared phage removed and transferred to a clean tube. The magnet was applied, and this step repeated two times to ensure no carryover of beads bound to phage to the next step. Biotinylated target (B-SUMO) was added to the precleared phage to 100 nM final concentration and incubated rotating 1 h. Block was removed from the second set of beads, and the phage/B-SUMO mix was added to the beads to precipitate the biotinylated target and bound phage. The beads were washed 8× with PBS-T, 1 ml each, vortexing between each step and applying the magnet. The washed beads were eluted with 800 μl 0.1 M glycine, pH 2.0, 10 min rotating, the magnet applied, and the eluate transferred to 72 μl 2 M Tris base to neutralize. The neutralized phage was then added to 9 ml XL1-blue E. coli, which had been grown to OD₆₀₀=0.435 and placed on ice. The cells were infected at 37° C., 45 min, 175 rpm, and then expanded to 100 ml 2×YT/Glu/CB and incubated overnight at 30° C., 250 rpm.

The overnight cultures were harvested by measuring the OD₆₀₀, centrifuging the cells at 10 k×g for 10 min and then resuspending the cells to an OD₆₀₀ of 75 in 2×YT/18% glycerol. To prepare phage for the next round of panning, 5 ml of 2×YT/Glu/CB was inoculated with 5 μl of the 75 OD₆₀₀ glycerol stock and incubated at 37° C., 250 rpm until the OD₆₀₀ reached 0.5. The cells were superinfected at 20:1 phage:cell, mixed well, and incubated at 37° C., 30 min, 150 rpm and then 30 min at 250 rpm. The cells were pelleted at 5500×g, 10 min, the glucose containing media discarded and the cells resuspended in 10 ml 2×YT/CB/KAN and incubated overnight at 30° C., 250 rpm.

The overnight phage prep was processed as described above. The phage was then prepared at A₂₆₈=0.8 in 2% M-PBS-T, and the panning and pre-clearing continued as described, except in the second and third rounds, the biotinylated target concentration was reduced 10× per round. Washes after phage-capture was also increased in the third round, to 12 washes. In round 2, neutravidin-coated magnetic beads (Spherotech) were used in place of streptavidin-beads to reduce enrichment for streptavidin binders.

Qualitative semELISA of Individual Clones following Panning.

At the end of the last panning round, individual colonies were plated on 2×YT/Glu/CB agar plates following the 45 min 150 rpm recovery at 37° C. of the infected XL1-blue cells with the eluted phage. The next day, 95 colonies were inoculated into 400 μl 2×YT/Glu/CB in a 96-deep-well culture plate, and grown overnight at 37° C., 300 rpm to generate a master plate, to which glycerol was added to 18% for storage at −80° C. To prepare an induction plate for the ELISA, 5 μl of each master-plate culture was inoculated into 400 μl fresh 2×YT/0.1% glucose/CB medium and incubated for 2.75 h at 37° C., 300 rpm. IPTG was then added to 0.5 mM and the plates incubated at 30° C. with 300 rpm shaking overnight. Because the phagemid contains an amber stop codon, some nanoCLAMP protein is produced without the pIII domain, even though XL1-blue is a suppressor strain, resulting in the periplasmic localization of some nanoCLAMP, of which some percentage is ultimately secreted to the media. The media can then be used directly in an ELISA assay (soluble expression-based monoclonal enzyme-linked immunosorbent assay: semELISA). After the overnight induction, the plates were centrifuged at 1200×g for 10 min to pellet the cells. Streptavidin coated microtiter plates (ThermoFisher) were rinsed 3 times with 200 μl PBS, and then coated with biotinylated target proteins at 2 μg/ml with 100 μl/well and incubated 1 h. For blank controls, a plate was incubated with 100 μl/well PBS. The coating solution was removed, and the plates blocked with 2% M-PBS-T. The block was removed and 50 μl of 4% M-PBS-T added to each well. At this point 50 μl of each induction plate supernatant was transferred to the blank and protein-coated wells and pipetted 10 times to mix and incubated 1 h. The plates were washed 4 times with 200 μl PBS-T and the plates dumped and slapped on paper towels in between washes. After the washes, 75 μl of 1:2000 dilution anti-FLAG-HRP (Sigma A8592) in 4% M-PBS-T was added to each well and incubated 1 h. The anti-FLAG-HRP was discarded, and the plates washed as before. The plates were developed by adding 75 μl TMB Ultra substrate (ThermoFisher) and analyzed for positive signals compared to controls. Positive clones were then grown up from the master plate by inoculating 1 ml 2×YT/2% glucose/100 μg/ml CB with 3 μl glycerol stock and incubated for at least 6 h at 37° C., 250 rpm. The cells were then pelleted, and the media was discarded. Plasmid DNA was prepared from the pellets using the Qiaprep Spin Miniprep Kit, and the sequences determined by Sanger sequencing at Genewiz (South Plainfield, NJ). The nanoCLAMP inserts from unique positive clones was amplified and cloned into the pET expression vector, described above.

Biolayer Interferometry of nanoCLAMPs

Kinetic analysis of interactions between nanoCLAMPs and Biotinylated SUMO was carried out on an OctetRed96 using SAX streptavidin coated sensor tips. The tips were transferred first to buffer (MBS, 1 mM CaCl₂), pH 6.5+1% BSA) for 300 sec, then to B-SUMO at 2 mg/ml in buffer for 180 sec, then to buffer for 300 sec, then to at least 4 dilutions of nanoCLAMPs in buffer (association) for 200 sec, then to buffer (dissociation) for 500 sec. The cells were constantly vortexed at 1000 rpm at rm temp. The kinetics were fit to a 1:1 model and Kd calculated using global fit analysis.

### Dynamic Binding Capacity of P2808 Resin and SMT3-A1 Resin

A packed volume of 0.6 ml of P2808 resin or SMT3-A1 resin was packed into a Tricorn 5/50 column (5 mm ID×3.06 cm height) and equilibrated in 20 mM NaH₂PO₄, 150 mM NaCl, pH 7.4 (PBS) at 0.5 ml/min for 5 CV. The load, a Sumo-GFP fusion protein (MW=41,559 g/mol) diluted to a concentration, c, of 0.2 mg/ml in PBS, was pumped through the system with the column on bypass and the eluate fluorescence measured to determine the total load fluorescence at Ex/Em 485/535 nm. The delay volume, V_{delay}, was measured for the configuration at 0.5 ml. The load was then directed to the column and the volume Vₓ measured, where Vₓ is the volume where the fluorescence of the eluate=10% of that of the total load. The dynamic binding capacity, in units of mg/(ml resin), was then calculated as follows: DBC=(Vₓ−V_{delay})*c/(Vol Resin).

Purification of Sumo-GFP from a Spiked E. coli Lysate by Affinity Chromatography with P2808 Resin

A cleared E. coli lysate was prepared by lysing a pellet of NEc1 E. coli (a derivative of BL21(DE3) with the C-terminal region of SlyD knocked out by recombineering, Nectagen, Inc) with BPER (Thermo) and removing insoluble material by centrifugation at 15 k×g, 20 min, 4° C. The cleared supernatant was diluted to a total protein concentration of roughly 3.3 mg/ml with PBS, pH 7.4 such that the BPER reagent was present at 20% vol/vol. The target protein SUMO-GFP (MW=41,559 g/mol) was spiked-in to a final concentration of either 0.2 mg/ml or 0.025 mg/ml. The spiked lysate was loaded onto the column at 0.5 ml/min for indicated times, washed with 20 CV PBS, pH 7.4, then eluted with 3 M Imidazole, pH 8. Fractions containing eluted target were pooled and desalted 2×on Zeba 7 MWCO columns and the protein quantified by A₂₈₀. Imidazole removal was verified by testing the A280 of elution buffer alone following 2× desalting. Spiked lysate, early wash fractions, and pooled elutions (post buffer exchange) were analyzed by NuPAGE SDS PAGE under reducing conditions, 12%, Bis-Tris in MES running buffer and stained with Gel Code Blue (Thermo).

### Repeated AC Purification Cycles Including Cleaning in Place (CIP) of Resins Using 0.1 M NaOH

Repeated affinity chromatography purifications were carried out on an FPLC with a small 50 μl (packed) column using running buffer 20 mM MOPS, 150 mM NaCl, 1 mM CaCl₂), pH 7.2. The Load consisted of Sumo-GFP spiked into a cleared E. coli lysate (described above in Purification of Sumo-GFP from a spiked E. coli lysate) at 0.1 mg/ml. The cycle consisted of a 2 ml equilibration in running buffer at 1 ml/min, 0.5 ml load of spiked lysate at 0.5 ml/min, 3 ml wash with running buffer at 0.5 ml/min, 2 ml elution with 3 M imidazole, pH 8 (collected) at 0.5 ml/min, a 0.5 ml wash with running buffer at 0.5 ml/min, a cleaning in place cycle of 1.5 ml NaOH at 1 ml/min and then 2 ml at 0.2 ml/min (total contact time 10 min), and finally a refolding step with 5 ml running buffer at 1 ml/min. The target concentration in the eluates was measured by fluorescence spectroscopy in duplicate on an iD5 plate reader (Molecular Dynamics) Ex/Em 485/535 nm. The eluates were analyzed by SDS-PAGE using NuPAGE gels as described above.

Repeated AC Purification Cycles with Low pH Elution and Short Cleaning in Place with NaOH

Repeated affinity chromatography purifications were carried out as described above, except the column was eluted with 0.1 M citrate, pH 2.5 instead of 3 M imidazole, pH 8. Also, the cleaning in place step with 0.1 M NaOH was shortened to 1 ml, at 1 ml/min (1 min contact time per cycle). Since the eluted SUMO-GFP was denatured by the low pH elution, the relative elution concentrations were compared using densitometry of the target bands on SDS PAGE.

### Determination of Effect of Autoclaving or DMF Incubation on SUMO Binding Resin Binding Capacity and Specificity

For each resin tested, three 10 μl aliquots (packed vol) of resin were loaded into 1.5 ml screw cap tubes. To one, 1 ml DMF was added, and the tube incubated at rm temp for 2 h. To the other two, 100 μl MBS, 1 mM CaCl₂), pH 7.2 was added. One of these was autoclaved with its cap left slightly loose, on a 30 min liquid cycle, which sterilizes at around 120° C. and 20 psi for 30 min, and then slowly drops the pressure and the temperature over the next 90 min. The other set of resin was left on ice as a control. After two hours, all of the resins were cooled to room temperature and centrifuged at 1 k×g, 1 min. The control and autoclaved resin were stored overnight at 4° C. The DMF treated resin was rinsed 3× with fresh MBS, 1 mM CaCl₂, pH 7.2 and then stored overnight at 4° C. The next day all three of the sets of beads were rinsed with fresh buffer, and then incubated with 1.3 ml of E. coli lysate (prepared as described above) spiked with SUMO-GFP at 0.2 mg/ml, for 1 h, 4° C., rotating. The resin was loaded into a small, tared column, rinsed 4×400 ml PBS, pH 7.4, then eluted 3×25 ml 3 M imidazole, pH 8. The fluorescence of the eluates was read on an iD5 plate reader in duplicate as described and the concentration determined by comparison with a standard curve of the target and compared to controls. The concentrations were normalized and analyzed by SDS PAGE as described above to assess purity.

### Example 2. Using the Protein Scaffold to Target Diverse Antigens

We selected protein scaffolds that bound to diverse protein targets. A summary of the protein scaffolds and their cognate targets is shown in Table 9 below. Table 9 contains a subset of a much larger set of target-specific nanoCLAMPs, the majority of which possess the loop lengths of 4, 7, and 5 residues for loops 1, 2, and 8, respectively, as designed in library NL-26 (see above). To demonstrate the protein scaffold's ability to tolerate various loop lengths, we only included those nanoCLAMPs in Table 9 that possess at least one loop with a different length than the designed length. In Table 10 we demonstrate the scaffold's ability to support vast loop diversity by tabulating the amino acid sequences of nanoCLAMPs specific to several targets and show the diversity of loop sequences to a single target in several cases.

TABLE 9
Sequences of binding scaffolds (nanoCLAMPs) with variable loop lengths

| nC | pET | | SEQ ID | | | SEQ ID | L1 | | SEQ ID | L2 | | SEQ ID | L8 |
|---|---|---|---|---|---|---|---|---|---|---|---|---|---|
| class | clone | Sequence | NO: | Target | L1 | NO: | length | L2 | NO: | length | L8 | NO: | length |
| nC-B | p2877 | GSSHHHHHHDPTLIHT | 58 | B- | WV | 59 | 3 | VHY | 60 | 7 | GP | 61 | 5 |
| | | PGWWVPGSEADLLD | | CTLA4 | P | | | PV | | | WH | | |
| | | GDDSTGVEYVHYPVQ | | | | | | QS | | | G | | |
| | | SSLAGEFIGLDLGEVV | | | | | | | | | | | |
| | | EVGGIHFVIGADGGGS | | | | | | | | | | | |
| | | SDKWTRFRLEYSLDG | | | | | | | | | | | |
| | | ESWTTIREYDHTGAPA | | | | | | | | | | | |
| | | GKDVIDEDFETPISAQ | | | | | | | | | | | |
| | | YIRLTNLEGPWHGLTF | | | | | | | | | | | |
| | | SEFAIVSDELEDYKDD | | | | | | | | | | | |
| | | DDKG* | | | | | | | | | | | |
| nC-B | p2871 | GSSHHHHHHDPTLIHT | 62 | B- | NV | 63 | 4 | YIT | 64 | 5 | EP | 65 | 5 |
| | | PGWNVTVGSEADLLD | | CTLA4 | TV | | | TS | | | GS | | |
| | | GDDSTGVEYYITTSSL | | | | | | | | | H | | |
| | | AGEFIGLDLGEVVEVG | | | | | | | | | | | |
| | | GIHFVIGADGGGSSDK | | | | | | | | | | | |
| | | WTRFRLEYSLDGESW | | | | | | | | | | | |
| | | TTIREYDHTGAPAGQD | | | | | | | | | | | |
| | | VIDEDFETPISAQYIRL | | | | | | | | | | | |
| | | TNLEEPGSHLTFSEFAI | | | | | | | | | | | |
| | | VSDELEDYKDDDDKG* | | | | | | | | | | | |
| nC-B | p2876 | HHHHHHDPTLIHTPG | 66 | B- | VV | 67 | 4 | PQF | 68 | 7 | AF | 69 | 5 |
| | | WVVAAGSEADLLDGD | | CTLA4 | AA | | | QH | | | QP | | |
| | | DSTGVEYPQFQHQVS | | | | | | QV | | | G | | |
| | | LAGEFIGLDLGEVVEV | | | | | | | | | | | |
| | | GGIHFVIGADGGGSSD | | | | | | | | | | | |
| | | KWTRFRLEYSLDGES | | | | | | | | | | | |
| | | WTTIREYDHTGAPAG | | | | | | | | | | | |
| | | QDVIDEDFETPITAQYI | | | | | | | | | | | |
| | | RLTNLEAFQPGLTFSE | | | | | | | | | | | |
| | | FAIVSDELEDYKDDDD | | | | | | | | | | | |
| | | KG* | | | | | | | | | | | |
| nC-A | p1212LC | NPSLIRSESWGNEANL | 70 | B-Trx | — | — | 0 | SD | 71 | 7 | YSF | 7 2 | 5 |
| | | LDGDDNTGVWYSDHP | | (Ex- | | | | HP | | | RF | | |
| | | WFNSLAGEFIGLDLGK | | cellgen, | | | | WF | | | | | |
| | | EIKLDGIRFVIGKNGGG | | EG-5) | | | | N | | | | | |
| | | SSDKWNKFKLEYSLD | | | | | | | | | | | |
| | | NESWTTIKEYDKTGAP | | | | | | | | | | | |
| | | AGKDVIEESFETPISAK | | | | | | | | | | | |
| | | YIRLTNLEYSFRFLTFS | | | | | | | | | | | |
| | | EFAIVSDDYKDDDDKG | | | | | | | | | | | |
| | | * | | | | | | | | | | | |
| nC-A | pC961 | GSSHHHHHHNPSLIRS | 73 | B-Trx | PV | 74 | 3 | YSS | 75 | 7 | LKY | 76 | 5 |
| | | ESWPVYGNEANLLDG | | | Y | | | GTY | | | YG | | |
| | | DDNTGVWYYSSGTYF | | | | | | F | | | | | |
| | | SLAGEFIGLDLGKEIKL | | | | | | | | | | | |
| | | DGIRFVIGKNGGGSSD | | | | | | | | | | | |
| | | KWNKFKLEYSLDNES | | | | | | | | | | | |
| | | WTTIKEYDKTGAPAGK | | | | | | | | | | | |
| | | DVIEESFETPISAKYIRL | | | | | | | | | | | |
| | | TNLELKYYGLTFSEFAI | | | | | | | | | | | |
| | | VSDDYKDDDDKG* | | | | | | | | | | | |
| nC-A | 32 | GSSHHHHHHNPSLIRS | 77 | B-HbS | LA | 78 | 3 | YDV | 79 | 15 | SVI | 80 | 5 |
| | pComb3 | ESWLAFGNEANLLDG | | | F | | | AWI | | | PI | | |
| | 1315 | DDNTGVWYYDVAWIS | | | | | | SED | | | | | |
| | | EDRNNRDASLAGEFIG | | | | | | RN | | | | | |
| | | LDLGKEIKLDGIRFVIG | | | | | | NR | | | | | |
| | | KNGGGSSDKWNKFKL | | | | | | DA | | | | | |
| | | EYSLDNESWTTIKEYD | | | | | | | | | | | |
| | | KTGAPAGKDVIEESFE | | | | | | | | | | | |
| | | TPISAKYIRLTNLESVIP | | | | | | | | | | | |
| | | ILTFSEFAIVSDG* | | | | | | | | | | | |
| nC-A | p1301 | NPSLIRSESWAENGN | 81 | B-Avi- | AE | 82 | 3 | NE | 83 | 7 | PR | 84 | 5 |
| | | EANLLDGDDNTGVWY | | Sumo- | N | | | DD | | | HD | | |
| | | NEDDHFGSLAGEFIGL | | TEV- | | | | HF | | | T | | |
| | | DLGKEIKLDGIRFVIGK | | rtTA3 | | | | G | | | | | |
| | | NGGGSSDKWNKFKLE | | (1269-1) | | | | | | | | | |
| | | YSLDNESWTTIKEYDK | | | | | | | | | | | |
| | | TGAPAGKDVIEESFET | | | | | | | | | | | |
| | | PISAKYIRLTNLEPRHD | | | | | | | | | | | |
| | | TLTFSEFAIVSDDYKD | | | | | | | | | | | |
| | | DDDKG* | | | | | | | | | | | |
| nC-A | p1304 | NPSLIRSESWVGKGN | 85 | B-Avi- | VG | 86 | 3 | DE | 87 | 7 | YQ | 88 | 5 |
| | | EANLLDGDDNTGVWY | | Sumo- | K | | | DN | | | TRE | | |
| | | DEDNHFGSLAGEFIGL | | TEV- | | | | HF | | | | | |
| | | DLGKEIKLDGIRFVIGK | | rtTA3 | | | | G | | | | | |
| | | NGGGSSDKWNKFKLE | | (1269-1) | | | | | | | | | |
| | | YSLDNESWTTIKEYDK | | | | | | | | | | | |
| | | TGAPAGKDVIEESFET | | | | | | | | | | | |
| | | PISAKYIRLTNLEYQTR | | | | | | | | | | | |
| | | ELTFSEFAIVSDDYKD | | | | | | | | | | | |
| | | DDDKG* | | | | | | | | | | | |
| nC-A | p1420 | NPSLIRSESWVASGNE | 89 | B-Avi- | VA | 90 | 3 | KFG | 91 | 7 | RK | 92 | 5 |
| | | ANLLDGDDNTGVWYK | | Sumo- | S | | | HE | | | YIF | | |
| | | FGHEWESLAGEFIGLD | | TEV- | | | | WE | | | | | |
| | | LGKEIKLDGIRFVIGKN | | 11x V5 | | | | | | | | | |
| | | GGGSSDKWNKFKLEY | | Suntag | | | | | | | | | |
| | | SLDNESWTTIKEYDKT | | (p1267-1) | | | | | | | | | |
| | | GAPAGKDVIEESFETPI | | | | | | | | | | | |
| | | SAKYIRLTNLERKYIFL | | | | | | | | | | | |
| | | TFSEFAIVSDDYKDDD | | | | | | | | | | | |
| | | DKG* | | | | | | | | | | | |
| nC-A | p1854 | NPSLIRSESWVFFGNE | 93 | B- | VF | 94 | 3 | FTA | 95 | 7 | EH | 96 | 5 |
| | | ANLLDGDDNTGVWYF | | hCD63 | F | | | GN | | | QYL | | |
| | | TAGNKQSLAGEFIGLD | | | | | | KQ | | | | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLEEHQYLL | | | | | | | | | | | |
| | | TFSEFAIVSDDYKDDD | | | | | | | | | | | |
| | | DKG* | | | | | | | | | | | |
| nC-A | p1867 | NPSLIRSESWVFFGNE | 97 | B- | VF | 98 | 3 | AG | 99 | 7 | TGF | 100 | 5 |
| | | ANLLDGDDNTGVWYA | | hCD63 | F | | | GH | | | NY | | |
| | | GGHADDSLAGEFIGLD | | | | | | AD | | | | | |
| | | LGKEIKLDGIRFVIGKN | | | | | | D | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLETGFNYL | | | | | | | | | | | |
| | | TFSEFAIVSDDYKDDD | | | | | | | | | | | |
| | | DKG* | | | | | | | | | | | |
| nC-A | p1886 | NPSLIRSESWVLVGNE | 101 | B- | VL | 102 | 3 | RAS | 103 | 7 | EIR | 104 | 5 |
| | | ANLLDGDDNTGVWYR | | hFetuinA | V | | | AW | | | SV | | |
| | | ASAWYNSLAGEFIGLD | | | | | | YN | | | | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLEEIRSVL | | | | | | | | | | | |
| | | TFSEFAIVSDDYKDDD | | | | | | | | | | | |
| | | DKG* | | | | | | | | | | | |
| nC-A | p1230LC | NPSLIRSESWPYSYGN | 105 | B-Trx | PY | 106 | 4 | N | 107 | 1 | AP | 108 | 4 |
| | | EANLLDGDDNTGVWY | | (Ex- | SY | | | | | | GN | | |
| | | NSLAGEFIGLDLGKEIK | | cellgen, | | | | | | | | | |
| | | LDGIRFVIGKNGGGSS | | EG-5) | | | | | | | | | |
| | | DKWNKFKLEYSLDNE | | | | | | | | | | | |
| | | SWTTIKEYDKTGAPAG | | | | | | | | | | | |
| | | KDVIEESFETPISAKYI | | | | | | | | | | | |
| | | RLTNLEAPGNLTFSEF | | | | | | | | | | | |
| | | AIVSDDYKDDDDKG* | | | | | | | | | | | |
| nC-A | p1796 | NPSLIRSESWDEWWG | 109 | B- | DE | 110 | 4 | YYD | 111 | 4 | DIK | 112 | 4 |
| | | NEANLLDGDDNTGVW | | 1631-5 | W | | | D | | | Y | | |
| | | YYYDDSLAGEFIGLDL | | (hMito | W | | | | | | | | |
| | | GKEIKLDGIRFVIGKNG | | NEET) | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEDIKYLTF | | | | | | | | | | | |
| | | SEFAIVSDDYKDDDDK | | | | | | | | | | | |
| | | G* | | | | | | | | | | | |
| nC-A | p1302 | NPSLIRSESWHKAHG | 113 | B-Avi- | HK | 114 | 4 | WA | 115 | 5 | YVE | 116 | 5 |
| | | NEANLLDGDDNTGVW | | Sumo- | AH | | | HKK | | | KE | | |
| | | YWAHKKSLAGEFIGLD | | TEV- | | | | | | | | | |
| | | LGKEIKLDGIRFVIGKN | | rtTA3 | | | | | | | | | |
| | | GGGSSDKWNKFKLEY | | (1269-1) | | | | | | | | | |
| | | SLDNESWTTIKEYDKT | | | | | | | | | | | |
| | | GAPAGKDVIEESFETPI | | | | | | | | | | | |
| | | SAKYIRLTNLEYVEKEL | | | | | | | | | | | |
| | | TFSEFAIVSDDYKDDD | | | | | | | | | | | |
| | | DKG* | | | | | | | | | | | |
| nC-A | p1884 | NPSLIRSESWLDNQG | 117 | B- | LD | 118 | 4 | EKN | 119 | 5 | TIIT | 120 | 5 |
| | | NEANLLDGDDNTGVW | | hITGAV/ | NQ | | | DY | | | E | | |
| | | YEKNDYSLAGEFIGLD | | ITGB5 | | | | | | | | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLETIITELT | | | | | | | | | | | |
| | | FSVFAIVSDDYKDDDD | | | | | | | | | | | |
| | | KG* | | | | | | | | | | | |
| nC-A | p2078 | NPSLIRSESWVIWVGN | 121 | human | VI | 122 | 4 | NFE | 123 | 5 | FVY | 124 | 5 |
| | | EANLLDGDDNTGVWY | | serum | WV | | | YY | | | GV | | |
| | | NFEYYSLAGEFIGLDL | | EV | | | | | | | | | |
| | | GKEIKLDGIRFVIGKNG | | | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEFVYGVLT | | | | | | | | | | | |
| | | FSEFAIVSDDYKDDDD | | | | | | | | | | | |
| | | KG* | | | | | | | | | | | |
| nC-A | p835 | GSSHHHHHHNPSLIRS | 125 | B- | EP | 126 | 4 | LGL | 127 | 6 | KVF | 128 | 5 |
| | | ESWEPWAGNEANLLD | | GFP- | WA | | | YG | | | IW | | |
| | | GDDNTGVWYLGLYGY | | avi | | | | Y | | | | | |
| | | SLAGEFIGLDLGKEIKL | | | | | | | | | | | |
| | | DGIRFVIGKNGGGSSD | | | | | | | | | | | |
| | | KWNKFKLEYSLDNES | | | | | | | | | | | |
| | | WTTIKEYDKTGAPAGK | | | | | | | | | | | |
| | | DVIEESFETPISAKYIRL | | | | | | | | | | | |
| | | TNLEKVFIWLTFSEFAI | | | | | | | | | | | |
| | | VSDDYKDDDDKG* | | | | | | | | | | | |
| nC-A | pC976- | MGCSHHHHHHNPSLI | 129 | Neutra | FIK | 130 | 4 | WL | 131 | 6 | THY | 132 | 5 |
| | T7 | RSESWFIKKGNEANLL | | vidin | K | | | PNY | | | FN | | |
| | | DGDDNTGVWYWLPN | | | | | | F | | | | | |
| | | YFSLAGEFIGLDLGKEI | | | | | | | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLETHYFNLTFSE | | | | | | | | | | | |
| | | FAIVSD* | | | | | | | | | | | |
| nC-A | p1157 | NPSLIRSESWQVFYG | 133 | B- | QV | 134 | 4 | YG | 135 | 6 | PW | 136 | 5 |
| | | NEANLLDGDDNTGVW | | GFP- | FY | | | KD | | | WS | | |
| | | YYGKDDWSLAGEFIGL | | avi | | | | DW | | | Y | | |
| | | DLGKEIKLDGIRFVIGK | | | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEPWW | | | | | | | | | | | |
| | | SYLTFSEFAIVSDDYK | | | | | | | | | | | |
| | | DDDDKG* | | | | | | | | | | | |
| nC-A | p1253LC- | MGSSHHHHHHNPSLI | 137 | B- | VR | 138 | 4 | AL | 139 | 6 | SQ | 140 | 5 |
| | T7 | RSESWVRWEGNEAN | | mCherry | WE | | | WN | | | YS | | |
| | | LLDGDDNTGVWYALW | | | | | | HL | | | D | | |
| | | NHLSLAGEFIGLDLGK | | | | | | | | | | | |
| | | EIKLDGIRFVIGKNGGG | | | | | | | | | | | |
| | | SSDKWNKFKLEYSLD | | | | | | | | | | | |
| | | NESWTTIKEYDKTGAP | | | | | | | | | | | |
| | | AGKDVIEESFETPISAK | | | | | | | | | | | |
| | | YIRLTNLESQYSDLTFS | | | | | | | | | | | |
| | | EFAIVSDGGGGSGGG | | | | | | | | | | | |
| | | GSGGGC* | | | | | | | | | | | |
| nC-A | p1363 | NPSLIRSESWPFIIGNE | 141 | B- | PFI | 142 | 4 | IPS | 143 | 6 | FKY | 144 | 5 |
| | | ANLLDGDDNTGVWYI | | Actin | I | | | HHF | | | TY | | |
| | | PSHHFSLAGEFIGLDL | | | | | | | | | | | |
| | | GKEIKLDGIRFVIGKNG | | | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEFKYTYLT | | | | | | | | | | | |
| | | FSEFAIVSDDYKDDDD | | | | | | | | | | | |
| | | KG* | | | | | | | | | | | |
| nC-A | p1373 | NPSLIRSESWIWAIGN | 145 | B- | IW | 146 | 4 | IPK | 147 | 6 | LQI | 148 | 5 |
| | | EANLLDGDDNTGVWY | | Actin | AI | | | EKK | | | SY | | |
| | | IPKEKKSLAGEFIGLDL | | | | | | | | | | | |
| | | GKEIKLDGIRFVIGKNG | | | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLELQISYLT | | | | | | | | | | | |
| | | FSEFAIVSDDYKDDDD | | | | | | | | | | | |
| | | KG* | | | | | | | | | | | |
| nC-A | p1810 | NPSLIRSESWKITWGN | 149 | B- | KIT | 150 | 4 | LNV | 151 | 6 | YE | 152 | 5 |
| | | EANLLDGDDNTGVWY | | 1632-5 | W | | | FKK | | | HS | | |
| | | LNVFKKSLAGEFIGLDL | | (hPDIA3) | | | | | | | E | | |
| | | GKEIKLDGIRFVIGKNG | | | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEYEHSELT | | | | | | | | | | | |
| | | FSEFAIVSDDYKDDDD | | | | | | | | | | | |
| | | KG* | | | | | | | | | | | |
| nC-A | p1857 | NPSLIRSESWIFWSGN | 153 | B- | IF | 154 | 4 | YKY | 155 | 6 | VVE | 156 | 5 |
| | | EANLLDGDDNTGVWY | | hCD63 | WS | | | PK | | | TW | | |
| | | YKYPKQSLAGEFIGLD | | | | | | Q | | | | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLEVVETW | | | | | | | | | | | |
| | | LTFSEFAIVSDDYKDD | | | | | | | | | | | |
| | | DDKG* | | | | | | | | | | | |
| nC-A | p1870 | NPSLIRSESWHVIVGN | 157 | B- | HV | 158 | 4 | ISE | 159 | 6 | GK | 160 | 5 |
| | | EANLLDGDDNTGVWY | | hCD63 | IV | | | FA | | | YVF | | |
| | | ISEFAWSLAGEFIGLDL | | | | | | W | | | | | |
| | | GKEIKLDGIRFVIGKNG | | | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEGKYVFLT | | | | | | | | | | | |
| | | FSEFAIVSDDYKDDDD | | | | | | | | | | | |
| | | KG* | | | | | | | | | | | |
| nC-A | p1892 | NPSLIRSESWFRDHG | 161 | B- | FR | 162 | 4 | FAL | 163 | 6 | HH | 164 | 5 |
| | | NEANLLDGDDNTGVW | | hCD81 | DH | | | VNT | | | FSD | | |
| | | YFALVNTSLAGEFIGL | | | | | | | | | | | |
| | | DLGKEIKLDGIRFVIGK | | | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEHHFS | | | | | | | | | | | |
| | | DLTFSEFAIVSDDYKD | | | | | | | | | | | |
| | | DDDKG* | | | | | | | | | | | |
| nC-A | p1900 | NPSXIRSESWPESFG | 165 | B- | PE | 166 | 4 | FAL | 167 | 6 | VN | 168 | 5 |
| | | NEANLLDGDDNTGVW | | hCD81 | SF | | | INS | | | ND | | |
| | | YFALINSSLAGEFIGLD | | | | | | | | | P | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLEVNNDP | | | | | | | | | | | |
| | | LTFSEFAIVSDDYKDD | | | | | | | | | | | |
| | | DDKG* | | | | | | | | | | | |
| nC-A | p1913 | NPSLIRSESWPTKFGN | 169 | B- | PT | 170 | 4 | FW | 171 | 6 | PSI | 172 | 5 |
| | | EANLLDGDDNTGVWY | | hCD81 | KF | | | GF | | | NY | | |
| | | FWGFWKSLAGEFIGL | | | | | | WK | | | | | |
| | | DLGKEIKLDGIRFVIGK | | | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEPSINY | | | | | | | | | | | |
| | | LTFSEFAIVSDDYKDD | | | | | | | | | | | |
| | | DDKG* | | | | | | | | | | | |
| nC-A | p1923 | NPSLIRSESWHENAG | 173 | B- | HE | 174 | 4 | FAL | 175 | 6 | RH | 176 | 5 |
| | | NEANLLDGDDNTGVW | | hCD81 | NA | | | ING | | | HQ | | |
| | | YFALINGSLAGEFIGLD | | | | | | | | | Q | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLERHHQQ | | | | | | | | | | | |
| | | LTFSEFAIVSDDYKDD | | | | | | | | | | | |
| | | DDKG* | | | | | | | | | | | |
| nC-A | p1928 | NPSLIRSESWGSGHG | 177 | B- | GS | 178 | 4 | YIVI | 179 | 6 | RG | 180 | 5 |
| | | NEANLLDGDDNTGVW | | hCD81 | GH | | | NK | | | VYV | | |
| | | YYIVINKSLAGEFIGLD | | | | | | | | | | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLERGVYV | | | | | | | | | | | |
| | | LTFSEFAIVSDDYKDD | | | | | | | | | | | |
| | | DDKG* | | | | | | | | | | | |
| nC-A | p1932 | NPSLIRSESWPRSNG | 181 | B- | PR | 182 | 4 | FAI | 183 | 6 | EQ | 184 | 5 |
| | | NEANLLDGDDNTGVW | | hCD81 | SN | | | LNI | | | AYT | | |
| | | YFAILNISLAGEFIGLDL | | | | | | | | | | | |
| | | GKEIKLDGIRFVIGKNG | | | | | | | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEEQAYTLT | | | | | | | | | | | |
| | | FSEFAIVSDDYKDDDD | | | | | | | | | | | |
| | | KG* | | | | | | | | | | | |
| nC-A | p2095_ | MGSSHHHHHHNPSLI | 185 | hPDIA3 | IIR | 186 | 4 | PKI | 187 | 6 | WR | 188 | 5 |
| | 1_ | RSESWIRDGNEANLL | | | D | | | FSH | | | GV | | |
| | 2019_ | DGDDNTGVWYPKIFS | | | | | | | | | A | | |
| | 05_23 | HSLAGEFIGLDLGKEIK | | | | | | | | | | | |
| | | LDGIRFVIGKNGGGSS | | | | | | | | | | | |
| | | DKWNKFKLEYSLDNE | | | | | | | | | | | |
| | | SWTTIKEYDKTGAPAG | | | | | | | | | | | |
| | | KDVIEESFETPISAKYI | | | | | | | | | | | |
| | | RLTNLEWRGVALTFSE | | | | | | | | | | | |
| | | FAIVSDGGGLNDIFEA | | | | | | | | | | | |
| | | QKIEWHE* | | | | | | | | | | | |
| nC-A | pC919 | GSSHHHHHHNPSLIRS | 189 | BgaI | ET | 190 | 4 | FKK | 191 | 8 | SHF | 192 | 5 |
| | | ESWETWTGNEANLLD | | | WT | | | NFH | | | RA | | |
| | | GDDNTGVWYFKKNFH | | | | | | VA | | | | | |
| | | VASLAGEFIGLDLGKEI | | | | | | | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLESHFRALTFSE | | | | | | | | | | | |
| | | FAIVSDDYKDDDDKG* | | | | | | | | | | | |
| nC-A | 1295 | NPSLIRSESWTYVIGN | 193 | B-Avi- | TY | 194 | 4 | VLN | 195 | 8 | EKF | 196 | 5 |
| | | EANLLDGDDNTGVWY | | Sumo- | VI | | | KLK | | | DN | | |
| | | VLNKLKKKSLAGEFIGL | | TEV- | | | | KK | | | | | |
| | | DLGKEIKLDGIRFVIGK | | rtTA3 | | | | | | | | | |
| | | NGGGSSDKWNKFKLE | | (1269-1) | | | | | | | | | |
| | | YSLDNESWTTIKEYDK | | | | | | | | | | | |
| | | TGAPAGKDVIEESFET | | | | | | | | | | | |
| | | PISAKYIRLTNLEEKFD | | | | | | | | | | | |
| | | NLTFSEFAIVSDDYKD | | | | | | | | | | | |
| | | DDDKG* | | | | | | | | | | | |
| nC-A | p1312 | NPSLIRSESWVFIIGNE | 197 | B-Avi- | VFI | 198 | 4 | LLH | 199 | 8 | HA | 200 | 5 |
| | | ANLLDGDDNTGVWYL | | Sumo- | I | | | KLT | | | KK | | |
| | | LHKLTKTSLAGEFIGLD | | TEV- | | | | KT | | | W | | |
| | | LGKEIKLDGIRFVIGKN | | rtTA3 | | | | | | | | | |
| | | GGGSSDKWNKFKLEY | | (1269-1) | | | | | | | | | |
| | | SLDNESWTTIKEYDKT | | | | | | | | | | | |
| | | GAPAGKDVIEESFETPI | | | | | | | | | | | |
| | | SAKYIRLTNLEHAKKW | | | | | | | | | | | |
| | | LTFSEFAIVSDDYKDD | | | | | | | | | | | |
| | | DDKG* | | | | | | | | | | | |
| nC-A | p1800 | NPSLIRSESWVVIDGN | 201 | B- | VVI | 202 | 4 | FTD | 203 | 8 | KTN | 204 | 5 |
| | | EANLLDGDDNTGVWY | | 1631-5 | D | | | YWI | | | EF | | |
| | | FTDYWIVESLAGEFIGL | | (hMito | | | | VE | | | | | |
| | | DLGKEIKLDGIRFVIGK | | NEET) | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEKTNE | | | | | | | | | | | |
| | | FLTFSEFAIVSDDYKD | | | | | | | | | | | |
| | | DDDKG* | | | | | | | | | | | |
| nC-A | p1859 | NPSLIRSESWAWWIG | 205 | B- | AW | 206 | 4 | ALK | 207 | 8 | PSL | 208 | 5 |
| | | NEANLLDGDDNTGVW | | hCD63 | WI | | | VFN | | | TD | | |
| | | YALKVFNYWSLAGEFI | | | | | | YW | | | | | |
| | | GLDLGKEIKLDGIRFVI | | | | | | | | | | | |
| | | GKNGGGSSDKWNKF | | | | | | | | | | | |
| | | KLEYSLDNESWTTIKE | | | | | | | | | | | |
| | | YDKTGAPAGKDVIEES | | | | | | | | | | | |
| | | FETPISAKYIRLTNLEP | | | | | | | | | | | |
| | | SLTDLTFSEFAIVSDDY | | | | | | | | | | | |
| | | KDDDDKG* | | | | | | | | | | | |
| nC-A | p1431 | NPSLIRSESWAQTNG | 209 | B-Avi- | AQ | 210 | 4 | VAV | 211 | 9 | HA | 212 | 5 |
| | | NEANLLDGDDNTGVW | | Sumo- | TN | | | VTH | | | YK | | |
| | | YVAVVTHIKFSLAGEFI | | TEV- | | | | IKF | | | N | | |
| | | GLDLGKEIKLDGIRFVI | | 11x V5 | | | | | | | | | |
| | | GKNGGGSSDKWNKF | | Suntag | | | | | | | | | |
| | | KLEYSLDNESWTTIKE | | (p1267-1) | | | | | | | | | |
| | | YDKTGAPAGKDVIEES | | | | | | | | | | | |
| | | FETPISAKYIRLTNLEH | | | | | | | | | | | |
| | | AYKNLTFSEFAIVSDD | | | | | | | | | | | |
| | | YKDDDDKG* | | | | | | | | | | | |
| nC-A | plate22- | GSSHHHHHHNPSLIRS | 213 | B- | SIY | 214 | 4 | TGK | 215 | 9 | FKT | 216 | 3 |
| | 624F | ESWSIYKGNEANLLDG | | Sumo | K | | | LDK | | | | | |
| | | DDNTGVWYTGKLDKS | | | | | | SYN | | | | | |
| | | YNSLAGEFIGLDLGKEI | | | | | | | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLEFKTLTFSEFAI | | | | | | | | | | | |
| | | VSD* | | | | | | | | | | | |
| nC-A | p1818 | NPSLIRSESWWRPTG | 217 | B- | W | 218 | 4 | AKI | 219 | 10 | RH | 220 | 5 |
| | | NEANLLDGDDNTGVW | | 1632-5 | RP | | | YFY | | | EKS | | |
| | | YAKIYFYYWDISLAGE | | (hPDIA3) | T | | | YW | | | | | |
| | | FIGLDLGKEIKLDGIRF | | | | | | DI | | | | | |
| | | VIGKNGGGSSDKWNK | | | | | | | | | | | |
| | | FKLEYSLDNESWTTIK | | | | | | | | | | | |
| | | EYDKTGAPAGKDVIEE | | | | | | | | | | | |
| | | SFETPISAKYIRLTNLE | | | | | | | | | | | |
| | | RHEKSLTFSEFAIVSD | | | | | | | | | | | |
| | | DYKDDDDKG* | | | | | | | | | | | |
| nC-A | p1386 | NPSLIRSESWDDVHG | 221 | B- | DD | 222 | 4 | FHY | 223 | 11 | KYS | 224 | 5 |
| | | NEANLLDGDDNTGVW | | tubulin | VH | | | YFR | | | LE | | |
| | | YFHYYFRSSKKHSLAG | | | | | | SSK | | | | | |
| | | EFIGLDLGKEIKLDGIR | | | | | | KH | | | | | |
| | | FVIGKNGGGSSDKWN | | | | | | | | | | | |
| | | KFKLEYSLDNESWTTI | | | | | | | | | | | |
| | | KEYDKTGAPAGKDVIE | | | | | | | | | | | |
| | | ESFETPISAKYIRLTNL | | | | | | | | | | | |
| | | EKYSLELTFSEFAIVSD | | | | | | | | | | | |
| | | DYKDDDDKG* | | | | | | | | | | | |
| nC-A | 22pC | GSSHHHHHHNPSLIRS | 225 | B-HbS | INL | 226 | 4 | GIL | 227 | 14 | KDT | 228 | 5 |
| | omb3 | ESWINLFGNEANLLDG | | | F | | | YTV | | | TF | | |
| | 1315 | DDNTGVWYGILYTVSL | | | | | | SLE | | | | | |
| | | EYSKIYSLAGEFIGLDL | | | | | | YSK | | | | | |
| | | GKEIKLDGIRFVIGKNG | | | | | | IY | | | | | |
| | | GGSSDKWNKFKLEYS | | | | | | | | | | | |
| | | LDNESWTTIKEYDKTG | | | | | | | | | | | |
| | | APAGKDVIEESFETPIS | | | | | | | | | | | |
| | | AKYIRLTNLEKDTTFLT | | | | | | | | | | | |
| | | FSEFAIVSDG* | | | | | | | | | | | |
| nC-A | mCherry | GSSHHHHHHNPSLIRS | 229 | B- | VG | 230 | 4 | PW | 231 | 15 | YVS | 232 | 5 |
| | E6-1 | ESWVGSKGNEANLLD | | mCherry | SK | | | FPK | | | VI | | |
| | | GDDNTGVWYPWFPK | | | | | | AIF | | | | | |
| | | AIFFKNREFGSLAGEFI | | | | | | FKN | | | | | |
| | | GLDLGKEIKLDGIRFVI | | | | | | REF | | | | | |
| | | GKNGGGSSDKWNKF | | | | | | G | | | | | |
| | | KLEYSLDNESWTTIKE | | | | | | | | | | | |
| | | YDKTGAPAGKDVIEES | | | | | | | | | | | |
| | | FETPISAKYIRLTNLEY | | | | | | | | | | | |
| | | VSVILTFSEFAIVSDG* | | | | | | | | | | | |
| nC-A | p1325 | NPSLIRSESWQFSNG | 233 | B-Avi- | QF | 234 | 4 | DN | 235 | 7 | VIY | 236 | 3 |
| | | NEANLLDGDDNTGVW | | Sumo- | SN | | | ND | | | | | |
| | | YDNNDHFGSLAGEFIG | | TEV- | | | | HF | | | | | |
| | | LDLGKEIKLDGIRFVIG | | rtTA3 | | | | G | | | | | |
| | | KNGGGSSDKWNKFKL | | (1269-1) | | | | | | | | | |
| | | EYSLDNESWTTIKEYD | | | | | | | | | | | |
| | | KTGAPAGKDVIEESFE | | | | | | | | | | | |
| | | TPISAKYIRLTNLEVIYL | | | | | | | | | | | |
| | | TFSEFAIVSDDYKDDD | | | | | | | | | | | |
| | | DKG* | | | | | | | | | | | |
| nC-A | pC962 | GSSHHHHHHNPSLIRS | 237 | B-Trx | HT | 238 | 4 | NN | 239 | 7 | AK | 240 | 4 |
| | | ESWHTYNGNEANLLD | | | YN | | | NS | | | NN | | |
| | | GDDNTGVWYNNNSW | | | | | | WF | | | | | |
| | | FSSLAGEFIGLDLGKEI | | | | | | S | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLEAKNNLTFSEF | | | | | | | | | | | |
| | | AIVSDDYKDDDDKG* | | | | | | | | | | | |
| nC-A | pC959 | GSSHHHHHHNPSLIRS | 241 | B-Trx | PG | 242 | 4 | EDK | 243 | 7 | AKY | 244 | 4 |
| | | ESWPGVIGNEANLLD | | | VI | | | IWF | | | N | | |
| | | GDDNTGVWYEDKIWF | | | | | | N | | | | | |
| | | NSLAGEFIGLDLGKEIK | | | | | | | | | | | |
| | | LDGIRFVIGKNGGGSS | | | | | | | | | | | |
| | | DKWNKFKLEYSLDNE | | | | | | | | | | | |
| | | SWTTIKEYDKTGAPAG | | | | | | | | | | | |
| | | KDVIEESFETPISAKYI | | | | | | | | | | | |
| | | RLTNLEAKYNLTFSEF | | | | | | | | | | | |
| | | AIVSDDYKDDDDKG* | | | | | | | | | | | |
| nC-A | pC969 | GSSHHHHHHNPSLIRS | 245 | B-Trx | PY | 246 | 4 | TG | 247 | 7 | AQ | 248 | 4 |
| | | ESWPYLDGNEANLLD | | | LD | | | RN | | | NN | | |
| | | GDDNTGVWYTGRNPF | | | | | | PFN | | | | | |
| | | NSLAGEFIGLDLGKEIK | | | | | | | | | | | |
| | | LDGIRFVIGKNGGGSS | | | | | | | | | | | |
| | | DKWNKFKLEYSLDNE | | | | | | | | | | | |
| | | SWTTIKEYDKTGAPAG | | | | | | | | | | | |
| | | KDVIEESFETPISAKYI | | | | | | | | | | | |
| | | RLTNLEAQNNLTFSEF | | | | | | | | | | | |
| | | AIVSDDYKDDDDKG* | | | | | | | | | | | |
| nC-A | pC960 | GSSHHHHHHNPSLIRS | 249 | B-Trx | PH | 250 | 4 | WD | 251 | 7 | PQ | 252 | 4 |
| | | ESWPHNEGNEANLLD | | | NE | | | RSV | | | YN | | |
| | | GDDNTGVWYWDRSV | | | | | | FN | | | | | |
| | | FNSLAGEFIGLDLGKEI | | | | | | | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLEPQYNLTFSEF | | | | | | | | | | | |
| | | AIVSDDYKDDDDKG* | | | | | | | | | | | |
| nC-A | pC968 | GSSHHHHHHNPSLIRS | 253 | B-Trx | KW | 254 | 4 | DG | 255 | 7 | SQ | 256 | 4 |
| | | ESWKWVSGNEANLLD | | | VS | | | NH | | | HN | | |
| | | GDDNTGVWYDGNHW | | | | | | WF | | | | | |
| | | FNSLAGEFIGLDLGKEI | | | | | | N | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLESQHNLTFSEF | | | | | | | | | | | |
| | | AIVSDDYKDDDDKG* | | | | | | | | | | | |
| nC-A | pC958 | GSSHHHHHHNPSLIRS | 257 | B-Trx | PS | 258 | 4 | ENK | 259 | 7 | TRY | 260 | 4 |
| | | ESWPSTSGNEANLLD | | | TS | | | SW | | | N | | |
| | | GDDNTGVWYENKSW | | | | | | FN | | | | | |
| | | FNSLAGEFIGLDLGKEI | | | | | | | | | | | |
| | | KLDGIRFVIGKNGGGS | | | | | | | | | | | |
| | | SDKWNKFKLEYSLDN | | | | | | | | | | | |
| | | ESWTTIKEYDKTGAPA | | | | | | | | | | | |
| | | GKDVIEESFETPISAKY | | | | | | | | | | | |
| | | IRLTNLETRYNLTFSEF | | | | | | | | | | | |
| | | AIVSDDYKDDDDKG* | | | | | | | | | | | |
| nC-A | p1209LC | NPSLIRSESWVIKKGN | 261 | B-Trx | VIK | 262 | 4 | SHK | 263 | 7 | VR | 264 | 4 |
| | | EANLLDGDDNTGVWY | | (Ex- | K | | | LW | | | DN | | |
| | | SHKLWFNSLAGEFIGL | | cellgen, | | | | FN | | | | | |
| | | DLGKEIKLDGIRFVIGK | | EG-5) | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEVRDN | | | | | | | | | | | |
| | | LTFSEFAIVSDDYKDD | | | | | | | | | | | |
| | | DDKG | | | | | | | | | | | |
| nC-A | p1210LC | NPSLIRSESWRGHPG | 265 | B-Trx | RG | 266 | 4 | RN | 267 | 7 | PS | 268 | 4 |
| | | NEANLLDGDDNTGVW | | (Ex- | HP | | | HH | | | NN | | |
| | | YRNHHVFSSLAGEFIG | | cellgen, | | | | VFS | | | | | |
| | | LDLGKEIKLDGIRFVIG | | EG-5) | | | | | | | | | |
| | | KNGGGSSDKWNKFKL | | | | | | | | | | | |
| | | EYSLDNESWTTIKEYD | | | | | | | | | | | |
| | | KTGAPAGKDVIEESFE | | | | | | | | | | | |
| | | TPISAKYIRLTNLEPSN | | | | | | | | | | | |
| | | NLTFSEFAIVSDDYKD | | | | | | | | | | | |
| | | DDDKG* | | | | | | | | | | | |
| nC-A | p1214LC | NPSLIRSESWPDLGG | 269 | B-Trx | PD | 270 | 4 | AEH | 271 | 7 | AR | 272 | 4 |
| | | NEANLLDGDDNTGVW | | (Ex- | LG | | | TVY | | | EN | | |
| | | YAEHTVYNSLAGEFIG | | cellgen, | | | | N | | | | | |
| | | LDLGKEIKLDGIRFVIG | | EG-5) | | | | | | | | | |
| | | KNGGGSSDKWNKFKL | | | | | | | | | | | |
| | | EYSLDNESWTTIKEYD | | | | | | | | | | | |
| | | KTGAPAGKDVIEESFE | | | | | | | | | | | |
| | | TPISAKYIRLTNLEARE | | | | | | | | | | | |
| | | NLTFSEFAIVSDDYKD | | | | | | | | | | | |
| | | DDDKG* | | | | | | | | | | | |
| nC-A | p1221LC | NPSLIRSESWVIDAGN | 273 | B-Trx | VI | 274 | 4 | AG | 275 | 7 | AG | 276 | 4 |
| | | EANLLDGDDNTGVWY | | (Ex- | DA | | | KN | | | VN | | |
| | | AGKNWFNSLAGEFIGL | | cellgen, | | | | WF | | | | | |
| | | DLGKEIKLDGIRFVIGK | | EG-5) | | | | N | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEAGVN | | | | | | | | | | | |
| | | LTFSEFAIVSDDYKDD | | | | | | | | | | | |
| | | DDKG* | | | | | | | | | | | |
| nC-A | p1223LC | NPSLIRSESWHGDAG | 277 | B-Trx | HG | 278 | 4 | DN | 279 | 7 | AR | 280 | 4 |
| | | NEANLLDGDDNTGVW | | (Ex- | DA | | | KN | | | QN | | |
| | | YDNKNWWNSLAGEFI | | cellgen, | | | | WW | | | | | |
| | | GLDLGKEIKLDGIRFVI | | EG-5) | | | | N | | | | | |
| | | GKNGGGSSDKWNKF | | | | | | | | | | | |
| | | KLEYSLDNESWTTIKE | | | | | | | | | | | |
| | | YDKTGAPAGKDVIEES | | | | | | | | | | | |
| | | FETPISAKYIRLTNLEA | | | | | | | | | | | |
| | | RQNLTFSEFAIVSDDY | | | | | | | | | | | |
| | | KDDDDKG* | | | | | | | | | | | |
| nC-A | p1226LC | NPSLIRSESWPDDQG | 281 | B- Trx | PD | 282 | 4 | WL | 283 | 7 | PR | 284 | 4 |
| | | NEANLLDGDDNTGVW | | (Ex- | DQ | | | KN | | | NN | | |
| | | YWLKNWYASLAGEFI | | cellgen, | | | | WY | | | | | |
| | | GLDLGKEIKLDGIRFVI | | EG-5) | | | | A | | | | | |
| | | GKNGGGSSDKWNKF | | | | | | | | | | | |
| | | KLEYSLDNESWTTIKE | | | | | | | | | | | |
| | | YDKTGAPAGKDVIEES | | | | | | | | | | | |
| | | FETPISAKYIRLTNLEP | | | | | | | | | | | |
| | | RNNLTFSEFAIVSDDY | | | | | | | | | | | |
| | | KDDDDKG* | | | | | | | | | | | |
| nC-A | p1227LC | NPSLIRSESWTVPWG | 285 | B-Trx | TV | 286 | 4 | NG | 287 | 7 | AEF | 288 | 4 |
| | | NEANLLDGDDNTGVW | | (Ex- | PW | | | KSI | | | N | | |
| | | YNGKSIFNSLAGEFIGL | | cellgen, | | | | FN | | | | | |
| | | DLGKEIKLDGIRFVIGK | | EG-5) | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEAEFN | | | | | | | | | | | |
| | | LTFSEFAIVSDDYKDD | | | | | | | | | | | |
| | | DDKG* | | | | | | | | | | | |
| nC-A | p1231LC | NPSLIRSESWTNALGN | 289 | B-Trx | TN | 290 | 4 | NKK | 291 | 7 | RG | 292 | 4 |
| | | EANLLDGDDNTGVWY | | (Ex- | AL | | | HW | | | QN | | |
| | | NKKHWFSSLAGEFIGL | | cellgen, | | | | FS | | | | | |
| | | DLGKEIKLDGIRFVIGK | | EG-5) | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLERGQN | | | | | | | | | | | |
| | | LTFSEFAIVSDDYKDD | | | | | | | | | | | |
| | | DDKG* | | | | | | | | | | | |
| nC-A | p1316 | NPSLIRSESWYANSG | 293 | B-Avi- | YA | 294 | 4 | DES | 295 | 7 | KPV | 296 | 4 |
| | | NEANLLDGDDNTGVW | | Sumo- | NS | | | DH | | | I | | |
| | | YDESDHYGSLAGEFIG | | TEV- | | | | YG | | | | | |
| | | LDLGKEIKLDGIRFVIG | | rtTA3 | | | | | | | | | |
| | | KNGGGSSDKWNKFKL | | (1269-1) | | | | | | | | | |
| | | EYSLDNESWTTIKEYD | | | | | | | | | | | |
| | | KTGAPAGKDVIEESFE | | | | | | | | | | | |
| | | TPISAKYIRLTNLEKPVI | | | | | | | | | | | |
| | | LTFSEFAIVSDDYKDD | | | | | | | | | | | |
| | | DDKG* | | | | | | | | | | | |
| nC-A | p1318 | NPSLIRSESWDRPTG | 297 | B-Avi- | DR | 298 | 4 | TDL | 299 | 7 | WG | 300 | 4 |
| | | NEANLLDGDDNTGVW | | Sumo- | PT | | | DHF | | | KI | | |
| | | YTDLDHFGSLAGEFIG | | TEV- | | | | G | | | | | |
| | | LDLGKEIKLDGIRFVIG | | rtTA3 | | | | | | | | | |
| | | KNGGGSSDKWNKFKL | | (1269-1) | | | | | | | | | |
| | | EYSLDNESWTTIKEYD | | | | | | | | | | | |
| | | KTGAPAGKDVIEESFE | | | | | | | | | | | |
| | | TPISAKYIRLTNLEWGK | | | | | | | | | | | |
| | | ILTFSEFAIVSDDYKDD | | | | | | | | | | | |
| | | DDKG* | | | | | | | | | | | |
| nC-A | p1323 | NPSLIRSESWGVNYG | 301 | B-Avi- | GV | 302 | 4 | NE | 303 | 7 | YD | 304 | 4 |
| | | NEANLLDGDDNTGVW | | Sumo- | NY | | | NN | | | DT | | |
| | | YNENNHYGSLAGEFIG | | TEV- | | | | HY | | | | | |
| | | LDLGKEIKLDGIRFVIG | | rtTA3 | | | | G | | | | | |
| | | KNGGGSSDKWNKFKL | | (1269-1) | | | | | | | | | |
| | | EYSLDNESWTTIKEYD | | | | | | | | | | | |
| | | KTGAPAGKDVIEESFE | | | | | | | | | | | |
| | | TPISAKYIRLTNLEYDD | | | | | | | | | | | |
| | | TLTFSEFAIVSDDYKD | | | | | | | | | | | |
| | | DDDKG* | | | | | | | | | | | |
| nC-A | p1332 | NPSLIRSESWRSPVG | 305 | B-Avi- | RS | 306 | 4 | NK | 307 | 7 | YFH | 308 | 4 |
| | | NEANLLDGDDNTGVW | | Sumo- | PV | | | ND | | | T | | |
| | | YNKNDHFGSLAGEFIG | | TEV- | | | | HF | | | | | |
| | | LDLGKEIKLDGIRFVIG | | rtTA3 | | | | G | | | | | |
| | | KNGGGSSDKWNKFKL | | (1269-1) | | | | | | | | | |
| | | EYSLDNESWTTIKEYD | | | | | | | | | | | |
| | | KTGAPAGKDVIEESFE | | | | | | | | | | | |
| | | TPISAKYIRLTNLEYFH | | | | | | | | | | | |
| | | TLTFSEFAIVSDDYKD | | | | | | | | | | | |
| | | DDDKG* | | | | | | | | | | | |
| nC-A | p1334 | NPSLIRSESWLLALGN | 309 | B-Avi- | LL | 310 | 4 | TD | 311 | 7 | AVK | 312 | 4 |
| | | EANLLDGDDNTGVWY | | Sumo- | AL | | | QD | | | W | | |
| | | TDQDHFGSLAGEFIGL | | TEV- | | | | HF | | | | | |
| | | DLGKEIKLDGIRFVIGK | | rtTA3 | | | | G | | | | | |
| | | NGGGSSDKWNKFKLE | | (1269-1) | | | | | | | | | |
| | | YSLDNESWTTIKEYDK | | | | | | | | | | | |
| | | TGAPAGKDVIEESFET | | | | | | | | | | | |
| | | PISAKYIRLTNLEAVKW | | | | | | | | | | | |
| | | LTFSEFAIVSDDYKDD | | | | | | | | | | | |
| | | DDKG* | | | | | | | | | | | |
| nC-A | p1803 | NPSLIRSESWLPQEGN | 313 | B- | LP | 314 | 4 | NYF | 315 | 7 | EIVI | 316 | 4 |
| | | EANLLDGDDNTGVWY | | 1631-5 | QE | | | DIA | | | | | |
| | | NYFDIAFSLAGEFIGLD | | (hMito | | | | F | | | | | |
| | | LGKEIKLDGIRFVIGKN | | NEET) | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLEEIVILTF | | | | | | | | | | | |
| | | SEFAIVSDDYKDDDDK | | | | | | | | | | | |
| | | G* | | | | | | | | | | | |
| nC-A | p1812 | NPSLIRSESWTQFIGN | 317 | B- | TQ | 318 | 4 | YDL | 319 | 7 | KIL | 320 | 4 |
| | | EANLLDGDDNTGVWY | | 1632-5 | FI | | | KH | | | P | | |
| | | YDLKHQYSLAGEFIGL | | (hPDIA3) | | | | QY | | | | | |
| | | DLGKEIKLDGIRFVIGK | | | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLEKILPL | | | | | | | | | | | |
| | | TFSEFAIVSDDYKDDD | | | | | | | | | | | |
| | | DKG* | | | | | | | | | | | |
| nC-A | p1816 | NPSLIRSESWPVYRG | 321 | B- | PV | 322 | 4 | NS | 323 | 7 | LEY | 324 | 4 |
| | | NEANLLDGDDNTGVW | | 1632-5 | YR | | | WA | | | G | | |
| | | YNSWAEKFSLAGEFIG | | (hPDIA3) | | | | EKF | | | | | |
| | | LDLGKEIKLDGIRFVIG | | | | | | | | | | | |
| | | KNGGGSSDKWNKFKL | | | | | | | | | | | |
| | | EYSLDNESWTTIKEYD | | | | | | | | | | | |
| | | KTGAPAGKDVIEESFE | | | | | | | | | | | |
| | | TPISAKYIRLTNLELEY | | | | | | | | | | | |
| | | GLTFSEFAIVSDDYKD | | | | | | | | | | | |
| | | DDDKG* | | | | | | | | | | | |
| nC-A | p1829 | NPSLIRSESWLVIYGN | 325 | B- | LVI | 326 | 4 | PKN | 327 | 7 | TVI | 328 | 4 |
| | | EANLLDGDDNTGVWY | | 1632-5 | Y | | | WS | | | L | | |
| | | PKNWSSYSLAGEFIGL | | (hPDIA3) | | | | SY | | | | | |
| | | DLGKEIKLDGIRFVIGK | | | | | | | | | | | |
| | | NGGGSSDKWNKFKLE | | | | | | | | | | | |
| | | YSLDNESWTTIKEYDK | | | | | | | | | | | |
| | | TGAPAGKDVIEESFET | | | | | | | | | | | |
| | | PISAKYIRLTNLETVILL | | | | | | | | | | | |
| | | TFSEFAIVSDDYKDDD | | | | | | | | | | | |
| | | DKG* | | | | | | | | | | | |
| nC-A | p1920 | NPSLIRSESWQLEGG | 329 | B- | QL | 330 | 4 | FAL | 331 | 7 | VKY | 332 | 4 |
| | | NEANLLDGDDNTGVW | | hCD81 | EG | | | IAIN | | | D | | |
| | | YFALIAINSLAGEFIGLD | | | | | | | | | | | |
| | | LGKEIKLDGIRFVIGKN | | | | | | | | | | | |
| | | GGGSSDKWNKFKLEY | | | | | | | | | | | |
| | | SLDNESWTTIKEYDKT | | | | | | | | | | | |
| | | GAPAGKDVIEESFETPI | | | | | | | | | | | |
| | | SAKYIRLTNLEVKYDLT | | | | | | | | | | | |
| | | FSEFAIVSDDYKDDDD | | | | | | | | | | | |
| | | KG* | | | | | | | | | | | |
| nC-A | plate | HHHHHHYPSLIRSES | 333 | B- | AR | 334 | 4 | HKK | 335 | 7 | VN | 336 | 4 |
| | 37- | WARPVGNEANLLDGD | | Sumo | PV | | | KSK | | | SP | | |
| | 624F | DNTGVWYHKKKSKISL | | | | | | I | | | | | |
| | | AGEFIGLDLGKEIKLDG | | | | | | | | | | | |
| | | IRFVIGKNGGGSSDKW | | | | | | | | | | | |
| | | NKFKLEYSLDNESWTT | | | | | | | | | | | |
| | | IKEYDKTGAPAGKDVI | | | | | | | | | | | |
| | | EESFETPISAKYIRLTN | | | | | | | | | | | |
| | | LEVNSPLTFSEFAIVSD | | | | | | | | | | | |
| | | * | | | | | | | | | | | |
| nC-A | SUM | GSSHHHHHHNPSLIRS | 337 | B- | AY | 338 | 4 | IYD | 339 | 7 | YY | 340 | 4 |
| | O-16- | ESWAYDTGNEANLLD | | Sumo | DT | | | SDE | | | HY | | |
| | 624F | GDDNTGVWYIYDSDE | | | | | | K | | | | | |
| | | KSLAGEFIGLDLGKEIK | | | | | | | | | | | |
| | | LDGIRFVIGKNGGGSS | | | | | | | | | | | |
| | | DKWNKFKLEYSLDNE | | | | | | | | | | | |
| | | SWTTIKEYDKTGAPAG | | | | | | | | | | | |
| | | KDVIEESFETPISAKYI | | | | | | | | | | | |
| | | RLTNLEYYHYLTFSEF | | | | | | | | | | | |
| | | AIVSD* | | | | | | | | | | | |
| nC-A | p817- | MGSSHHHHHHNPSLI | 341 | B- | AL | 342 | 4 | KFK | 343 | 7 | TEY | 344 | 6 |
| | 1-T7 | RSESWALVYGNEANL | | MBP- | VY | | | YW | | | KW | | |
| | | LDGDDNTGVWYKFKY | | avi | | | | AV | | | G | | |
| | | WAVSLAGEFIGLDLGK | | | | | | | | | | | |
| | | EIKLDGIRFVIGKNGGG | | | | | | | | | | | |
| | | SSDKWNKFKLEYSLD | | | | | | | | | | | |
| | | NESWTTIKEYDKTGAP | | | | | | | | | | | |
| | | AGKDVIEESFETPISAK | | | | | | | | | | | |
| | | YIRLTNLETEYKWGLT | | | | | | | | | | | |
| | | FSEFAIVSDGGGLNDI | | | | | | | | | | | |
| | | FEAQKIEWHE* | | | | | | | | | | | |
| nC-A | pC894 | GSSHHHHHHNPSLIRS | 345 | BgaI | TP | 346 | 4 | KTL | 347 | 7 | YVL | 348 | 6 |
| | | ESWTPYDGNEANLLD | | | YD | | | NG | | | RW | | |
| | | GDDNTGVWYKTLNGD | | | | | | DT | | | W | | |
| | | TSLAGEFIGLDLGKEIK | | | | | | | | | | | |
| | | LDGIRFVIGKNGGGSS | | | | | | | | | | | |
| | | DKWNKFKLEYSLDNE | | | | | | | | | | | |
| | | SWTTIKEYDKTGAPAG | | | | | | | | | | | |
| | | KDVIEESFETPISAKYI | | | | | | | | | | | |
| | | RLTNLEYVLRWWLTF | | | | | | | | | | | |
| | | SEFAIVSDDYKDDDDK | | | | | | | | | | | |
| | | G* | | | | | | | | | | | |
| nC-A | p1875 | NPSLIRSESWVVRFGN | 349 | B- | VV | 350 | 4 | ING | 351 | 7 | NT | 352 | 6 |
| | | EANLLDGDDNTGVWY | | hCD63 | RF | | | WW | | | RV | | |
| | | INGWWWSSLAGEFIG | | | | | | WS | | | HY | | |
| | | LDLGKEIKLDGIRFVIG | | | | | | | | | | | |
| | | KNGGGSSDKWNKFKL | | | | | | | | | | | |
| | | EYSLDNESWTTIKEYD | | | | | | | | | | | |
| | | KTGAPAGKDVIEESFE | | | | | | | | | | | |
| | | TPISAKYIRLTNLENTR | | | | | | | | | | | |
| | | VHYLTFSEFAIVSDDY | | | | | | | | | | | |
| | | KDDDDKG* | | | | | | | | | | | |
| nC-A | p1863 | NPSLIRSESWVWFAIG | 353 | B- | VW | 354 | 5 | FTK | 355 | 7 | LFN | 356 | 5 |
| | | NEANLLDGDDNTGVW | | hCD63 | FAI | | | TW | | | NP | | |
| | | YFTKTWSESLAGEFIG | | | | | | SE | | | | | |
| | | LDLGKEIKLDGIRFVIG | | | | | | | | | | | |
| | | KNGGGSSDKWNKFKL | | | | | | | | | | | |
| | | EYSLDNESWTTIKEYD | | | | | | | | | | | |
| | | KTGAPAGKDVIEESFE | | | | | | | | | | | |
| | | TPISAKYIRLTNLELFN | | | | | | | | | | | |
| | | NPLTFSEFAIVSDDYK | | | | | | | | | | | |
| | | DDDDKG* | | | | | | | | | | | |

*denotes a stop codon

TABLE 10
Loop sequence variety of nC-B nanoCLAMPs binding diverse targets

| | | SEQ | | SEQ | | SEQ | | SEQ | |
|---|---|---|---|---|---|---|---|---|---|
| | | ID | | ID | | ID | | ID | |
| Clone | Sequence | NO: | L1 | NO: | L2 | NO: | L8 | NO: | Target |
| p2803 | GSSHHHHHHDPTLIHTPGWAVGDGS | 357 | AVG | 358 | VIVWQ | 359 | VHIQ | 360 | B- |
| | EADLLDGDDSTGVEYVIVWQWDSLAG | | D | | WD | | G | | Sumo |
| | EFIGLDLGEVVEVGGIHFVIGADGGGS | | | | | | | | |
| | SDKWTRFRLEYSLDGESWTTIREYDH | | | | | | | | |
| | TGAPAGQDVIDEDFETPISAQYIRLTNL | | | | | | | | |
| | EVHIQGLTFSEFAIVSDELEDYKDDDD | | | | | | | | |
| | KG* | | | | | | | | |
| p2804 | GSSHHHHHHDPTLIHTPGWDVIAGSE | 361 | DVIA | 362 | PIPYQG | 363 | WAG | 364 | B- |
| | ADLLDGDDSTGVEYPIPYQGWSLAGE | | | | W | | DN | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDGIDEDFETPISAQYIRLTNL | | | | | | | | |
| | EWAGDNLTFSEFAIVSDELEDYKDDD | | | | | | | | |
| | DKG* | | | | | | | | |
| p2805 | GSSHHHHHHDPTLIHTPGWDVYGGS | 365 | DVY | 366 | VVIWEV | 367 | GQT | 368 | B- |
| | EADLLDGDDSTGVEYVVIWEVGSLAG | | G | | G | | QQ | | Sumo |
| | EFIGLDLGEVVEVGGIHFVIGADGGGS | | | | | | | | |
| | SDKWTRFRLEYSLDGESWTTIREYDH | | | | | | | | |
| | TGAPAGQDVIDEDFETPISAQYIRLTNL | | | | | | | | |
| | EGQTQQLTFSEFAIVSDELEDYKDDD | | | | | | | | |
| | DKG* | | | | | | | | |
| p2806 | GSSHHHHHHDPTLIHTPGWEVSIGSE | 369 | EVSI | 370 | VVIWEV | 371 | YVIS | 372 | B- |
| | ADLLDGDDSTGVEYVVIWEVNSLAGE | | | | N | | Q | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | YVISQLTFSEFAIVSDELEDYKDDDDK | | | | | | | | |
| | G* | | | | | | | | |
| p2807 | SSHHHHHHDPTLIHTPGWIVDDGSEA | 373 | IVDD | 374 | FAYPAY | 375 | ELDT | 376 | B- |
| | DLLDGDDSTGVEYFAYPAYISLAGEFI | | | | I | | E | | Sumo |
| | GLDLGEVVEVGGIHFVIGADGGGSSD | | | | | | | | |
| | KWTRFRLEYSLDGESWITIREYDHTGA | | | | | | | | |
| | PAGQDVIDEDFETPISAQYIRLTNLEEL | | | | | | | | |
| | DTELTFSEFAIVSDELEDYKDDDDKG* | | | | | | | | |
| p2808 | GSSHHHHHHDPTLIHTPGWLVIPGSE | 377 | LVIP | 45 | IIVEEFT | 46 | YNFI | 47 | B- |
| | ADLLDGDDSTGVEYIIVEEFTSLAGEFI | | | | | | D | | Sumo |
| | GLDLGEVVEVGGIHFVIGADGGGSSD | | | | | | | | |
| | KWTRFRLEYSLDGESWTTIREYDHTG | | | | | | | | |
| | APAGQDVIDEDFETPISAQYIRLTNLEY | | | | | | | | |
| | NFIDLTFSEFAIVSDELEDYKDDDDKG | | | | | | | | |
| p2809 | GSSHHHHHHDPTLIHTPGWNVEQGS | 378 | NVE | 48 | ILVEEFP | 49 | NEV | 50 | B- |
| | EADLLDGDDSTGVEYILVEEFPSLAGE | | Q | | | | PQ | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | NEVPQLTFSEFAIVSDELEDYKDDDDK | | | | | | | | |
| | G* | | | | | | | | |
| p2810 | GSSHHHHHHDPTLIHTPGWNVSDGSE | 379 | NVS | 380 | VLIWQ | 381 | QQQ | 382 | B- |
| | ADLLDGDDSTGVEYVLIWQWDSLAGE | | D | | WD | | DE | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | QQQDELTFSEFAIVSDELEDYKDDDD | | | | | | | | |
| | KG* | | | | | | | | |
| p2811 | GSSHHHHHHDPTLIHTPGWPVVTGSE | 383 | PVV | 51 | IVLTDQ | 52 | LAQ | 53 | B- |
| | ADLLDGDDSTGVEYIVLTDQASLAGEF | | T | | A | | DP | | Sumo |
| | IGLDLGEVVEVGGINFVIGADGGGSSD | | | | | | | | |
| | KWTRFRLEYSLDGESWTTIREYDHTG | | | | | | | | |
| | APAGQDVIDEDFETPISAQYIRLTNLEL | | | | | | | | |
| | AQDPLTFSEFAIVSDELEDYKDDDDKG | | | | | | | | |
| | * | | | | | | | | |
| p2812 | HHHHHHDPTLIHTPGWQVFNGSEADL | 384 | QVF | 385 | PHWTP | 386 | DYQ | 387 | B- |
| | LDGDDSTGVEYPHWTPHQSLAGEFIG | | N | | HQ | | EI | | Sumo |
| | LDLGEVVEVGGIHFVIGADGGGSSDK | | | | | | | | |
| | WTRFRLEYSLDGESWTTIREYDHKGP | | | | | | | | |
| | CRARCYRRRFQTPISAQYIRLTNLEDY | | | | | | | | |
| | QEILTFSEFAIVSDELEDYKDDDDKG* | | | | | | | | |
| p2813 | GSSHHHHHHDPTLIHTPGWQVGDGS | 388 | QVG | 389 | ITWIWL | 390 | WDQ | 391 | B- |
| | EADLLDGDDSTGVEYITWIWLDSLAGE | | D | | D | | ES | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | WDQESLTFSEFAIVSDELEDYKDDDD | | | | | | | | |
| | KG* | | | | | | | | |
| p2814 | GSSHHHHHHDPTLIHTPGWRSNLGSE | 392 | RSN | 393 | IIITDYE | 394 | TAYF | 395 | B- |
| | ADLLDGDDSTGVEYIIITDYESLAGEFI | | L | | | | T | | Sumo |
| | GLDLGEVVEVGGINFVIGADGGGSSD | | | | | | | | |
| | KWTRFRLEYSLDGESWTTIREYDHTG | | | | | | | | |
| | APAGQDVIDEDFETPISAQYIRLTNLET | | | | | | | | |
| | AYFTLTFSEFAIVSDELEDYKDDDDKG | | | | | | | | |
| | * | | | | | | | | |
| p2815 | GSSHHHHHHDPTLIHTPGWSVAGGSE | 396 | SVA | 397 | PQPLVG | 398 | WAA | 399 | B- |
| | ADLLDGDDSTSVEYPQPLVGTSLAGE | | G | | T | | DA | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | WAADALTFSEFAIVSDELEDYKDDDDK | | | | | | | | |
| | G* | | | | | | | | |
| p2816 | HHHHHHDPTLIHTPGWTVAEGSEADL | 400 | TVA | 401 | VIIHEFT | 402 | HDP | 403 | B- |
| | LDGDDSTGVEYVIIHEFTSLAGEFIGLD | | E | | | | GV | | Sumo |
| | LGEVVEVGGIHFVIGADGGGSSDKWT | | | | | | | | |
| | RFRLEYSLDGESWTTIREYDHTGAPA | | | | | | | | |
| | GQDVIDEDFETPISAQYIRLTNLEHDP | | | | | | | | |
| | GVLTFSEFAIVSDELEDYKDDDDKG* | | | | | | | | |
| p2817 | GSSHHHHHHDPTLIHTPGWTVDIGSE | 404 | TVDI | 405 | PPATPY | 406 | SHG | 407 | B- |
| | ADLLDGDDSTGVEYPPATPYASLAGE | | | | A | | DI | | Sumo |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | SHGDILTFSEFAIVSDELEDYKDDDXK | | | | | | | | |
| | G* | | | | | | | | |
| p2818 | GSSHHHHHHDPTLIHTPGWYVAAGSE | 408 | YVA | 409 | VIIWEP | 410 | YIHN | 411 | B- |
| | ADLLDGDDSTGVEYVIIWEPQSLAGEF | | A | | Q | | W | | Sumo |
| | IGLDLGEVVEVGGIHFVIGADGGGSSD | | | | | | | | |
| | KWTRFRLEYSLDGESWTTIREYDHTG | | | | | | | | |
| | APAGQDVIDEDFETPISAQYIRLTNLEY | | | | | | | | |
| | IHNWLTFSEFAIVSDELEDYKDDDDKG | | | | | | | | |
| | * | | | | | | | | |
| p2819 | HHHHHHDPTLIHTPGWYVVTGSEADL | 412 | YVV | 413 | PYIIQGL | 414 | YHQ | 415 | B- |
| | LDGDDSTGVEYPYIIQGLSLAGEFIGLD | | T | | | | FH | | Sumo |
| | LGEVVEVGGIHFVIGADGGGSSDKWT | | | | | | | | |
| | RFRLEYSLDGESWTTIREYDHTGAPA | | | | | | | | |
| | GQDVIDEDFETPISAQYIRLTNLEYHQF | | | | | | | | |
| | HLTFSEFAIVSDELEDYKDDDDKG* | | | | | | | | |
| p2821 | GSSHHHHHHDPTLIHTPGWAVIHGSE | 416 | AVIH | 417 | WWLSV | 418 | FYH | 419 | B- |
| | ADLLDGDDSTGVEYWWLSVAVSLAG | | | | AV | | PY | | PDL1 |
| | EFIGLDLGEVVEVGGIHFVIGADGGGS | | | | | | | | |
| | SDKWTRFRLEYSLDGESWTTIREYDH | | | | | | | | |
| | TGAPAGQDVIDEDFETPISAQYIRLTNL | | | | | | | | |
| | EFYHPYLTFSEFAIVSDELEDYKDDDD | | | | | | | | |
| | KG* | | | | | | | | |
| p2822 | SSHHHHHHDPTLIHTPGWAVYVGSEA | 420 | AVY | 421 | WWTAT | 422 | NVD | 423 | B- |
| | DLLDGDDSTGVEYWWTATGDSLAGE | | V | | GD | | QS | | PDL1 |
| | FIGLDLGKVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | NVDQSLXFSEFAIVSDELEDYKDDDDK | | | | | | | | |
| | G* | | | | | | | | |
| p2823 | GSSHHHHHHDPTLIHTPGWEVNYGSE | 424 | EVN | 425 | FTWAY | 426 | NSH | 427 | B- |
| | ADLLDGDDSTGVEYFTWAYGTSLAGE | | Y | | GT | | WW | | PDL1 |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | NSHWWLTFSEFAIVSDELEDYKDDDD | | | | | | | | |
| | KG* | | | | | | | | |
| p2824 | GSSHHHHHHDPTLIHTPGWFVHWGS | 428 | FVH | 429 | WVWHS | 430 | HQG | 431 | B- |
| | EADLLDGDDSTGVEYWVWHSFTSLA | | W | | FT | | DA | | PDL1 |
| | GEFIGLDLGEVVEVGGIHFVIGADGGG | | | | | | | | |
| | SSDKWTRFRLEYSLDGESWTTIREYD | | | | | | | | |
| | HTGAPAGQDVIDEDFETPISAQYIRLT | | | | | | | | |
| | NLEHQGDALTFSEFAIVSDELEDYKDD | | | | | | | | |
| | DDKG* | | | | | | | | |
| p2825 | GSSHHHHHHDPTLIHTPGWFVSDGSE | 432 | FVS | 433 | IYPYSY | 434 | PTH | 435 | B- |
| | ADLLDGDDSTGVEYIYPYSYLSLAGEFI | | D | | L | | WI | | PDL1 |
| | GLDLGEVVEVGGIHFVIGADGGGSSD | | | | | | | | |
| | KWTRFRLEYSLDGESWTTIREYDHTG | | | | | | | | |
| | APAGQDVIDEDFETPISAQYIRLTNLEP | | | | | | | | |
| | THWILTFSEFAIVSDELEDYKDDDDKG | | | | | | | | |
| | * | | | | | | | | |
| p2826 | GSSHHHHHHDPTLIHTPGWFVVPGSE | 436 | FVV | 437 | FLXPG | 438 | DPP | 439 | B- |
| | ADLLDGDDSTGVEYFLXPGWTSLAGE | | P | | WT | | VW | | PDL1 |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | DPPVWLTFSEFAIVSDELEDYKDDDDK | | | | | | | | |
| | G* | | | | | | | | |
| p2827 | GSSHHHHHHDPTLIHTPGWIVVAGSE | 440 | IVVA | 441 | FQVPG | 442 | DPP | 443 | B- |
| | ADLLDGDDSTGVEYFQVPGWTSLAGE | | | | WT | | VW | | PDL1 |
| | FIGLDLGEVVEVGGIHFVIGADGGGSS | | | | | | | | |
| | DKWTRFRLEYSLDGESWTTIREYDHT | | | | | | | | |
| | GAPAGQDVIDEDFETPISAQYIRLTNLE | | | | | | | | |
| | DPPVWLTFSEFAIVSDELEDYKDDDDK | | | | | | | | |
| | G* | | | | | | | | |
| p2867 | HHHHHHDPTLIHTPGWDVNFGSEADL | 444 | DVN | 445 | PQIGHY | 446 | SFV | 447 | B- |
| | LDGDDSTGVEYPQIGHYISLAGEFIGL | | F | | I | | HP | | CTLA4 |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLESFV | | | | | | | | |
| | HPLTFSEFAIVSDELEDYKDDDDKG* | | | | | | | | |
| p3137 | HHHHHHDPTLIHTPGWLVANGSEADL | 448 | LVA | 449 | PFAHH | 450 | SFIY | 451 | B- |
| | LDGDDSTGVEYPFAHHQISLAGEFIGL | | N | | QI | | P | | CTLA4 |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLESFI | | | | | | | | |
| | YPLTFSEFAIVSDELEDYKDDDDKG* | | | | | | | | |
| p3138 | HHHHHHDPTLIHTPGWNVAPGSEADL | 452 | NVA | 453 | FHIGHHI | 454 | PFT | 455 | B- |
| | LDGDDSTGIEYFHIGHHISLAGEFIGLD | | P | | | | WP | | CTLA4 |
| | LGEVVEVGGIHFVIGADGGGSSDKWT | | | | | | | | |
| | RFRLEYSLDGESWTTIREYDHTGAPA | | | | | | | | |
| | GQDVIDEDFETPISAQYIRLTNLEPFTW | | | | | | | | |
| | PLTFSEFAIVSDELEDYKDDDDKG* | | | | | | | | |
| p2871- | HHHHHHDPTLIHTPGWNVTVGSEADL | 456 | NVT | 457 | YITTS | 458 | EPG | 459 | B- |
| 1 | LDGDDSTGVEYYITTSSLAGEFIGLDL | | V | | | | SH | | CTLA4 |
| | GEVVEVGGIHFVIGADGGGSSDKWTR | | | | | | | | |
| | FRLEYSLDGESWTTIREYDHTGAPAG | | | | | | | | |
| | QDVIDEDFETPISAQYIRLTNLEEPGSH | | | | | | | | |
| | LTFSEFAIVSDELEDYKDDDDKG* | | | | | | | | |
| p2847- | HHHHHHDPTLIHTPGWPVSFGSEADL | 460 | PVS | 461 | TFHGH | 462 | HFV | 463 | B- |
| 1 | LDGDDSTGVEYTFHGHHVSLAGEFIG | | F | | HV | | YP | | CTLA4 |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEHF | | | | | | | | |
| | VYPLTFSEFAIVSDELEDYKDDDDKG* | | | | | | | | |
| p313 | HHHHHHDPTLIHTPGWQVNDGSEADL | 464 | QVN | 465 | PVYQH | 466 | DFQ | 467 | B- |
| 9 | LDGDDSTGVEYPVYQHHVSLAGEFIG | | D | | HV | | YP | | CTLA4 |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPIPAQYIRLTNLEDF | | | | | | | | |
| | QYPLTFSEFAIVSDELEDYKDDDDKG* | | | | | | | | |
| p2874- | HHHHHHDPTLIHTPGWTVAIGSEADLL | 468 | TVAI | 469 | PQYAHT | 470 | QFIY | 471 | B- |
| 1 | DGDDSTGVEYPQYAHTISLAGEFIGLD | | | | I | | P | | CTLA4 |
| | LGEVVEVGGIHFVIGADGGGSSDKWT | | | | | | | | |
| | RFRLEYSLDGESWTTIREYDHTGAPA | | | | | | | | |
| | GQDVIDEDFETPISAQYIRLTNLEQFIY | | | | | | | | |
| | PLTFSEFAIVSDELEDYKDDDDKG* | | | | | | | | |
| p2875- | HHHHHHDPTLIHTPGWTVSPGSEADL | 472 | TVS | 473 | PLGWH | 474 | PFH | 475 | B- |
| 1 | LDGDDSTGVEYPLGWHAVSLAGEFIG | | P | | AV | | PG | | CTLA4 |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEPF | | | | | | | | |
| | HPGLTFSEFAIVSDELEDYKDDDDKG* | | | | | | | | |
| p2878- | HHHHHHDPTLIHTPGWYVAGGSEADL | 476 | YVA | 477 | PHYLHT | 478 | SFTY | 479 | B- |
| 1 | LDGDDSTGVEYPHYLHTISLAGEFIGL | | G | | I | | P | | CTLA4 |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLESFT | | | | | | | | |
| | YPLTFSEFAIVSDELEDYKDDDDKG* | | | | | | | | |
| p2880 | HHHHHHDPTLIHTPGWYVGTGSEADL | 480 | YVG | 481 | PHIAHTI | 482 | EFQ | 483 | B- |
| | LDGDDSTGVEYPHIAHTISLAGEFIGLD | | T | | | | YP | | CTLA4 |
| | LGEVVEVGGIHFVIGADGGGSSDKWT | | | | | | | | |
| | RFRLEYSLDGESWTTIREYDHTGAPA | | | | | | | | |
| | GQDVIDEDFETPISAQYIRLTNLEEFQY | | | | | | | | |
| | PLTFSEFAIVSDELEDYKDDDDKG* | | | | | | | | |
| p3140 | HHHHHHDPTLIHTPGWAVTTGSEADL | 484 | AVTT | 485 | PFVHH | 486 | HFIF | 487 | B- |
| | LDGDDSTGVEYPFVHHQVSLAGEFIG | | | | QV | | P | | CTLA4 |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEHF | | | | | | | | |
| | IFPLTFSEFAIVSDELEDYKDDDDKG* | | | | | | | | |
| p3142 | HHHHHHDPTLIHTPGWEVEIGSEADLL | 488 | EVEI | 489 | PNYDH | 490 | QFQ | 491 | B- |
| | DGDDSTGVEYPNYDHQISLAGEFIGLD | | | | QI | | PG | | CTLA4 |
| | LGEVVEVGGIHFVIGADGGGSSDKWT | | | | | | | | |
| | RFRLEYSLDGESWTTIREYDHTGAPA | | | | | | | | |
| | GQDVIDEDFETPISAQYIRLTNLEQFQP | | | | | | | | |
| | GLTFSEFAIVSDELEDYKDDDDKG* | | | | | | | | |
| p3143 | HHHHHHDPTLIHTPGWFVHPGSEADL | 492 | FVH | 493 | VDTYQL | 494 | SFP | 495 | B- |
| | LDGDDSTGVEYVDTYQLYSLAGEFIGL | | P | | Y | | SH | | CTLA4 |
| | DLGEVVEVGGIHFVSGADGGGSSDK | | | | | | | | |
| | WTRFRLEYSLDGESWTTIREYDHTGA | | | | | | | | |
| | PAGQDVIDEDFETPITAQYIRLTNLESF | | | | | | | | |
| | PSHLTFSEFAIVSDELEDYKDDDDKG* | | | | | | | | |
| p2845- | HHHHHHDPTLIHTPGWLVHNGSEADL | 496 | LVH | 497 | VPWTAL | 498 | ASQ | 499 | B- |
| 1 | LDGDDSTGVEYVPWTALSSLAGEFIGL | | N | | S | | SY | | CTLA4 |
| | DLGEVVEVGGIHFVTDGGGSSDKWTR | | | | | | | | |
| | FRLEYSLDGESWTTIREYDHTGAPAG | | | | | | | | |
| | QDVIDEDFETPISAQYIRLTNLEASQSY | | | | | | | | |
| | LTFSEFAIVSDELEDYKDDDDKG* | | | | | | | | |
| p3145 | HHHHHHDPTLIHTPGWPVLIGSEADLL | 500 | PVLI | 501 | PFHGH | 502 | YFV | 503 | B- |
| | DGDDSTGVEYPFHGHHLSLAGEFIGL | | | | HL | | HP | | CTLA4 |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEYFV | | | | | | | | |
| | HPLTFSEFAIVSDELEDYKDDDDKG* | | | | | | | | |
| p2849- | HHHHHHDPTLIHTPGWQVHNGSEADL | 504 | QVH | 505 | LHSEPL | 506 | DATI | 507 | B- |
| 1 | LDGDDSTGVEYLHSEPLESLAGEFIGL | | N | | E | | S | | CTLA4 |
| | DLGEVVEVGGIHFVSGADGGGSSDK | | | | | | | | |
| | WTRFRLEYSLDGESWTTIREYDHTGA | | | | | | | | |
| | PAGQDVIDEDFETPISAQYIRLTNLEDA | | | | | | | | |
| | TISLTFSEFAIVSDELEDYKDDDDKG* | | | | | | | | |
| p3146 | HHHHHHDPTLIHTPGWQVHPGSEADL | 508 | QVH | 509 | PQVHH | 510 | DFTF | 511 | B- |
| | LDGDDSTGVEYPQVHHHLSLAGEFIG | | P | | HL | | P | | CTLA4 |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEDF | | | | | | | | |
| | TFPLTFSEFAIVSDELEDYKDDDDKG* | | | | | | | | |
| p3147 | HHHHHHDPTLIHTPGWQVITGSEADLL | 512 | QVIT | 513 | WNWAT | 514 | NQV | 515 | B- |
| | DGDDSTGVEYWNWATQISLAGEFIGL | | | | QI | | ST | | CTLA4 |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLENQV | | | | | | | | |
| | STLTFSEFAIVSDELEDYKDDDDKG* | | | | | | | | |
| p2852- | HHHHHHDPTLIHTPGWSVYEGSEADL | 516 | SVY | 517 | PQFHH | 518 | DFV | 519 | B- |
| 1 | LDGDDSTGVEYPQFHHSISLAGEFIGL | | E | | SI | | YP | | CTLA4 |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEDFV | | | | | | | | |
| | YPLTFSEFAIVSDELEDYKDDDDKG* | | | | | | | | |
| p3148 | HHHHHHDPTLIHTPGWVVDPGSEADL | 520 | VVD | 521 | TFHGH | 522 | HFV | 523 | B- |
| | LDGDDSTGVEYTFHGHHVSLAGEFIG | | P | | HV | | YP | | CTLA4 |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEHF | | | | | | | | |
| | VYPLTFSEFAIVSDELEDYKDDDDKG* | | | | | | | | |
| p2854- | HHHHHHDPTLIHTPGWEVTVGSEADL | 524 | EVT | 525 | LFIHDH | 526 | FLYI | 527 | B- |
| 1 | LDGDDSTGVEYLFIHDHVSLAGEFIGL | | V | | V | | H | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEFLYI | | | | | | | | |
| | HLTFSEFAIVSDELEDYKDDDDKG* | | | | | | | | |
| p2848- | HHHHHHDPTLIHTPGWLVLSGSEADL | 528 | LVLS | 529 | VWNDW | 530 | ILTD | 531 | B- |
| 1 | LDGDDSTGVEFVWNDWGQSLAGEFI | | | | GQ | | H | | TIGIT |
| | GLDLGEVVEVGGIHFVIGADGGGSSD | | | | | | | | |
| | KWTRFRLEYSLDGESWTTIREYDHTG | | | | | | | | |
| | APAGQHVIDEDFETAISAQYIRLTNLEIL | | | | | | | | |
| | TDHLTFSELAIVSDELEDYKDDDDKG* | | | | | | | | |
| p3149 | HHHHHHDPTLIHTPGWNVQEGSEADL | 532 | NVQ | 533 | QVLHIH | 534 | AYL | 535 | B- |
| | LDGDDSTGVEYQVLHIHVSLAGEFIGL | | E | | V | | QP | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEAYL | | | | | | | | |
| | QPLTFSEFAIVSDELEDYKDDDDKG* | | | | | | | | |
| p2857- | HHHHHHDPTLIHTPGWVVYGGSEADL | 536 | VVY | 537 | LVPYLS | 538 | GYS | 539 | B- |
| 1 | LDGDDSTGVEYLVPYLSWSLAGEFIGL | | G | | W | | AY | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEGYS | | | | | | | | |
| | AYLTFSEFAIVSDELEDYKDDDDKG* | | | | | | | | |
| p2972- | HHHHHHDPTLIHTPGWYVPFGSEADL | 540 | YVP | 541 | YPTLHN | 542 | FYIG | 543 | B- |
| 1 | LDGDDSTGVEYYPTLHNVSLAGEFIGL | | F | | V | | Y | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEFYI | | | | | | | | |
| | GYLTFSEFAIVSDELEDYKDDDDKG* | | | | | | | | |
| p3151 | HHHHHHDPTLIHTPGWYVPFGSEADL | 544 | YVP | 545 | YPTLRS | 546 | FYIG | 547 | B- |
| | LDGDDSTGVEYYPTLRSVSLAGEFIGL | | F | | V | | Y | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEFYI | | | | | | | | |
| | GYLTFSEFAIVSDELEDYKDDDDKG* | | | | | | | | |
| p2979- | HHHHHHDPTLIHTPGWEVWFGSEADL | 548 | EVW | 549 | FTAQAQ | 550 | GSV | 551 | B- |
| 1 | LDGDDSTGVEYFTAQAQPSLAGEFIGL | | F | | P | | WY | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEGSV | | | | | | | | |
| | WYLTFSEFAIVSDELEDYKDDDDKG* | | | | | | | | |
| p2855- | HHHHHHDPTLIHTPGWFVFAGSEADL | 552 | FVFA | 553 | TWVNW | 554 | GHF | 555 | B- |
| 1 | LDGDDSTGVEYTWVNWPVSLAGEFIG | | | | PV | | TS | | TIGIT |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEGH | | | | | | | | |
| | FTSLTFSEFAIVSDELEDYKDDDDKG* | | | | | | | | |
| p2975- | HHHHHHDPTLIHTPGWIVISGSEADLL | 556 | IVIS | 557 | YAFDY | 558 | FTTY | 559 | B- |
| 1 | DGDDSTGVEYYAFDYWNSLAGEFIGL | | | | WN | | 1 | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEFTT | | | | | | | | |
| | YILTFSEFAIVSDELEDYKDDDDKG* | | | | | | | | |
| p2976- | HHHHHHDPTLIHTPGWLVLSGSEADL | 560 | LVLS | 561 | WTIQSQ | 562 | FSG | 563 | B- |
| 1 | LDGDDSTGVEYWTIQSQPSLAGEFIGL | | | | P | | VI | | TIGIT |
| | DLGEVVEVGGIHFVIGADGGGSSDKW | | | | | | | | |
| | TRFRLEYSLDGESWTTIREYDHTGAP | | | | | | | | |
| | AGQDVIDEDFETPISAQYIRLTNLEFSG | | | | | | | | |
| | VILTFSEFAIVSDELEDYKDDDDKG* | | | | | | | | |
| p2970- | HHHHHHDPTLIHTPGWNVAAGSEADL | 564 | NVA | 565 | YSAHW | 566 | HVV | 567 | B- |
| 1 | LDGDDSTGVEYYSAHWWVSLAGEFIG | | A | | WV | | GF | | TIGIT |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLEHV | | | | | | | | |
| | VGFLTFSEFAIVSDELEDYKDDDDKG* | | | | | | | | |
| p2974- | HHHHHHDPTLIHTPGWWVFQGSEAD | 568 | WVF | 569 | FHPHLP | 570 | TTFF | 571 | B- |
| 1 | LLDGDDSTGVEYFHPHLPFSLAGEFIG | | Q | | F | | Q | | TIGIT |
| | LDLGEVVEVGGIHFVIGADGGGSSDK | | | | | | | | |
| | WTRFRLEYSLDGESWTTIREYDHTGA | | | | | | | | |
| | PAGQDVIDEDFETPISAQYIRLTNLETT | | | | | | | | |
| | FFQLTFSEFAIVSDELEDYKDDDDKG* | | | | | | | | |

### Example 3. Terbium Binding

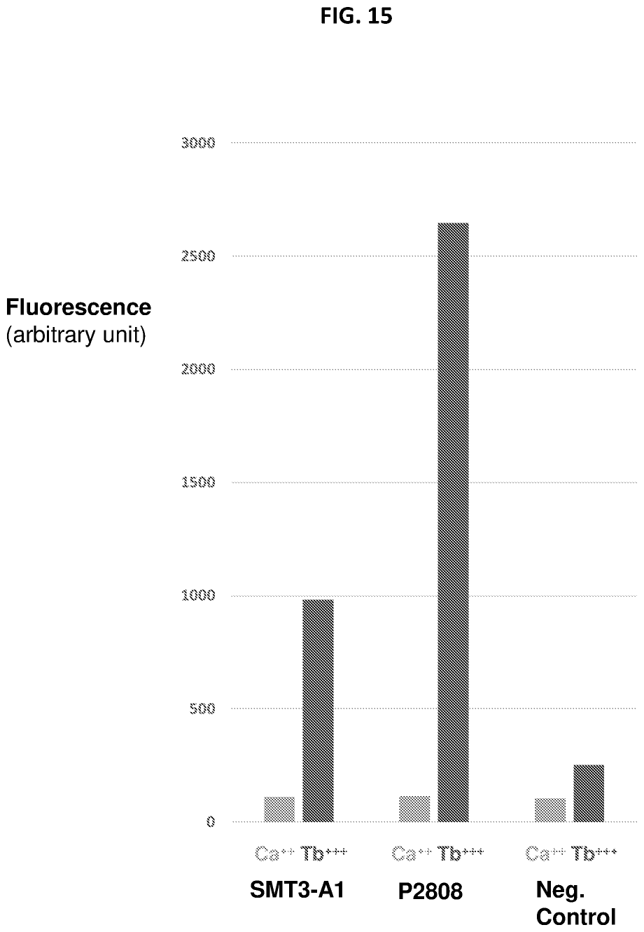

Lanthanide binding to the protein scaffold was demonstrated by incubating the proteins with terbium, removing unbound terbium by buffer exchange, and measuring time resolved fluorescence. Proteins were prepared at 30 μM in 20 mM MOPS, 150 mM NaCl (MBS), pH 6.5 and buffer exchanged to remove any unbound Ca. SMT3-A1 (nC-A), P2808 (nC-B), and a negative control protein (recombinant SMT3) were added to a 140 μl reaction in the same buffer so their final concentrations were 8.57 μM, and either CaCl₂ or TbCl₃ added to 300 μM. The reactions were incubated at 4° C. for 16 h, and then buffer exchanged with Zeba 7 MWCO desalting columns into MBS, pH 6.5. The proteins were diluted to 0.5 μM in MBS, pH 6.5 and then 200 μl analyzed in duplicate on iD5 (Molecular Devices) plate reader using time-resolved fluorescence with Ex/Em: 350/544 nm, 200 micro-sec delay. As shown in FIG. 15, P972 (nC-A) exhibited 9× greater fluorescence when incubated with terbium instead of calcium, and P2808 (nC-B) exhibited 23× greater fluorescence when incubated with terbium instead of calcium.

### Example 4. Loop Length Modelling

We next undertook modelling experiments to ascertain the ability of the protein scaffold to maintain its secondary and tertiary structure while varying loop lengths of each of L1-L8. For each loop, the plasticity was modeled with P2808 as follows. To explore long length, each loop was replaced by a flexible 15-amino acid (G₄S)₃ linker (sequence: GGGGSGGGGSGGGGS (SEQ ID NO: 41). The protein fold was modeled in AlphaFold mmseq without relaxation. The top result was aligned with P2808 in Swiss PDB Viewer with the MagicFit function.

The loop, the flanking N-terminal, and the flanking C-terminal amino acid are shown in different shades. The structure was assessed qualitatively for the maintenance of the overall beta-sheet structures. Structures that maintained the overall beta-sheet structure were considered to maintain the overall fold. To explore short loop lengths, each loop was completely deleted and modeled as above. If the complete deletion did not impact the overall fold, no additional constructs were modeled. The complete deletion was aligned with Swiss PDB Viewer with the MagicFit function and then assessed qualitatively.

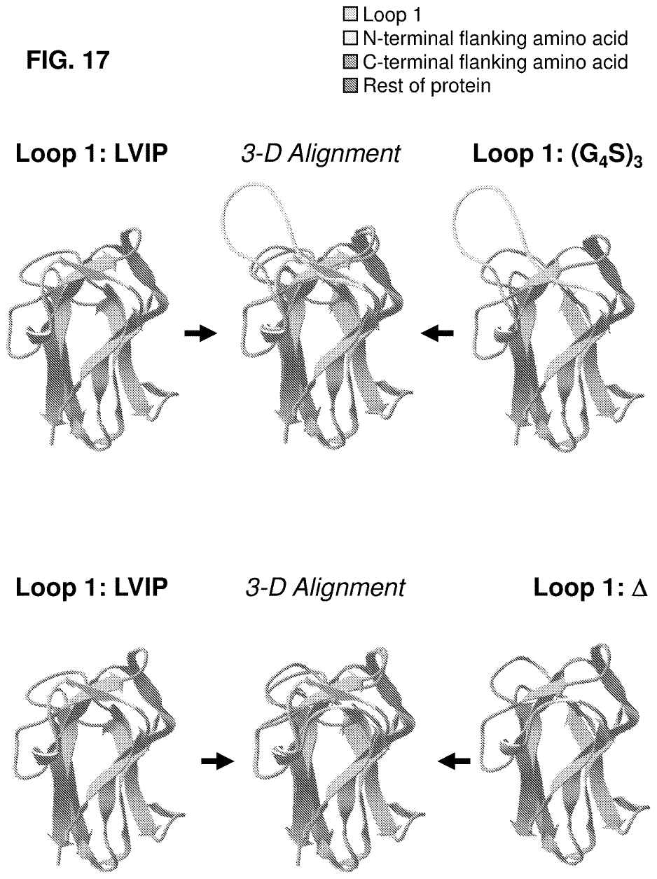

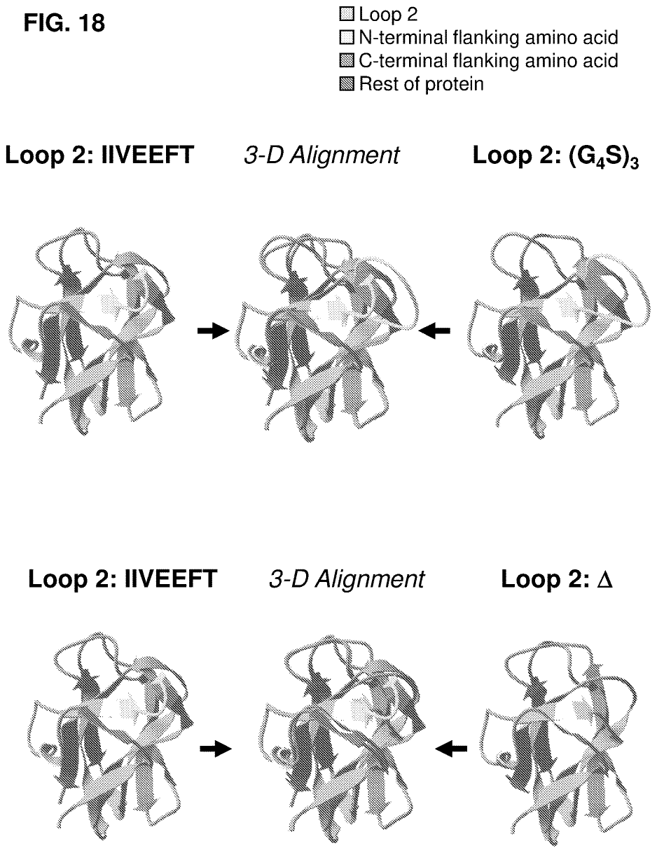

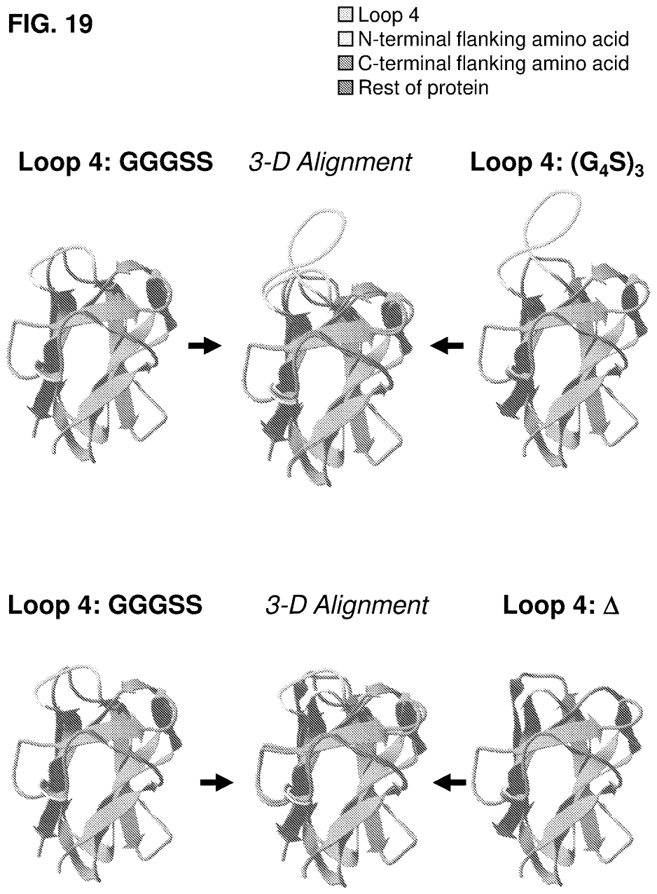

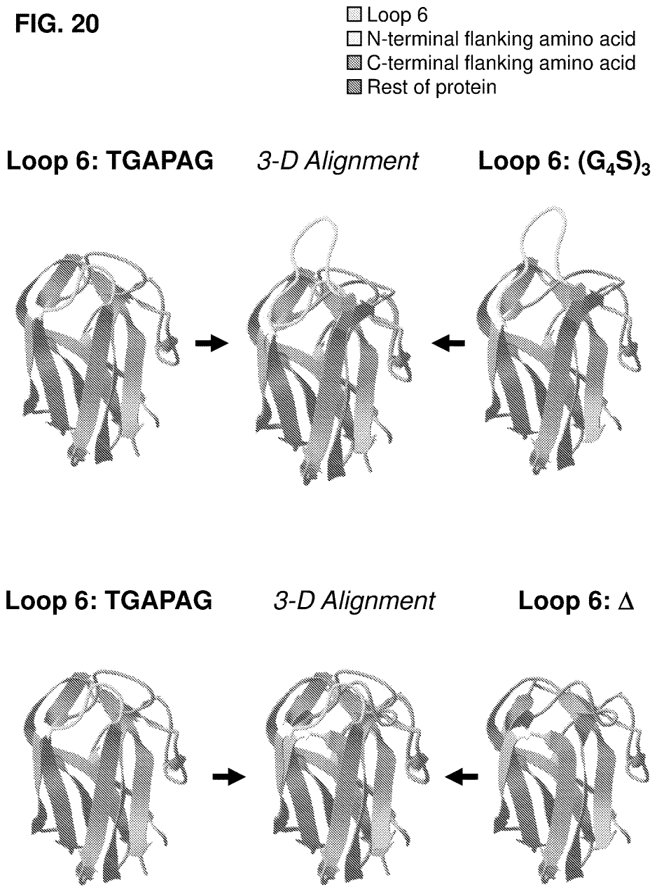

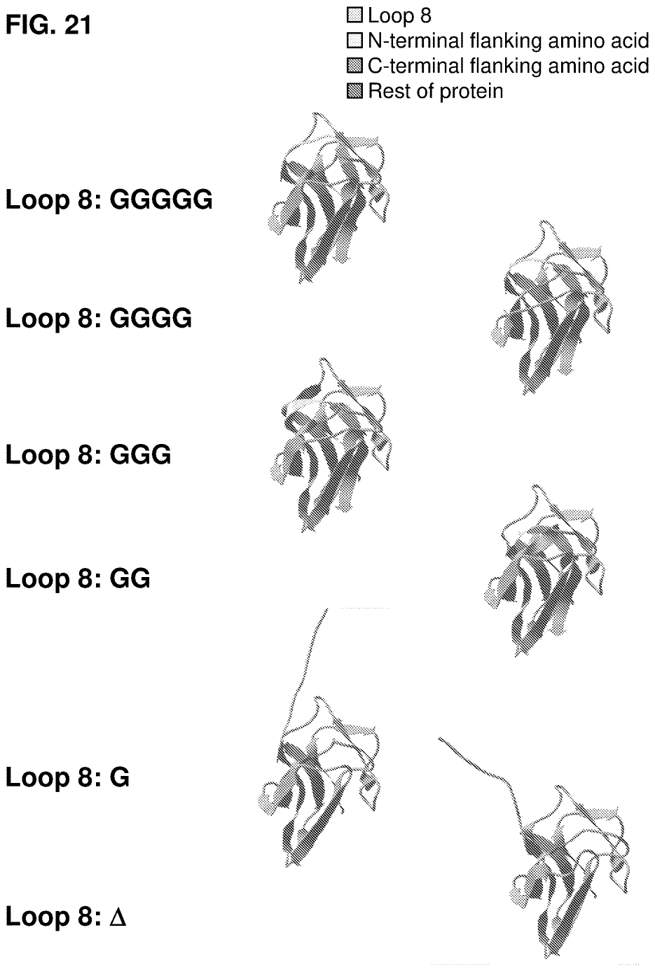

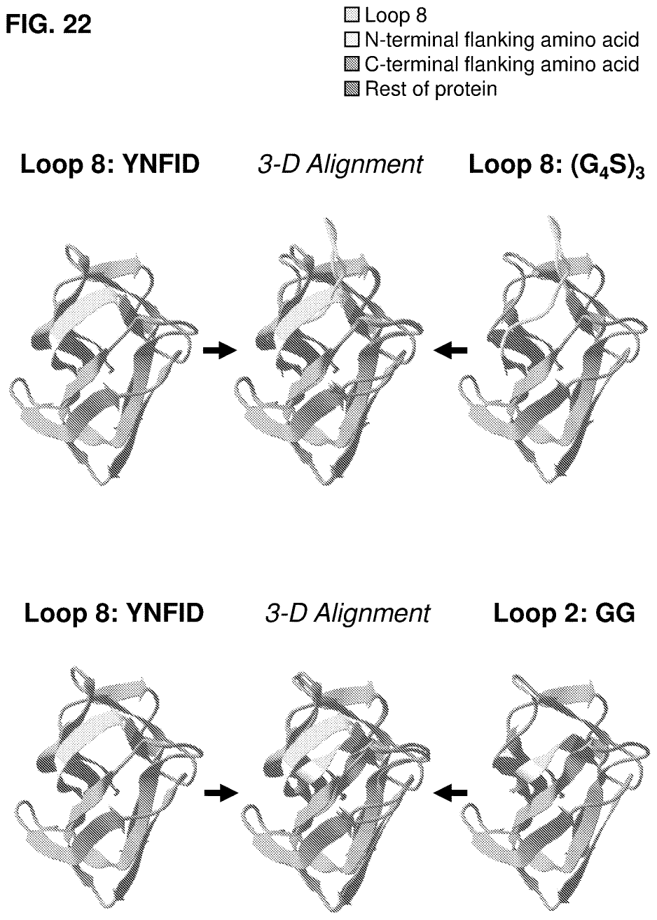

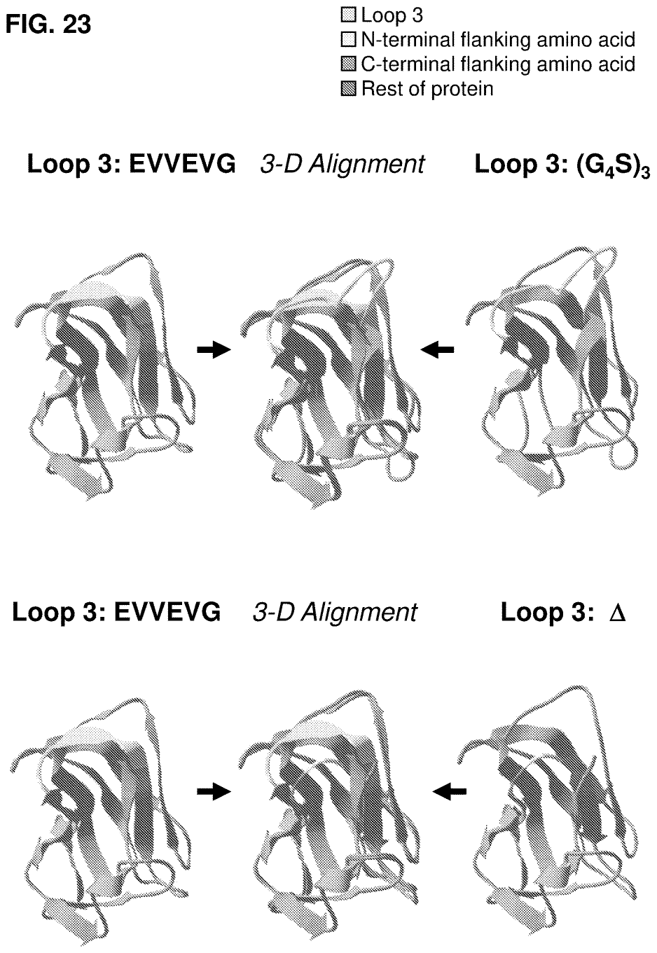

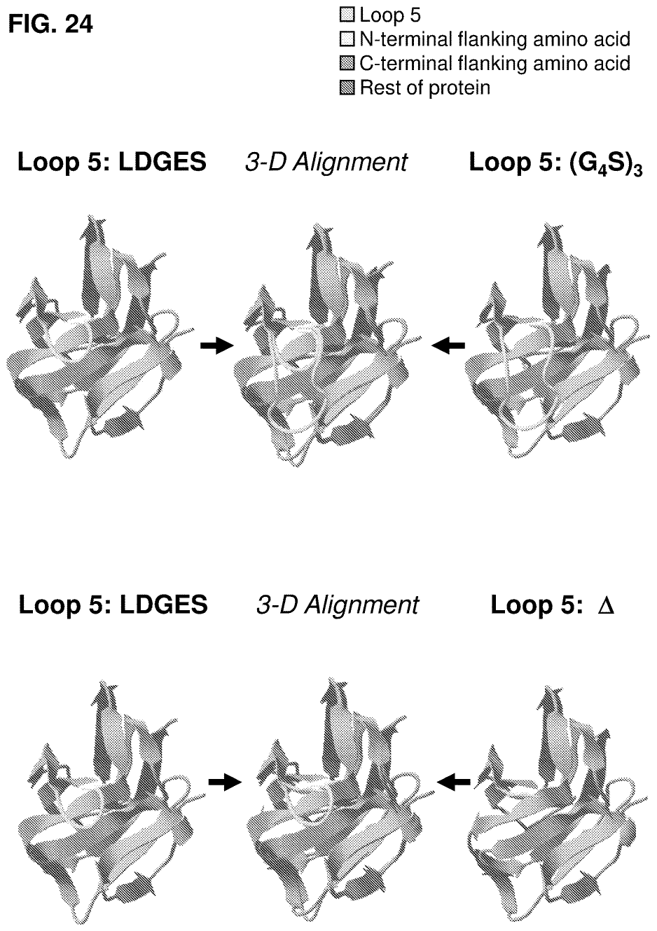

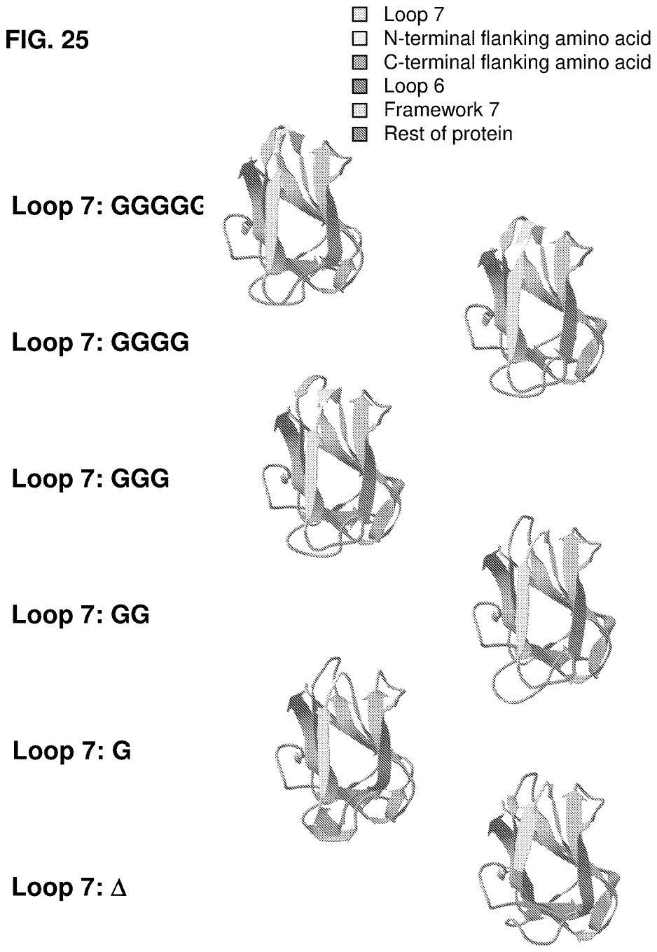

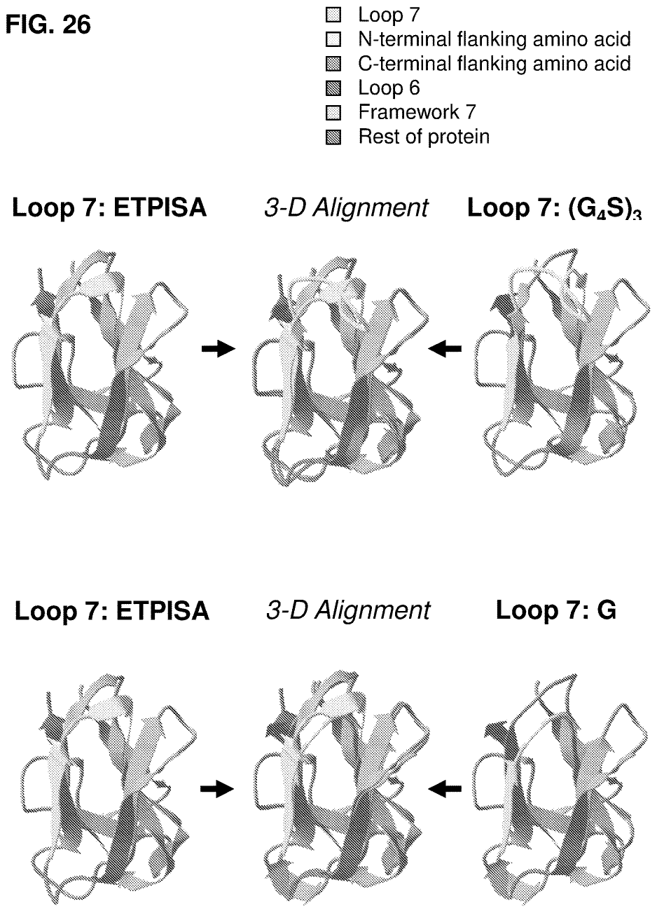

A deletion was considered to result in disruption of the overall beta-sheet structure if a beta-strand secondary structure assignment was converted to a coil assignment or one or more beta strands lost association with an adjacent beta-strand. If the complete deletion resulted in a disruption of the overall beta-sheet structure, a deletion series was made starting with each wild-type amino acid replaced by G and then removing one G at a time. The construct in the deletion series with the shortest loop length that maintained the fold qualitatively was aligned and assessed. The results from these modelling experiments are shown in FIGS. 17-26.

Our database of nanoCLAMP binders was searched for any clones whose loops deviated from the standard lengths of Loop 1: 4 amino acids, Loop 2: 7 amino acids and Loop 8: 5 amino acids. The resulting clones are listed in Table 9 above. Tables 11 and 12 below summarize the lengths for clones observed with the nC-B and nC-A scaffolds. Table 11 shows top loops and Table 12 shows bottom loops.

The third column indicates the length diversity observed across orthologs. The observation of variation correlates roughly with the modeling data.

TABLE 11
Lengths observed for top loops in isolated nanoCLAMP clones
or in alignment with NagH CBM32-2 in different species

| | | | Length of exemplars |
|---|---|---|---|
| | Length of exemplars | Length of exemplars | observed in NagH |
| | of the nC-B class | of the nC-A class | CBM32-2 alignment |
| Loop | (no. of residues) | (no. of residues) | (no. of residues) |
| L1 | 2, 3, 4 | 0, 3, 4, 5 | 4, 5 |
| L2 | 5, 6, 7 | 1, 4, 5, 6, 7, 8, 9, 10, | 7, 8, 9, 14 |
| | | 11, 14, 15, 16 | |
| L4 | 3, 5 | 5 | 0, 1, 2, 3, 5 |
| L6 | 6 | 6 | 3, 4, 6 |
| L8 | 5 | 4, 5, 6 | 5 |

TABLE 12
Lengths observed for bottom loops in alignment
with NagH CBM32-2 in different species

| | Length of exemplars |
|---|---|
| | observed in NagH |
| | CBM32-2 alignment |
| Loop | (no. of residues) |
| L3 | 6 |
| L5 | 5 |
| L7 | 4, 5 |

Taken together, the modeling results, the observed loop lengths in isolated nanoCLAMPs, and natural variation in loop length across species indicate that the length and sequence of each of L1-L8 can be independently varied without disrupting the core fold of the protein.

### Example 5. Introduction of Artificial Disulfides into the nC-B Scaffold to Improve Stability

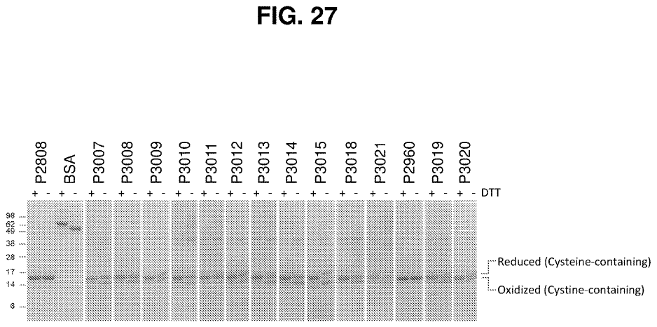

The well characterized SUMO binder P2808 was modeled with AlphaFold to rationally select adjacent residues on neighboring beta strands for substitution with Cys residues, with the aim of further stabilizing the protein by introducing a disulfide bond. Substitution mutations were chosen by visual inspection of the structure to identify residues whose side chains were located in the core of the protein, whose side were oriented towards each other, and whose alpha carbons were approximately the same distance apart as observed with natural disulfide bonds. AlphaFold modeling predicted that several of the selected substitution mutations would form disulfide bonds and that a few that would not (Table 13). We cloned, expressed, and purified 14 mutants predicted to form disulfides (12 mutants of P2808 and 2 mutants of a P2808 mutant, P2960, which has DGGGSS871-876GDT and DHTGAP900-905SST X and Y loops from C. celatum). The proteins were immobilized on Ni Sepharose 6 Fast Flow beads under denaturing and reducing conditions and refolded by reducing concentration of denaturant over time, in the presence of reduced and oxidized glutathione to aid in formation of disulfide bonds. The refolded proteins were then eluted from the resin and tested for the presence of disulfide bonds by mobility shift on SDS PAGE under reducing vs oxidizing conditions. Because proteins possessing intramolecular disulfides remain more compact than those that do not, proteins with disulfides typically run faster in SDS-PAGE due to their smaller hydrodynamic radius. Thirteen of the 14 proteins predicted by AlphaFold to form disulfide bonds migrated faster on SDS PAGE in sample buffer lacking reducing agent than in sample buffer containing reducing agent. This observation is consistent with the agent reducing disulfide bonds in those proteins (FIG. 27). The proteins that appeared to possess disulfide bonds also had a band that ran similarly to reduced form. This observation may indicate a mixed population of proteins with and without disulfides. These preparations also contained a small percentage of higher molecular weight species, likely intermolecular disulfide bonded multimers, which largely disappeared upon reduction. The control proteins, P2808 and P2960, which contain no cysteines, migrated at the same rate in both the reducing and non-reducing buffer. Further, an unrelated disulfide-containing control protein, bovine serum albumin (BSA), showed the expected decrease in electrophoretic mobility in reducing buffers. Taken together, these results indicate that disulfide bonds can be successfully introduced into nC-B scaffold in the twelve positive constructs in Table 13. Clones P3007, P3008, and P3009 contain no lysines. The absence of lysines is expected to reduce susceptibility to trypsin and increase the specificity of labeling with amine-reactive reagents. These scaffolds have only a single primary amine located the N-terminal alpha amino group and are expected to be modified specifically at this position by amine-reactive reagents. Clones P3013 and P3014 contain no asparagines, common sites of deamidation, so are expected to be less susceptible to deamidation.

TABLE 13
nanoCLAMPs with introduced pairs of Cysteines for disulfide formation

| | | | | | | Disulfides |
|---|---|---|---|---|---|---|
| | | AlphaFold | Tₘ | Tₘ | | detected? |
| | | predicted | (oxidizing) | (reducing) | Neutral or | (SDS |
| Clone | Description | disulfide? | ° C. | ° C. | better Tₘ* | PAGE) |
| P2808 | Basis of mutants | n.a. | 76 | 78 | n.a. | no |
| | P3007 - P3018 | | | | | |
| P3007¹ | P2808 with K878C | yes | 72 | 74 | yes | yes |
| | G907C | | | | | |
| P3008¹ | P2808 with K878C | yes | 72 | 76 | yes | yes |
| | A904C | | | | | |
| P3009¹ | P2808 with K878C | yes | 73 | 77 | yes | yes |
| | A904C P905Δ | | | | | |
| P3010 | P2808 with V861C | yes | 62 | 64 | no | yes |
| | I943C | | | | | |
| P3011 | P2808 with P905C | yes | 58 | 62 | no | no |
| | L855C | | | | | |
| P3012¹ | P2808 with S845C | yes | 75 | 79 | yes | yes |
| | L936C | | | | | |
| P3013¹ | P2808 with W879C | yes | 74 | 75 | yes | yes |
| | N928C | | | | | |
| P3014¹ | P2808 with W879C | yes | 70 and 90 | 68 | yes | yes |
| | N928C L926W | | | | | |
| P3015¹ | P2808 with L884C | yes | 82 | 73 | yes | yes |
| | L926C | | | | | |
| P3017 | P2808 with V804S | yes | 72 | 77 | no | yes |
| | E805E F806C | | | | | |
| | L948C | | | | | |
| P3018¹ | P2808 with V858C | yes | 72 | 70 | yes | yes |
| | L888C | | | | | |
| P3021 | P2808 with K878C | yes | n.a. | 61 | no | yes |
| | A904C P905Δ | | | | | |
| | P808C V861C | | | | | |
| | I943C L855C | | | | | |
| | S845C L936C | | | | | |
| P2960 | Basis of mutants | yes | 73 | 74 | n.a. | no |
| | P3019, P3020 | | | | | |
| P3019¹ | P2960 with K878C | yes | 73 | 74 | yes | yes |
| | G907C | | | | | |
| P3020¹ | P2960 with K878C | yes | 73 | 77 | yes | yes |
| | A906GC | | | | | |
| M1 | P2808 with S845C | no | n.a. | n.a. | n.a. | n.a. |
| | N928C | | | | | |
| M2 | P2808 with K878C | no | n.a. | n.a. | n.a. | n.a. |
| | A904C P905Δ, | | | | | |
| | A906Δ | | | | | |
| M3 | P2808 with K878C | no | n.a. | n.a. | n.a. | n.a. |
| | A904C P905Δ and | | | | | |
| | add P808C 1943C | | | | | |
| M4 | P2808 with K878C | no | n.a. | n.a. | n.a. | n.a. |
| | A904C P905Δ | | | | | |
| | V861C I924C | | | | | |

*Neutral or better Tm is defined by Tm of basis, +/− 4C.
¹Clones with disulfide bonds and good thermal stability

To ensure that the thermal stability was not negatively affected by the mutations, we measured the melting temperature of the mutants and the parents using differential scanning fluorimetry (DSF). We defined neutral or better results as a Tₘ not more than 4° C. below that of the parent. Clones P3010, P3011, P3017 and P3021 had deleterious effects on thermal stability and were not pursued further. Of the 14 proteins tested, 10 had neutral or better effect on the melting temperature and were at least as thermally stable as the parent constructs (Table 13). One clone, P3015 with a disulfide bond between residues 884 and 926, showed an 8° C. improvement in thermal stability in oxidized vs. reduced conditions (FIG. 27).

### Materials and Methods

### AlphaFold Modeling of Introduced Disulfides

The 2808 sequence was modeled in AlphaFold with pairs of cysteine substitutions. The version was mmseq, and the modeling was performed with relaxation.

### Analysis of Disulfides by SDS PAGE

nanoCLAMPs were purified as described above, under denaturing conditions, except the refolding step was modified. Briefly, nanoCLAMPs were bound to Ni Sepharose 6 Fast Flow (Cytivia) in 6 M GuHCl, 20 mM Tris, pH 8 (QCB)+5 mM TCEP. The resins were washed with 5 column volumes (CV) QCB+1 mM TCEP, then 5 CV QCB (no TCEP). The proteins were then gradually refolded by washing with 10 CV QCB+2 mM GSH/1 mM GSSG, then steps of 5 CVs each stepping the GuHCl down from 4, 3, 2, 1, and finally 0 M GuHCl by diluting with 20 mM MOPS, 150 mM NaCl (MBS), 1 mM CaCl₂), 2 mM GSH/1 mM GSSG, pH 8. Each refolding step of 5 CVs was incubated for 30 min. The refolded protein was then washed in 10 CV MBS, pH 8, 1 mM CaCl₂), and finally eluted with MBS, pH 8, 1 mM CaCl₂), 250 mM imidazole. Proteins were normalized to 1 mg/ml in MBS, 1 mM CaCl₂), pH 6.5. The proteins were diluted 10× into SDS sample buffer containing 50 mM DTT (reducing) and SDS sample buffer lacking reducing agent. The proteins were heated to 95° C. for 5 min, cooled and 1 μg separated on 12% NuPAGE BisTris gel (Thermo) with MES running buffer. Gels were stained with GelCode Blue (Thermo).

### Differential Scanning Fluorimetry (DSF)

DSF was performed as described previously, except in the reducing case, TCEP was included in the DSC cocktail at 50 mM (final). The DSC program was performed as described, and the Tₘ measured at the inflection point of the curve.

### Example 6. Identification of Framework Variants that Maintain Binding Activity

Phage library NL-26 contains clones of nanoCLAMPs with the wild-type nC-B framework as well as mutations resulting from errors in gene synthesis, PCR and phage propagation. The library was screened and analyzed to identify nanoCLAMPs that maintain the ability to bind their intended target and that contain one or more mutations in the framework regions. The analysis resulted in the identification of 105 nanoCLAMP variants, each recognizing one of four target antigens and each containing one or more mutations in the framework regions. The number of mutations identified in each framework and their position are summarized in Tables 14 and 15. A listing of individual enriched clones, targets and framework mutations is shown in Table 16. The identification of these variants in this non-exhaustive analysis indicates that each framework region can tolerate one or more mutations while maintaining the ability to be displayed on the phage surface and mediate binding to its target.

### Methods

The NL-26 phage library was panned against recombinant GFPMut2, Human Serum Albumin, mCherry, and TEV protease, as described above. Phage were enriched in two rounds, and approximately 200,000 clones from each round were DNA sequenced by next generation DNA sequencing with an Illumina MiSeq system. The sequencing reads were processed with PipeBio software to cluster and count like-sequences. Clones were identified that met the criteria of 1) showing greater than two-fold normalized enrichment from Round 1 to Round 2 and 2) having one or more mutations in the framework region.

TABLE 14
Tolerance of Frameworks 1-9 to Mutations

| Framework | No. of enriched clones | No. of enriched clones |
|---|---|---|
| (length in | identified with 1 | identified with 2 or more |
| amino acids) | mutation in framework | mutations in framework |
| Framework 1 (8 aa) | 6 | 1 |
| Framework 2 (17 aa) | 29 | 6 |
| Framework 3 (12 aa) | 17 | 0 |
| Framework 4 (9 aa) | 10 | 0 |
| Framework 5 (11 aa) | 6 | 1 |
| Framework 6 (9 aa) | 7 | 2 |
| Framework 7 (8 aa) | 17 | 1 |
| Framework 8 (9 aa) | 6 | 0 |
| Framework 9 (10 aa) | 3 | 0 |

TABLE 15
Positions of Mutations in Frameworks 1-9

| | Position of mutations | | |
|---|---|---|---|
| | identified in enriched | | Position number of |
| | clones (bolded | SEQ | mutations identified |
| Framework | and underlined) | ID NO: | in enriched clones |
| Framework 1 | TLIHTPGW | 22 | 809, 810, 811, 813, |
| | | | 814, 816 |
| Framework 2 | GSEADLLDGDDSTGV | 23 | 821, 822, 823, 824, |
| | EY | | 825, 826, 827, 828, |
| | | | 829, 830, 831, 832, |
| | | | 833, 834, 837 |
| Framework 3 | SLAGEFIGLDLG | 24 | 845, 847, 848, 849, |
| | | | 851, 852, 853, 854, |
| | | | 855, 856 |
| Framework 4 | GIHFVIGAD | 25 | 863, 865, 870, 871 |
| Framework 5 | DKWTRFRLEYS | 26 | 878, 882, 883, 885, 886 |
| Framework 6 | WTTIREYDH | 27 | 897, 898, 899, 900, 901 |
| Framework 7 | QDVIDEDF | 28 | 908, 909, 910, 911, |
| | | | 912, 913, 914, 915 |
| Framework 8 | QYIRLTNLE | 29 | 922, 923, 925, 928 |
| Framework 9 | LTFSEFAIVS | 30 | 936, 937, 939 |

TABLE 16
Listing of individual enriched clones, target and framework mutations

| Clone ID | Target | Framework Mutation(s) |
|---|---|---|
| NGSNJ- | GFP | E885Q |
| 086:985:GW230514000:1:2210:7410:22811 | | |
| NGSNJ- | GFP | V910A |
| 086:985:GW230514000:1:2234:18882:11052 | | |
| NGSNJ- | GFP | D830N |
| 086:985:GW230514000:1:2141:30924:12774 | | |
| NGSNJ- | GFP | G848R, E913G |
| 086:985:GW230514000:1:2170:13684:23030 | | |
| NGSNJ- | GFP | L855I |
| 086:985:GW230514000:1:2115:31250:25864 | | |
| NGSNJ- | GFP | L827F |
| 086:985:GW230514000:1:2214:6008:9518 | | |
| NGSNJ- | GFP | L827F |
| 086:985:GW230514000:1:2237:10511:34381 | | |
| NGSNJ- | GFP | D825N |
| 086:985:GW230514000:1:2210:20166:26459 | | |
| NGSNJ- | GFP | D871G |
| 086:985:GW230514000:1:2273:3721:13917 | | |
| NGSNJ- | GFP | Q922R |
| 086:985:GW230514000:1:2237:7491:8202 | | |
| NGSNJ- | GFP | E849G |
| 086:985:GW230514000:1:2254:27552:20776 | | |
| NGSNJ- | GFP | F882V |
| 086:985:GW230514000:1:2177:30373:29543 | | |
| NGSNJ- | GFP | D914N |
| 086:985:GW230514000:1:2272:24632:21136 | | |
| NGSNJ- | HSA | R897, E898L, Y899N, D900T, |
| 086:985:GW230514000:1:2110:13259:8234 | | H901T, Q908G, D909K, V910I, |
| | | I911Y, D912R, E913R, D914R |
| NGSNJ- | HSA | D828G, D830E, D831I, S832P, |
| 086:985:GW230514000:1:2101:16152:30718 | | T833 |
| NGSNJ- | HSA | E885, Y886 |
| 086:985:GW230514000:1:2148:15628:26897 | | |
| NGSNJ- | HSA | T809Y, I811N |
| 086:985:GW230514000:1:2120:9082:6574 | | |
| NGSNJ- | HSA | F915S |
| 086:985:GW230514000:1:2143:13286:22811 | | |
| NGSNJ- | HSA | D912N |
| 086:985:GW230514000:1:2230:15203:32111 | | |
| NGSNJ- | HSA | D828Y, S939N |
| 086:985:GW230514000:1:2126:1606:30326 | | |
| NGSNJ- | HSA | D828G, Y899 |
| 086:985:GW230514000:1:2247:25084:29622 | | |
| NGSNJ- | HSA | D900E |
| 086:985:GW230514000:1:2204:30816:28432 | | |
| NGSNJ- | HSA | R925H |
| 086:985:GW230514000:1:2167:25156:15468 | | |
| NGSNJ- | HSA | D912N |
| 086:985:GW230514000:1:2230:8024:9157 | | |
| NGSNJ- | HSA | D909N |
| 086:985:GW230514000:1:2224:3640:17848 | | |
| NGSNJ- | HSA | E823K |
| 086:985:GW230514000:1:2141:1768:26757 | | |
| NGSNJ- | HSA | H865Y |
| 086:985:GW230514000:1:2157:18141:16125 | | |
| NGSNJ- | HSA | D831 |
| 086:985:GW230514000:1:2171:30752:8093 | | |
| NGSNJ- | HSA | D830G |
| 086:985:GW230514000:1:2270:15429:6605 | | |
| NGSNJ- | HSA | R925H |
| 086:985:GW230514000:1:2243:8332:17143 | | |
| NGSNJ- | HSA | I851V |
| 086:985:GW230514000:1:2134:8495:7529 | | |
| NGSNJ- | HSA | I911N |
| 086:985:GW230514000:1:2272:21242:29919 | | |
| NGSNJ- | HSA | G863D |
| 086:985:GW230514000:1:2108:10158:9439 | | |
| NGSNJ- | HSA | L826, L827, D828 |
| 086:985:GW230514000:1:2104:18819:27696 | | |
| NGSNJ- | HSA | G852E |
| 086:985:GW230514000:1:2244:28438:17581 | | |
| NGSNJ- | HSA | V910G |
| 086:985:GW230514000:1:2253:8748:25974 | | |
| NGSNJ- | HSA | Y923H |
| 086:985:GW230514000:1:2256:18430:25520 | | |
| NGSNJ- | HSA | A870T |
| 086:985:GW230514000:1:2175:23601:36323 | | |
| NGSNJ- | HSA | G848E |
| 086:985:GW230514000:1:2208:13774:24846 | | |
| NGSNJ- | HSA | A870V |
| 086:985:GW230514000:1:2103:17164:12211 | | |
| NGSNJ- | HSA | S845F |
| 086:985:GW230514000:1:2262:32081:30154 | | |
| NGSNJ- | HSA | L827F |
| 086:985:GW230514000:1:2169:21847:26835 | | |
| NGSNJ- | HSA | E913G |
| 086:985:GW230514000:1:2215:10122:33927 | | |
| NGSNJ- | HSA | T813A |
| 086:985:GW230514000:1:2137:3278:29121 | | |
| NGSNJ- | HSA | G834S |
| 086:985:GW230514000:1:2238:3992:18145 | | |
| NGSNJ- | HSA | D854N |
| 086:985:GW230514000:1:2148:25165:5525 | | |
| NGSNJ- | HSA | P814L |
| 086:985:GW230514000:1:2265:12527:28197 | | |
| NGSNJ- | HSA | G856S |
| 086:985:GW230514000:1:2242:13322:15890 | | |
| NGSNJ- | HSA | G834D |
| 086:985:GW230514000:1:2149:22281:17597 | | |
| NGSNJ- | HSA | Y837F |
| 086:985:GW230514000:1:2109:19804:3380 | | |
| NGSNJ- | HSA | G829E |
| 086:985:GW230514000:1:2237:23267:6026 | | |
| NGSNJ- | HSA | R883H |
| 086:985:GW230514000:1:2259:25780:10191 | | |
| NGSNJ- | mCherry | F882V |
| 086:985:GW230514000:1:2120:16324:7341 | | |
| NGSNJ- | mCherry | V910D |
| 086:985:GW230514000:1:2212:31756:4726 | | |
| NGSNJ- | mCherry | L827F |
| 086:985:GW230514000:1:2111:11831:5134 | | |
| NGSNJ- | mCherry | R897L, E898N, Y899T, D900P, |
| 086:985:GW230514000:1:2118:20329:16376 | | H901Y, V910G |
| NGSNJ- | mCherry | D828E, I911N |
| 086:985:GW230514000:1:2220:5828:26083 | | |
| NGSNJ- | mCherry | R897C |
| 086:985:GW230514000:1:2153:7753:7999 | | |
| NGSNJ- | mCherry | T937A |
| 086:985:GW230514000:1:2204:13973:36965 | | |
| NGSNJ- | mCherry | R897C |
| 086:985:GW230514000:1:2234:23140:21778 | | |
| NGSNJ- | mCherry | Y837F, R897C |
| 086:985:GW230514000:1:2113:9290:36432 | | |
| NGSNJ- | mCherry | G829, D830E, D831R |
| 086:985:GW230514000:1:2121:13738:3740 | | |
| NGSNJ- | mCherry | G863S |
| 086:985:GW230514000:1:2256:28311:9157 | | |
| NGSNJ- | mCherry | D831V, S832, T833P |
| 086:985:GW230514000:1:2202:2220:25817 | | |
| NGSNJ- | mCherry | A847T |
| 086:985:GW230514000:1:2272:21260:5494 | | |
| NGSNJ- | mCherry | K878R |
| 086:985:GW230514000:1:2112:4291:1564 | | |
| NGSNJ- | mCherry | D830G |
| 086:985:GW230514000:1:2272:11876:5462 | | |
| NGSNJ- | mCherry | L827F |
| 086:985:GW230514000:1:2140:9498:1125 | | |
| NGSNJ- | mCherry | V910A |
| 086:985:GW230514000:1:2222:23827:29136 | | |
| NGSNJ- | mCherry | D830, D831A, S832I, T833P |
| 086:985:GW230514000:1:2209:3595:16642 | | |
| NGSNJ- | mCherry | Q922R |
| 086:985:GW230514000:1:2238:11577:15186 | | |
| NGSNJ- | mCherry | V910I |
| 086:985:GW230514000:1:2237:6271:25661 | | |
| NGSNJ- | mCherry | V910G |
| 086:985:GW230514000:1:2222:4273:10927 | | |
| NGSNJ- | mCherry | R883C |
| 086:985:GW230514000:1:2121:23330:18411 | | |
| NGSNJ- | mCherry | Y899N |
| 086:985:GW230514000:1:2177:21866:18067 | | |
| NGSNJ- | TEV | H865N |
| 086:985:GW230514000:1:2205:20636:33692 | | |
| NGSNJ- | TEV | D828G |
| 086:985:GW230514000:1:2273:14362:13839 | | |
| NGSNJ- | TEV | H865N |
| 086:985:GW230514000:1:2119:16215:19805 | | |
| NGSNJ- | TEV | G852R |
| 086:985:GW230514000:1:2170:23059:20729 | | |
| NGSNJ- | TEV | D830G |
| 086:985:GW230514000:1:2116:5186:34773 | | |
| NGSNJ- | TEV | H865N |
| 086:985:GW230514000:1:2165:10682:18302 | | |
| NGSNJ- | TEV | W816L |
| 086:985:GW230514000:1:2227:22950:22200 | | |
| NGSNJ- | TEV | D831E |
| 086:985:GW230514000:1:2204:14877:3051 | | |
| NGSNJ- | TEV | D830G |
| 086:985:GW230514000:1:2103:2356:6230 | | |
| NGSNJ- | TEV | L810S |
| 086:985:GW230514000:1:2169:3938:10441 | | |
| NGSNJ- | TEV | D825G |
| 086:985:GW230514000:1:2169:17047:33426 | | |
| NGSNJ- | TEV | H865Y |
| 086:985:GW230514000:1:2107:15872:3677 | | |
| NGSNJ- | TEV | I851L |
| 086:985:GW230514000:1:2143:5828:32690 | | |
| NGSNJ- | TEV | D900N |
| 086:985:GW230514000:1:2170:27064:31798 | | |
| NGSNJ- | TEV | S822 |
| 086:985:GW230514000:1:2263:9733:22608 | | |
| NGSNJ- | TEV | P814S |
| 086:985:GW230514000:1:2168:16468:4272 | | |
| NGSNJ- | TEV | G852R |
| 086:985:GW230514000:1:2263:16920:17895 | | |
| NGSNJ- | TEV | E849G |
| 086:985:GW230514000:1:2258:28863:19476 | | |
| NGSNJ- | TEV | G821W |
| 086:985:GW230514000:1:2171:26304:12195 | | |
| NGSNJ- | TEV | I851V |
| 086:985:GW230514000:1:2204:20175:35399 | | |
| NGSNJ- | TEV | D828G |
| 086:985:GW230514000:1:2240:10321:26193 | | |
| NGSNJ- | TEV | D828G |
| 086:985:GW230514000:1:2123:19732:20165 | | |
| NGSNJ- | TEV | I851 |
| 086:985:GW230514000:1:2108:19886:24878 | | |
| NGSNJ- | TEV | S822P, E823K, A824P, D825N |
| 086:985:GW230514000:1:2134:13810:10160 | | |
| NGSNJ- | TEV | N928D |
| 086:985:GW230514000:1:2123:19018:28354 | | |
| NGSNJ- | TEV | L853S |
| 086:985:GW230514000:1:2257:29342:8844 | | |
| NGSNJ- | TEV | I811V |
| 086:985:GW230514000:1:2216:5005:12446 | | |
| NGSNJ- | TEV | V910A |
| 086:985:GW230514000:1:2240:27398:26459 | | |
| NGSNJ- | TEV | L936S |
| 086:985:GW230514000:1:2225:17101:5024 | | |
| NGSNJ- | TEV | D831 |
| 086:985:GW230514000:1:2117:22896:14497 | | |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A protein scaffold comprising the structure: 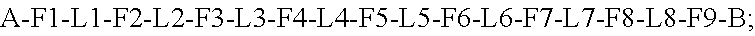
wherein:
F1-F9 correspond to framework regions 1-9;
L1-L8 correspond to loop regions 1-8;
A and B are each independently, absent or comprise at least one amino acid;
F1 comprises the sequence of: (T/S)-LI-(H/R)-(T/S)-(P/E)-(G/S)-W (SEQ ID NO: 4) or a sequence having one or two amino acid insertions, deletions, substitution mutations, or a combination thereof relative to SEQ ID NO: 4;
L1 is absent or comprises at least one amino acid;
F2 comprises the sequence of: G-(S/N/T)-E-(A/S)-(D/N/S/A)-LLDGDD-(S/N/T)-TGV-(E/W/A)-Y (SEQ ID NO: 5) or a sequence having one or two amino acid insertions, deletions, substitution mutations, or a combination thereof relative to SEQ ID NO: 5;
L2 is absent or comprises at least one amino acid;
F3 comprises the sequence of: S-(L/V)-AGEFIGLDLG (SEQ ID NO: 6) or a sequence having one or two amino acid insertions, deletions, substitution mutations, or a combination thereof relative to SEQ ID NO: 6;
L3 is absent or comprises at least one amino acid;
F4 comprises the sequence of: G-(I/V)-(H/R/Y/N)-FVIG-(A/K/R)-(D/N) (SEQ ID NO: 7) or a sequence having one or two amino acid insertions, deletions, substitution mutations, or a combination thereof relative to SEQ ID NO: 7;
L4 is absent or comprises at least one amino acid;
F5 comprises the sequence of: DKW-(T/N/S)-(R/K)-F-(R/K)-LEYS (SEQ ID NO: 8) or a sequence having one or two amino acid insertions, deletions, substitution mutations, or a combination thereof relative to SEQ ID NO: 8;
L5 is absent or comprises at least one amino acid;
F6 comprises the sequence of: WTTI-(R/K/H/Q)-EYD-(H/K/R/Q) (SEQ ID NO: 9) or a sequence having one or two amino acid insertions, deletions, substitution mutations, or a combination thereof relative to SEQ ID NO: 9;
L6 is absent or comprises at least one amino acid;
F7 comprises the sequence of: (Q/K)-DVI-(D/E)-E-(D/S)-F (SEQ ID NO: 10) or a sequence having one or two amino acid insertions, deletions, substitution mutations, or a combination thereof relative to SEQ ID NO: 10;
L7 is absent or comprises at least one amino acid;
F8 comprises the sequence of: (Q/K/R)-YIRLTNLE (SEQ ID NO: 11) or a sequence having one or two amino acid insertions, deletions, substitution mutations, or a combination thereof relative to SEQ ID NO: 11;
L8 is absent or comprises at least one amino acid; and
F9 comprises the sequence of: LTFSEFA-(I/V)-VS (SEQ ID NO: 12) or a sequence having one or two amino acid insertions, deletions, substitution mutations, or a combination thereof relative to SEQ ID NO: 12; and
wherein the protein scaffold comprises at least one mutation selected from the group consisting of N807X, S809X, R812X, S813X, E814X, S815X₁, D818X, N822X, N825X, N832X, W836X, K857X, E858X, I859X, K860X₂, L861X, D862X, R865X, K870X, N871X, N880X, K881X, K883X, N890X, K897X, K901X, K908X, E912X, S914X, and K922X₃ relative to SEQ ID NO: 1, wherein:
X is any amino acid except the amino acid in the equivalent position in SEQ ID NO: 1;
X₁ is any amino acid except R or S;
X₂ is any amino acid except P or K; and
X₃ is any amino acid except R or K.

2. The protein scaffold of claim 1, wherein:
F1 comprises the sequence of: (T/S)-LI-(H/R)-(T/S)-(P/E)-(G/S)-W (SEQ ID NO: 4) or a sequence having one amino acid insertion, deletion, or substitution mutation relative to SEQ ID NO: 4;
F2 comprises the sequence of: G-(S/N/T)-E-(A/S)-(D/N/S/A)-LLDGDD-(S/N/T)-TGV-(E/W/A)-Y (SEQ ID NO: 5) or a sequence having one amino acid insertion, deletion, or substitution mutation relative to SEQ ID NO: 5;
F3 comprises the sequence of: S-(L/V)-AGEFIGLDLG (SEQ ID NO: 6) or a sequence having one amino acid insertion, deletion, or substitution mutation relative to SEQ ID NO: 6;
F4 comprises the sequence of: G-(I/V)-(H/R/Y/N)-FVIG-(A/K/R)-(D/N) (SEQ ID NO: 7) or a sequence having one amino acid insertion, deletion, or substitution mutation relative to SEQ ID NO: 7;
F5 comprises the sequence of: DKW-(T/N/S)-(R/K)-F-(R/K)-LEYS (SEQ ID NO: 8) or a sequence having one amino acid insertion, deletion, or substitution mutation relative to SEQ ID NO: 8;
F6 comprises the sequence of: WTTI-(R/K/H/Q)-EYD-(H/K/R/Q) (SEQ ID NO: 9) or a sequence having one amino acid insertion, deletion, or substitution mutation relative to SEQ ID NO: 9;
F7 comprises the sequence of: (Q/K)-DVI-(D/E)-E-(D/S)-F (SEQ ID NO: 10) or a sequence having one amino acid insertion, deletion, or substitution mutation relative to SEQ ID NO: 10;
F8 comprises the sequence of: (Q/K/R)-YIRLTNLE (SEQ ID NO: 11) or a sequence having one amino acid insertion, deletion, or substitution mutation relative to SEQ ID NO: 11; and
F9 comprises the sequence of: LTFSEFA-(I/V)-VS (SEQ ID NO: 12) or a sequence having one amino acid insertion, deletion, or substitution mutation relative to SEQ ID NO: 12.

3. The protein scaffold of claim 2, wherein:
F1 comprises the sequence of: (T/S)-LI-(H/R)-(T/S)-(P/E)-(G/S)-W (SEQ ID NO: 4);
F2 comprises the sequence of: G-(S/N/T)-E-(A/S)-(D/N/S/A)-LLDGDD-(S/N/T)-TGV-(E/W/A)-Y (SEQ ID NO: 5);
F3 comprises the sequence of: S-(L/V)-AGEFIGLDLG (SEQ ID NO: 6);
F4 comprises the sequence of: G-(I/V)-(H/R/Y/N)-FVIG-(A/K/R)-(D/N) (SEQ ID NO: 7);
F5 comprises the sequence of: DKW-(T/N/S)-(R/K)-F-(R/K)-LEYS (SEQ ID NO: 8);
F6 comprises the sequence of: WTTI-(R/K/H/Q)-EYD-(H/K/R/Q) (SEQ ID NO: 9);
F7 comprises the sequence of: (Q/K)-DVI-(D/E)-E-(D/S)-F (SEQ ID NO: 10);
F8 comprises the sequence of: (Q/K/R)-YIRLTNLE (SEQ ID NO: 11); and
F9 comprises the sequence of: LTFSEFA-(I/V)-VS (SEQ ID NO: 12).

4. The protein scaffold of any one of claims 1-3, wherein the protein scaffold comprises at least one mutation selected from the group consisting of N807X, S809X, R812X, S813X, E814X, S815X, D818X, N822X, N825X, N832X, W836X, K857X, E858X, I859X, K860X, L861X, D862X, R865X, K870X, N871X, N880X, K881X, K883X, N890X, K897X, K901X, K908X, E912X, S914X, and K922X relative to SEQ ID NO: 1, wherein X is any amino acid.

5. The protein scaffold of claim 4, wherein the protein scaffold comprises at least one mutation selected from the group consisting of N807D, S809T, R812H, S813T, E814P, S815G, D818V, N822S, N825D, N832S, W836E, K857E, E858V, I859V, K860E, L861V, D862G, R865H, K870A, N871D, N880T, K881R, K883R, N890G, K897R, K901H, K908Q, E912D, S914D, and K922Q relative to SEQ ID NO: 1.

6. The protein scaffold of claim 4, wherein at least one mutation is K870X and/or N890X.

7. The protein scaffold of claim 6, wherein at least one mutation is K870A and/or N890G.

8. The protein scaffold of any one of claims 1-7, comprising at least 3 fewer lysines relative to SEQ ID NO: 1.

9. The protein scaffold of claim 8, comprising at least 6 fewer lysines relative to SEQ ID NO: 1.

10. The protein scaffold of claim 9, comprising 9 fewer lysines relative to SEQ ID NO: 1.

11. The protein scaffold of claim 9, comprising no lysines.

12. The protein scaffold of any one of claims 1-11, comprising at least 3 fewer asparagines relative to SEQ ID NO: 1.

13. The protein scaffold of claim 12, comprising at least 5 fewer asparagines relative to SEQ ID NO: 1.

14. The protein scaffold of claim 13, comprising 7 fewer asparagines relative to SEQ ID NO: 1.

15. The protein scaffold of claim 13, comprising no asparigines.

16. The protein scaffold of any one of claims 1-15, wherein A and B are each independently, absent or from 1 amino acid to 20 amino acids.

17. The protein scaffold of any one of claims 1-16, wherein each of L1-L8 is, independently, from 1 amino acid to 20 amino acids.

18. The protein scaffold of claim 17, wherein each of L1-L8 is, independently, from 1 amino acid to 10 amino acids.

19. The protein scaffold of claim 18, wherein each of L1-L8 is, independently, from 3 amino acids to 10 amino acids.

20. The protein scaffold of claim 19, wherein each of L1-L8 is, independently, from 3 amino acids to 8 amino acids.

21. The protein scaffold of claim 20, wherein L1 is 4 amino acids, L2 is 7 amino acids, and/or L8 is 5 amino acids.

22. The protein scaffold of claim 21, wherein L1 comprises the sequence of: X₁X₂X₃X₄ (SEQ ID NO: 13), wherein each of X₁-X₄ is, independently, any amino acid.

23. The protein scaffold of claim 21 or 22, wherein L2 comprises the sequence of: X₁X₂X₃X₄X₅X₆X₇ (SEQ ID NO: 14), wherein each of X₁-X₇ is, independently, any amino acid.

24. The protein scaffold do of any one of claims 21-23, wherein L8 comprises the sequence of:
X₁X₂X₃X₄X₅ (SEQ ID NO: 15), wherein each of X₁-X₅ is, independently, any amino acid.

25. The protein scaffold of any one of claims 1-23, wherein L4 comprises the sequence of:
X₁X₂X₃X₄X₅X₆X₇ (SEQ ID NO: 14), wherein each of X₁-X₇ is, independently, any amino acid.

26. The protein scaffold of any one of claims 1-25, wherein L6 comprises the sequence of: X₁X₂X₃X₄X₅X₆ (SEQ ID NO: 16), wherein each of X₁-X₆ is, independently, any amino acid.

27. The protein scaffold of any one of claims 1-26, wherein L8 comprises at least two amino acids.

28. The protein scaffold of any one of claims 1-27, wherein L4 comprises the sequence of: (G/D)-GGSS (SEQ ID NO: 17) or GDT or a sequence having one or two amino acid insertions, deletions, substitution mutations, or a combination thereof relative to SEQ ID NO: 17 or GDT.

29. The protein scaffold of any one of claims 1-28, wherein L6 comprises the sequence of TGAPAG (SEQ ID NO: 18) or a sequence having one or two amino acid insertions, deletions, substitution mutations, or a combination thereof relative to SEQ ID NO: 18.

30. The protein scaffold of any one of claims 1-29, wherein:
L4 comprises the sequence of: (G/D)-GGSS (SEQ ID NO: 17) or GDT; and
L6 comprises the sequence of TGAPAG (SEQ ID NO: 18).

31. The protein scaffold of any one of claims 1-30, wherein L3 comprises the sequence of: (E/K/S)-(V/E)-(V/I/T)-(E/K/P/S)-(V/L)-(G/D) (SEQ ID NO: 19) or a sequence having one or two amino acid insertions, deletions, substitution mutations, or a combination thereof relative to SEQ ID NO: 19.

32. The protein scaffold of any one of claims 1-31, wherein L5 comprises the sequence of: LD-(G/N)-(E/S)-S(SEQ ID NO: 20) or a sequence having one or two amino acid insertions, deletions, substitution mutations, or a combination thereof relative to SEQ ID NO: 20.

33. The protein scaffold of any one of claims 1-32, wherein L7 comprises at least one amino acid.

34. The protein scaffold of any one of claims 1-33, wherein L7 comprises the sequence of ETPI-(S/E)-A (SEQ ID NO: 21) or a sequence having one or two amino acid insertions, deletions, substitution mutations, or a combination thereof relative to SEQ ID NO: 21.

35. The protein scaffold of any one of claims 32-34, wherein:
L3 comprises the sequence of: (E/K/S)-(V/E)-(V/I/T)-(E/K/P/S)-(V/L)-(G/D) (SEQ ID NO: 19) or a sequence having one or two amino acid insertions, deletions, substitution mutations, or a combination thereof relative to SEQ ID NO: 19;
L5 comprises the sequence of: LD-(G/N)-(E/S)-S(SEQ ID NO: 20) or a sequence having one or two amino acid insertions, deletions, substitution mutations, or a combination thereof relative to SEQ ID NO: 20; and
L7 comprises the sequence of ETPI-(S/E)-A (SEQ ID NO: 21) or a sequence having one or two amino acid insertions, deletions, substitution mutations, or a combination thereof relative to SEQ ID NO: 21.

36. The protein scaffold of any one of claims 1-35, wherein A comprises the sequence of (D/N/H)-P.

37. The protein scaffold of claim 36, wherein A comprises the sequence of DP.

38. The protein scaffold of any one of claims 1-37, wherein B comprises the sequence of DELE (SEQ ID NO: 35).

39. The protein scaffold of any one of claims 1-38, wherein:
F1 comprises the sequence of: TLIHTPGW (SEQ ID NO: 22) or a sequence having one or two amino acid insertions, deletions, substitution mutations, or a combination thereof relative to SEQ ID NO: 22;
L1 is absent or comprises at least one amino acid;
F2 comprises the sequence of: GSEADLLDGDDSTGVEY (SEQ ID NO: 23) or a sequence having one or two amino acid insertions, deletions, substitution mutations, or a combination thereof relative to SEQ ID NO: 23;
L2 is absent or comprises at least one amino acid;
F3 comprises the sequence of: SLAGEFIGLDLG (SEQ ID NO: 24) or a sequence having one or two amino acid insertions, deletions, substitution mutations, or a combination thereof relative to SEQ ID NO: 24;
L3 comprises the sequence of: EVVEVG (SEQ ID NO: 31) or a sequence having one or two amino acid insertions, deletions, substitution mutations, or a combination thereof relative to SEQ ID NO: 31;
F4 comprises the sequence of: GIHFVIGAD (SEQ ID NO: 25) or a sequence having one or two amino acid insertions, deletions, substitution mutations, or a combination thereof relative to SEQ ID NO: 25;
L4 comprises the sequence of: GGGSS (SEQ ID NO: 32) or a sequence having one or two amino acid insertions, deletions, substitution mutations, or a combination thereof relative to SEQ ID NO: 32;
F5 comprises the sequence of: DKWTRFRLEYS (SEQ ID NO: 26) or a sequence having one or two amino acid insertions, deletions, substitution mutations, or a combination thereof relative to SEQ ID NO: 26;
L5 comprises the sequence of: LDGES (SEQ ID NO: 33) or a sequence having one or two amino acid insertions, deletions, substitution mutations, or a combination thereof relative to SEQ ID NO: 33;
F6 comprises the sequence of: WTTIREYDH (SEQ ID NO: 27) or a sequence having one or two amino acid insertions, deletions, substitution mutations, or a combination thereof relative to SEQ ID NO: 27;
L6 comprises the sequence of: TGAPAG (SEQ ID NO: 18) or a sequence having one or two amino acid insertions, deletions, substitution mutations, or a combination thereof relative to SEQ ID NO: 18;
F7 comprises the sequence of: QDVIDEDF (SEQ ID NO: 28) or a sequence having one or two amino acid insertions, deletions, substitution mutations, or a combination thereof relative to SEQ ID NO: 28;
L7 comprises the sequence of: ETPISA (SEQ ID NO: 34) or a sequence having one or two amino acid insertions, deletions, substitution mutations, or a combination thereof relative to SEQ ID NO: 34;
F8 comprises the sequence of: QYIRLTNLE (SEQ ID NO: 29) or a sequence having one or two amino acid insertions, deletions, substitution mutations, or a combination thereof relative to SEQ ID NO: 29;
L8 is absent or comprises at least one amino acid; and
F9 comprises the sequence of: LTFSEFAIVS (SEQ ID NO: 30) or a sequence having one or two amino acid insertions, deletions, substitution mutations, or a combination thereof relative to SEQ ID NO: 30.

40. The protein scaffold of claim 39, wherein:
F1 comprises the sequence of: TLIHTPGW (SEQ ID NO: 22) or a sequence having one amino acid insertion, deletion, or substitution mutation relative to SEQ ID NO: 22;
L1 is absent or comprises at least one amino acid;
F2 comprises the sequence of: GSEADLLDGDDSTGVEY (SEQ ID NO: 23) or a sequence having one amino acid insertion, deletion, or substitution mutation relative to SEQ ID NO: 23;
L2 is absent or comprises at least one amino acid;
F3 comprises the sequence of: SLAGEFIGLDLG (SEQ ID NO: 24) or a sequence having one amino acid insertion, deletion, or substitution mutation relative to SEQ ID NO: 24;
L3 comprises the sequence of: EVVEVG (SEQ ID NO: 31) or a sequence having one amino acid insertion, deletion, or substitution mutation relative to SEQ ID NO: 31;
F4 comprises the sequence of: GIHFVIGAD (SEQ ID NO: 25) or a sequence having one amino acid insertion, deletion, or substitution mutation relative to SEQ ID NO: 25;
L4 comprises the sequence of: GGGSS (SEQ ID NO: 32) or a sequence having one amino acid insertion, deletion, or substitution mutation relative to SEQ ID NO: 32;
F5 comprises the sequence of: DKWTRFRLEYS (SEQ ID NO: 26) or a sequence having one amino acid insertion, deletion, or substitution mutation relative to SEQ ID NO: 26;
L5 comprises the sequence of: LDGES (SEQ ID NO: 33) or a sequence having one amino acid insertion, deletion, or substitution mutation relative to SEQ ID NO: 33;
F6 comprises the sequence of: WTTIREYDH (SEQ ID NO: 27) or a sequence having one amino acid insertion, deletion, or substitution mutation relative to SEQ ID NO: 27;
L6 comprises the sequence of: TGAPAG (SEQ ID NO: 18) or a sequence having one amino acid insertion, deletion, or substitution mutation relative to SEQ ID NO: 18;
F7 comprises the sequence of: QDVIDEDF (SEQ ID NO: 28) or a sequence having one amino acid insertion, deletion, or substitution mutation relative to SEQ ID NO: 28;
L7 comprises the sequence of: ETPISA (SEQ ID NO: 34) or a sequence having one amino acid insertion, deletion, or substitution mutation relative to SEQ ID NO: 34;
F8 comprises the sequence of: QYIRLTNLE (SEQ ID NO: 29) or a sequence having one amino acid insertion, deletion, or substitution mutation relative to SEQ ID NO: 29;
L8 is absent or comprises at least one amino acid; and
F9 comprises the sequence of: LTFSEFAIVS (SEQ ID NO: 30) or a sequence having one amino acid insertion, deletion, or substitution mutation relative to SEQ ID NO: 30.

41. The protein scaffold of any one of claim 40, wherein:
F1 comprises the sequence of: TLIHTPGW (SEQ ID NO: 22);
L1 is absent or comprises at least one amino acid;
F2 comprises the sequence of: GSEADLLDGDDSTGVEY (SEQ ID NO: 23);
L2 is absent or comprises at least one amino acid;
F3 comprises the sequence of: SLAGEFIGLDLG (SEQ ID NO: 24);
L3 comprises the sequence of: EVVEVG (SEQ ID NO: 31);
F4 comprises the sequence of: GIHFVIGAD (SEQ ID NO: 25);
L4 comprises the sequence of: GGGSS (SEQ ID NO: 32);
F5 comprises the sequence of: DKWTRFRLEYS (SEQ ID NO: 26);
L5 comprises the sequence of: LDGES (SEQ ID NO: 33);
F6 comprises the sequence of: WTTIREYDH (SEQ ID NO: 27);
L6 comprises the sequence of: TGAPAG (SEQ ID NO: 18);
F7 comprises the sequence of: QDVIDEDF (SEQ ID NO: 28);
L7 comprises the sequence of: ETPISA (SEQ ID NO: 34);
F8 comprises the sequence of: QYIRLTNLE (SEQ ID NO: 29);
L8 is absent or comprises at least one amino acid; and
F9 comprises the sequence of: LTFSEFAIVS (SEQ ID NO: 30).

42. The protein scaffold of any one of claim 41, wherein:
F1 comprises the sequence of: TLIHTPGW (SEQ ID NO: 22);
L1 comprises the sequence of: X₁X₂X₃X₄ (SEQ ID NO: 13), wherein each of X₁-X₄ is, independently, any amino acid;
F2 comprises the sequence of: GSEADLLDGDDSTGVEY (SEQ ID NO: 23);
L2 comprises the sequence of: X₁X₂X₃X₄X₅X₆X₇ (SEQ ID NO: 14), wherein each of X₁-X₇ is, independently, any amino acid;
F3 comprises the sequence of: SLAGEFIGLDLG (SEQ ID NO: 24);
L3 comprises the sequence of: EVVEVG (SEQ ID NO: 31);
F4 comprises the sequence of: GIHFVIGAD (SEQ ID NO: 25);
L4 comprises the sequence of: GGGSS (SEQ ID NO: 32);
F5 comprises the sequence of: DKWTRFRLEYS (SEQ ID NO: 26);
L5 comprises the sequence of: LDGES (SEQ ID NO: 33);
F6 comprises the sequence of: WTTIREYDH (SEQ ID NO: 27);
L6 comprises the sequence of: TGAPAG (SEQ ID NO: 18);
F7 comprises the sequence of: QDVIDEDF (SEQ ID NO: 28);
L7 comprises the sequence of: ETPISA (SEQ ID NO: 34);
F8 comprises the sequence of: QYIRLTNLE (SEQ ID NO: 29);
L8 comprises the sequence of: X₁X₂X₃X₄X₅ (SEQ ID NO: 15), wherein each of X₁-X₅ is, independently, any amino acid; and
F9 comprises the sequence of: LTFSEFAIVS (SEQ ID NO: 30).

43. The protein scaffold of any one of claim 42, wherein:
A comprises the sequence of: DP;
F1 comprises the sequence of: TLIHTPGW (SEQ ID NO: 22);
L1 comprises the sequence of: X₁X₂X₃X₄ (SEQ ID NO: 13), wherein each of X₁-X₄ is, independently, any amino acid;
F2 comprises the sequence of: GSEADLLDGDDSTGVEY (SEQ ID NO: 23);
L2 comprises the sequence of: X₁X₂X₃X₄X₅X₆X₇ (SEQ ID NO: 14), wherein each of X₁-X₇ is, independently, any amino acid;
F3 comprises the sequence of: SLAGEFIGLDLG (SEQ ID NO: 24);
L3 comprises the sequence of: EVVEVG (SEQ ID NO: 31);
F4 comprises the sequence of: GIHFVIGAD (SEQ ID NO: 25);
L4 comprises the sequence of: GGGSS (SEQ ID NO: 32);
F5 comprises the sequence of: DKWTRFRLEYS (SEQ ID NO: 26);
L5 comprises the sequence of: LDGES (SEQ ID NO: 33);
F6 comprises the sequence of: WTTIREYDH (SEQ ID NO: 27);
L6 comprises the sequence of: TGAPAG (SEQ ID NO: 18);
F7 comprises the sequence of: QDVIDEDF (SEQ ID NO: 28);
L7 comprises the sequence of: ETPISA (SEQ ID NO: 34);
F8 comprises the sequence of: QYIRLTNLE (SEQ ID NO: 29);
L8 comprises the sequence of: X₁X₂X₃X₄X₅ (SEQ ID NO: 15), wherein each of X₁-X₅ is, independently, any amino acid;
F9 comprises the sequence of: LTFSEFAIVS (SEQ ID NO: 30); and
B comprises the sequence of: DELE (SEQ ID NO: 35).

44. The protein scaffold of claim 42 or 43, wherein L1 comprises the sequence of X₁X₂X₃X₄ (SEQ ID NO: 13), wherein each of X₁, X₃, and X₄ is, independently, any amino acid, and X₂ is V.

45. A protein scaffold comprising a polypeptide having at least 80% sequence identity to SEQ ID NO: 3.

46. The protein scaffold of claim 45, wherein the polypeptide has at least 85%, 90%, 95%, 97%, or 99% sequence identity to SEQ ID NO: 3.

47. The protein scaffold of claim 45 or 46, wherein the polypeptide comprises at least one mutation selected from the group consisting of N807X, S809X, R812X, S813X, E814X, S815X₁, D818X, N822X, N825X, N832X, W836X, K857X, E858X, I859X, K860X₂, L861X, D862X, R865X, K870X, N871X, N880X, K881X, K883X, N890X, K897X, K901 X, K908X, E912X, S914X, and K922X₃ relative to SEQ ID NO: 1,
wherein:
X is any amino acid except the amino acid in the equivalent position in SEQ ID NO: 1;
X₁ is any amino acid except R or S;
X₂ is any amino acid except P or K; and
X₃ is any amino acid except R or K.

48. The protein scaffold of claim 47, wherein the protein scaffold comprises at least one mutation selected from the group consisting of N807X, S809X, R812X, S813X, E814X, S815X, D818X, N822X, N825X, N832X, W836X, K857X, E858X, I859X, K860X, L861X, D862X, R865X, K870X, N871X, N880X, K881X, K883X, N890X, K897X, K901 X, K908X, E912X, S914X, and K922X relative to SEQ ID NO: 1, wherein X is any amino acid.

49. The protein scaffold of claim 48, wherein the protein scaffold comprises at least one mutation selected from the group consisting of N807D, S809T, R812H, S813T, E814P, S815G, D818V, N822S, N825D, N832S, W836E, K857E, E858V, I859V, K860E, L861V, D862G, R865H, K870A, N871D, N880T, K881R, K883R, N890G, K897R, K901H, K908Q, E912D, S914D, and K922Q relative to SEQ ID NO: 1.

50. A protein scaffold comprising framework regions and loop regions, the protein scaffold comprising at least 7 framework regions from the following structure: 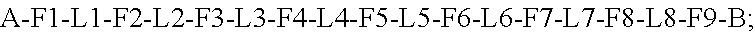
wherein:
F1-F9 correspond to framework regions 1-9;
L1-L8 correspond to loop regions 1-8 that are each independently, absent or comprises one or more amino acids;
A and B are each independently, absent or comprise at least one amino acid;
F1 comprises the sequence of: (T/S)-LI-(H/R)-(T/S)-(P/E)-(G/S)-W (SEQ ID NO: 4) or a sequence having one or two amino acid insertions, deletions, substitution mutations, or a combination thereof relative to SEQ ID NO: 4;
F2 comprises the sequence of: G-(S/N/T)-E-(A/S)-(D/N/S/A)-LLDGDD-(S/N/T)-TGV-(E/W/A)-Y (SEQ ID NO: 5) or a sequence having one or two amino acid insertions, deletions, substitution mutations, or a combination thereof relative to SEQ ID NO: 5;
F3 comprises the sequence of: S-(L/V)-AGEFIGLDLG (SEQ ID NO: 6) or a sequence having one or two amino acid insertions, deletions, substitution mutations, or a combination thereof relative to SEQ ID NO: 6;
F4 comprises the sequence of: G-(I/V)-(H/R/Y/N)-FVIG-(A/K/R)-(D/N) (SEQ ID NO: 7) or a sequence having one or two amino acid insertions, deletions, substitution mutations, or a combination thereof relative to SEQ ID NO: 7;
F5 comprises the sequence of: DKW-(T/N/S)-(R/K)-F-(R/K)-LEYS (SEQ ID NO: 8) or a sequence having one or two amino acid insertions, deletions, substitution mutations, or a combination thereof relative to SEQ ID NO: 8;
F6 comprises the sequence of: WTTI-(R/K/H/Q)-EYD-(H/K/R/Q) (SEQ ID NO: 9) or a sequence having one or two amino acid insertions, deletions, substitution mutations, or a combination thereof relative to SEQ ID NO: 9;
F7 comprises the sequence of: (Q/K)-DVI-(D/E)-E-(D/S)-F (SEQ ID NO: 10) or a sequence having one or two amino acid insertions, deletions, substitution mutations, or a combination thereof relative to SEQ ID NO: 10;
F8 comprises the sequence of: (Q/K/R)-YIRLTNLE (SEQ ID NO: 11) or a sequence having one or two amino acid insertions, deletions, substitution mutations, or a combination thereof relative to SEQ ID NO: 11; and
F9 comprises the sequence of: LTFSEFA-(I/V)-VS (SEQ ID NO: 12) or a sequence having one or two amino acid insertions, deletions, substitution mutations, or a combination thereof relative to SEQ ID NO: 12.

51. The protein scaffold of claim 50, comprising at least 7 of the framework regions, wherein
F1 comprises the sequence of: TLIHTPGW (SEQ ID NO: 22) or a sequence having one or two amino acid insertions, deletions, substitution mutations, or a combination thereof relative to SEQ ID NO: 22;
F2 comprises the sequence of: GSEADLLDGDDSTGVEY (SEQ ID NO: 23) or a sequence having one or two amino acid insertions, deletions, substitution mutations, or a combination thereof relative to SEQ ID NO: 23;
F3 comprises the sequence of: SLAGEFIGLDLG (SEQ ID NO: 24) or a sequence having one or two amino acid insertions, deletions, substitution mutations, or a combination thereof relative to SEQ ID NO: 24;
F4 comprises the sequence of: GIHFVIGAD (SEQ ID NO: 25) or a sequence having one or two amino acid insertions, deletions, substitution mutations, or a combination thereof relative to SEQ ID NO: 25;
F5 comprises the sequence of: DKWTRFRLEYS (SEQ ID NO: 26) or a sequence having one or two amino acid insertions, deletions, substitution mutations, or a combination thereof relative to SEQ ID NO: 26;
F6 comprises the sequence of: WTTIREYDH (SEQ ID NO: 27) or a sequence having one or two amino acid insertions, deletions, substitution mutations, or a combination thereof relative to SEQ ID NO: 27;
F7 comprises the sequence of: QDVIDEDF (SEQ ID NO: 28) or a sequence having one or two amino acid insertions, deletions, substitution mutations, or a combination thereof relative to SEQ ID NO: 28;
F8 comprises the sequence of: QYIRLTNLE (SEQ ID NO: 29) or a sequence having one or two amino acid insertions, deletions, substitution mutations, or a combination thereof relative to SEQ ID NO: 29; and
F9 comprises the sequence of: LTFSEFAIVS (SEQ ID NO: 30) or a sequence having one or two amino acid insertions, deletions, substitution mutations, or a combination thereof relative to SEQ ID NO: 30.

52. The protein scaffold of claim 51, comprising at least 7 of the framework regions, wherein
F1 comprises the sequence of: TLIHTPGW (SEQ ID NO: 22);
F2 comprises the sequence of: GSEADLLDGDDSTGVEY (SEQ ID NO: 23);
F3 comprises the sequence of: SLAGEFIGLDLG (SEQ ID NO: 24);
F4 comprises the sequence of: GIHFVIGAD (SEQ ID NO: 25);
F5 comprises the sequence of: DKWTRFRLEYS (SEQ ID NO: 26);
F6 comprises the sequence of: WTTIREYDH (SEQ ID NO: 27);
F7 comprises the sequence of: QDVIDEDF (SEQ ID NO: 28);
F8 comprises the sequence of: QYIRLTNLE (SEQ ID NO: 29); and
F9 comprises the sequence of: LTFSEFAIVS (SEQ ID NO: 30).

53. The protein scaffold of any one of claims 50-52, wherein the protein scaffold comprises at least 8 of the framework regions F1-F9.

54. The proteins scaffold of any one of claims 50-53, wherein the protein scaffold comprises at least 80%, 85%, 90%, 95%, 97%, or 99% sequence identity to the framework regions F1-F9 over one or more regions of alignment.

55. The protein scaffold of any one of claims 1-54, further comprising a substitution mutation that adds a cysteine residue.

56. The protein scaffold of claim 55, wherein the protein scaffold comprises a first substitution mutation that adds a first cysteine residue and a second substitution mutation that adds a second cysteine residue.

57. The protein scaffold of claim 56, wherein the first cysteine residue and the second cysteine residue form a disulfide bond under oxidizing conditions.

58. The protein scaffold of any one of claims 55-57, wherein the protein scaffold comprises at least one mutation selected from the group consisting of F806C, P808C, S845C, L855C, V858C, V861C, K878C, W879C, L884C, L888C, A904C, P905C, A906GC, G907C, I924C, L926C, N928C, L936C, I943C, L948C.

59. The protein scaffold of claim 58, wherein the protein scaffold comprises at least two or more mutations selected from the group consisting of F806C, P808C, S845C, L855C, V858C, V861C, K878C, W879C, L884C, L888C, A904C, P905C, A906GC, G907C, I924C, L926C, N928C, L936C, I943C, L948C.

60. The proteins scaffold of claim 59, wherein the protein scaffold comprises a pair of cysteine mutations selected from the group consisting of K878C and G907C, K878C and A904C, V861C and I943C, P905C and L855C, S845C and L936C, W879C and N928C, L884C and L926C, F806C and L948C, V858C and L888C, K878C and G907C, K878C and A906GC, S845C and N928C, K878C and A904C, P808C and I943C, V861C and I924C, P808C and V861C, and I943C and L855C.

61. The protein scaffold of claim 60, wherein the pair of cysteine mutations is selected from the group consisting of K878C and G907C, K878C and A904C, S845C and L936C, W879C and N928C, W879C and N928C, L884C and L926C, V858C and L888C, K878C and G907C, and K878C and A906GC.

62. The protein scaffold of any one of claims 1-61, further comprising a tag covalently attached to the scaffold.

63. The protein scaffold of claim 62, wherein the tag is an affinity tag.

64. The protein scaffold of claim 62 or 63, wherein the tag is attached to the N-terminus or the C-terminus of the scaffold.

65. The protein scaffold of any one of claims 1-64, wherein the scaffold is conjugated to a functional group.

66. The protein scaffold of claim 65, wherein the functional group comprises biotin, streptavidin or a derivative of streptavidin, a polyethylene glycol moiety, a fluorescent dye, an enzyme, a radioactive moiety, a lanthanide, or a lanthanide binding motif.

67. The protein scaffold of claim 66, wherein the lanthanide is terbium.

68. The protein scaffold of claim 66, wherein the radioactive moiety is an α or β emitter.

69. The protein scaffold of any one of claims 65-68, wherein the functional group is conjugated to sulfhydryl group or a primary amine.

70. A polynucleotide encoding the protein scaffold of any one of claims 1-69.

71. The polynucleotide of claim 70, wherein the polynucleotide is a ribonucleotide.

72. The polynucleotide of claim 70, wherein the polynucleotide is a deoxyribonucleotide.

73. A vector comprising the polynucleotide of claim 71 or 72.

74. A cell comprising the polynucleotide of any one of claims 70-72 or the vector of claim 73.

75. A method of producing the protein scaffold of any one of claims 1-69 comprising:
(a) providing a cell transformed with the polynucleotide of any one of claims 70-72 or the vector of claim 73;
(b) culturing the transformed cell under conditions for expressing the polynucleotide, wherein the culturing results in expression of the protein scaffold; and
(c) isolating the protein scaffold.

76. A particle comprising the protein scaffold of any one of claims 1-69.

77. The particle of claim 76, wherein the particle is a magnetic particle.

78. A resin comprising a plurality of the particles of claim 76 or 77.

79. A column comprising the resin of claim 78.

80. A method of purifying a target molecule from a plurality of molecules, the method comprising:
(a) providing a sample comprising a mixture of the target molecule and the plurality of molecules;
(b) contacting the sample with the protein scaffold of any one of claims 1-69, wherein the scaffold specifically binds to the target molecule; and
(c) separating the target molecule bound to the protein scaffold from the plurality of molecules.

81. The method of claim 80, wherein the step of separating comprises immobilizing the protein scaffold.

82. The method of claim 81, wherein the protein scaffold is conjugated to a particle.

83. The method of claim 82, wherein the particle comprises a magnetic bead.

84. The method of claim 82 or 83, wherein the protein scaffold is conjugated to a resin or monolith comprising a plurality of the particles.
